(19) United States
(12) Patent Application Publication
(10) Pub. No.: US 2005/0032695 A1
(43) Pub. Date: Feb. 10, 2005

(54) Method for alleviating pain using protein associated with MYC and related compounds, and assays for identifying such compounds

(75) Inventors: Martin Michaelis, Frankfurt (DE); Gerd Geisslinger, Bad Soden (DE); Klaus Scholich, Dreieich (DE); Irmgard Tegeder, Frankfurt (DE)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma Deutschland GmbH, Frankfurt (DE)

(21) Appl. No.: 10/850,591

(22) Filed: May 20, 2004

Related U.S. Application Data
(60) Provisional application No. 60/520,587, filed on Nov. 17, 2003.

(30) Foreign Application Priority Data
May 30, 2003 (EP) 03012388.9

(51) Int. Cl. A61K 38/52

(52) U.S. Cl. 514/12

(57) ABSTRACT

Use of PAM or functional fragments or derivatives thereof for the preparation of pharmaceutical compounds.

### RELATED APPLICATIONS

This application claims the benefit of priority under 35 U.S.C. § 119 of EP Application No. 03012388.9 filed May 30, 2003, and of U.S. Provisional Application No. 60/520,587 filed on Nov. 17, 2003, the contents of both of which are incorporated by reference.

### BACKGROUND OF THE INVENTION

The invention concerns the use of PAM (Protein Associated with Myc). Other aspects of the invention concern a method for screening pharmaceuticals and methods for the treatment of pain.

Pain is a complex subjective sensation reflecting real or potential tissue damage and the affective response to it. Acute pain is a physiological signal indicating a potential or actual injury. Chronic pain can either be somatogenetic (organic) or psychogenic. Chronic pain is frequently accompanied or followed by vegetative signs, which often result in depression.

Somatogenetic pain may be of nociceptive origin, inflammatory or neuropathic. Nociceptive pain is judged to be commensurate with ongoing activation of somatic or visceral pain-sensitive nerve fibers. Neuropathic pain results from dysfunction in the nervous system; it is believed to be sustained by aberrant somatosensory processes in the peripheral nervous system, the CNS, or both. (For an overview of pain mechanisms, see for example Scholz and Woolf, 2002; Julius and Basbaum, 2001, Woolf and Mannion, 1999; Wood, J. D., 2000; Woolf and Salter, 2000.) Chronic pain results in individual suffering and social economic costs of tremendous extent. Existing pharmacological pain therapies are widely unsatisfying both in terms of efficacy and of safety.

Up to now, two classes of analgesics are mainly employed for the treatment of pain: Non-opioid analgesics, mostly acetaminophen and NSAIDS (non-steroidal anti-inflammatory drugs) and opioid (narcotic) agonists (wherein “opioid” is a generic term for natural or synthetic substances that bind to specific opioid receptors in the CNS, producing an agonist action). Unfortunately both analgesic classes, opioids and non-opioids, have several unwanted side effects. The most serious side effects of opioids are the possibility of inhibition of the respiratory system and after long-term treatment the possibility of addiction (Schaible H. G., Vanegas H., 2000). NSAIDs, a major class of non-opioids, on the other hand, can induce a variety of gastrointestinal complications such as ulcers and bleeding, but also kidney damage (Schaible H. G., Vanegas H., 2000). It has been estimated that in the U.S.A. about 16.000 patients die every year because of severe gastrointestinal complications caused by conventional NSAIDs.

In light of the severe drawbacks connected with state of the art pain treatments, there is a great need for novel classes of pain alleviating drugs. Especially in light of the vast gap between the fast advancing understanding of the neurobiology of pain and the unmet clinical need to provide effective treatments without the drawbacks of state of the art treatments, efforts need to be directed to the discovery of new targets for novel classes of analgesics. Thus, it is the object of the present invention to provide a new means for the development and provision of a new class of pain alleviating drugs.

This object is solved by the use of PAM or functional fragments or derivatives thereof for the preparation of pharmaceutical compounds that alleviate pain.

### DESCRIPTION OF THE FIGURES

FIG. 1: PAM is highly expressed in spinal cord neurons. In situ hybridization using horizontal sections of spinal cords hybridized with sense or antisense probes against rat PAM.

FIG. 2: PAM is expressed in DRG neurons as well as in neuronal cells in rat spinal cord.

FIG. 2A: Immunhistochemical analysis of rat spinal cord sections. The sections were stained with anti-PAM antibody (green) and anti-NeuN or anti-GFAP (red) to visualize neurons or glia cells, respectively. The overlay of both signals is presented in the right panels. The objects were magnified 20×.

FIG. 2B: Immunhistochemical analysis of rat DRGs sections. The sections were stained with anti-PAM antibody (green) and anti-Neu68, anti-Histon or anti-GFAP (red) to visualize neurons, nuclei or glia cells, respectively. The overlay of both signals is presented in the right panels. The objects were magnified 40× except for the Histon staining, which was magnified 63×.

FIG. 3: PAM is differentially expressed in DRGs and spinal cord during different developmental stages. Quantitative RT-PCR (Taqman™) was used to detect PAM in RNA (40 ng) of spinal cord, and DRGs of embryonic rats day 16 (E16), postnatal day 0.5 (P0.5), 3 (P3), 5 (P5), 9 (P9) and adult rats. The mean±SEM of at least 3 determinations is shown.

FIG. 4: PAM is upregulated in the rat spinal cord after zymosan and formalin treatment.

Panel A: RT-PCR analysis with RNA (40 ng) of spinal cords from control animals or animals treated with zymosan after 24 h, 48 h and 96 h. The lower panel shows the mean±SEM of 7 experiments. Student T test: * p<0.001.

Panel B: Western blot analysis using a 7% SDS-PAGE gel with rat spinal cord lysates of control animals or animals treated with zymosan after 24 h, 48 h and 96 h (40 μg) with anti-PAM antibody and anti-ERK1/2.

Panel C: Quantitative RT-PCR analysis with RNA (40 ng) of spinal cords from control animals or animals treated with formalin for 1 hour.

FIG. 5: PAM inhibits Gαs-stimulated AC activity in spinal cord lysates.

Panel A: RT-PCR was used to determine AC isoform expression in spinal cord and DRG RNA (40 ng).

Panel B: Lysates of spinal cords or DRG (10 μg) were assayed for AC activity in the presence of 80 nM Gas as described in Material and Methods. The mean±SEM of at least 3 determinations done in triplicates is shown.

FIG. 6: Intrathecal application of antisense ODNs against PAM increases nociceptive behavior.

Panel A: Adult rats were given intrathecal sense and antisense ODN as described. After formalin treatment, the spinal cord was removed and subjected to immunhistological analysis using anti-PAM antibodies (green) or anti NeuN (red).

Panel B: Formalin assay of animals treated with sense or antisense ODNs as described in the material section. The total amount of flinches over 1 hour is shown. The mean±SE of at least 4 determinations is shown.

Panel C: Formalin assay of animals treated with sense or antisense ODNs as described in the material section. The number of flinches during 1 hour is shown. The mean±SE of at least 4 determinations is shown.

FIG. 7: Protein, genomic and coding nucleotide sequence of human PAM according to NCBI accession numbers AAC39928 (protein sequence; SEQ ID No.1), AF075587 (coding sequence; SEQ ID No.2). Human PAM is located on Chromosome 13q22; its genomic sequence is publicly available under NT_{—}024524.11 (Start: position 24679861; Stop: position 24962245; SEQ ID No 3); FIG. 7C shows the contiguous sequence from position 24679861 to position 24962245.

FIG. 8: EST-clone coding sequences for rat PAM:

FIG. 8A: AW921303 (corresponds to bp 960-1394 of hs cDNA; SEQ ID No. 4)

FIG. 8B: AW918711 (corresponds to bp 8188-8632 of hs cDNA; SEQ ID No. 5)

FIG. 8C: BQ201485 (corresponds to bp 8966-9633 of hs cDNA; SEQ ID No. 6)

FIG. 8D: BE112881 (corresponds to bp 10311-10830 of hs cDNA; SEQ Id No. 7)

FIG. 8E: AW441131 (corresponds to bp 13569-14152 of hs cDNA; SEQ ID No. 8)

FIG. 8F: BF409872 (corresponds to bp 13569-14807 of hs CDNA). (SEQ ID No. 9)

FIG. 9: Overview of domain structure of human PAM according to Guo et al., 1988/Grossberger et al., JBC 1999 and Scholich et al., JBC 2001)

FIG. 10: PCR Primers for rat PAM RT PCR.

FIG. 11: Antisense Oligodesoxynucleotides for inhibiting rat PAM expression and control oligonucleotide.

FIG. 12: Different PAM hs polypeptides for the use in the context of present invention. The polypeptides are fragments derived from the polypeptide according to SEQ ID No.2.

FIG. 13: Different PAM hs polynucleotides for the use in the context of present invention.

FIG. 13A: cDNA sequence coding for the protein fragment according to SEQ ID No.17 comprising nucleotide positions 1317 to 4366 of hs Pam cDNA (SEQ ID No. 24);

FIG. 13B: cDNA sequence coding for the protein fragment according to SEQ ID No.18 comprising nucleotide positions 1482 to 3332 of hs Pam cDNA (SEQ ID No. 25);

FIG. 13C: cDNA sequence coding for the protein fragment according to SEQ ID No.19 comprising nucleotide positions 1641 to 3341 of hs Pam cDNA (SEQ ID No. 26);

FIG. 13D: cDNA sequence coding for the protein fragment according to SEQ ID No.20 comprising nucleotide positions 3142 to 4046 of hs Pam cDNA (SEQ ID No. 27);

FIG. 13E: cDNA sequence coding for the protein fragment according to SEQ ID No.21 comprising nucleotide positions 3142 to 3446 of hs Pam cDNA (SEQ ID No. 28);

FIG. 13F: cDNA sequence coding for the protein fragment according to SEQ ID No.22 comprising nucleotide positions 3228 to 3839 of hs Pam cDNA (SEQ ID No. 29);

FIG. 13G: cDNA sequence coding for the protein fragment according to SEQ ID No.23 comprising nucleotide positions 3228 to 3341 of hs Pam cDNA (SEQ ID No. 30);

FIG. 14: PAM signaling according to the above findings.

### DETAILED DESCRIPTION OF THE INVENTION

The invention is based on findings of the inventors that demonstrate for the first time the implication of PAM in nociceptive processing and its ability to decrease pain (a U.S. Patent Application filed May 20, 2004 listing the same inventors, entitled “Method for alleviating pain using sphingosine-1-phosphate and related compounds, and assays for identifying such compounds,” the contents of which are incorporated herein by reference, describes a similar role for sphingosine-1-phosphate). The term functional refers to the ability of PAM to lower intracellular cAMP levels or to interact with AC; more preferably it refers to its ability to inhibit AC activity and even more preferably to its ability to decrease pain.

A fragment of PAM can be any polypeptide or polynucleotide that is shorter than the corresponding wild type, e.g. shorter than homo sapiens (hs) PAM according to one of the sequences according the accession numbers ACC 39928 (SEQ ID No. 1), NP_{—}055872, NM_{—}015057, AF075587 (SEQ ID No.2) or shorter than a polynucleotide from position 24679861 to position 24962245 of the sequence according to accession number NT_{—}024524.11 (SEQ ID No.3).

A derivative of PAM or of a PAM fragment can be any modification of the amino acid or nucleotide sequence having PAM function or any other kind of modification, such as a chemical or biological modification e.g. leading to the stabilization of the polypeptide or polynucleotide (such as phosphoorothioate modifications), or enabling a specific targeting of the polypeptide or polynucleotide to certain cells or facilitating its entry into or uptake by cells (such as cell-permeant phosphopeptides orthe coupling to cell-permeant peptide vectors, e.g. based on the antennapedia/penetratin, TAT, and signal-peptide based sequences; or coupling to parts of ligands for specific transporters or importers).

PAM or fragments or derivatives thereof may either be used in the form of a polypeptide or a polynucleotide. Useful are suitable modifications or additives for ensuring or facilitating its targeting to the site of need and its entering the cell. On the other hand, a local application, such as an intraspinal application using suitable catheters, etc. or the like is possible for ensuring its targeting to the spinal cord. Other useful additives include salts, buffers or the like for its stabilization, etc.

The PAM polynucleotide or fragment or derivative thereof can e.g. be put into a suitable vector that ensures its intracellular expression and, preferably also its targeting into the cell. A cell type specific expression can be ensured using appropriate promoters/enhancers of neuron-specific genes which are known in the art The use of PAM oligonucleotides is also possible.

The present invention is based on studies of the inventors, that demonstrate for the first time the surprising implication of PAM in sensitisation mechanisms within the spinal cord and dorsal root ganglia (DRGs).

PAM (Protein Associated with Myc) is a giant protein of 510 kDa. The protein, genomic and coding polynucleotide sequences of PAM are known in the state of the art and are, e. g. publicly available from the NCBI (National Centre for Biotechnology Information; National Library of Medicine, Building 38A, Bethesda, Md. 20894, USA; www.ncbi.nhm.nih.gov) data base under the accession numbers AAC39928 (coding sequence; SEQ ID No.1), AF075587 (protein sequence; SEQ ID No.2). Human PAM is located on Chromosome 13q22; its genomic sequence is publicly available under NT_{—}024524.11 (Start: position 24679861; Stop: position 24962245; SEQ ID No.3). Alternatively, the protein and coding sequence are publicly available under KIM0916 protein Accession NP_{—}055872 (protein sequence) and NM_{—}015057 (coding sequence).

For rat PAM, the following EST-clone coding sequences are publicly available:

AW921303 (corresponds to bp 960-1394 of hs cDNA; SEQ ID No.4)

AW918711 (corresponds to bp 8188-8632 of hs cDNA; SEQ ID No.5)

BQ201485 (corresponds to bp 8966-9633 of hs cDNA; SEQ ID No.6)

BE112881 (corresponds to bp 10311-10830 of hs cDNA; SEQ ID No.7)

AW441131 (corresponds to bp 13569-14152 of hs cDNA; SEQ ID No.8)

BF409872 (corresponds to bp 13569-14807 of hs cDNA; SEQ ID No.9).

PAM was originally identified by its ability to interact specifically with the transcriptional activating domain in the N-Terminus of Myc (Guo Q., et al., 1998). PAM has recently been described as a powerful inhibitor of AC activity (Scholich K., Pierre S., Patel T. B.: Protein associated with myc (PAM) is a potent inhibitor of adenylyl cyclase. J. Biol. Chem. 2001, Dec. 14;276(50):47583-9.), but there has been no evidence of its function in nociceptive processing and sensitisation, so far.

Rather, PAM is believed to be playing a role in presynaptic growth regulation: PAM mRNA has been known to be highly expressed in specific anatomical regions, including hippocampus, dentate gyrus and cerebellum. Both PAM and Myc expression in the brain of adult rats and mice is confined to the maturing Purkinje cells in the cerebellum and granule and pyramidal cells in the hippocampus (Ruppert C., et al., 1986; Yang H. et al., 2002). None of these cell types, however is known to be involved in pain processing and sensitisation.

PAM homologues in Drosophila (highwire) and C. elegans (rpm-1) have been shown to play a crucial role in presynaptic terminal organization (Zhen et al., 2000), the regulation of synaptic growth (Wan et al., 2000), synaptogenesis, and neurite growth and targeting (Schaefer et al., 2000). These findings led to the assumption that highwire, rpm-1 and their mammalian homolog PAM might act as negative regulators of synaptic growth (Chang et al., 2000; Jin Y. 2002). Accordingly, a dramatic increase in PAM expression in the cerebellum, hippocampus and dentate gyrus was found during the major synaptogenic period in these structures (Yang et al., 2002).

During brain development in rodents, PAM expression is turned on shortly after birth, up-regulated during the first two weeks, and, thereafter, PAM expression remains elevated during adulthood (Yang et al., 2002). So far, nothing has been known about the expression and regulation of PAM in the spinal cord and DRGs and its function in sensitisation mechanisms and regulation of pain.

Previously, it has been demonstrated that human PAM is a potent regulator of cyclic AMP (cAMP)-signaling and inhibits the enzyme activity of several adenylyl cyclase (AC; E.C.4.6.1.1) isoforms at nanomolar concentrations (Scholich et al. 2001).

The ubiquitous cyclic AMP (cAMP) second messenger system is one of different signal transduction mechanisms translating extracellular stimuli to intracellular signals and responses. Upon extracellular stimulation, G-protein coupled receptors (GPCRs) modulate plasma-membrane bound enzymes or ion channels via trimeric GTP-binding regulatory proteins (G-proteins). One of the enzymes modulated in its activity by GPCRs is the adenylyl cyclase (AC), a cAMP generating enzyme. Thus, the incoming extracellular stimuli influence the intracellular concentration of the intracellular mediator cyclic AMP. A rise in cAMP levels affects the cell by stimulating protein kinase A (PKA), which phosphorylates specific intracellular target proteins and thereby alters their activity.

Each type of cell has characteristic sets of GPCRs, enzymes modulated by those GPCRs, specific subsets of adenylyl cyclase (AC) and target proteins, that, acting together with more unspecific or generally occurring players (such as the ubiquitous cAMP), enable each cell to make its own distinctive response to incoming extracellular signals. It is for example known that the cyclic AMP (cAMP)-second messenger plays a major role in the regulation of synaptic plasticity (Bailey et al., 1996; Xia et al., 1997; Brandon et al., 1997); on the other hand it is involved in metabolic processes and cellular proliferation. Thus, the role of the ubiquitous cAMP messenger system and its different components varies according to different specializations of different tissue and cell types.

A further aspect of the invention concerns the use of PAM or functional fragments or derivatives thereof for the alleviation of pain. The term “alleviate,” as applied to pain, means the ability to prevent, lessen, or abolish pain, or to otherwise make pain more bearable.

Moreover, the use of PAM or functional fragments of derivatives thereof for identifying compounds that alleviate pain is encompassed by present invention. The alleviating compounds are preferably PAM activating compounds. More preferably they have the ability to alleviate pain.

The compounds can for example be identified by their ability to

a) activate or enhance PAM function (i.e. its ability to lower intracellular cAMP levels, to interact with other factors like AC, especially with AC, to inhibit AC or its ability to lower the pain perception) or

b) activate PAM on RNA level (i.e. by activation of PAM transcription or transcript stabilisation) or

c) activate PAM on protein level (i.e. by activation of PAM translation or its posttranslational processing; by modulation of PAM posttranslational modification or by activation of its stabilisation or inhibition of its degradation).

In this aspect, PAM (or its fragment or derivatives thereof) can either be used as a polypeptide or oligo- or polynucleotide.

Another aspect of present invention regards a method of alleviating pain comprising administering a sufficient amount of PAM or a functional fragment or derivative thereof to an individual.

Administration should suitably be performed in a way that allows for targeting of PAM to the site of action (DRG or spinal cord), e.g. by systemical administration of PAM derivatives or formulations targeting themselves to site of need or local (e.g. intraspinal) application of PAM or its fragments or derivatives thereof.

Another aspect of the invention concerns a method of screening for pharmaceuticals useful for alleviating pain, comprising the steps
a. Providing two samples
b. contacting one sample containing PAM or a functional fragment or derivative thereof with a compound,
c. determining the PAM activity in the presence of compound,
d. determining the PAM activity in the absence of compound, and
e. comparing the PAM activity according to c) with that according to d).

PAM can be derived from any sequence available that allows for its specific purpose according to the different aspects of the present invention. Preferably, PAM is human PAM.

For the different aspects of present invention it is also preferred, if PAM is an isolated polypeptide or a oligo- or polynucleotide,. Isolated in the context of the different aspects of present invention means at least partially purified from a natural source or, recombinant PAM (which can, of course, also be purified or partially purified).

An assay is any type of analytical method to monitor a biological process. For the use in drug screening, the assay needs to be reproducible and is preferably also scalable and robust. The assay is preferably suitable for high throughput screening of chemical substances for their ability of alleviating pain. The type of assay depends e.g. on the type of PAM used (either polypeptide or polynucleotide) and the “read out”, i.e. the way in which PAM activity is determined (see below).

Different types of such assays are commonly known in the state of the art and commercially available from commercial suppliers. Suitable assays encompass radioisotopic or fluorescent assays, for example fluorescence polarization assays (such as those offered commercially by Panvera, Perkin-Elmer life sciences (e.g. LANCE) or Packard BioScience (e.g. HTRF or ALPHAscreen™)) for measuring the interaction of a labeled member with a non-labeled member (e.g. PAM or fragments thereof could be labeled and their interaction with AC could be monitored).

More examples include cell based assays, wherein a cell line stably (inducibly or not; chromosomal or episomal) or transiently expresses a recombinant protein of interest. These assays comprise e.g. reporter gene assays, wherein the regulation of a certain promotor or a signal transduction pathway of a member of a signal transduction cascade is measured according to the activity of a reporter enzyme, the expression of which is under the control of said certain promotor. For this type of assay, a recombinant cell line has to be constructed containing the reporter gene under the control of a defined promotor that is to be investigated itself or that is regulated by the signaling cascade under investigation. Suitable reporter enzymes are commonly known within the state of the art and comprise firefly luciferase, renilla luciferase (e.g. commercially available by Packard reagents), β-Galactosidase. Suitable cell lines depend on the aim of the assay but comprise mostly cell lines that are easy to transfect and easy to cultivate, such as, e.g. HeLA, COS, CHO, NIH-3T3, etc.

Assays for measuring the intracellular ion level comprise e.g. FLIPR (fluorometric imaging plate reader, commercially available from Molecular Devices) assays, wherein an argon laser light source combined with a cooled CCD camera allows for parallel measurements in 384 well plates transient ion signals (such as Ca²⁺, etc) within cells (e.g. neuronal cells or other cells (e.g. cells recombinantly or naturally expressing certain ion channels). FLIPR assays allow e.g. for monitoring of intracellular calcium using certain fluorochromes, such as Fluo-3, Fluo-4, or monitoring intracellular pH using BCECF or BCPCF pr specific FLIPR assay kits, or detecting membrane potential changes using e.g. DiBAC or specific FLIPR assay kits, or monitoring of membrane polarization. For the monitoring of other intracellular ions, e.g. zinc or sodium, other dyes known in the state of the art can be used. Other types of assays and other types of read outs are commonly known to persons with skills in the art.

For the measurement of cAMP levels, e.g. AlphaScreen, fluorescence polarization or HTRF technology is suitable.

For the determination of ion channel activity (which control e.g. intracellular ion concentrations and can thus be employed for measurement of intracellular ion concentrations) e.g. membrane potential sensitive assays and dyes can be used such as DiBAC or Molecular Devices membrane potential assay kit on FLIPR technology; mitochondrial membrane polarization measuring JC-1 dye with FLIPR technology; ion sensitive dyes such as Fluo-3, Fluo-4 or Molecular Devices calcium assay kit for intracellular calcium concentration measurement; sodium sensitive dye e.g. from Molecular Probes for measurement of intracellular sodium; assays based on patch-clamping or atomic adsorption spectroscopy-based Rubidium ion efflux measurement for determining of intracellular potassium concentrations, and so on. Further automatical devices and analytical methods for detecting certain changes and states within cells are known to the person of skill in the art and comprise, e.g. the Acumen detector (florescence-based laser scanning reader that allows for 3dimensional reconstitution of distribution of suitably labeled objects) by ACUMEN bioscience.

The PAM polypeptide is preferably a polypeptide that comprises or consists of the sequence according to SEQ ID No 2 or is encoded by a polynucleotide comprising or consisting of the sequence according to SEQ ID No 1 or 3.

The PAM polynucleotide is preferably a polynucleotide comprising or consisting of the sequence according to SEQ ID No 1 or 3 or a polynucleotide comprising or consisting of a sequence that is able to hybridize with the above polynucleotides under stringent conditions.

Stringency describes reaction conditions that influence the specificity of hybridisation or annealing of two single stranded nucleic acid molecules. Stringency, and thus specificity of a reaction depends, inter alia, of the temperature and buffer-conditions used for a reaction: Stringency, and thus specificity, can e.g. be increased by increasing the reaction temperature and/or lowering the ion strength of the reaction-buffer. Conditions of low stringence (and thus low reaction and hybridisation specificity) exist for example, if a hybridisation is performed at room temperature in 2×SSC-solution. Conditions of high stringency comprise e.g. a hybridisation reaction at 68° C. in 0.1×SSC and 0,1% SDS solution.

Hybridisation under conditions of stringency within the different aspects of present invention is preferably understood to be:
1) Hybridising a labelled probe with a nucleic acid sample to be analysed at 65° C., or in the case of oligonucleotide probes, at 5° C. below the annealing or melting temperature of the duplex consisting of oligonucleotide and sample (annealing and melting temperature are in the following understood to be synonyms) over night in 50 mM Tris pH 7,5, 1M Nacl, 1% SDS, 10% Dextran Sulfate, 0.5 mg/ml denatured salmon or herring sperm DNA.
2) Washing for 10 minutes in 2×SSC at room temperature.
3) Washing for 30 minutes in 1×SSC/0.1% SDS at 65° C. (or in the case of oligonucleotides: 5° C. below the annealing temperature).
4) Washing for 30 minutes in 0.1×SSC/0.1% SDS at 65° C. (or in the case of oligonucleotides: 5° C. below the annealing temperature).

Oligonucleotides for the use as hybridisation probes are polynucleotide and preferably DNA-fragments having a length of 15 to 30, preferably 20 nucleotides. The annealing temperature is determined according to the formula Tm=2×(number of A+T) +4×(number of G+C)° C.

For preparing a 2×SSC or a 0.1×SSC (or any other kind of SSC dilution), e.g. a 20×SSC solution is diluted accordingly. 20×SSC consists of 3M NaCl/0.3 M Na-Citrate×2H₂O.

Before performing a hybridisation reaction, the polynucleotides are, if wanted after performing electrophoretic separation (then: Southern Blot (DNA) or Northern Blot (RNA)) or without electrophoretic separation (then: slot or dot Blot), transferred to a suitable membrane, e.g. a nylon or nitrocellulose membrane. Hybridisation is performed using a suitably labelled probe. Suitable labelling techniques are e.g. radioactive labelling or labelling using fluorescence dyes. The probe is a single stranded polyribo- or polydesoxyribonucleotide being single stranded naturally or being usually double stranded and having been made single stranded by denaturation. This probe binds to the DNA or RNA sample (which is also in single stranded state) by means of base pairing.

The PAM fragments are preferably fragments comprised within the above sequences ID No. 1, 2 or 3 and the derivatives are preferably derived from the above sequences ID No. 1, 2 or 3 or from fragments thereof.

The functional fragments or derivatives thereof are preferably capable of inhibiting adenylyl cyclase (AC) activity, more preferably that of AC Type I, V or VI.

According to a preferred embodiment of the different aspects of present invention, the functional fragments or derivatives thereof comprise or consist of amino acids 400 to 1400, preferably 446 to 1062, 499 to 1065 or 1028 to 1231, and more preferably 1000 to 1300 and even more preferably 1000 to 1100 and even more preferably 1028 to 1065 of the human PAM sequence, preferably of the human PAM sequence according to SEQ ID No. 2, or if they are encoded by the respective polynucleotide fragments, especially if comprised within the sequences according to SEQ ID No.2 or 3.

If the functional fragments or derivatives thereof are polynucleotides, it is preferred, if they comprise or consist of polynucleotides encoding the above polypeptide fragments. More specifically, it is preferred if they comprise or consist of positions 1482 to 3332 (encoding amino acids 446 to 1062) or 1641 to 3341 (encoding amino acids 498 to 1066) or 3228 to 3839 (encoding amino acids 1038 to 1231) of the human PAM cds. It is even more preferred, if the human PAM cds from which the fragments are derived has the sequence according to SEQ ID No.2.

According to one preferred embodiment of present method for identifying pain alleviating compounds, a cell expressing, PAM, preferably recombinant PAM is used. The cell can be any type of cell, e.g. a eukaryotic or prokaryotic single cell organism (such as bacteria, e.g. e. coli, or yeast, e.g. s. pombe or s. cerevisiae) or cell lines derived from multicellular organisms (such as HeLa, COS, NIH-3T3, CHO, etc), wherein mammalian cell lines are preferred.

According to another preferred embodiment, a modified cell, having a lower PAM activity as compared to its unmodified state, is used. This way, it can be tested, if the chemical compounds to be tested for their ability of alleviating pain, are able to enhance or restore the lowered or totally abolished PAM activity.

The modification can be any type of modification (stable or transient, preferably stable), that leads to a decrease of PAM activity (i.e. its ability to lower intracellular cAMP levels, to interact with other factors like AC, especially with AC, to inhibit AC or its ability to lower the pain perception), PAM transcript steady state level (i.e. by activation of PAM transcription or transcript stabilization) or PAM protein steady state level (i.e. by activation of PAM translation or its posttranslational processing; by modulation of PAM posttranslational modification or by activation of its stabilization or by inhibition of its degradation). This can for example be achieved by using dominant negative mutants of PAM, antisense oligonucleotides, RNAi constructs of PAM, by generating functional or genomic PAM knock outs (which can e.g. be inducible) or other suitable techniques known within the state of the art. For an overview of the above techniques, see for example: Current protocols in Molecular biology (2000) J. G. Seidman, Chapter 23, Supplement 52, John Wiley and Sons, Inc.; Gene Targeting: a practical approach (1995), Editor: A. L. Joyner, IRL Press; Genetic Manipulation of Receptor Expression and Function, 2000; Antisense Therapeutics, 1996; Scherr et al, 2003.

According to a preferred embodiment, a PAM knock-out cell is used. Suitable cell lines for the generation of knock-outs are well known in the state of the art and comprise e.g. Current protocols in Molecular Biology (2000) J. G. Seidman, Chapter 23, Supplement 52, John Wiley and Sons, Inc; or Gene Targeting a practical approach. (1995) Ed. A. L. Joyner, IRL Press.

The PAM activity can either be determined directly, e.g. by its ability (or the ability of its fragments and derivatives) to interact with AC or inactivate AC, or it can be determined indirectly, e.g. by its ability (or the ability of its fragments and derivatives) to lower intracellular cAMP levels, to modulate ion concentrations within the neurons or its ability to alleviate pain perception. Suitable techniques for measuring the above parameters are well known in the state of the art (see also above): The cAMP levels can e.g. be measured by HTRF or ALPHAscreen™, the ion concentrations can e.g. be estimated by patch clamping or suitable dyes, the pain perception can e.g. be measured by means of the formalin test or tests of mechanical or thermal hyperalgesia, or the hot plate test etc.

Another aspect of present invention concerns a method of identifying a compound that alleviates pain comprising
a) Selecting a compound that modulates the activity of PAM as a test compound, and
b) Administering said test compound to a subject to determine whether the pain is alleviated.

The subject can be any subject with the ability of perceiving pain, preferably it is a mammal, either a non-human mammal or a human (i.e. within a patient study).

In the following, the invention is illustrated in more detail by means of examples and figures. However, the examples are not meant to limit the scope of the invention.

### EXAMPLES

### Investigation of PAM Expression Pattern and Function

1. Preparation of Animal Sections:

Wild type Sprague Dawley rats were purchased from Charles River Wiga GmbH (Sulzfeld, Germany). The animals had free access to food and water prior to the experiments. They were maintained in climate- and light-controlled rooms (24±0.5° C.). Each animal was used at one occasion only. In all experiments the ethics guidelines for investigations in conscious animals were obeyed, and the procedures were approved by the local Ethics Committee. After killing, adult rats were fixed by perfusion with 4% paraformaldehyde in 0.1 M phosphate buffered saline (PBS, pH 7.2) for one hour. Tissues were cryostat-sectioned in the horizontal plane at a thickness of 14-16 μm. Sections were mounted on Superfrost Plus Slides (Fisher Scientific Co., Pittsburgh, Pa.) and stored at −80° C. until use.

2. Preparation of Riboprobes:

The riboprobes were generated as described previously (Yang et al., 2002). Antisense and sense riboprobes of rat PAM were obtained with T7 and T3 polymerases, after linearizing the plasmid with Hind III (antisense) and BamHI (sense), respectively (see Yang et al., 2002). In vitro transcription was performed in the presence of [35S] UTP-αS (ICN, Irvine Calif.), linearized PAM cDNA, NTP at 37° C. for 1 hour according to the manufacturer's recommendation (Promega, Madison, Wis.). The RNA transcripts were purified using RNA Probe Purification Kit (Pequlab, Erlangen, Germany).

3. In situ Hybridization:

In situ hybridization was performed as described earlier (Yang et al. 2002): Sections were fixed in 4% paraformadehyde in 0.1 M phosphate-buffered saline (pH 7.2), pretreated with 0.25% acetic anhydride and 0.1 M triethanolamine, rinsed with 0.2×SSC and dehydrated with serially increasing concentrations of alcohol. Sections were prehybridized with prehybridization solution (50% deionized formamide, 0.6 M sodium chloride, 10 mM Tris-HCl (pH 7.6), 50 mM EDTA, 0.025% sodium pyrophosphate, 0.02% Ficoll, 0.02% BSA, 0.02% polyvinyl pyrrolidone, 10 mM DTT, and heat-denatured, heterologous nucleic acids (0.005% yeast tRNA, type X, 0.05% yeast total RNA, type I, 0.05% salmon testes DNA, type III)) for 2 hours at room temperature; hybridized with riboprobes in hybridization solution (2.5×106 cpm/section), 50% deionized formamide and 50% hybridization buffer containing 0.6 M sodium chloride, 10 mM Tris-HCl (pH 7.6), 50 mM EDTA, 0.025% sodium pyrophosphate, 0.02% Ficoll, 0.02% BSA, 0.02% polyvinyl pyrrolidone, heat-denatured, heterologous nucleic acids (0.005% yeast tRNA, type X, 0.005% yeast total RNA, type I, 0.05% salmon testes DNA, type III), 100 mM DTT, 0.0005% polyadenylic acid, 10% dextran sulfate) at 50° C. overnight. Sections were rinsed with 2×, 1×, 0.5×SSC at RT (room temperature). After digestion in 20 μg/ml RNase A (Sigma, St. Louis, Mo.), sections were washed in 1× RNase buffer, 2×, 1×, 0.5×SSC at room temperature, and in 0.1×SSC overnight at 45° C. Sections were dehydrated with serially increasing concentrations of alcohol, exposed to Kodak Biomax MR film (Kodak, Rochester, N.Y.) for 3-7 days at −80° C.

4. Antibody Generation and Immunofluorescence Staining:

Antisera were raised commercially in rabbits using peptides consisting of amino acid residues 135-153 and 4601-4614 of human PAM corresponding to the SEQ ID No.1, respectively (BioTrend, Cologne, Germany). The antiserum was commercially produced by BioTrend, Cologne, Germany, according to standard procedures. To monitor the distribution of PAM in spinal cord and DRG slices, the slices were permeablized in 0.1% Triton X-100 for 5 minutes. The slices were blocked for 1 hour in 3% BSA in PBS and then incubated for 1 hour with anti-PAM antiserum (1:50 dilution). This was followed by incubation with FITC-labeled goat anti-rabbit antibody in PBS containing 3% BSA. The slices were then washed with PBS and mounted using fluoromount™.

5. RT-PCR:

Total RNA from rat spinal cords and DRGs was isolated by guanidinium isothiocyanate/phenol/chloroform extraction (Chomczynski and Sacchi 1987). 2 μg of total RNA were annealed with 0.6 μM of each of oligo (dT) primer and reverse-transcribed using reverse transcriptase (Promega, Madison, Wis.) for 30 minutes at 37° C. The cDNA was then immediately used for amplification. Oligonucleotide primers used for the amplification of rat GADPH were 5′-GAAGGGTGGGGCCAAAAG-3′ (sense; SEQ ID No.10) and 5′-GGATGCAGGGATGATGTTCT-3′ (antisense; SEQ ID No.11; Trajkovic et al. 2000). Primers for the amplification of AC isoforms were chosen as published by Bek et al. (Bek et al. 2001). Primers for rat PAM were 5′-GGTGGTGAAGCTCGCTGTGATGCT-3′ (sense; SEQ ID No.12) and 5′-CGTGTGAGCATTTCTGCACAC TCC-3′ (antisense; SEQ ID No.13). The PCR product corresponds to the human PAM CDNA nucleotides 13692-14064. The corresponding rat sequence was derived from the EST clone AW441131 (SEQ ID No.8). For semiquantitative PCR, SAWDAY DNA Polymerase (Peqlab, Erlangen, Germany) was used. After an initial denaturation step at 95° C. for 5 minutes, 30 cycles were performed with 1 minute at 95° C., 30 seconds at 55° C., and 10 seconds at 72° C., followed by a final 10-minute extension step at 72° C. Quantitative PCR was performed using the TaqMan™ system and reagents (Applied Biosystems, Weiterstadt, Germany) according to the instructions of the manufacturer.

6. Purification of Full Length PAM:

PAM purification was performed with some modifications as published previously (Scholich et al. 2001). Shortly, HeLa cells were grown in DMEM medium with 10% fetal bovine serum and 1% penicillin/streptomycin. Confluent cells of forty 150 mm dishes were harvested with 1×PBS, 1 mM EDTA and pelleted for 5 minutes at 400×g. The cells were resuspended in TED buffer (50 mM Tris-HCl, pH 8.0, 1 mM EDTA, 1 mM DTT) containing 125 mM NaCl, 20 μg/ml of aprotinin, 20 μg/ml leupeptin, 1 mM benzamidine, 5 μg/ml soybean trypsin inhibitor and lysed by 2×5 seconds of sonication. The homogenate was centrifuged at 27000×g for 30 min at 4° C., and the supernatant was loaded on a Q-Sepharose XK16 column (Amersham, Pharmacia, Piscataway, N.J.) and eluted with a gradient of 150-350 mM NaCl in TED according to instructions of the manufacturer. The fractions were analyzed by Western blotting according to standard procedures; positive fractions were pooled and the NaCl concentration adjusted to 1 M. The protein was then loaded on a Phenyl-Sepharose XK16 column (Amersham, Pharmacia, Piscataway, N.J.) and washed with 300 mM NaCl in TED according to instructions of the manufacturer. The flow through and wash fractions contained PAM. They were pooled and the buffer exchanged for the aforementioned TED buffer containing 100 mM NaCl using Centricon 50 (Amicon, Beverly, Mass.) according to the manufacturer's instructions. The protein was then loaded on a MonoS 5/5 FPLC column (Amersham, Pharmacia, Piscataway, N.J.) and washed with the loading buffer (100 mM NaCl in TED) according to instructions of the manufacturer. The flow through was collected and applied to a Mono Q 5/5 FPLC column (Amersham, Pharmacia, Piscataway, N.J.). The protein was eluted with a gradient from 150-400 mM NaCl in TED. Positive fractions were pooled and the buffer exchanged for 50 mM Tris-HCl, pH 8.0, 1 mM DTT using Centricon 50 (Amicon, Beverly, Mass.) and stored at −80° C. The stored PAM was used within 3 weeks.

7. Expression and Purification of Recombinant Gsα:

The hexahistidyl tagged constitutively active Q213L mutant of Gsα (Gsα*) was expressed and purified as described in Graziano et al., 1991. To ensure maximal activation of the Gsα*, the G protein was incubated with 1 μM GTPγS in the presence of MgCl2 (25 mM) for 30 minutes prior to use in AC activity assays.

8. Adenylyl Cyclase Activity Assays (AC Activity Assays):

Spinal cords were lysed in 25 mM Hepes, pH 7.4, 1 mM EGTA and cell membranes were prepared as described by Kassis and Fishman. Aliquots were stored at −80° C. until use. AC activity assays were performed in a volume of 100 μl for 15 min at room temperature in the presence of 100 μM MgCl₂ as previously described (Patel et al., 2002). Gsα* (80 nM) or forskolin (100 μM) were used to stimulate AC enzyme activity in membranes (10 μg protein).

9. Spinal Delivery of PAM Antisense and Sense Oligonucleotides:

Rats were anesthetized with ketamine (60 mg/kg i.p.) and midazolam (0.5-1 mg/kg i.p.). The skin was incised above the vertebral column from vertebrae Th13 up to L3. Muscle tissue around L2-3 was cleared away. The processus spinosus of L3 was removed and a laminectomy was done at L2. Polyethylene catheters (ID 0.28 mm, OD 0.61 mm) were then inserted into the peridural space so that the tip of the catheter reached Th9-10. The catheter was fixed with cyanacrylate glue, externalized in the neck region, and the skin was sutured.

10. Infusion of PAM Oligonucleotides:

The sequences of the oligodeoxynucleotides (ODNS) were chosen from the rat PAM sequence as follows. Sense: 5′-GACTGGTTTAGCAATGGC-3′(SEQ ID No.14), antisense: 5′-GCCATTGCTAAACCAGTC-3′ (SEQ ID No.15), and antisense ODN harboring three mutations (3M-as; mutations are underlined): 5′-GCAATTGCTAAATCAGTA-3′ (SEQ ID No.16). Three days after surgery, rats were placed into a “freely moving system” (CMA, Stockholm, Sweden) and antisense (n=5) or sense (n=5) oligonucleotides (2.5 mg/ml in artificial cerebrospinal fluid) were infused through the catheter at a flow rate of 0.05-0.1 μl/min for 100 hours using a microinfusion pump (CMA, Stockholm, Sweden).

11. Formalin Test:

Within 15 min after stopping the infusion, the formalin test was performed. 50 μl of a 5% formaldehyde solution was injected subcutaneously (s.c.) into the dorsal surface of one hind paw. Flinches were counted in one-minute intervals up to 60 min starting right after formalin injection. Flinches of 5 min intervals were summarized as mean flinches per minute. To compare the nociceptive behavior between groups, the sum of flinches during the two phases of the one-hour observation period were submitted to the Students t-test. α was set at 0.05.

At the end of the formalin test, the rats were killed, the lumbar spinal cord and dorsal root ganglia (DRGs) were excised, snap frozen in liquid nitrogen and stored at −80° C. until further analysis. To determine PAM expression, spinal cord slices were analyzed immunhistochemically using the above anti PAM antibodies.

12. Zymosan-Evoked Inflammation:

For induction of an inflammation, 2.5 mg zymosan A (Sigma, St. Louis, Mo.) suspended in 30 μl 0.03 M phosphate buffered saline (PBS, pH 7.5) was injected subcutaneously into the midplantar region of the right hindpaw. Such intraplantar zymosan injection is known to induce a reliable model of thermal and mechanical hyperalgesia rats (Meller and Gebhart 1997). Rats were killed by cardiac puncture under deep isoflurane anesthesia 24-96 hours after the Zymosan injection. The lumbar spinal cord and dorsal root ganglia (DRGs) were excised, snap frozen in liquid nitrogen and stored at −80° C. until further analysis.

13 Results:

The above experiments of the inventors using RT-PCR, immunhistochemistry and in situ hybridisation, demonstrate for the first time that PAM is expressed in sensory neurons of the spinal cord as well as in dorsal root ganglia (DRGs) of adult rats. PAM mRNA was detected by RT-PCR at similar levels in the spinal cord and dorsal root ganglia throughout development (E14-adult). PAM-expression is up-regulated 24-48 hours after zymosan treatment of rats as shown by western blot and RT-PCR.

The major adenylyl cyclase isoforms, which are expressed in the spinal cord and DRGs, are AC type 5 and 6 and AC type 4 and 6, respectively. No major changes in AC isoform expression were observed after zymosan treatment in spinal cord. Hence, Gαs stimulated AC activity in membrane preparations from spinal cord and DRG was inhibited by PAM. Consequently, it was found that treatment with antisense but not sense oligonucleotides against PAM increased formalin induced paw flinching in adult rats. Accordingly cAMP accumulation in the spinal cord of rats treated with antisense oligonucleotides to PAM was elevated as compared to control rats.

Addition of purified PAM to spinal cord lysates resulted in an inhibition of Gαs-stimulated AC activity of spinal cord lysates from control and zymosan treated animals (FIG. 5b). At 30 nM Gas-stimulated AC activity was decreased by 50% in spinal cord lysates of control animals and 70% in lysates derived from rats treated with zymosan for 96 hours.

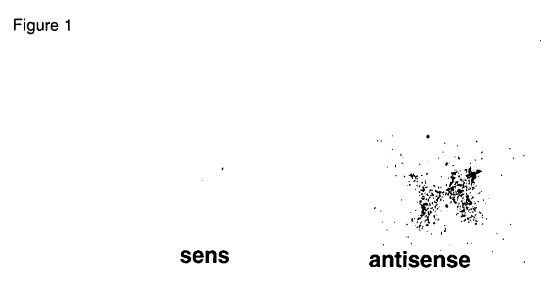

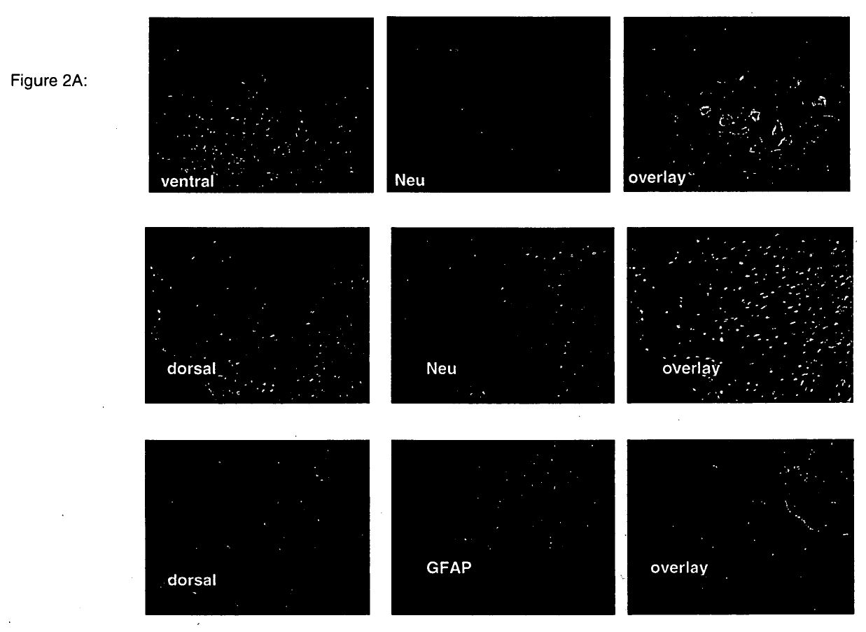

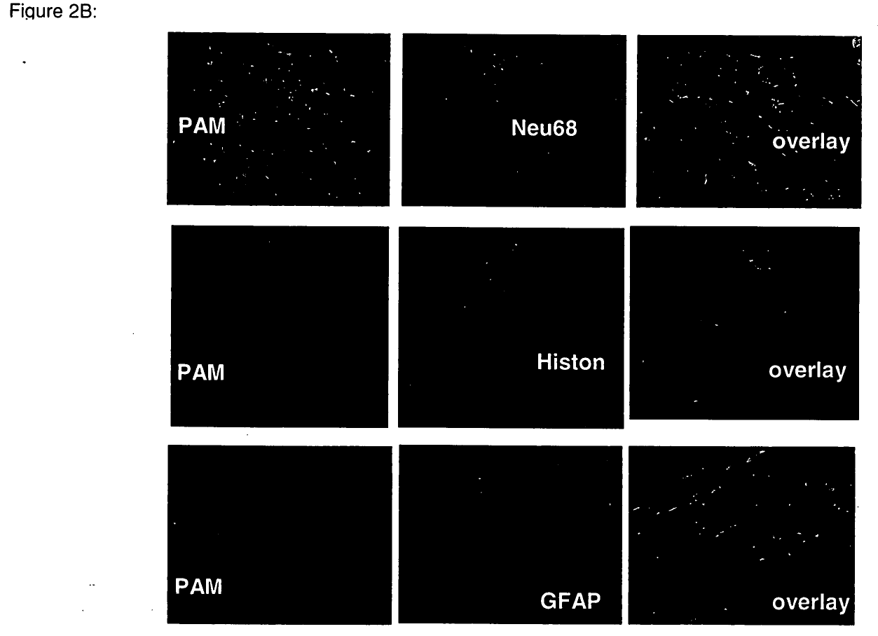

To determine if PAM is expressed in the spinal cord, first in situ hybridization was performed. This led to the detection of a clear signal for PAM mRNA throughout the gray matter but not in the white matter of the spinal cord of adult rats (FIG. 1). To define more precisely the cell populations that express PAM in the spinal cord as well as in DRGs, antibodies against PAM using peptides corresponding to the amino acid residues 135-153 and 4601-4614 of human PAM were generated. The immunhistochemical analysis revealed that PAM was co-localized with anti-NeuN but not with anti-GFAP immunoreactivity (FIG. 2a). More specifically, PAM expression was detected predominantly in dorsal horn neurons (FIG. 2a) while non-neuronal cell populations exhibited very little PAM expression. Especially high PAM expression could be detected in DRG neurons (FIG. 2b). Here, PAM immunoreactivity was located in the axons as well as in the cell body of both large- and small-diameter neurons (FIG. 2b). Interestingly, no PAM was detected in the nuclei of the cells as demonstrated by co-staining with anti-Histone antibody (FIG. 2b). As could also be seen in the spinal cord, PAM expression was not detected in GFAP expressing cells.

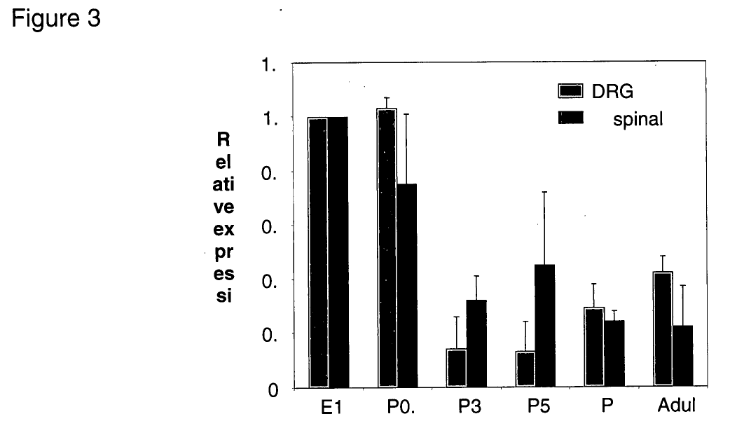

Since PAM expression in the brain is differentially regulated during development in rats and mice (Yang et al. 2002), the inventors investigated if PAM mRNA expression also changes during development in rat spinal cord and DRG. To this end, PAM mRNA expression was determined using quantitative RT-PCR. PAM mRNA was found to be highly expressed in the spinal cord and DRG in late embryonic stages (E16) until shortly after birth (P0.5; FIG. 3). Interestingly, shortly after birth the expression declined to 30-40% of the embryonic expression and then remained constant throughout adulthood (FIG. 3).

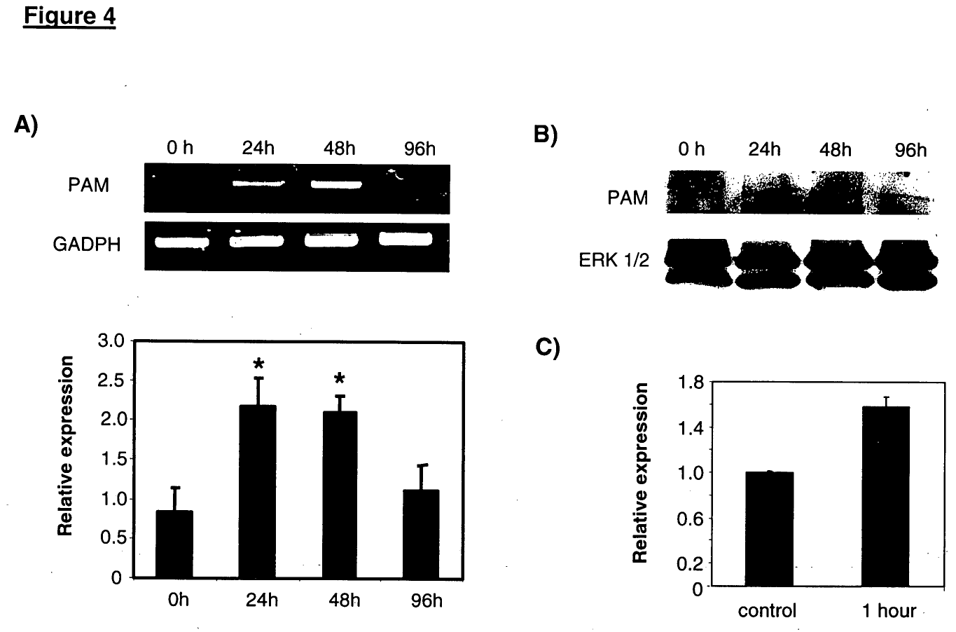

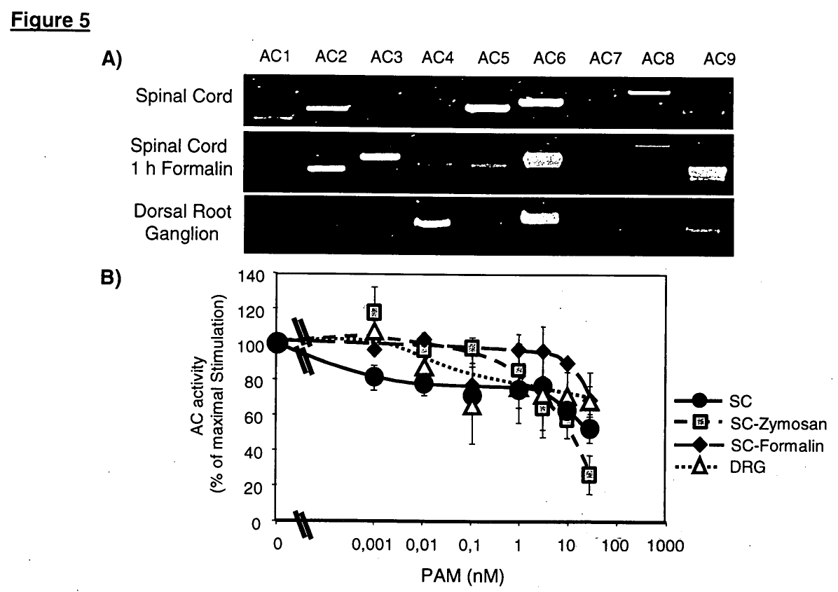

Next, it was examined if PAM expression in the spinal cord is regulated by nociceptive stimuli. Therefore, spinal PAM expression was monitored after zymosan and formalin injection in the hind paws of adult rats. PAM mRNA was up-regulated about two-fold at 24 and 48 hours after zymosan treatment (FIG. 4a). Accordingly, PAM expression was up-regulated at the protein level 24 hours after zymosan injection and stayed elevated for 96 hours (FIG. 4b). 96 hours after zymosan injection, PAM mRNA expression declined. This reduction in PAM mRNA expression was not reflected at the protein level (comp. FIG. 4a and b). Notably, PAM mRNA was also upregulated in the spinal cord of rats 1 hour after formalin injection (FIG. 4c).

Since PAM is known to be a powerful inhibitor of adenylyl cyclases type 1, 5 and 6 (Scholich et al. 2001) it was investigated if PAM is able to inhibit AC activity in spinal cord and DRG lysates. Two major AC isoforms are detected by semi-quantitative RT-PCR in the spinal cord. These AC isoforms are type 5 and 6 (FIG. 5a). Notably, both isoforms are inhibited at nanomolar concentrations of PAM (Scholich et al. 2001). The AC isoform expression pattern in the spinal cord was not significantly altered 24-96 hours after zymosan injection. Interestingly, 1 hour after formalin injection a shift in AC isoform expression was detected (FIG. 5a). The mRNA of AC type 5 is down-regulated and the mRNA of AC type 3 and 9 are up-regulated.

As could be shown by the above experiments, addition of purified PAM to spinal cord lysates resulted in inhibition of Gas-stimulated AC activity in spinal cord preparations. Addition of 30 nM PAM decreased Gαs-stimulated AC activity by 49% in spinal cord lysates of control animals (FIG. 5b). Gαs-stimulated AC activity in spinal cord lysates derived from rats 96 hours after zymosan injection exhibited higher sensitivity to PAM inhibition as compared to untreated animals (70% inhibition at 30 nM; FIG. 5b). In contrast, inhibition of AC activity by PAM in spinal cord lysates from animals treated for 1 hour with formalin was inhibited to a lesser extent (25% inhibition at 30 nM; FIG. 5b).

In DRGs the predominantly expressed AC isoforms are AC type 4 and 6 (FIG. 5a). Gαs-stimulated AC activity in DRG lysates was decreased by 32% in presence of 30 nM PAM in contrast to spinal cord lysates (FIG. 5b).

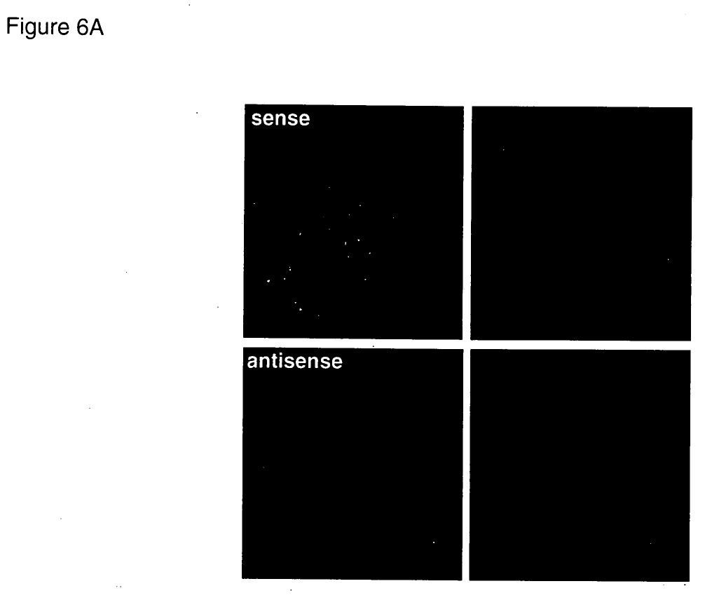

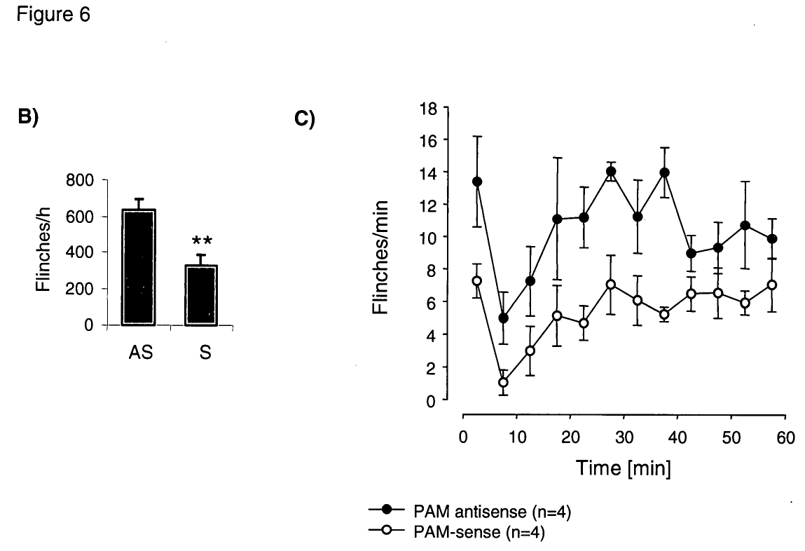

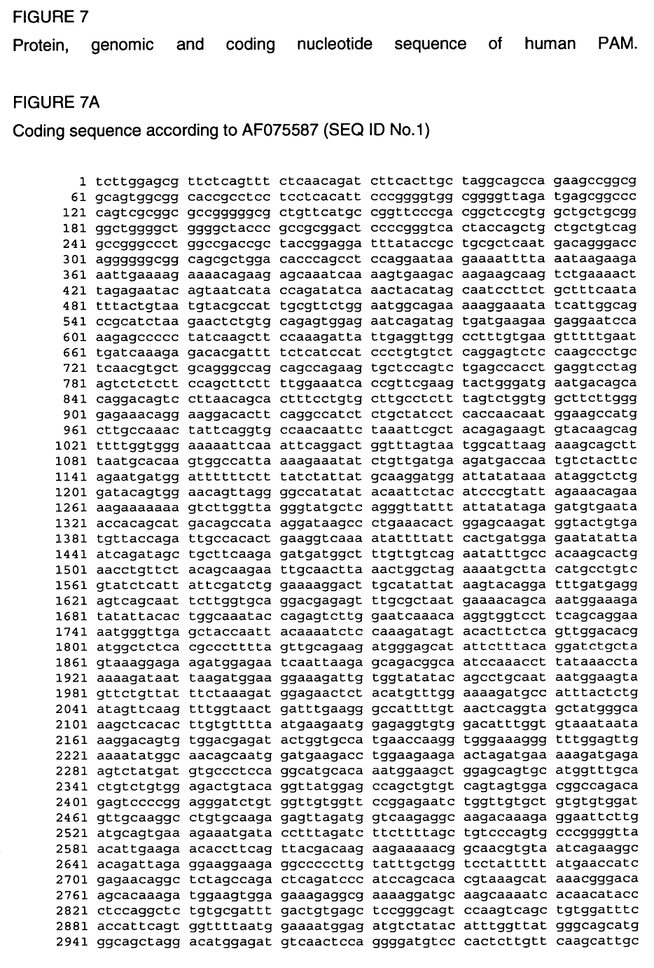

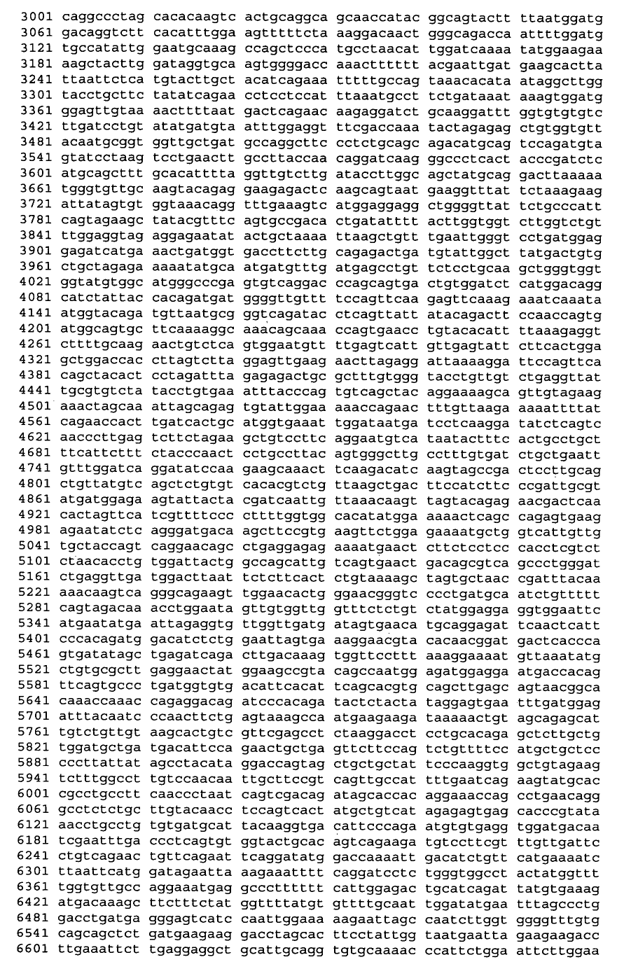

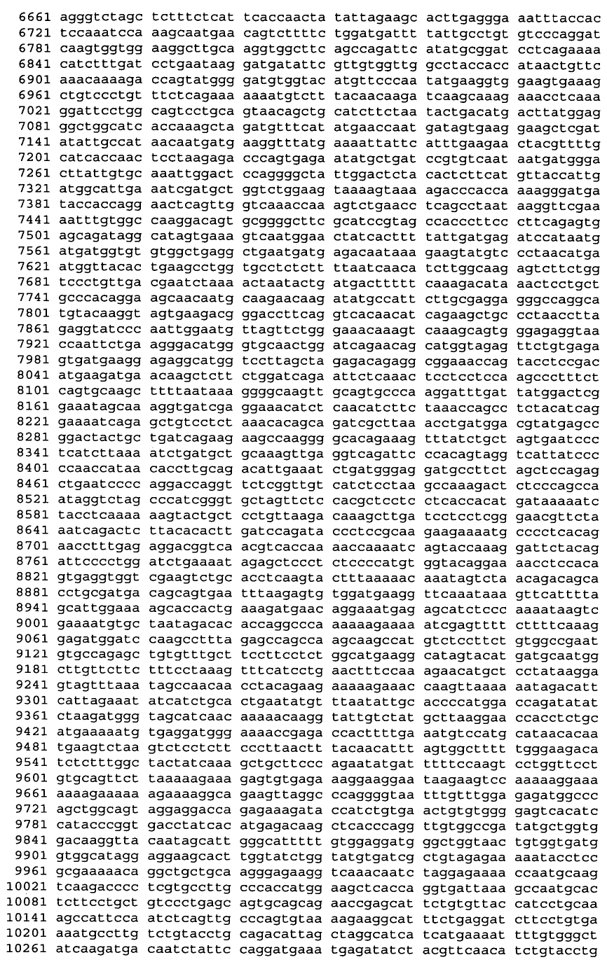

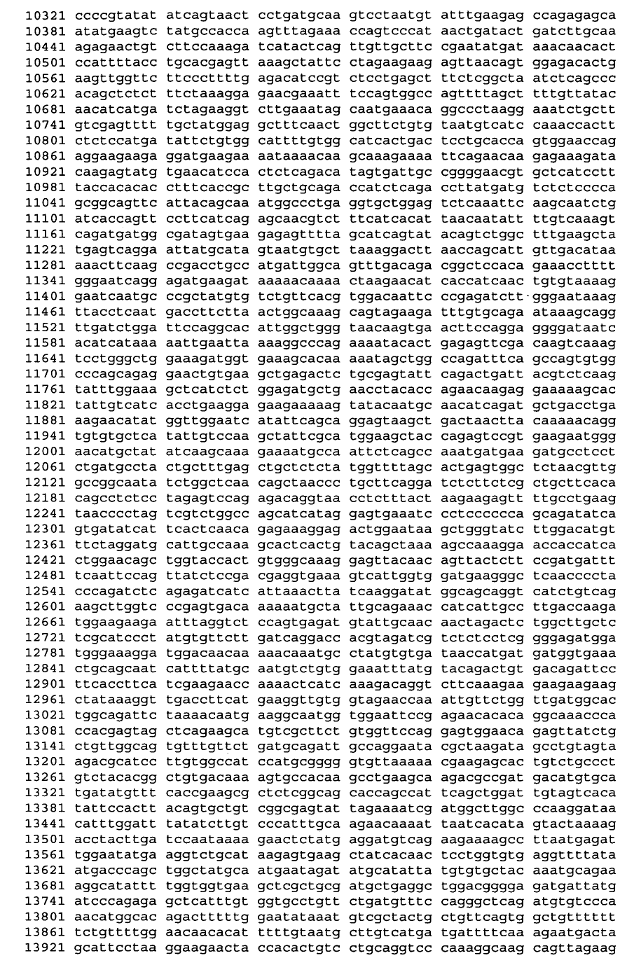

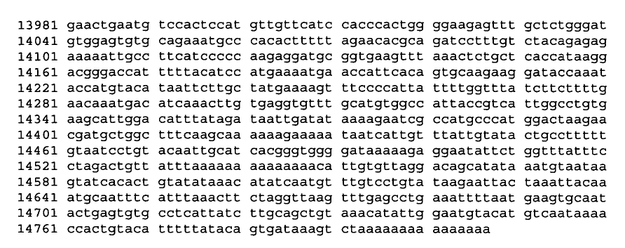

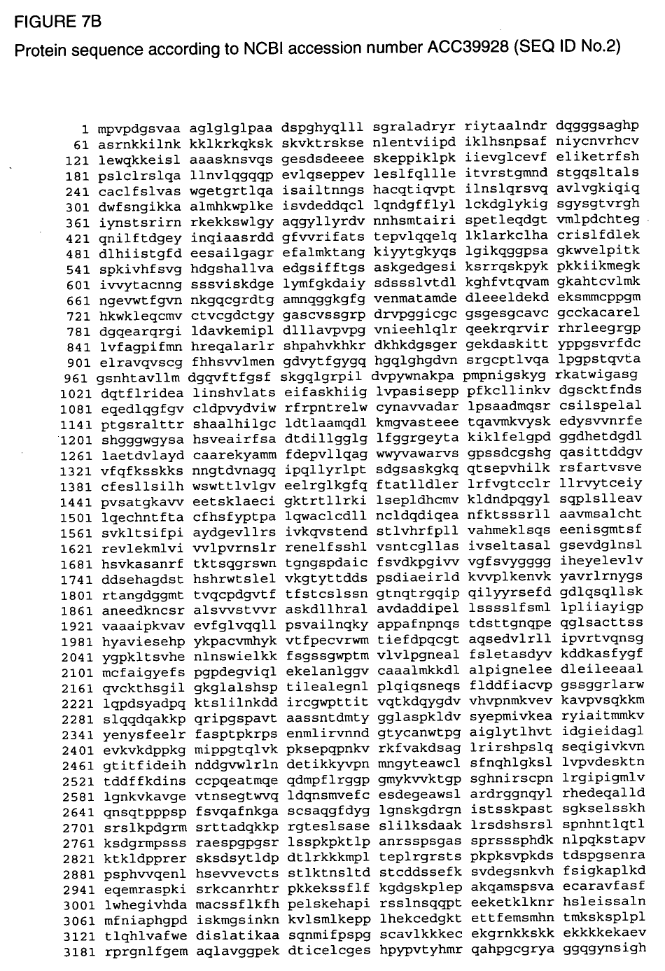

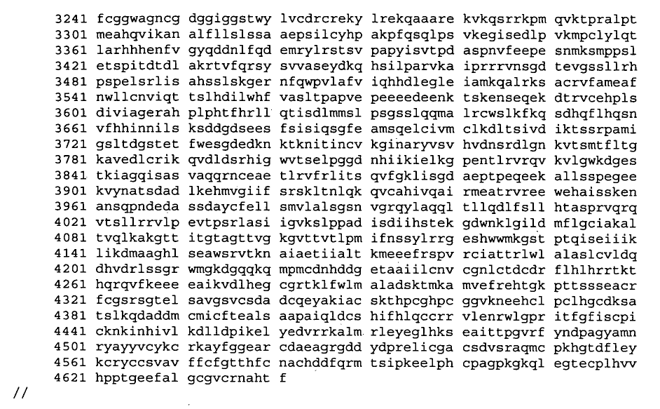

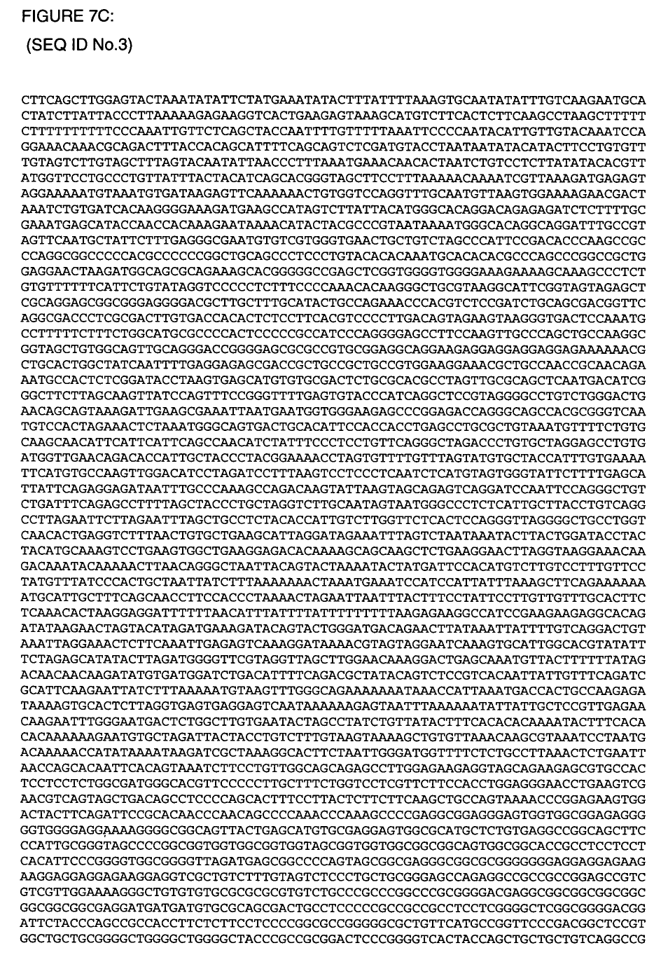

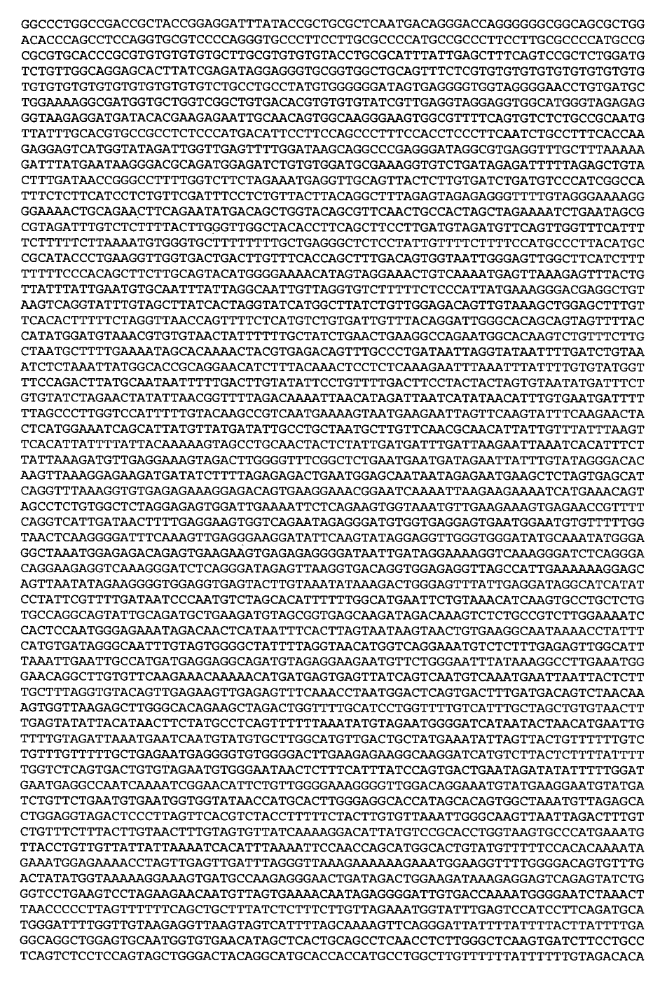

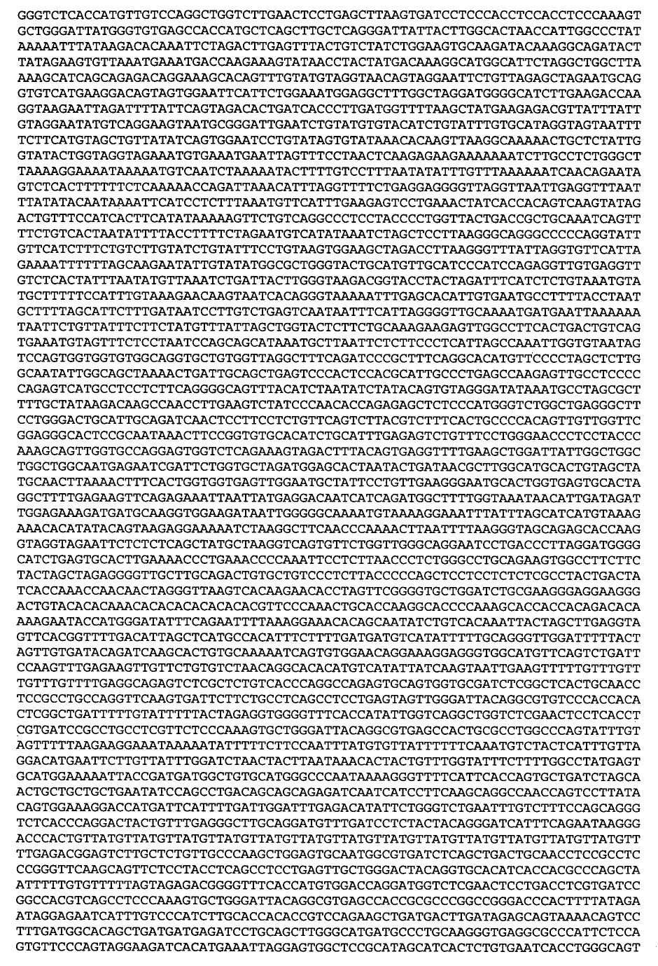

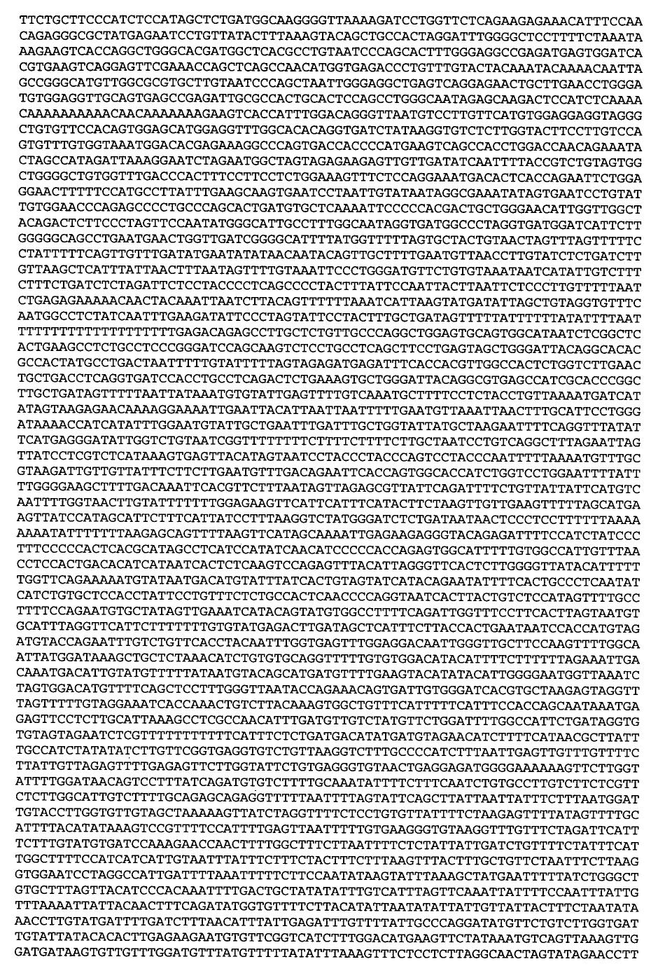

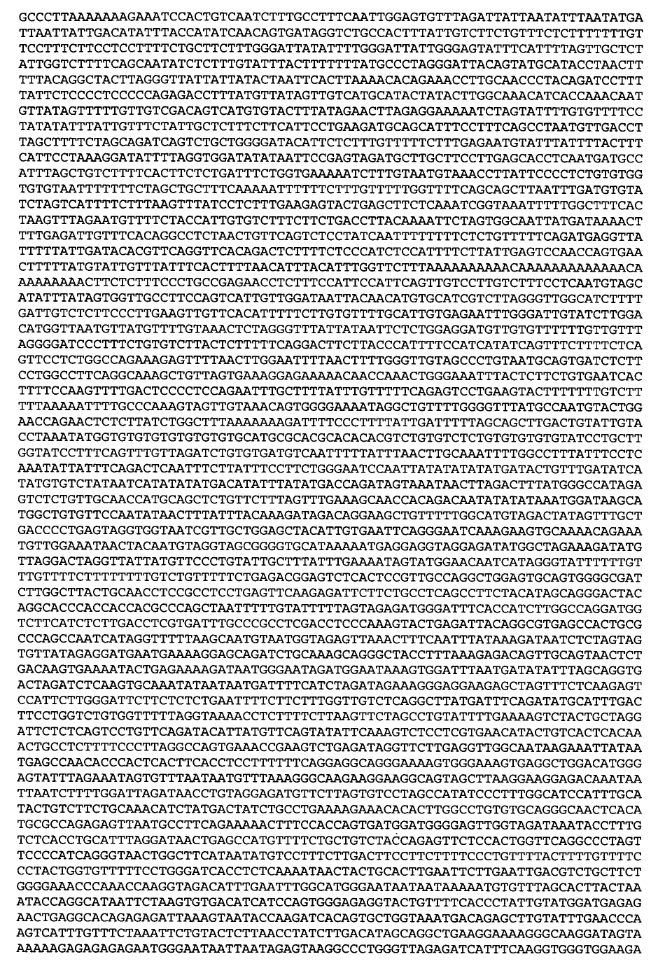

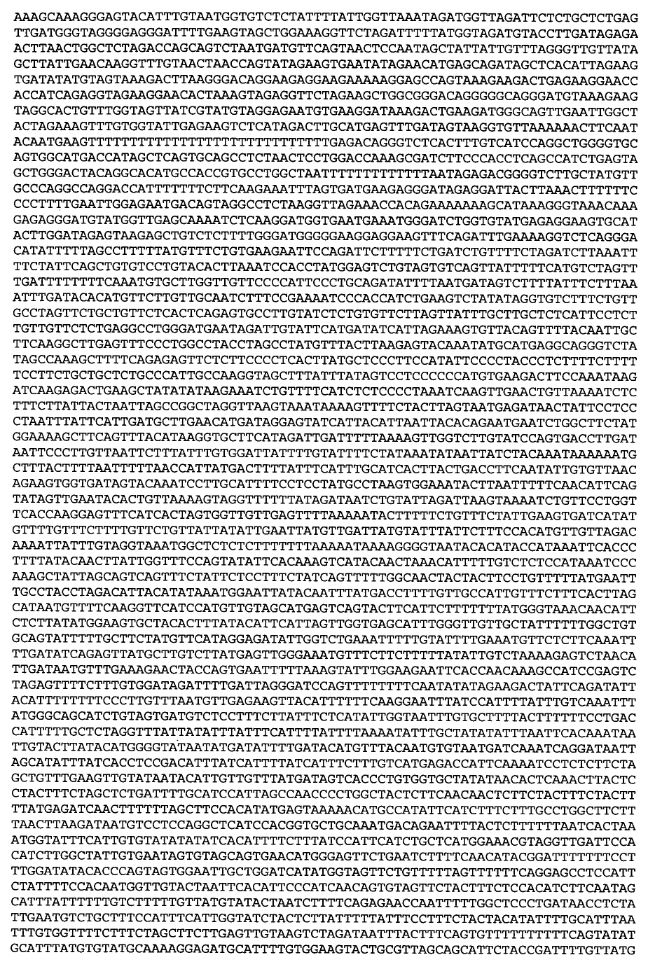

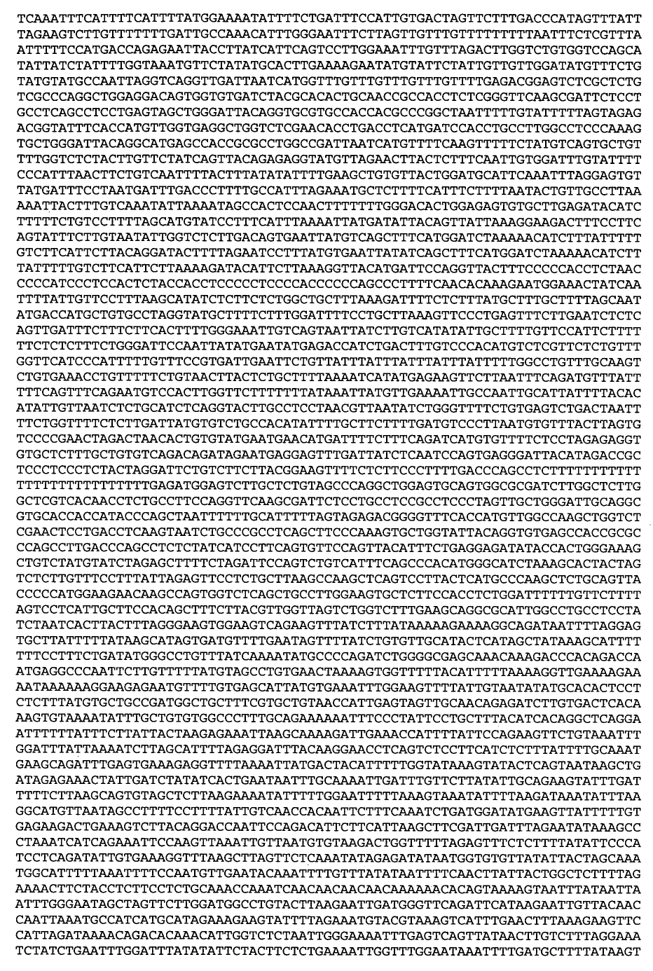

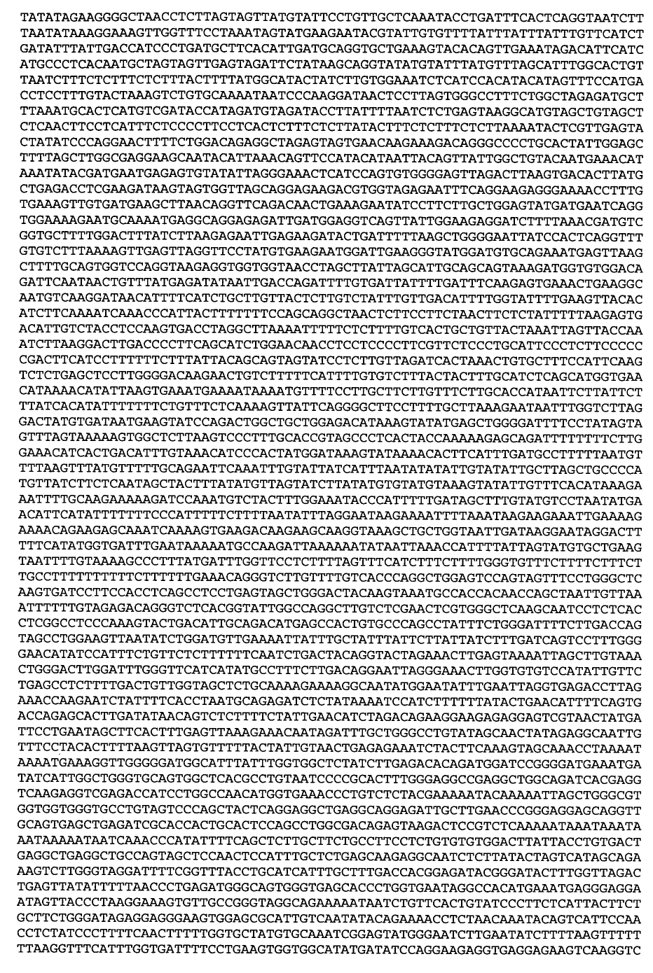

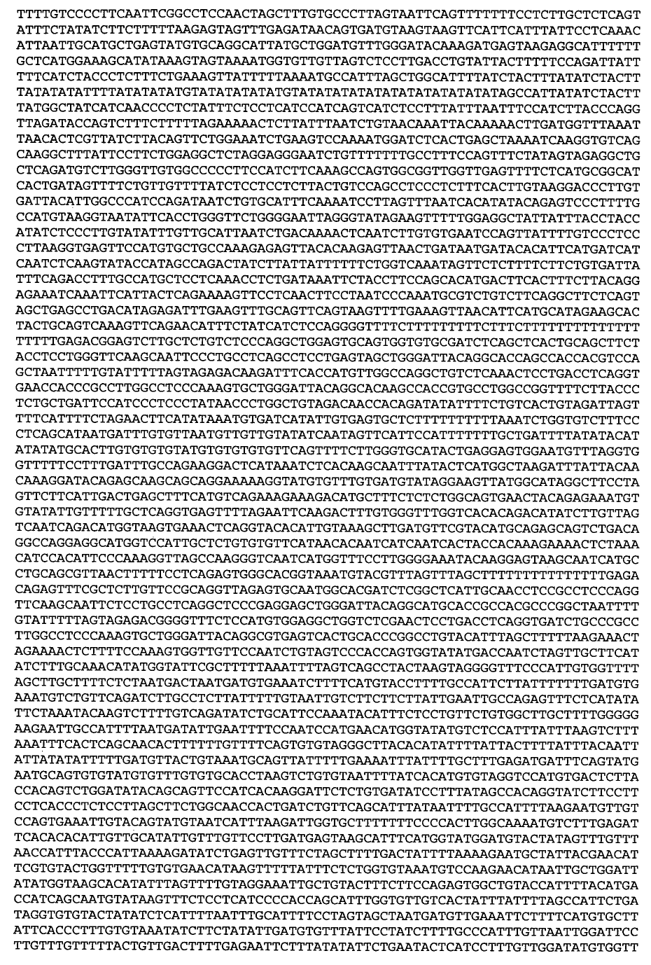

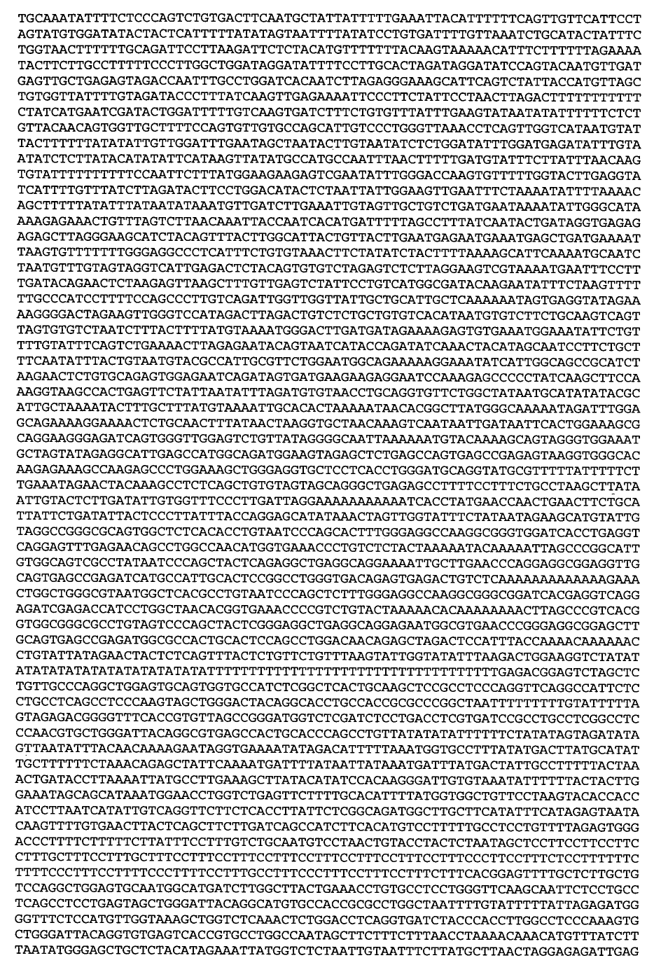

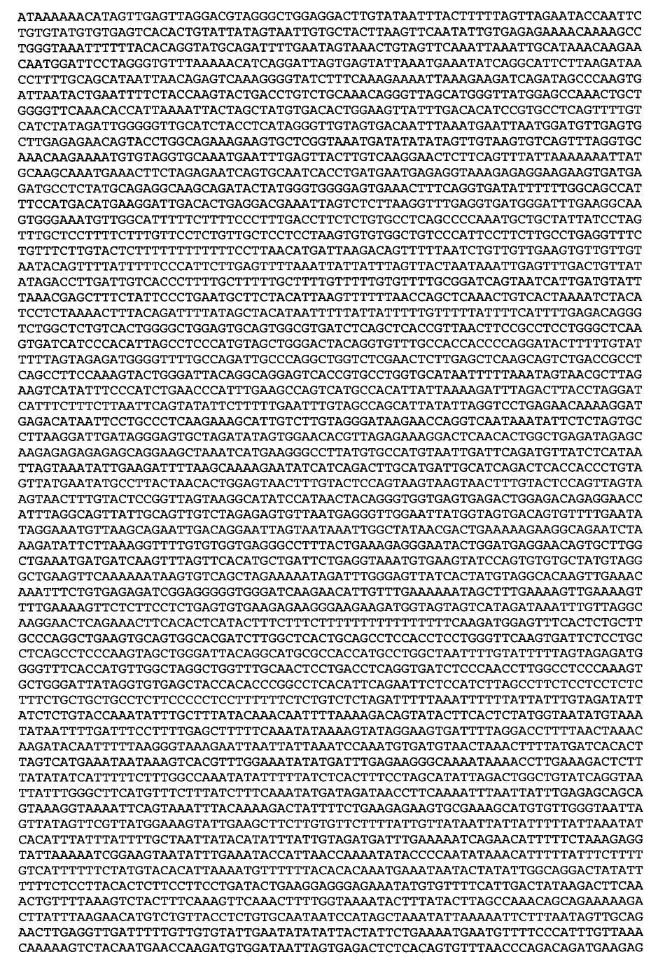

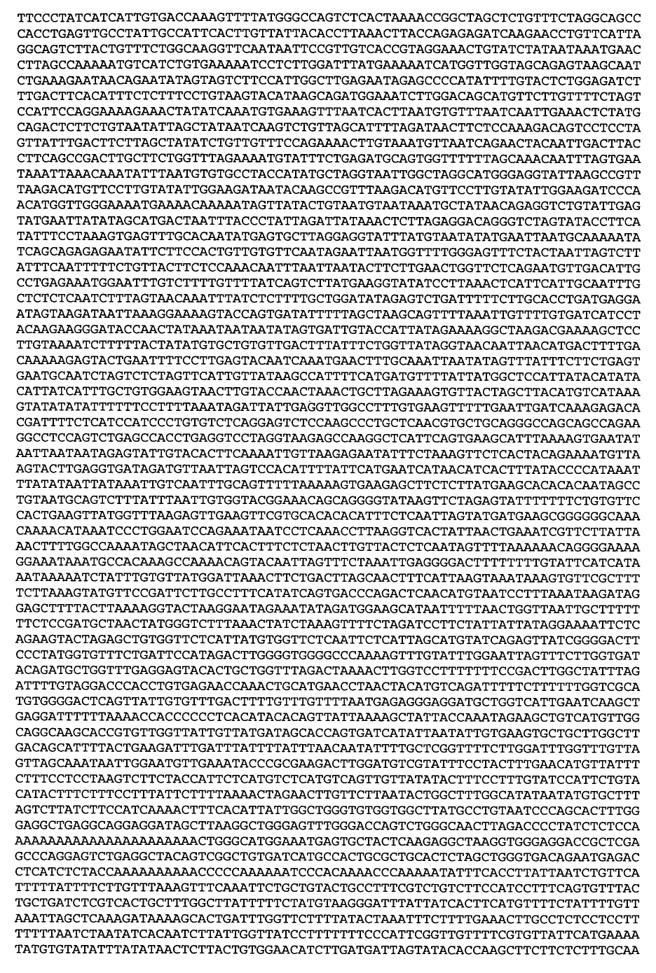

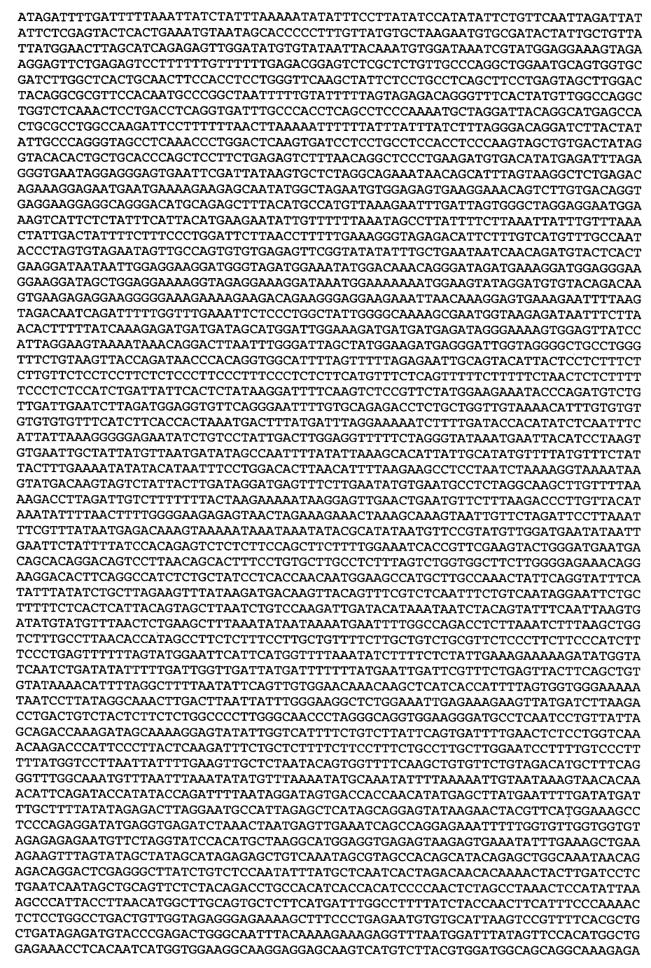

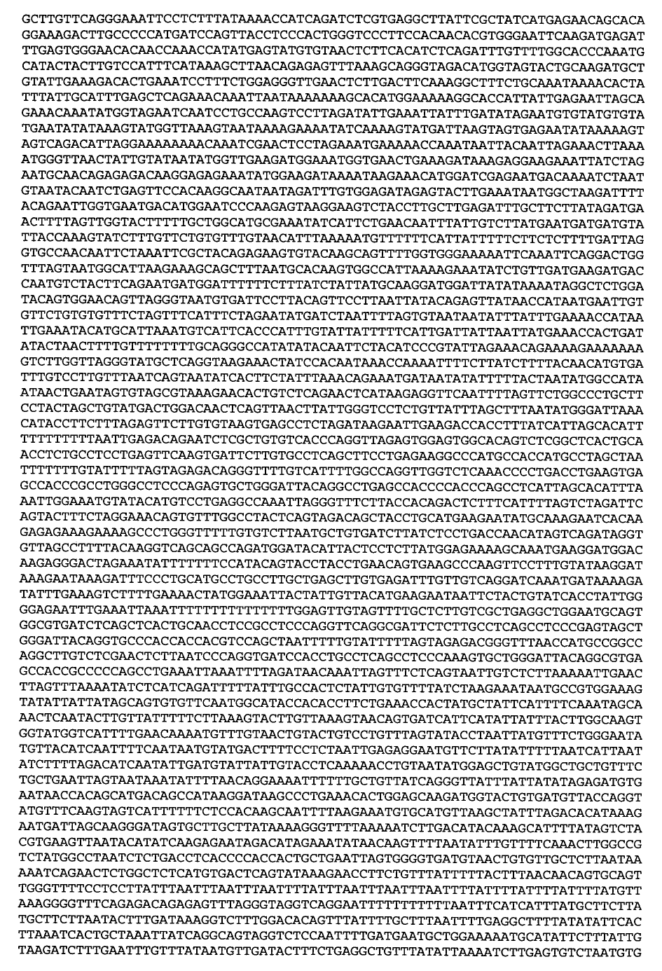

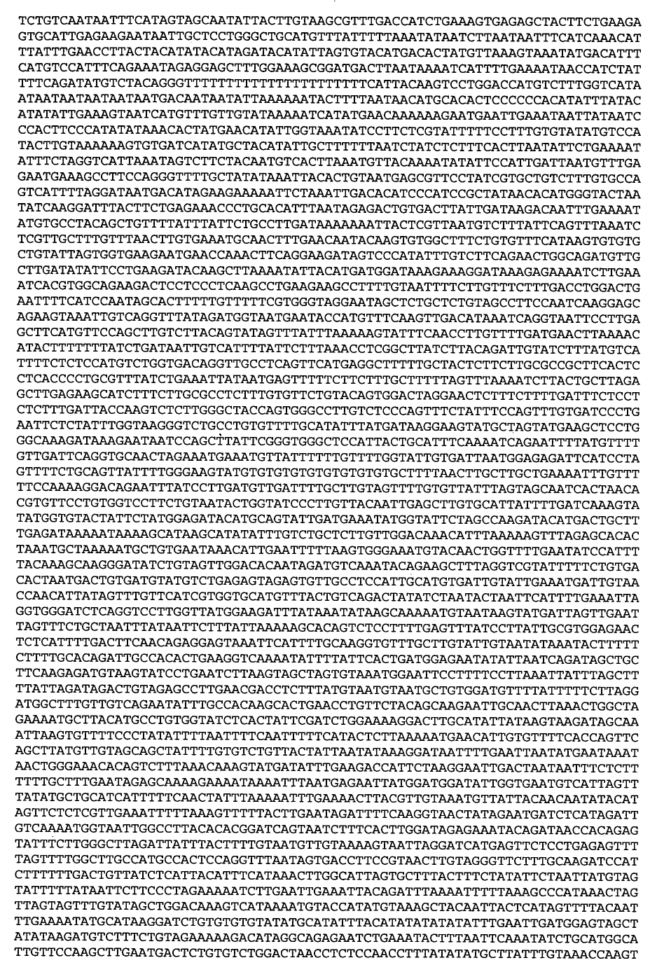

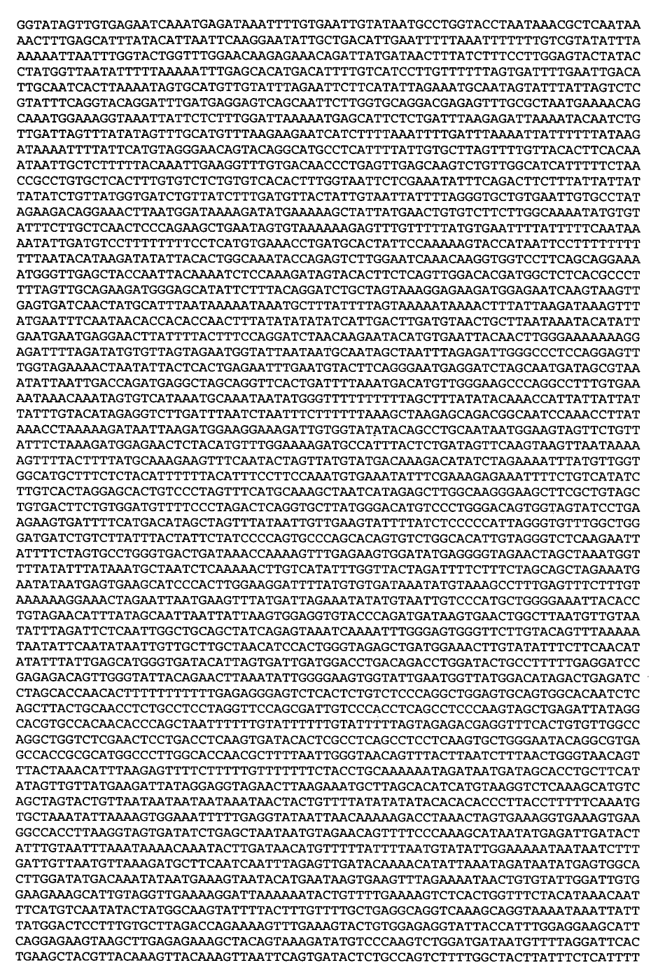

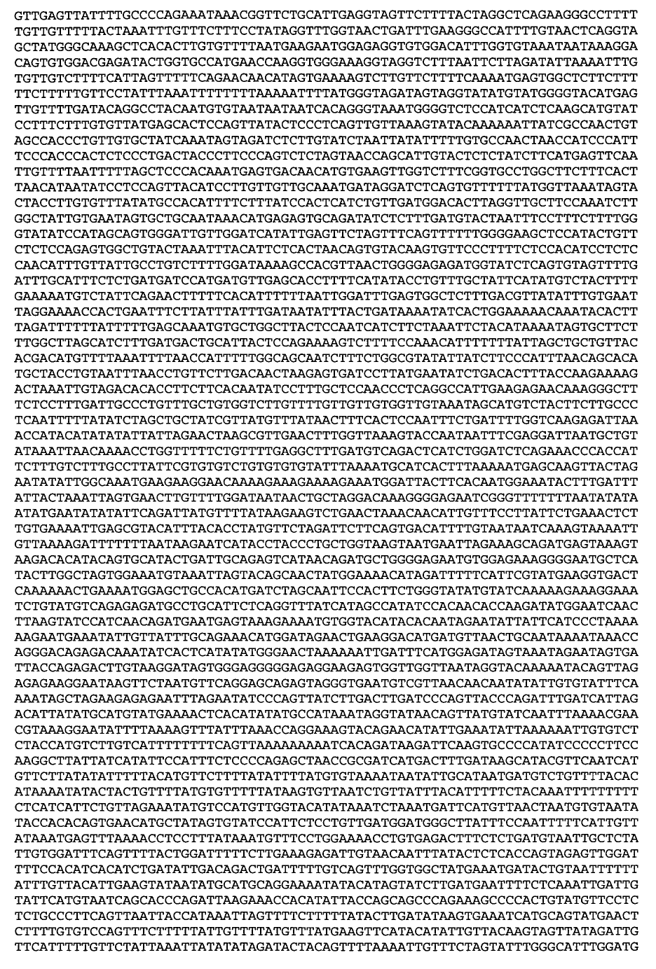

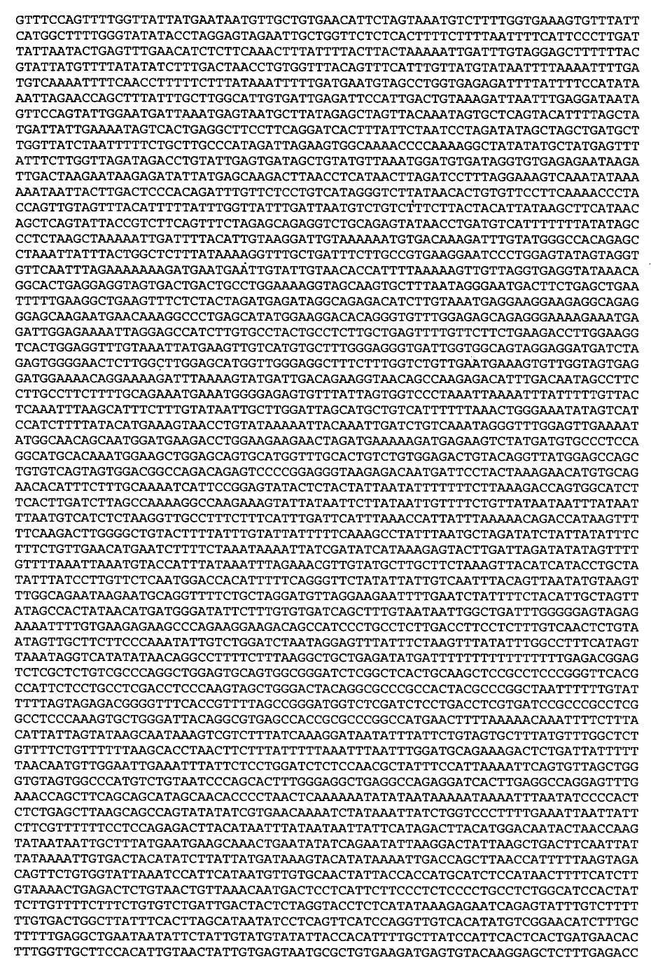

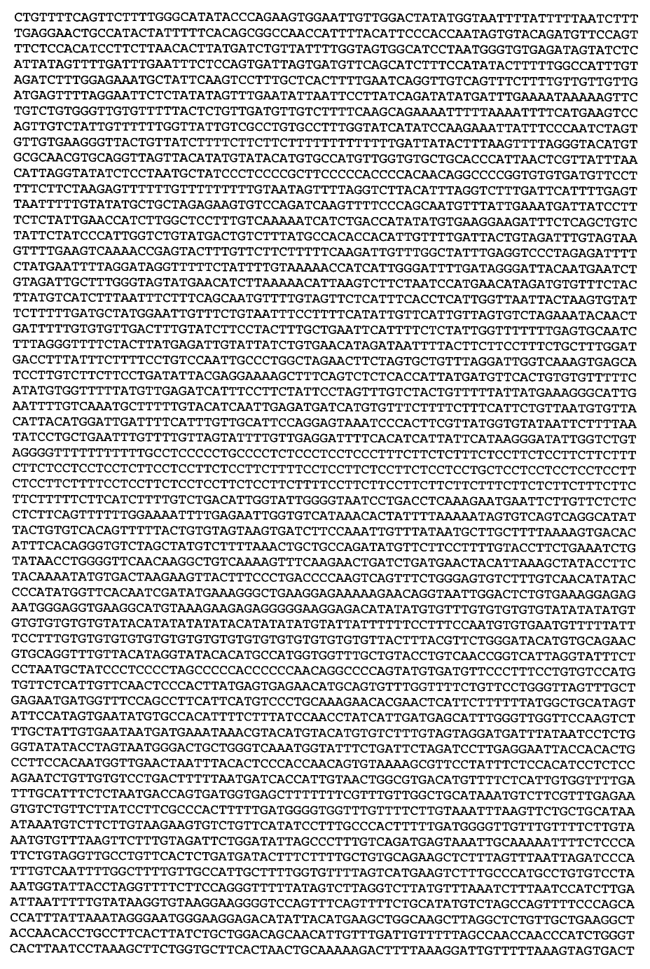

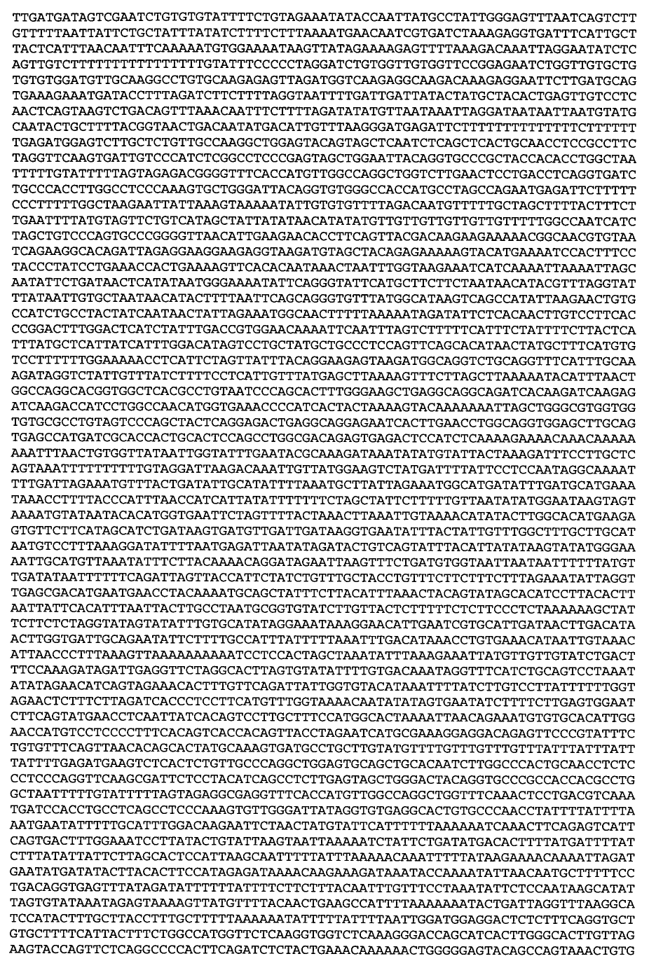

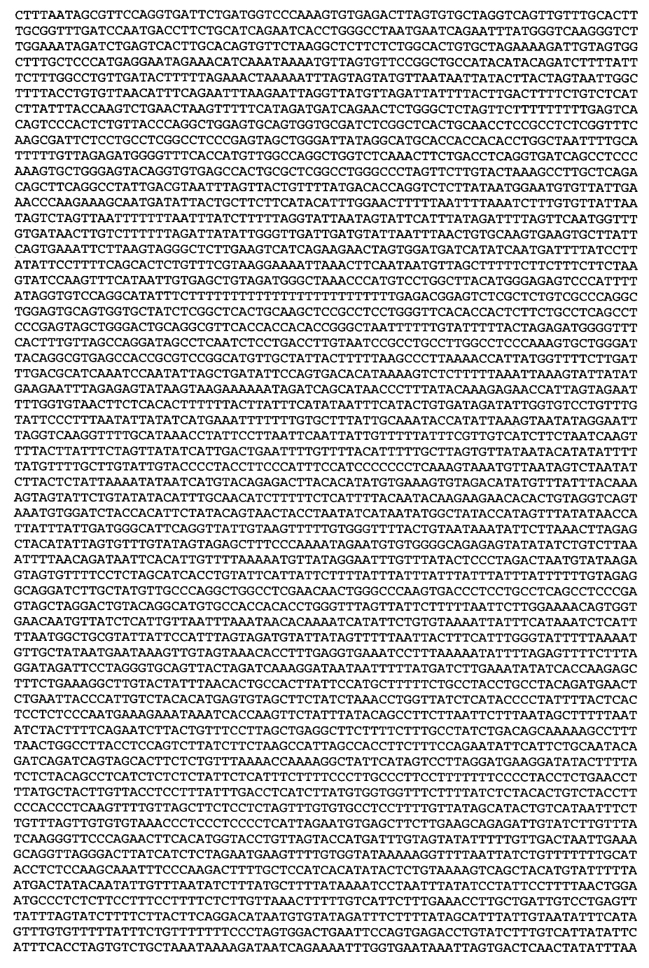

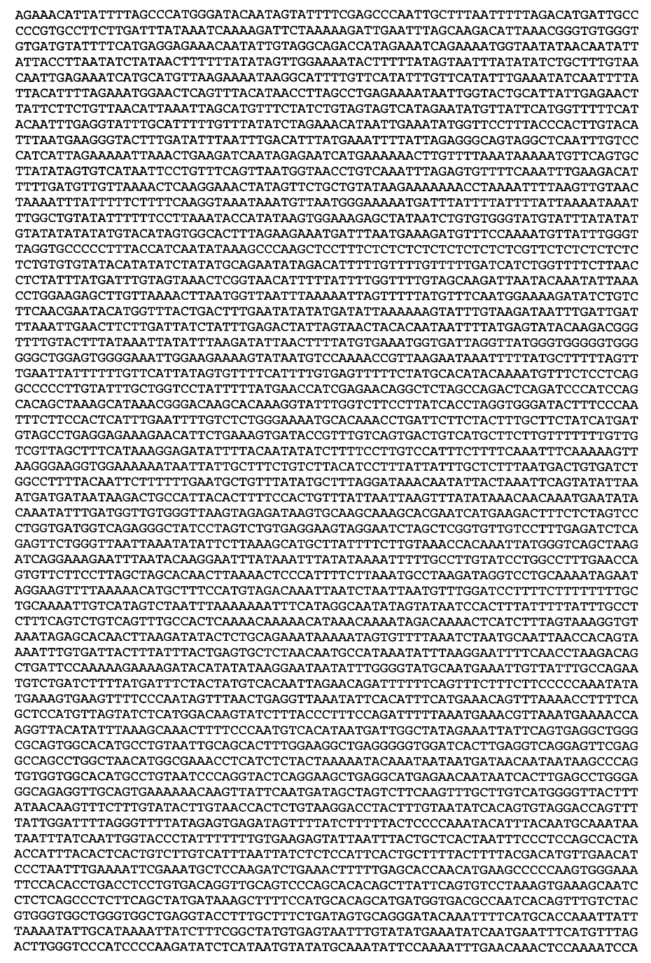

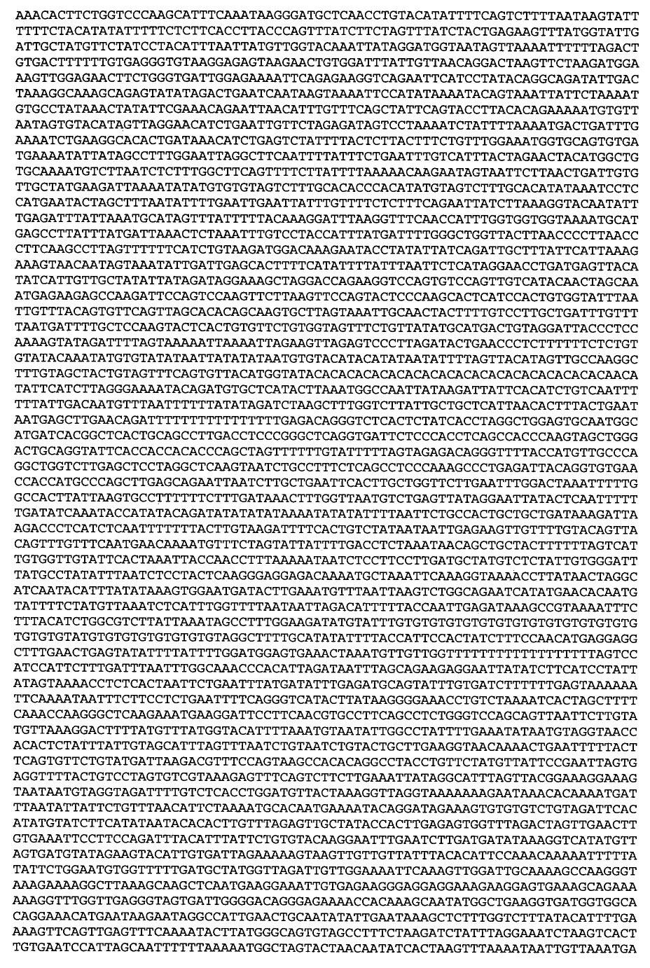

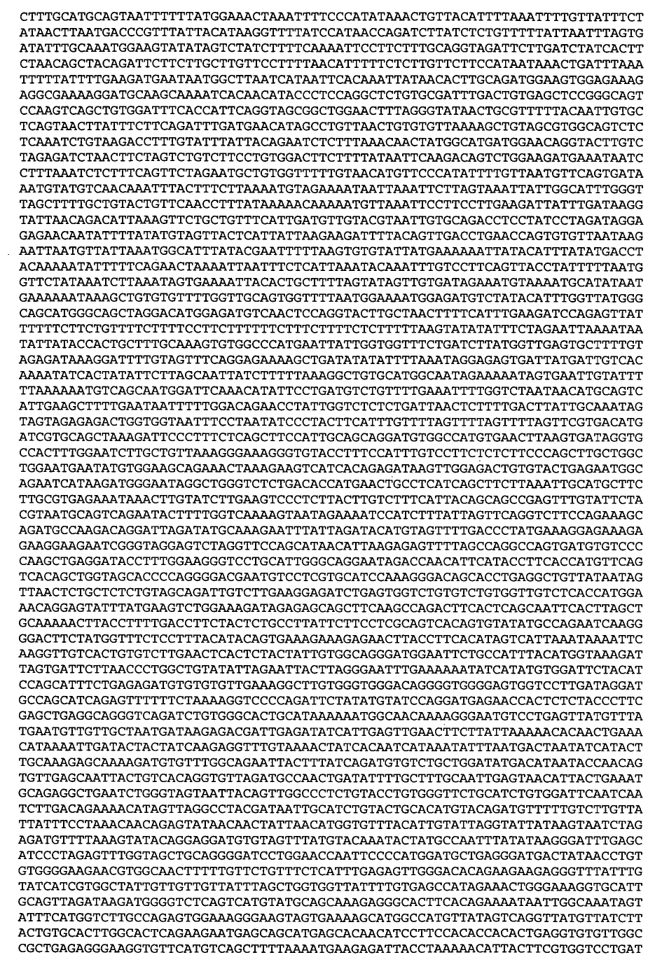

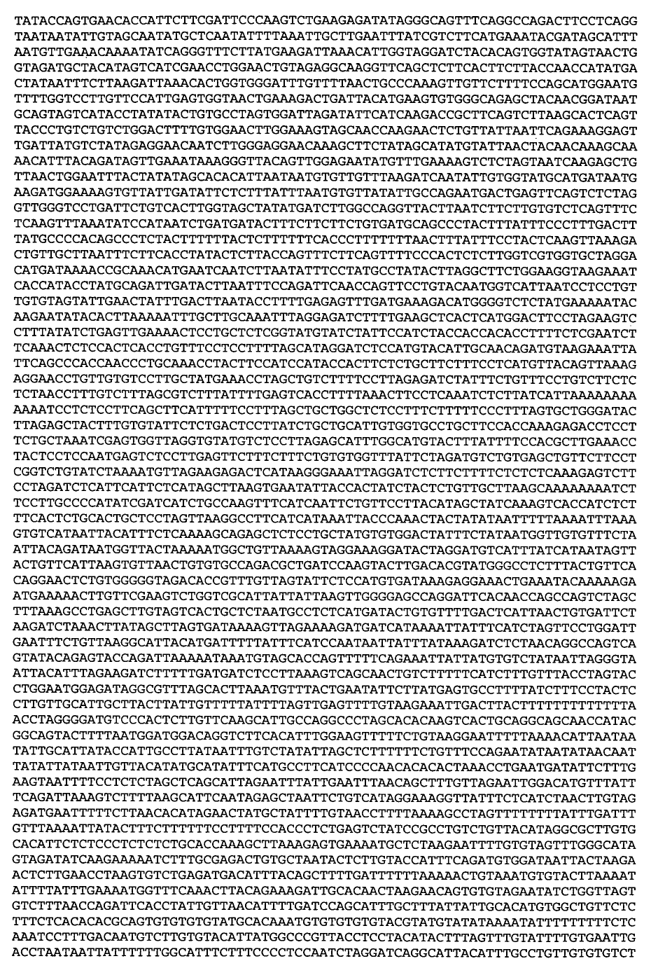

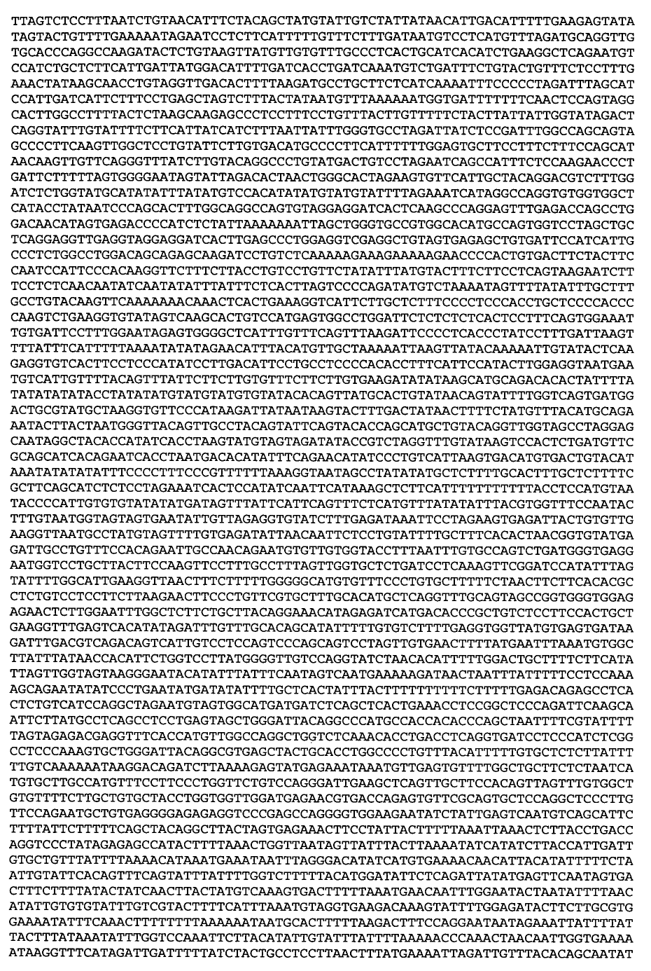

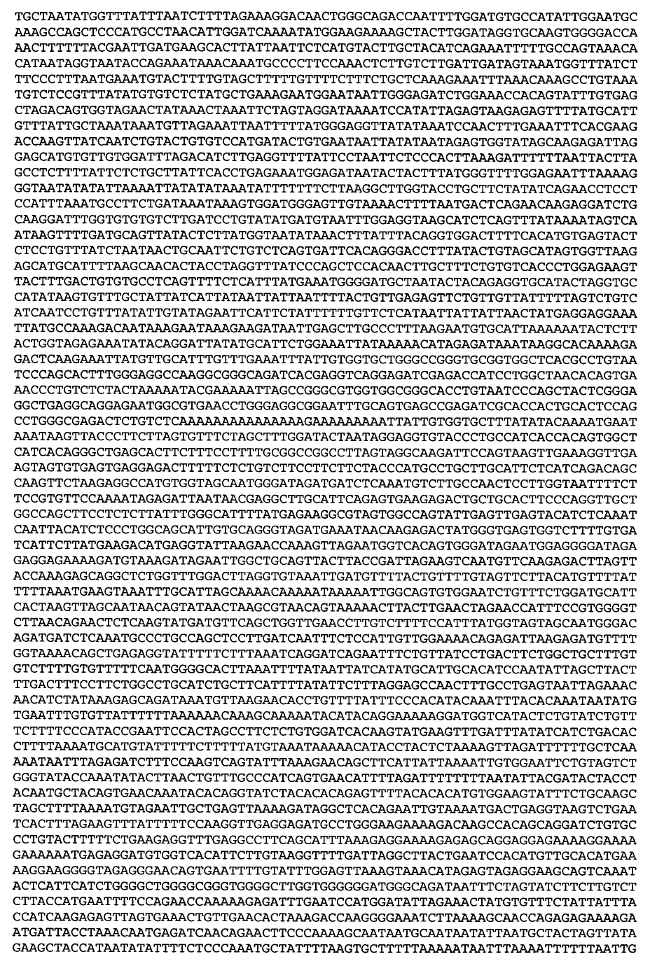

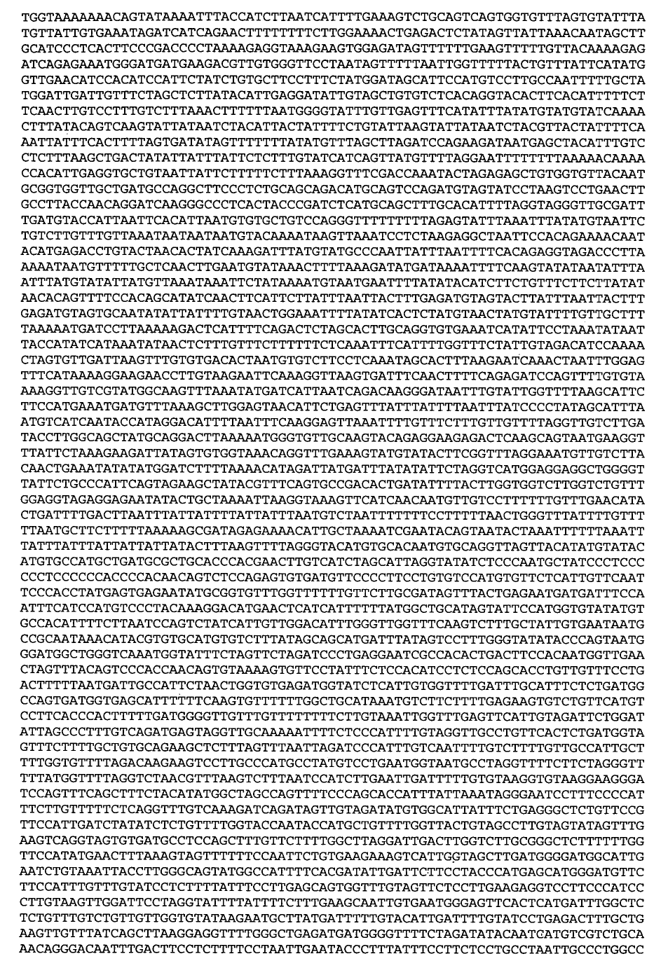

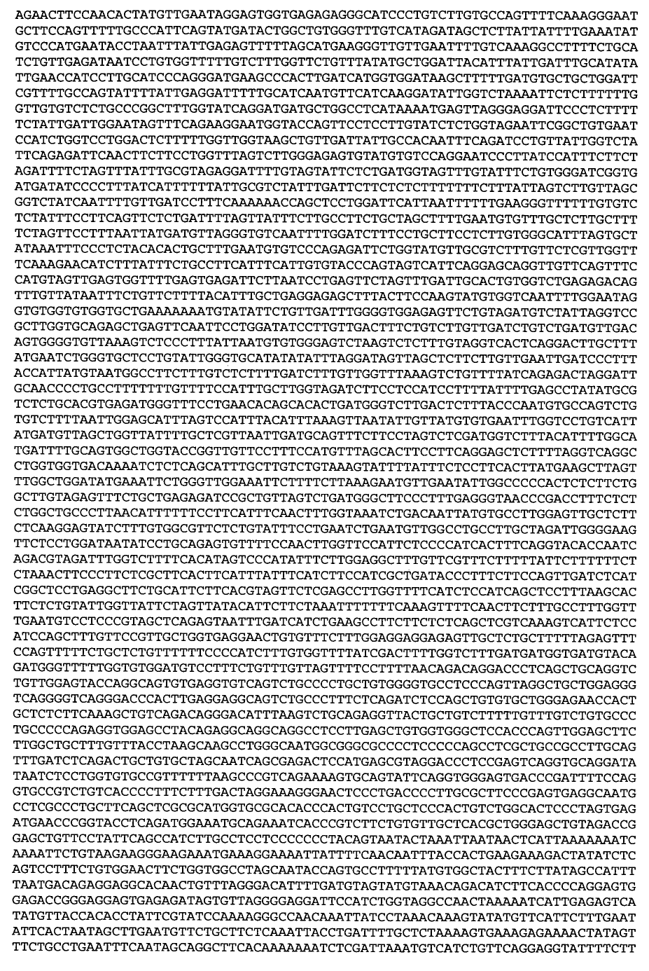

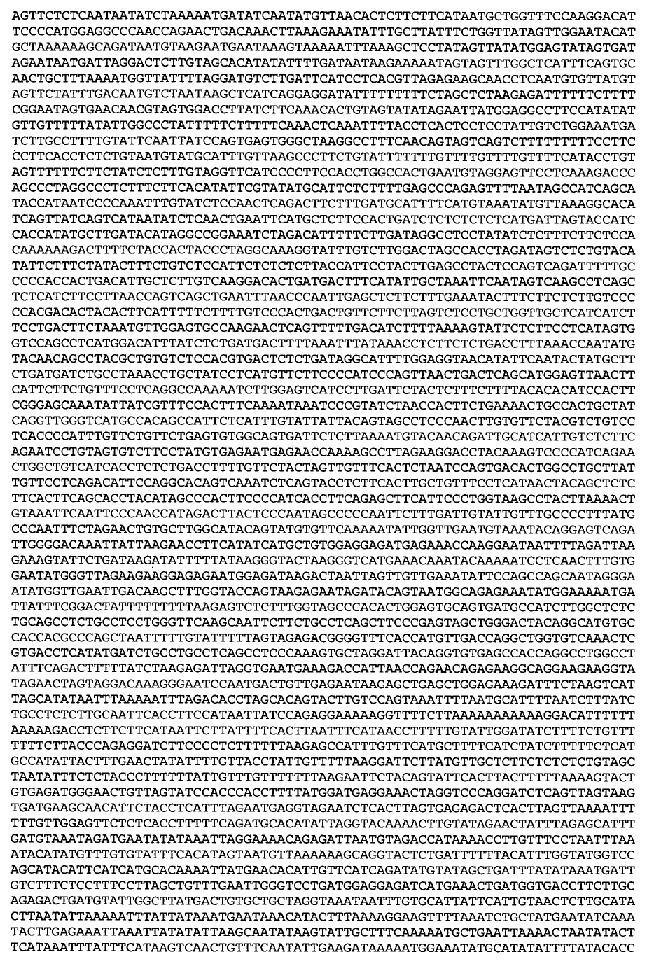

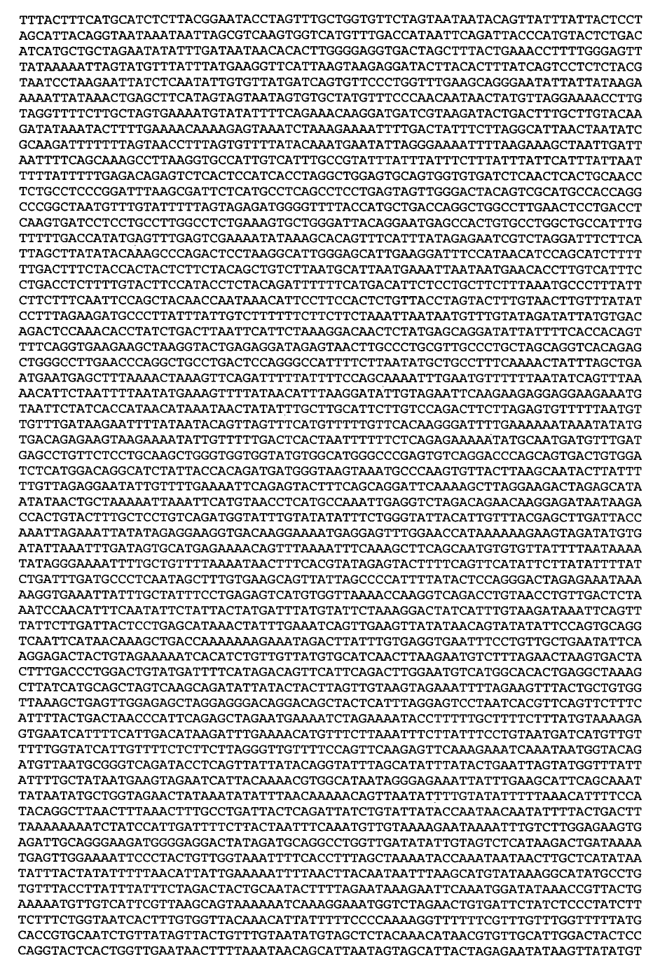

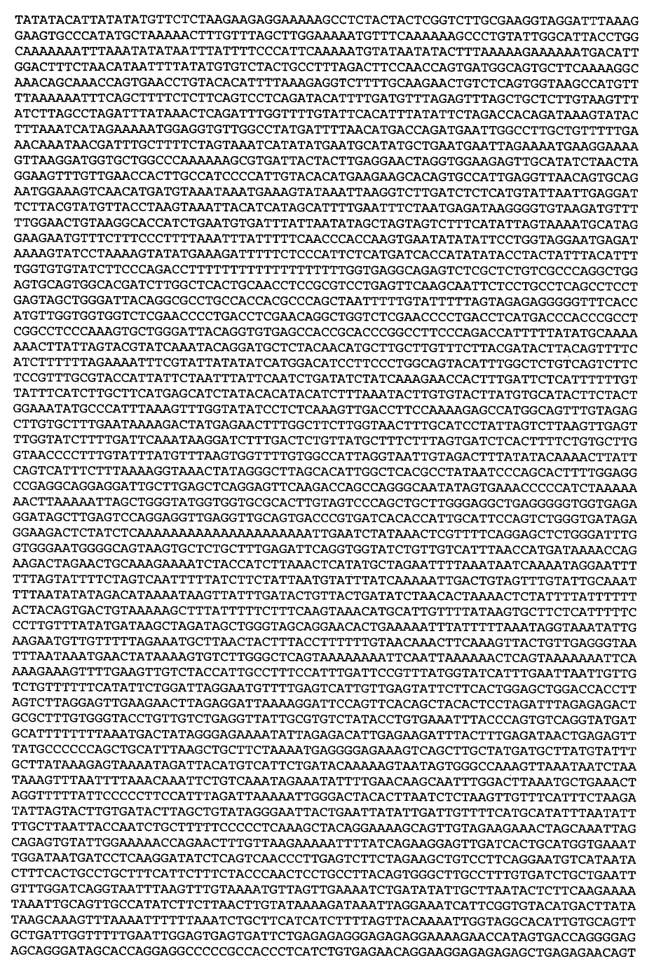

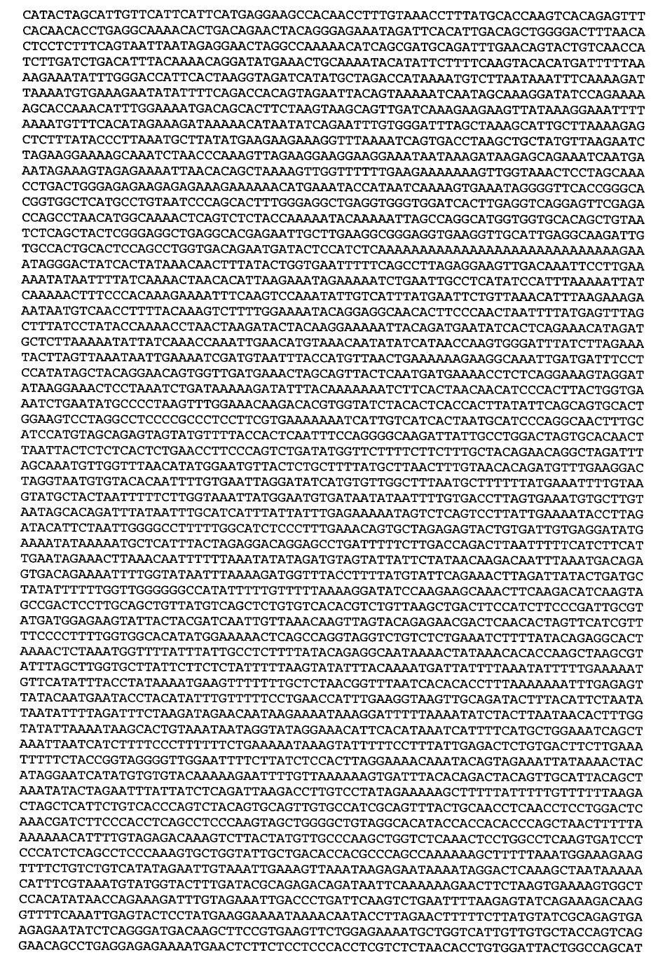

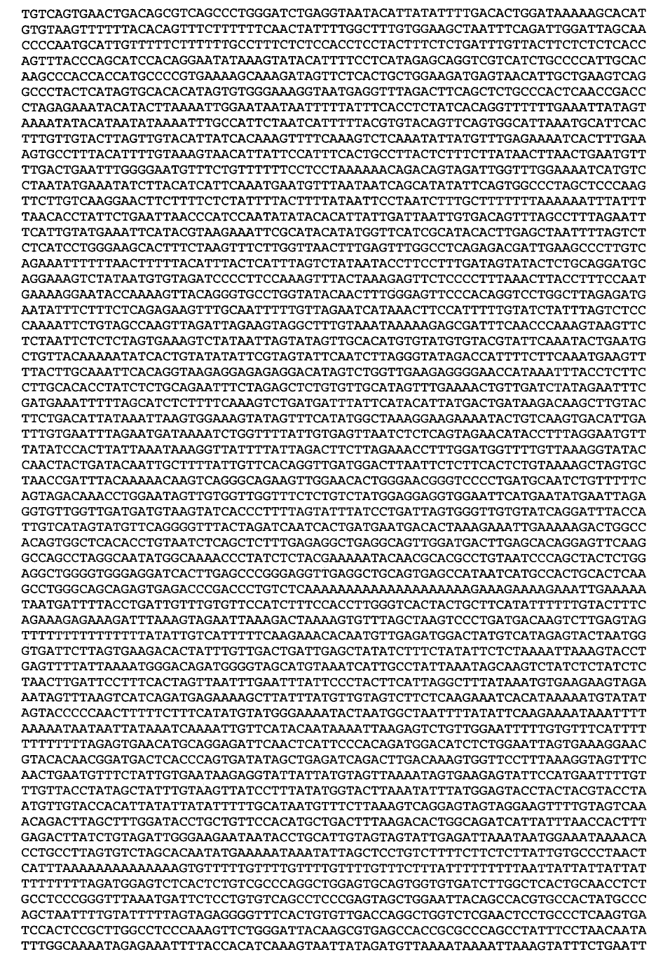

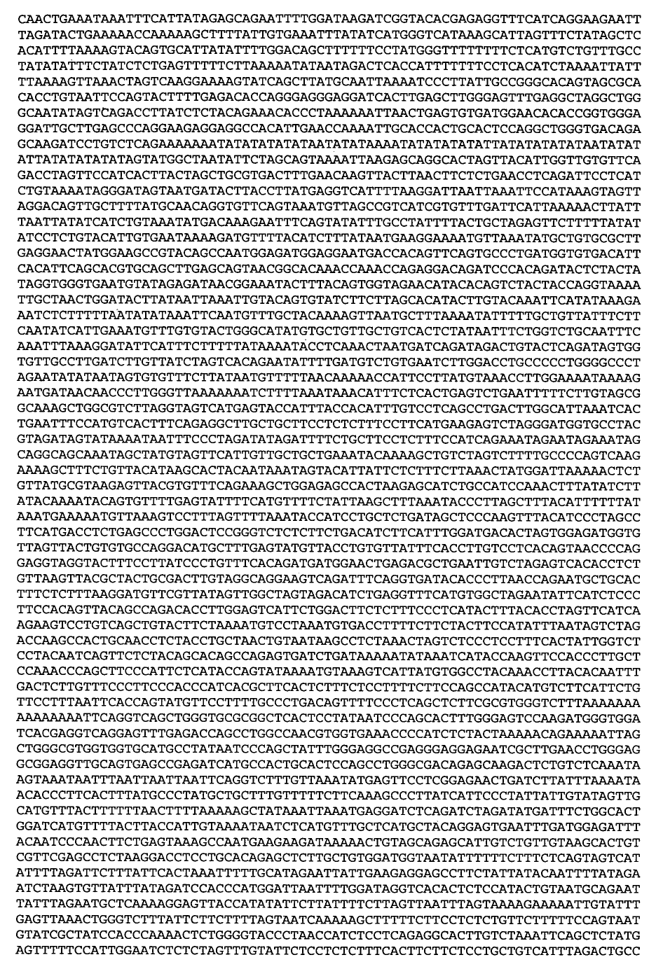

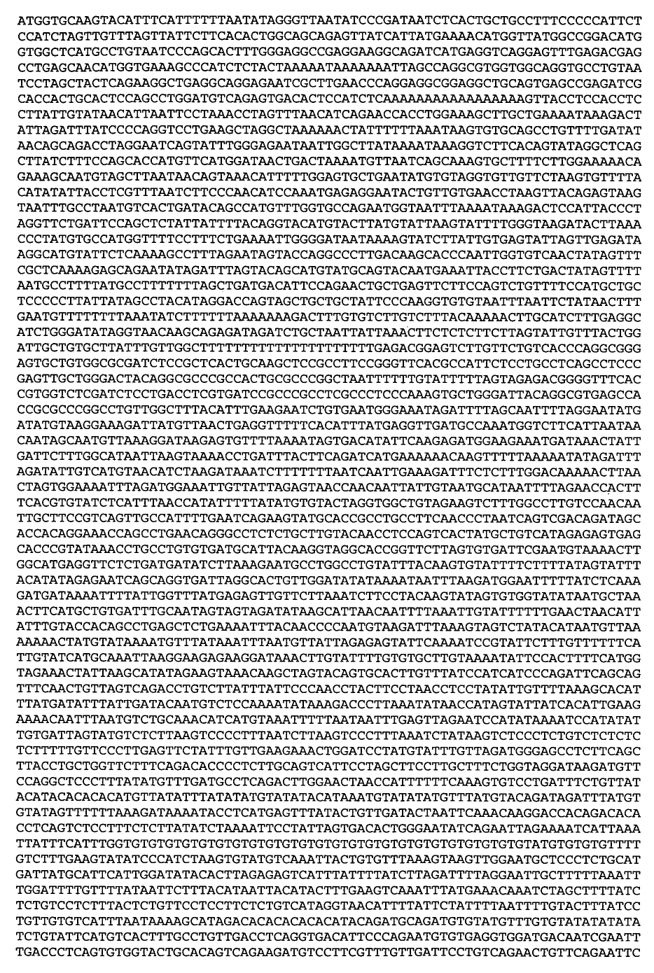

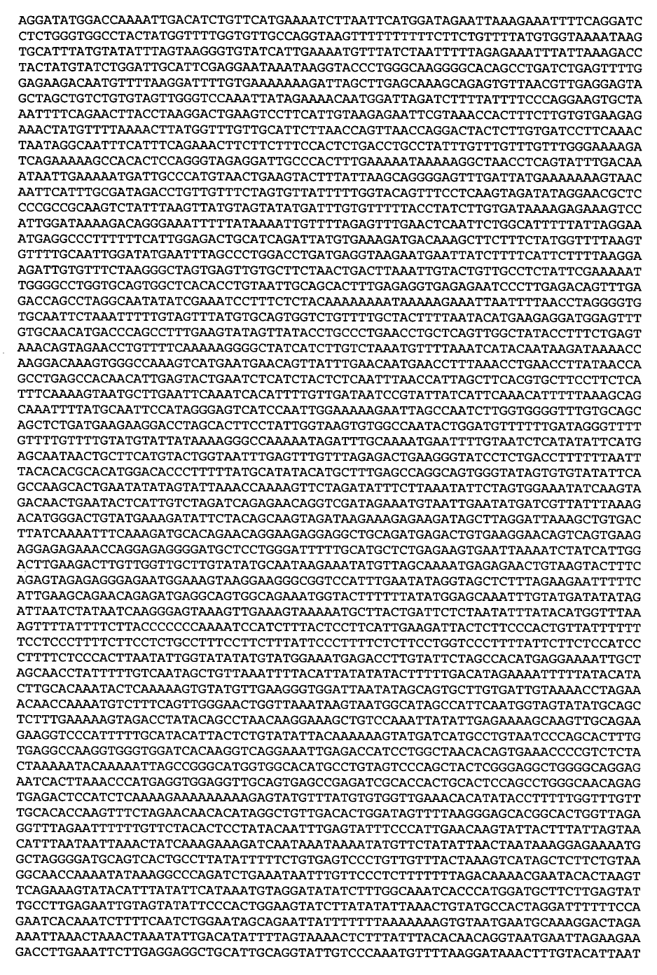

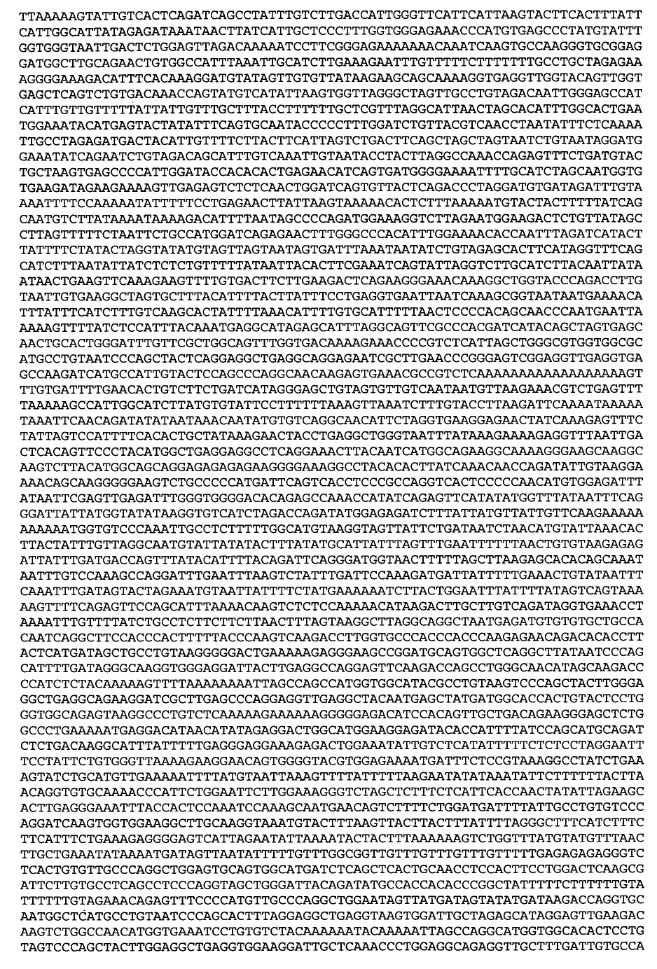

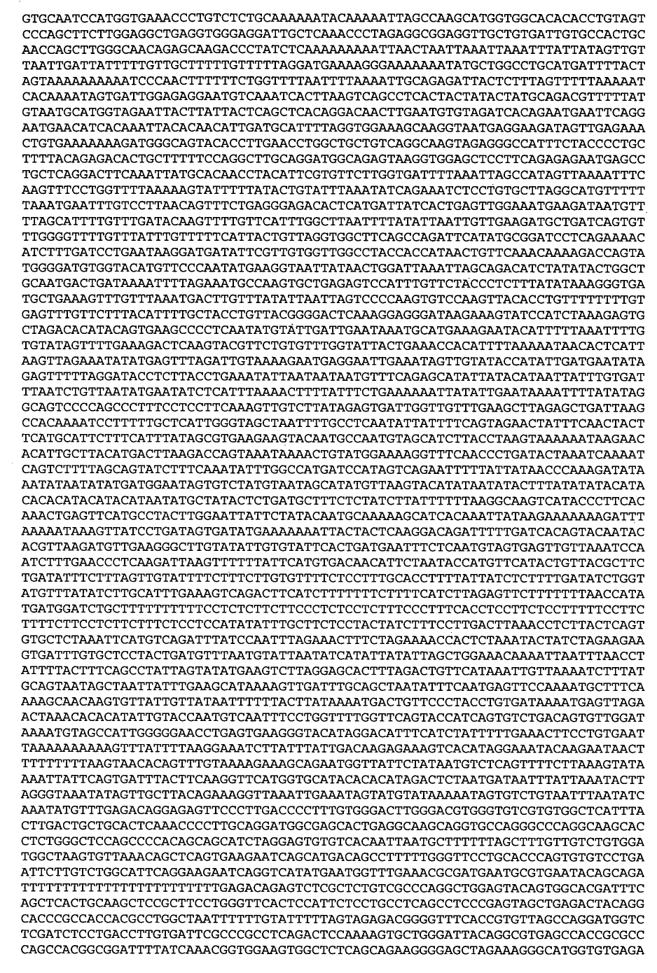

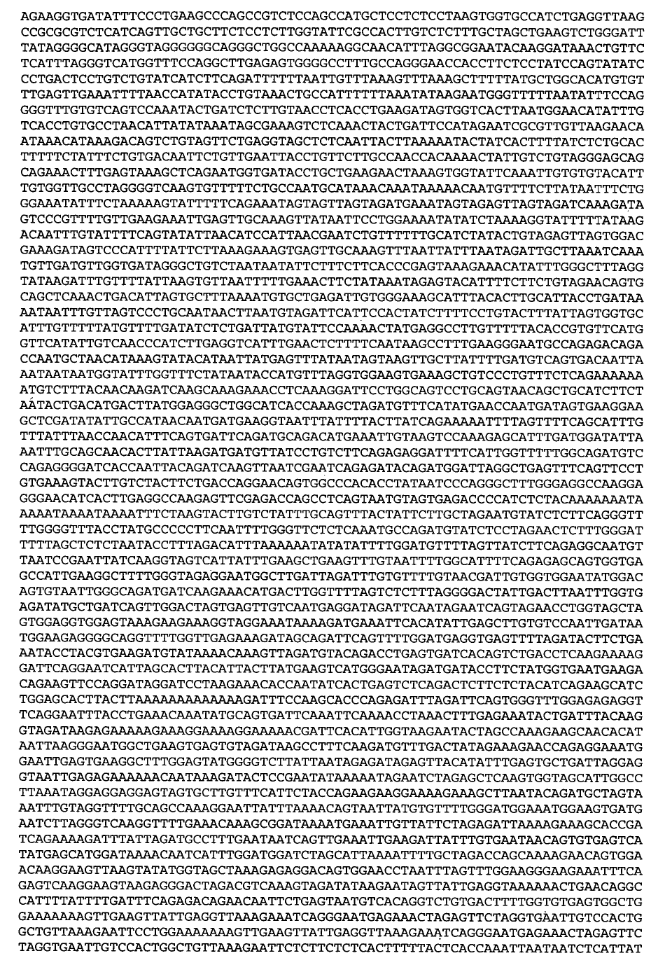

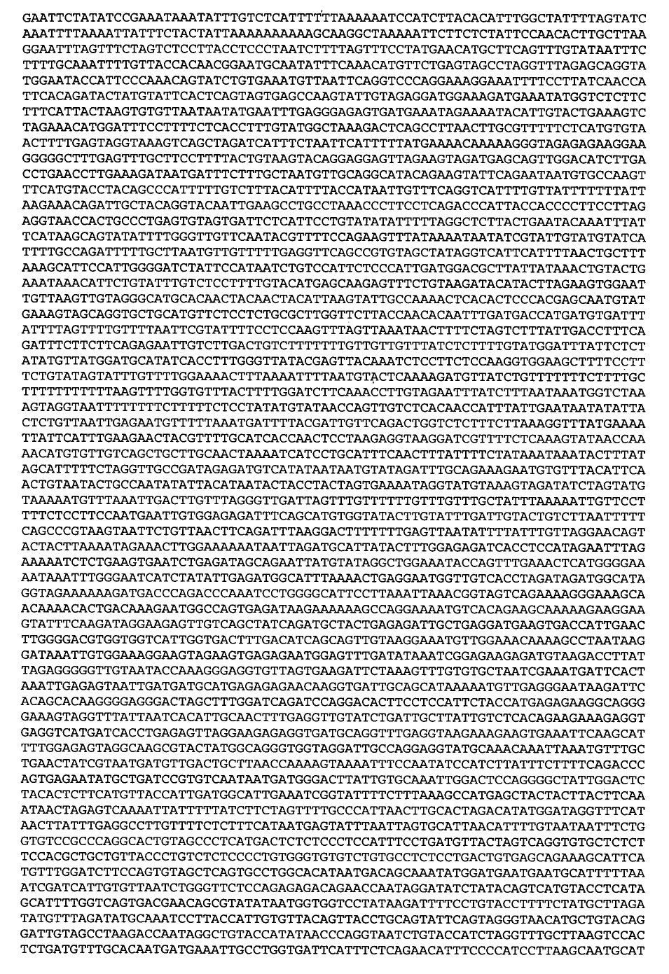

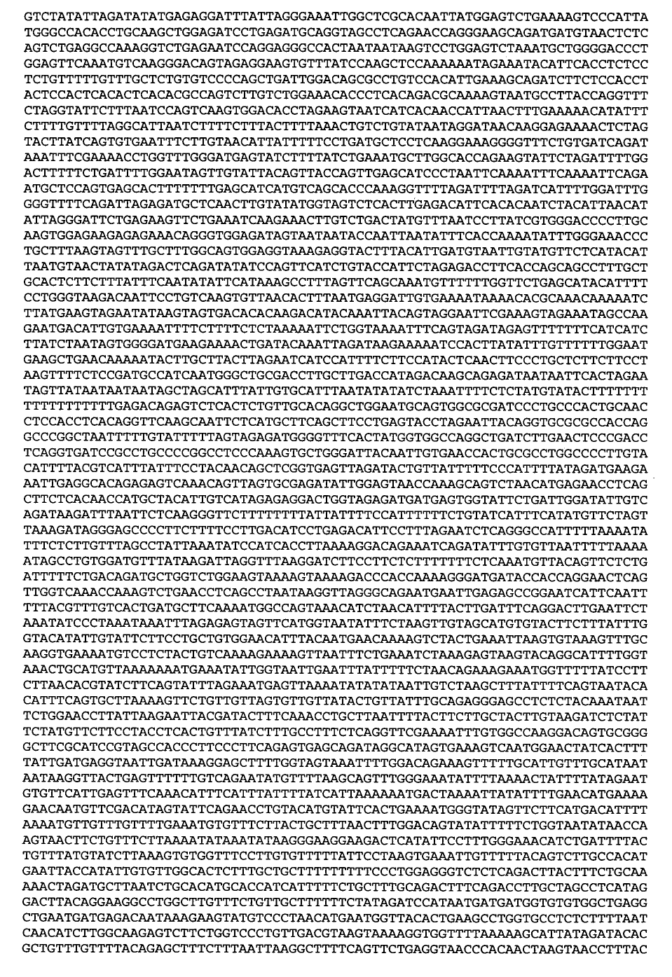

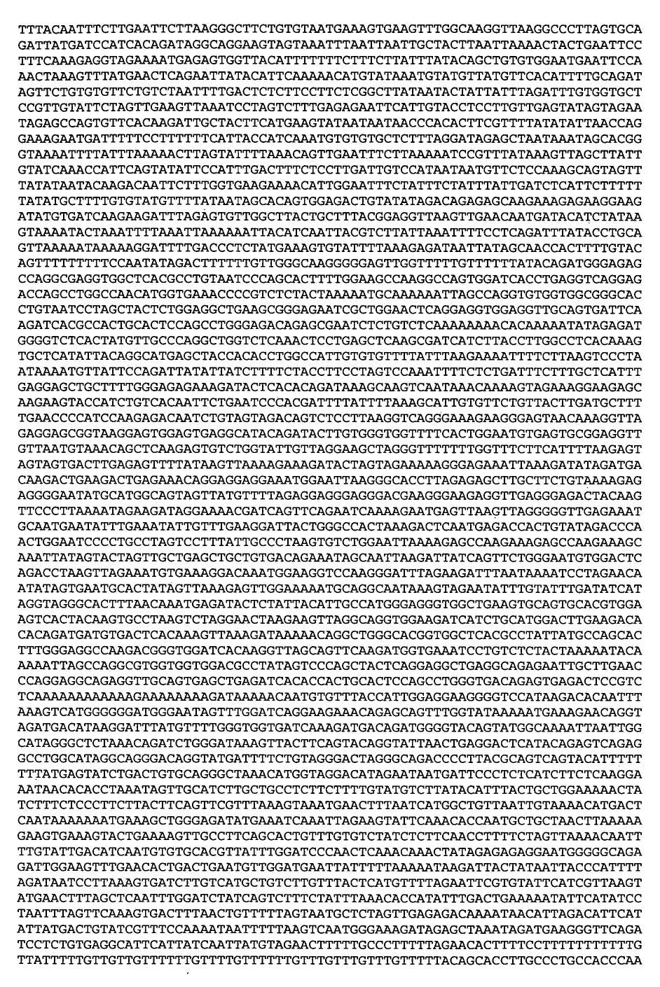

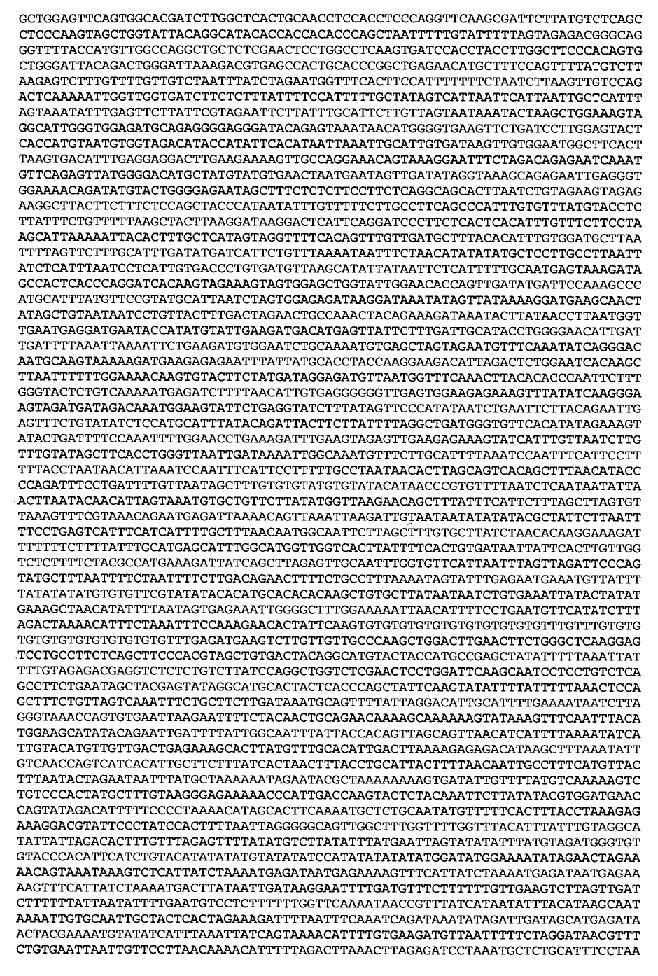

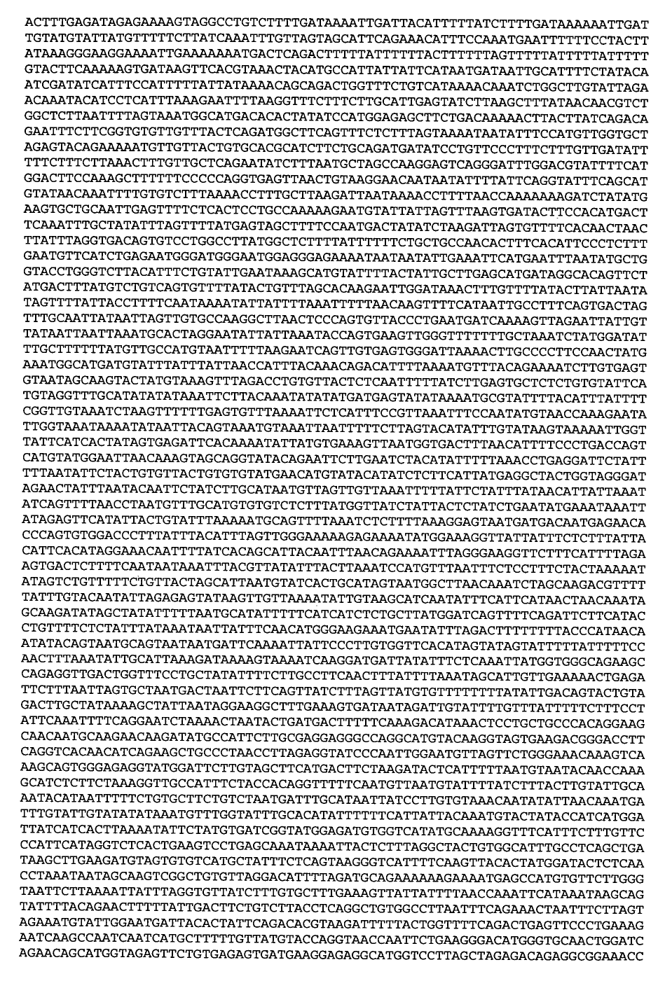

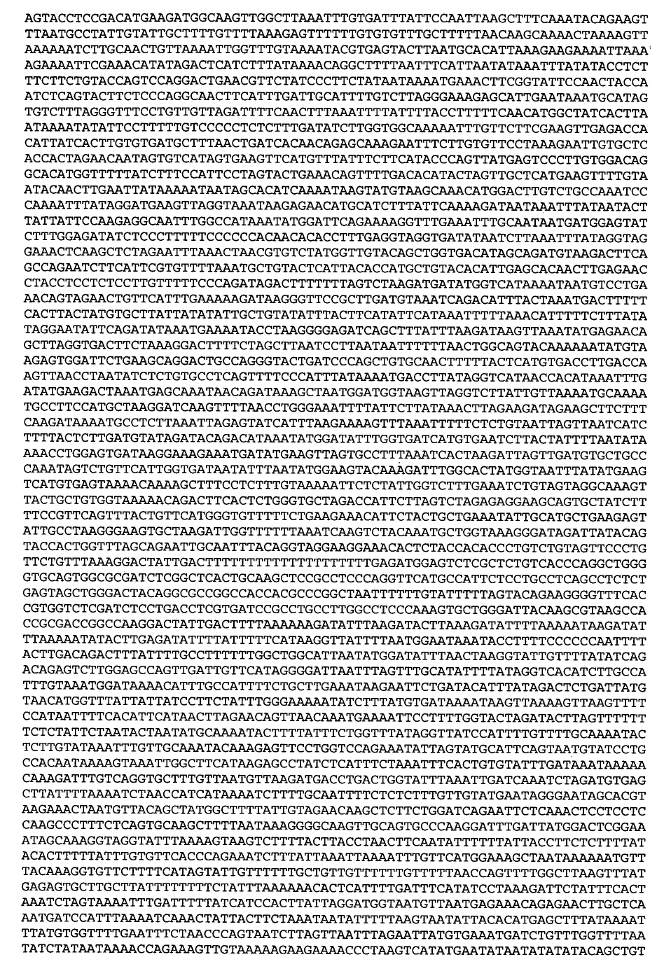

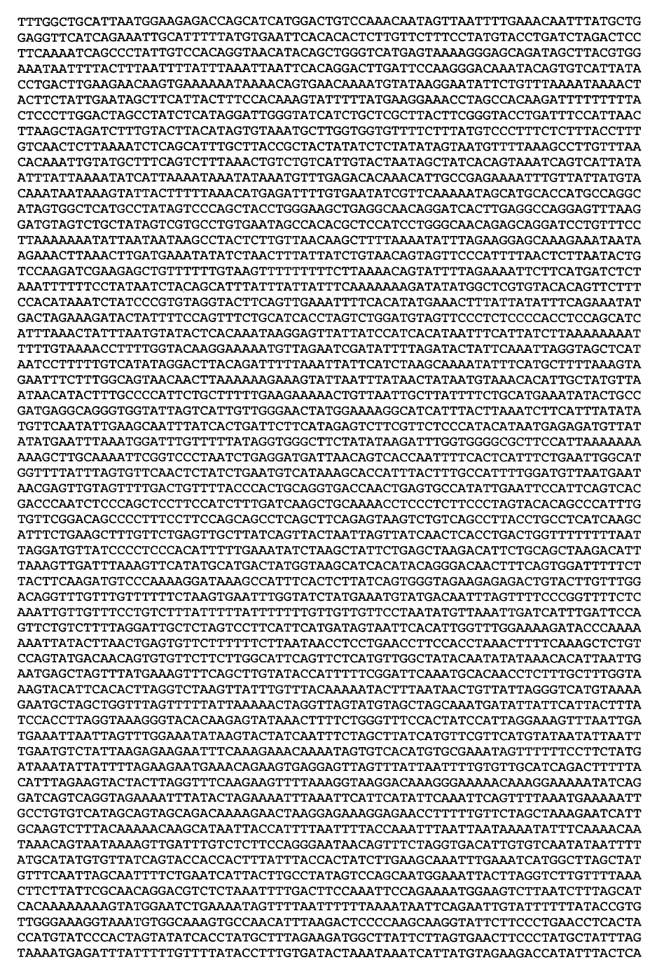

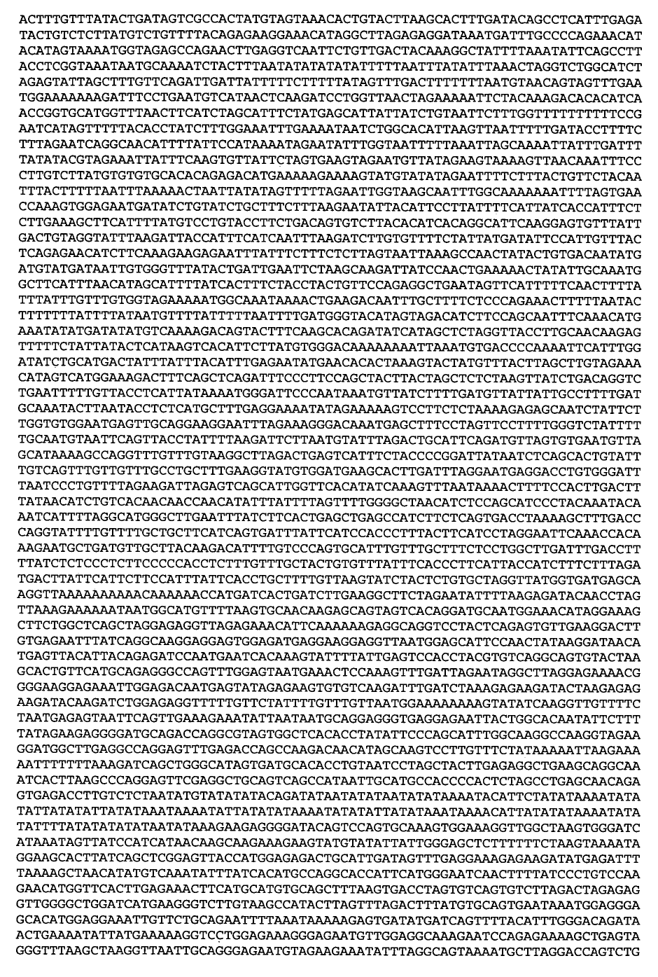

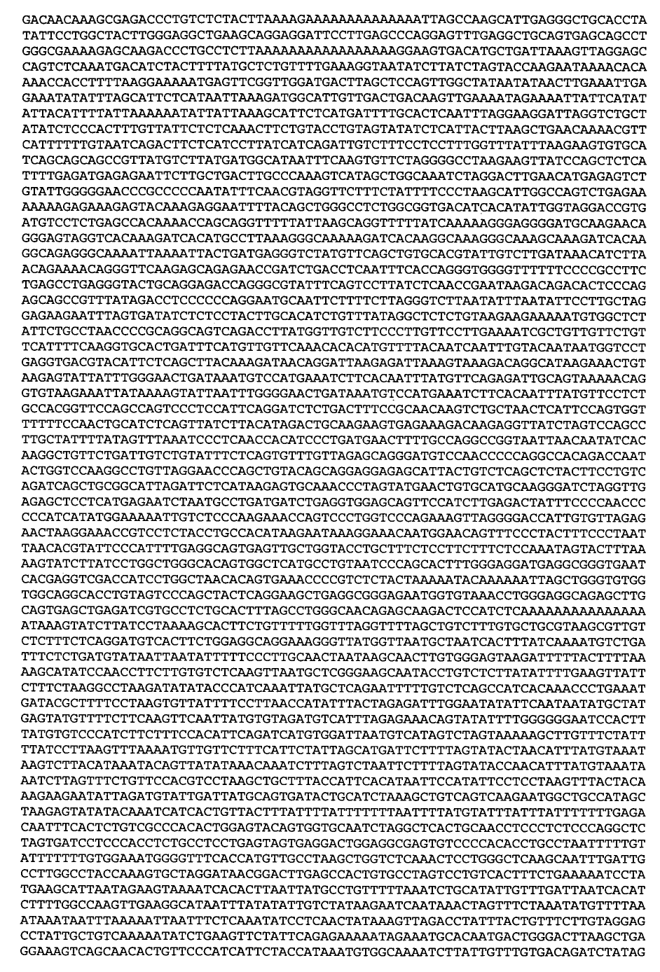

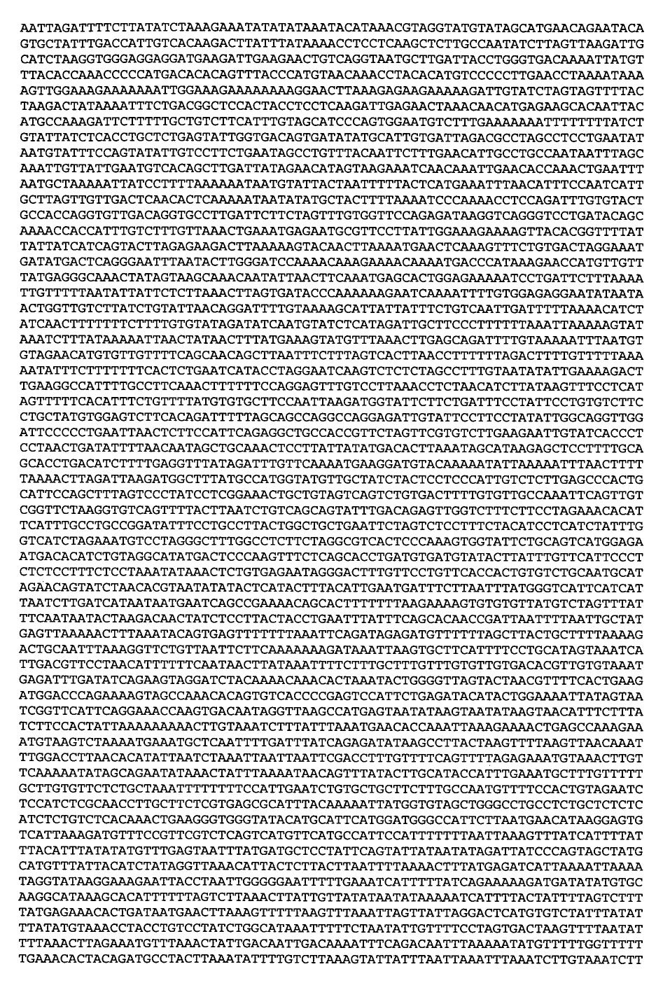

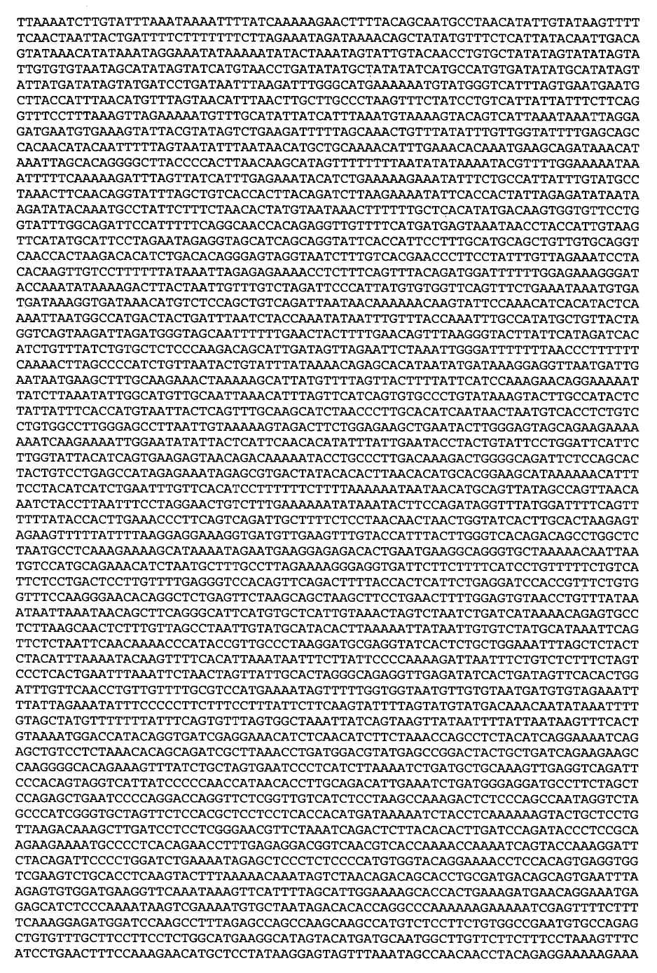

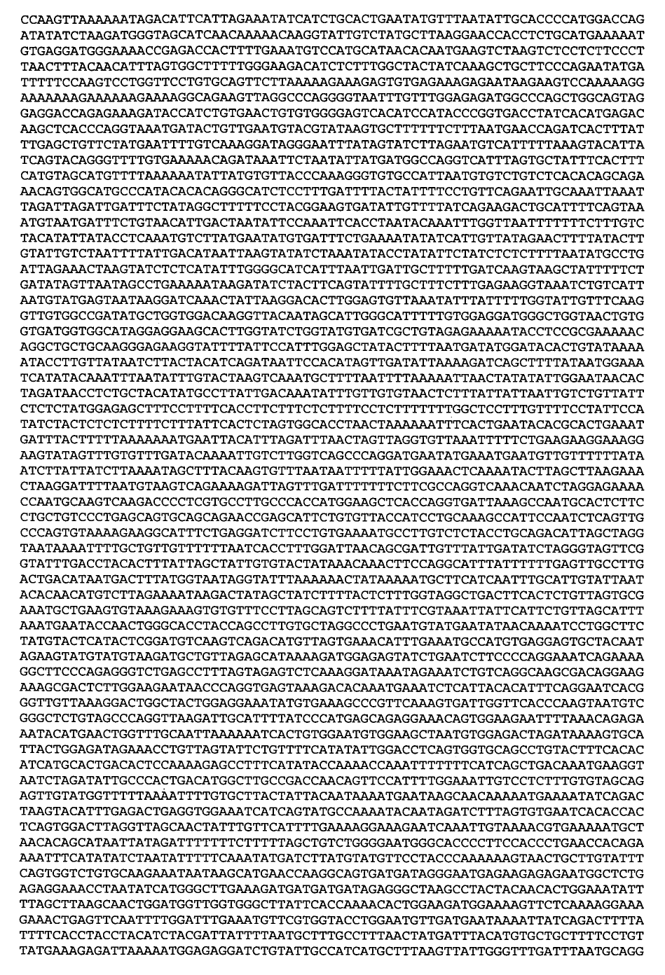

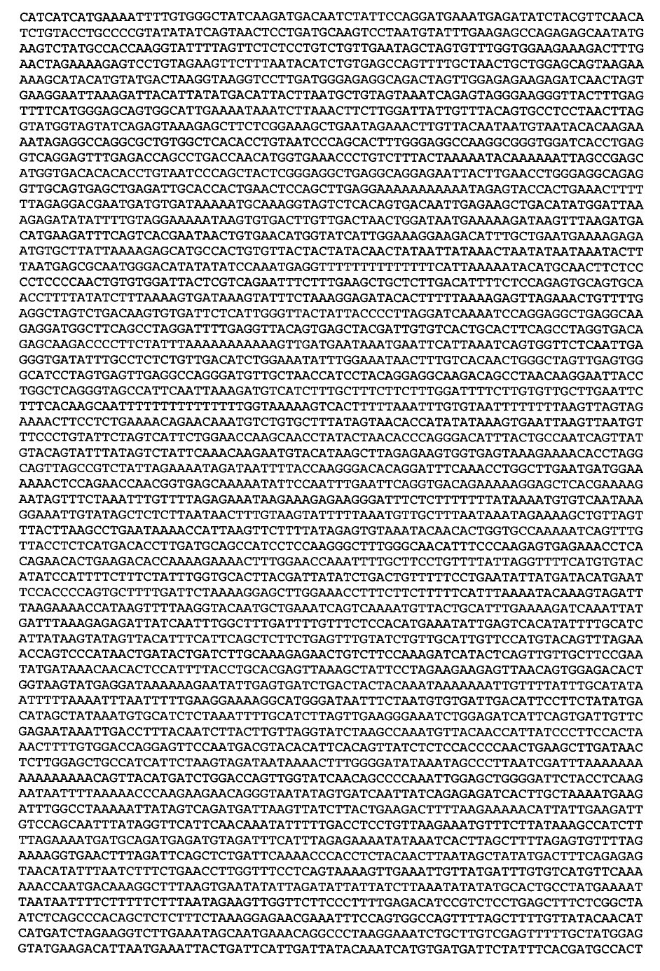

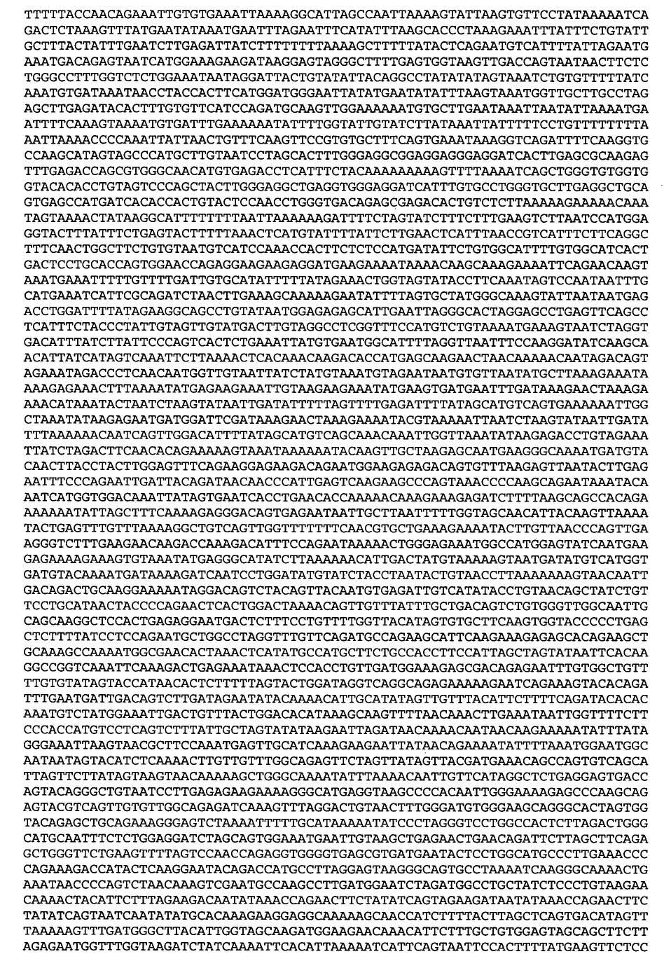

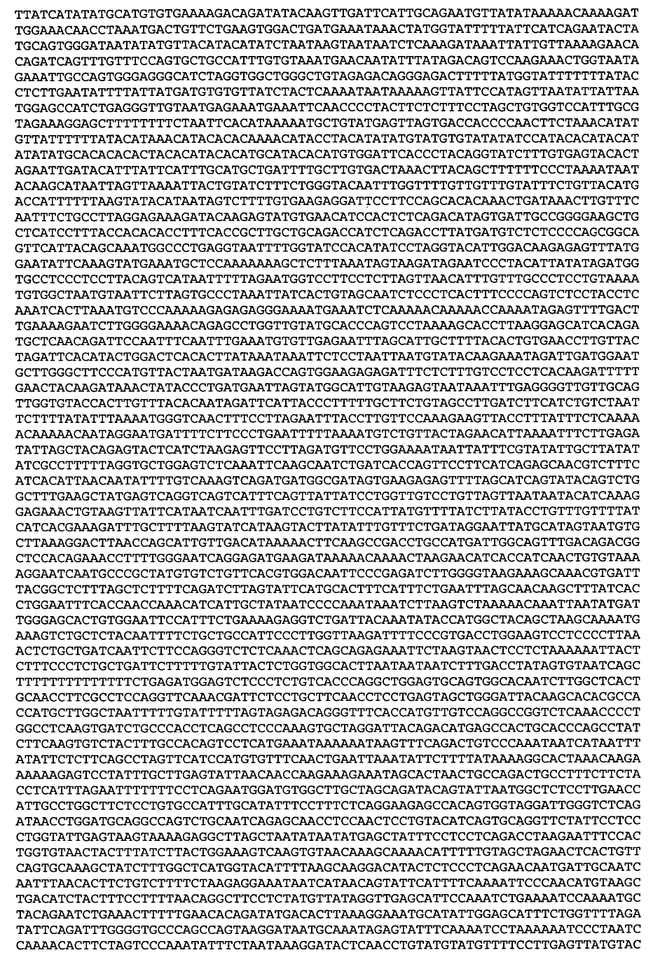

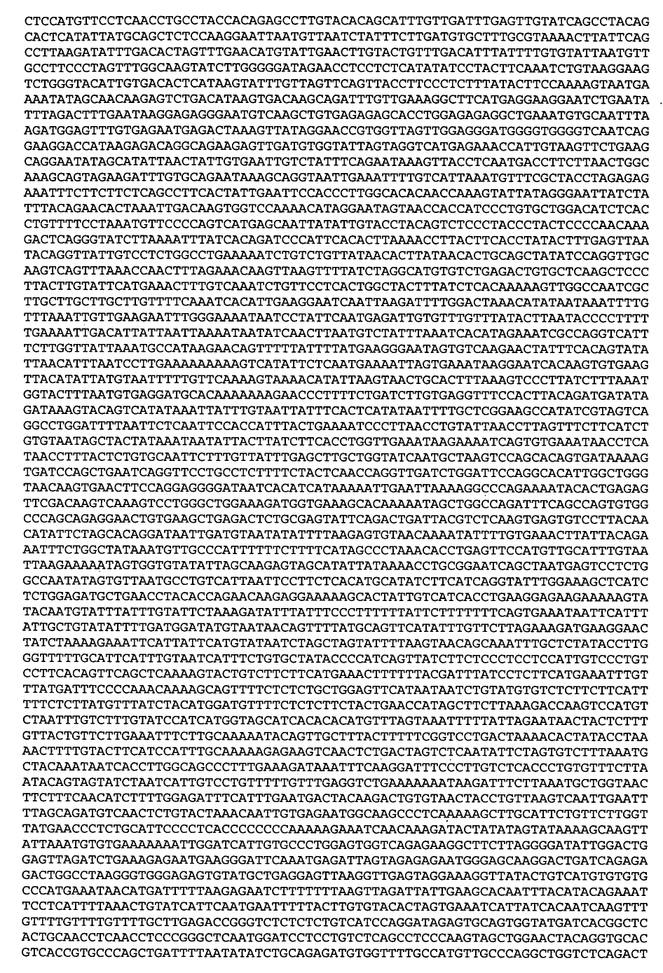

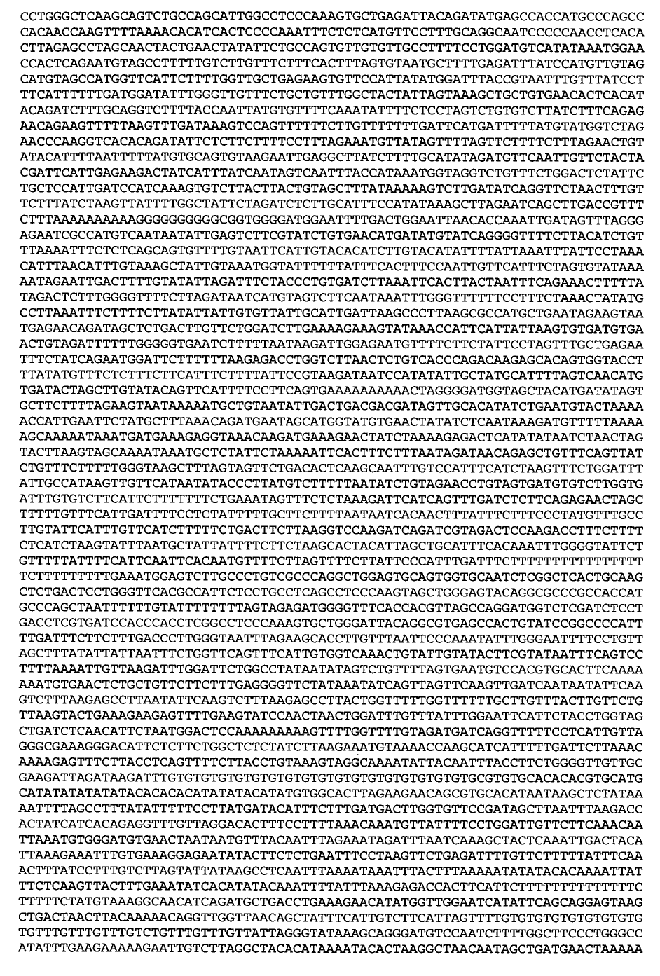

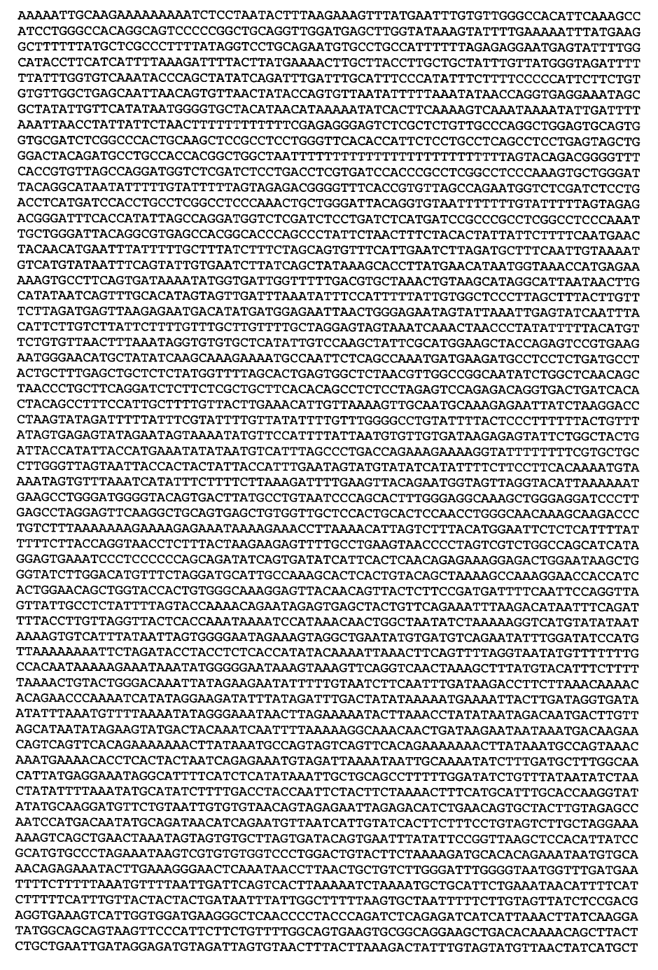

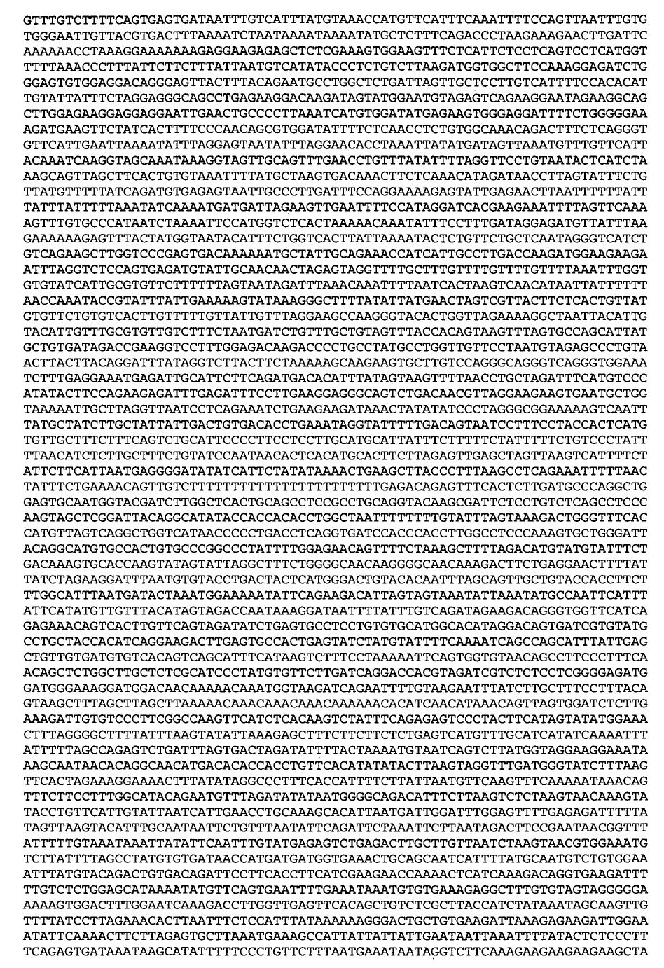

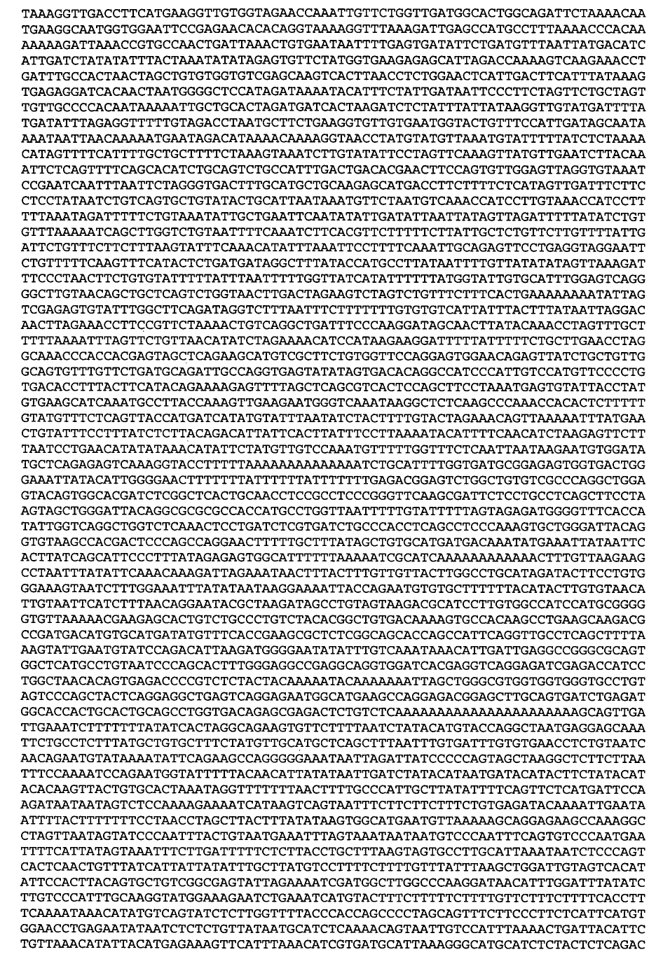

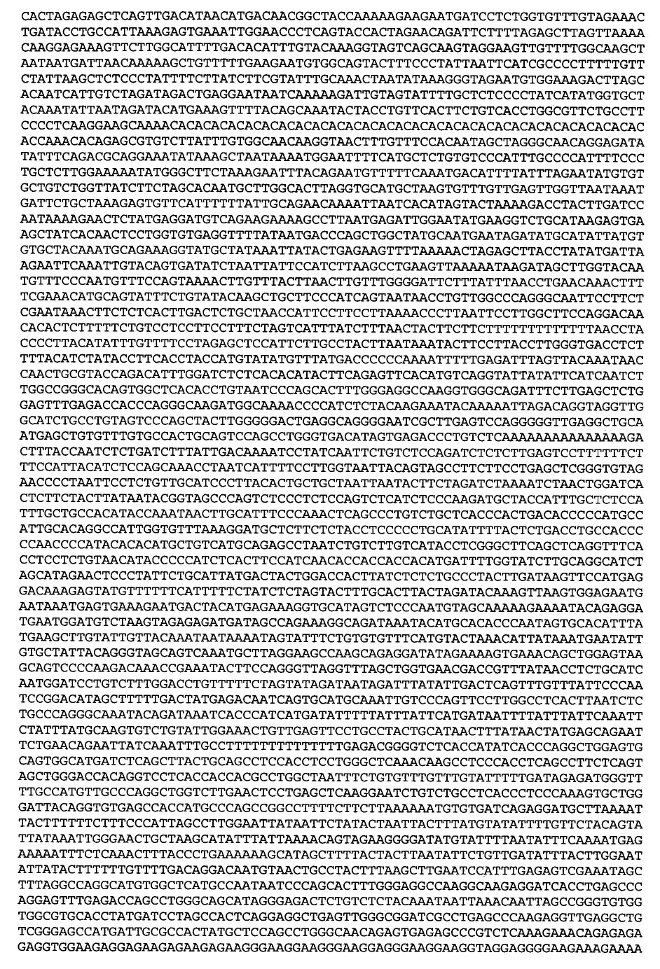

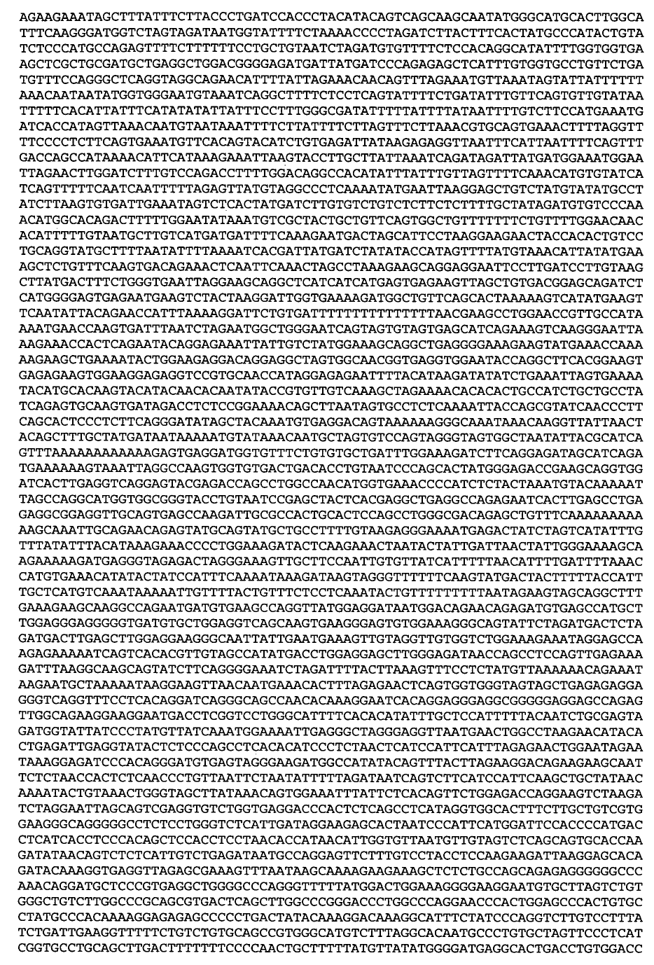

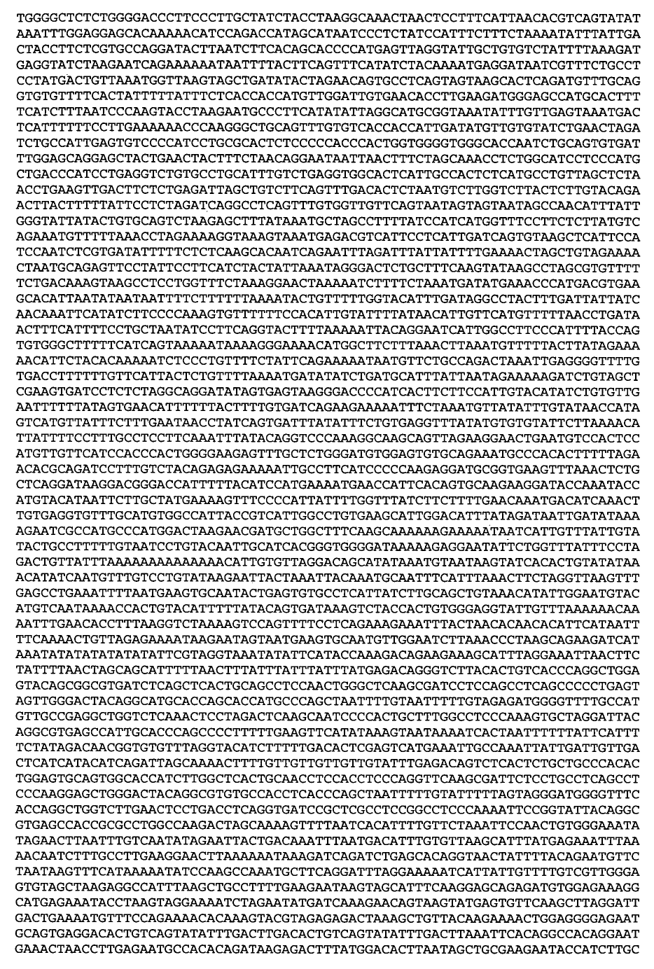

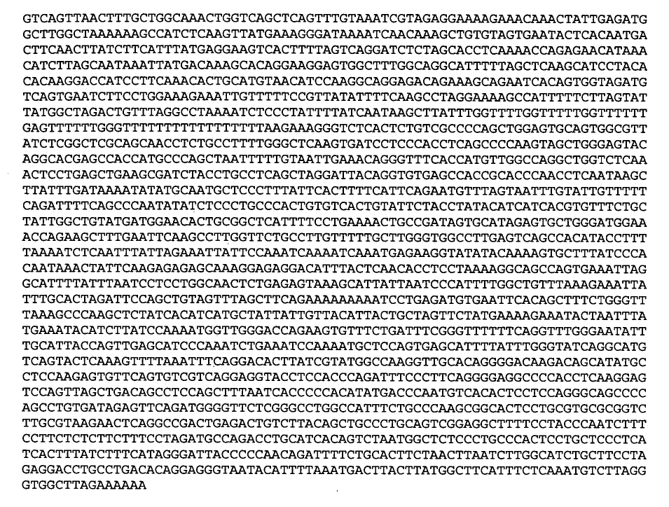

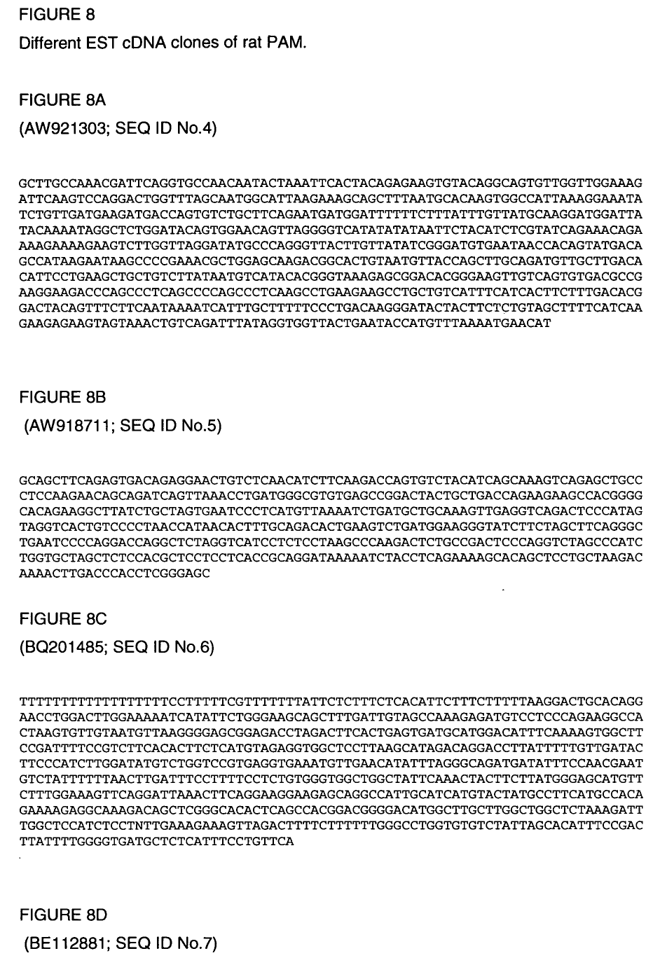

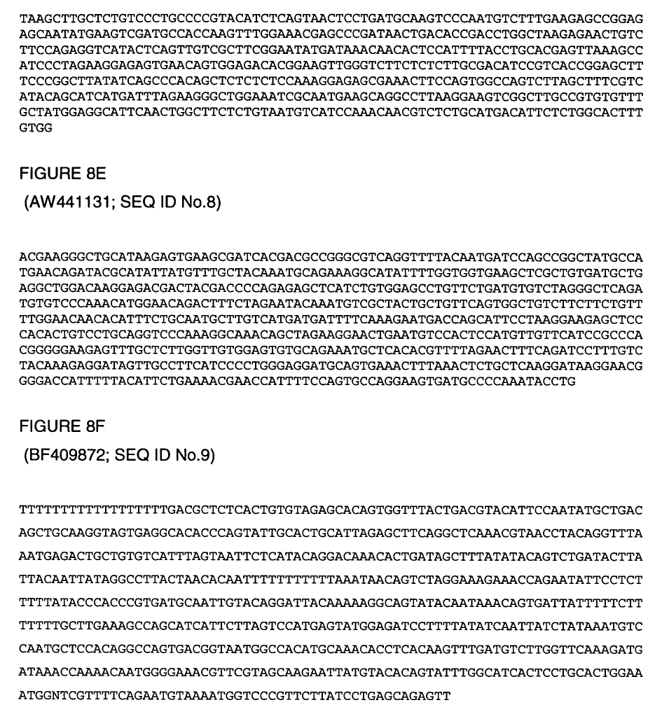

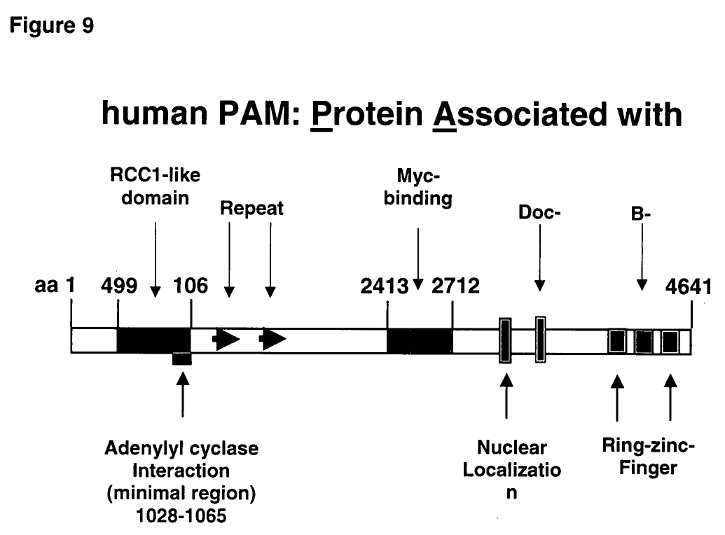

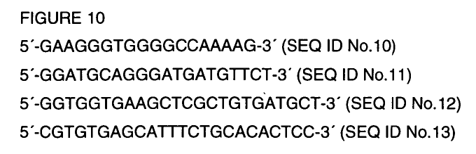

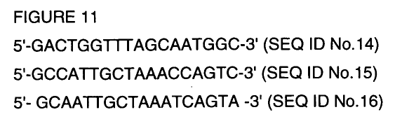

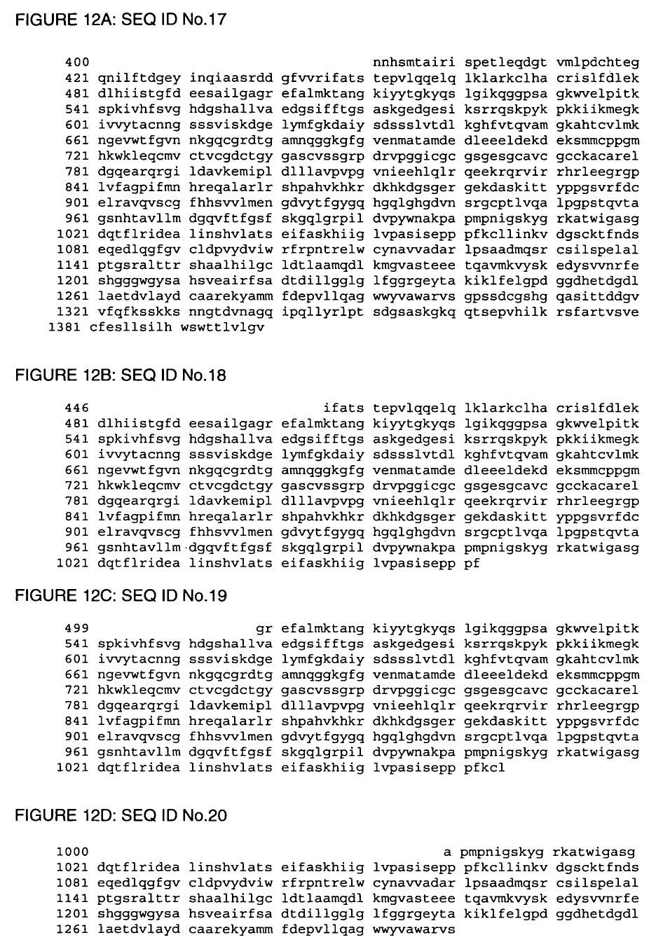

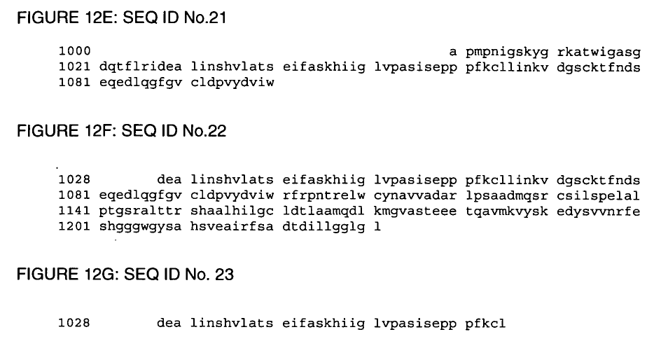

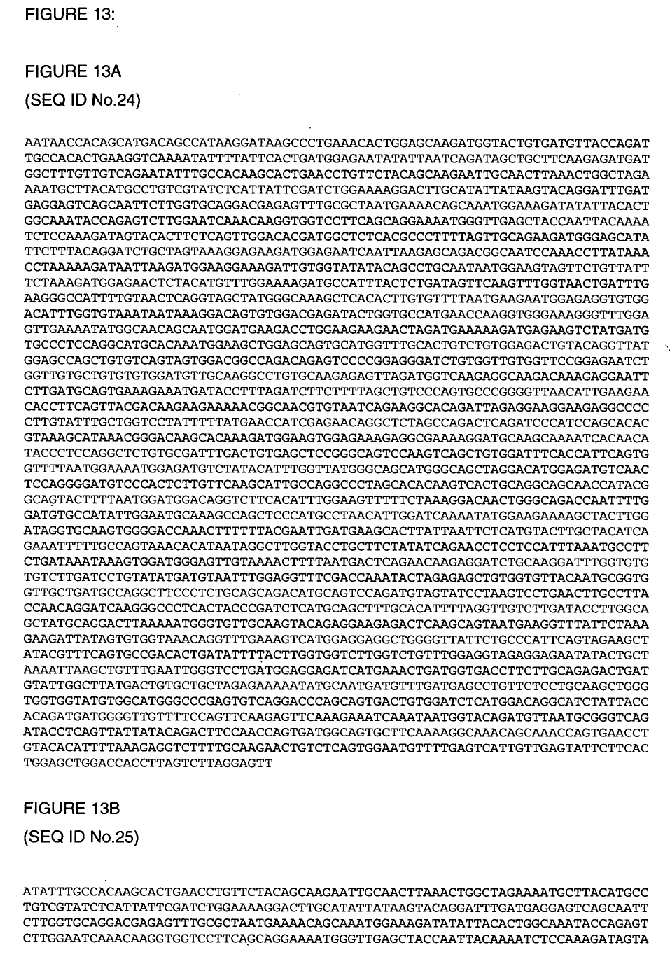

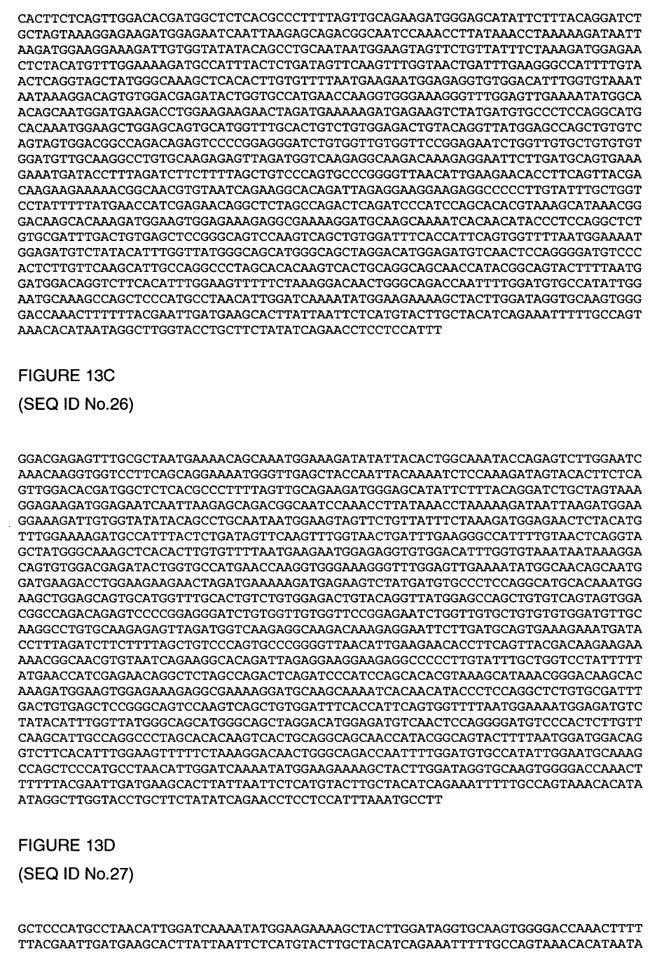

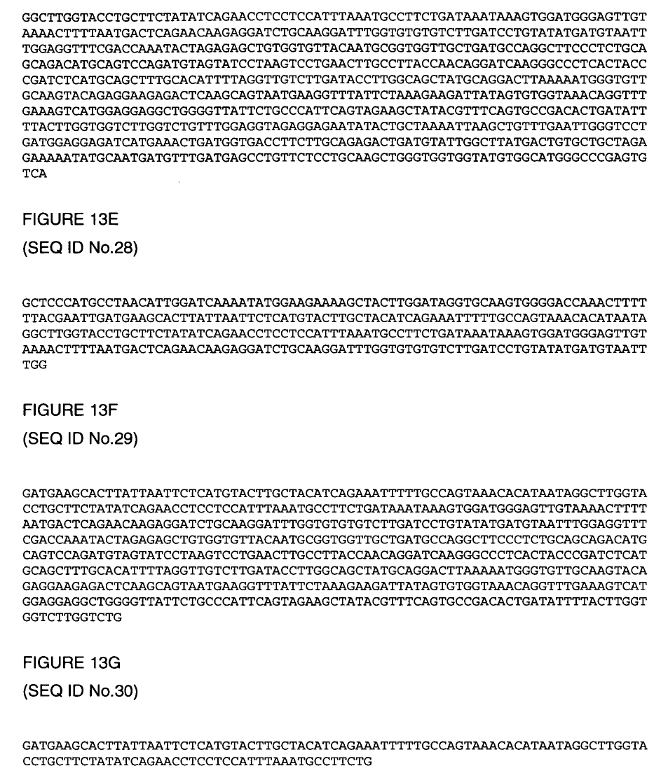

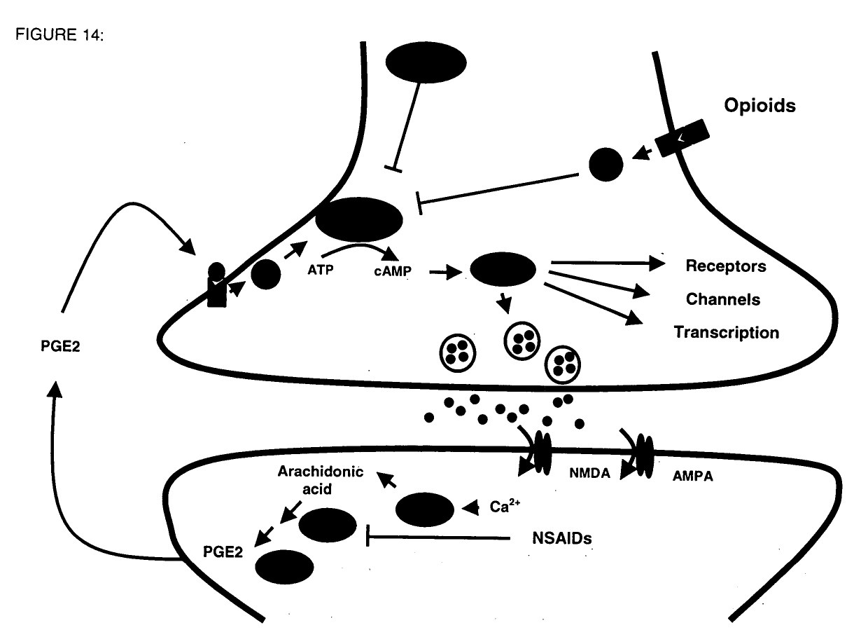

To examine a possible role of PAM in spinal nociceptive transmission, animals were infused with PAM sense and antisense oligonucleotides by lumbar intrathecal catheters before performing a formalin assay. PAM expression was decreased in spinal cord neurons as observed by immunhistochemistry (FIG. 6a). Infusion of PAM antisense oligonucleotides caused a significant increase of the nociceptive response following formalin injection as compared to PAM sense treatment (p=0.007; FIG. 6b and c). The hyperalgesia in PAM antisense-treated rats was accompanied by increased licking and biting behavior. Since PAM is an inhibitor of AC activity (Scholich et al. 2001), basal, Gαs- and forskolin-stimulated AC activities were determined in spinal cord lysates of sense and antisense-ODN treated rats. The experiments showed a significant increase (20.7%) of the basal AC activity in antisense treated rats (Table 1). In contrast, no significant changes were detected in Gαs- and forskolin-stimulated AC activities indicating that the total amount of AC was not altered by the ODN treatment (Table 1).

The above experiments showed that PAM is localized in the cell body and axons of both spinal cord and DRG neurons (FIG. 2a,b). Only very little immunreactivity was detectable in the nucleus suggesting different functions for PAM in neurons and cancer cell lines.

Central sensitization after prolonged nociceptive stimuli is based on neuronal and synaptic changes in the spinal cord (Woolf and Costigan 1999; Woolf and Salter 2000; Ji and Woolf 2001). The finding of the inventors that Pam is expressed in sensory neurons of the spinal cord and DRGs led to the newly formed hypothesis, that PAM could be implicated in synaptic changes during spinal nociceptive processing. The above findings that PAM was up-regulated after nociceptive stimuli (FIG. 4a) supported this hypothesis that PAM may play a role in synaptic changes during spinal nociceptive processing.

Furthermore, the surprising finding of the inventors, that PAM is expressed in sensory nervous of the spinal cord and DRGs led to the question whether PAM is capable of inhibiting AC activity in spinal cord and DRG, as well.

The above experiments showed for the first time that PAM is a potent inhibitor of Gαs-stimulated AC activity in spinal cord preparations (FIG. 5b). AC activity was decreased by 50% after addition of 30 nM PAM. To achieve comparable inhibition using the α-subunit of the inhibitory G-protein, Gαi, 200-800 nM Gαi has to be used (Wittpoth et al. 1999). The inhibitory action of PAM was even stronger in spinal cord preparations of animals treated for 96 hours with zymosan (FIG. 5b) and could be explained by the elevated amounts of endogenous PAM in the spinal cord after zymosan injection (FIG. 4a,b). Inhibition of Gαs-stimulated AC activity in spinal cord preparations from formalin-treated animals (25% inhibition) was less pronounced as compared to control or zymosan-treated animals (50% and 75%, respectively).

Notably, in animals treated with formalin for 1 hour a shift in AC isoform expression was observed (FIG. 5a). AC of type 3 and 9 are up-regulated while AC type 5 is down-regulated. To date it is not known if PAM is an inhibitor of AC type 3 and 9. Therefore, these isoforms may not be inhibited by PAM or the tested PAM concentrations were too low to achieve an inhibitory effect. Since PAM is a giant protein of 510 kDa, it is technically not possible to test PAM concentrations greater than 30 nM. Nonetheless, according to the dose response curves shown in FIG. 5b, higher PAM concentrations might result in a stronger inhibition of Gαs-stimulated AC activity in the tested spinal cord preparations.

Interestingly PAM was a less effective inhibitor of AC enzyme activity in DRG than in spinal cord preparations. The different inhibitory efficiencies of PAM in spinal cord and DRG preparations are most likely due to the observed differences in AC isoform expression. The major AC isoforms that are expressed in the spinal cord are type 5 and 6 that are both strongly inhibited by PAM (FIG. 5a; (Scholich et al. 2001)). In DRGs AC type 4 and 6 are the dominant AC isoforms (FIG. 5a). Since it is unknown if PAM inhibits AC type 4 either this isoform is not inhibited by PAM or, again, the tested PAM concentrations were too low to achieve the inhibitory effect. However, according to the dose response curve shown in FIG. 5bit is seems likely that higher concentrations of PAM would result in a stronger inhibition of Gαs-stimulated AC activity in the DRG preparations.

Most surprising, however, were the findings, that PAM activity had an influence on the nociceptive behavior of the test animals: This could be demonstrated for the first time by experiments of the inventors showing a significant increase in basal AC activity (Table 1) and—more important—a significant increase of the nociceptive response following formalin injection as compared to PAM sense treatment (FIG. 6b and c) when endogenous PAM expression in the spinal cord was decreased by infusing animals with PAM antisense oligonucleotides (FIG. 6a).

14. Determination of the Analgesic Effect of PAM

The above-listed evidence for the analgesic effect of PAM could for example be supported by the following hypothetic experiment: The analgesic effect of PAM, e.g. in the formalin model of acute pain, could be determined directly by intrathecal application of e.g. a peptide corresponding to amino acid residues 1028 to 1065. This peptide represents the minimal region found to be capable of mediating PAM-adenylyl cyclase interactions as determined by the yeast-two-hybrid system and AC activity assays. The peptide could be applied in a complex with the bioporter lipofection reagent (commercially available at Peqlab, Germany). This approach would allow the peptide to enter the tissue and mimic the actions of physiological PAM towards ACs.

Literature

Bailey C. H., Bartsch D., Kandel E. R.: Toward a molecular definition of long-term memory storage. Proc. Natl. Acad. Sci. U.S.A. 1996 Nov. 26;93(24):13445-52;
Brandon E. P., Idzerda R. L., McKnight G. S.: PKA isoforms, neural pathways, and behaviour: making the connection. Curr. Opin. Neurobiol. 1997 June;7(3):397-403;
Bek M. J., Zheng S., Xu J., Yamaguchi I., Asico L. D., Sun X. G. and Jose P. A. (2001) Differential expression of adenylyl cyclases in the rat nephron. Kidney Int 60, 890-899;
Chang Q. and Balice-Gordon R. J. (2000) Highwire, rpm-1, and futsch: balancing synaptic growth and stability. Neuron 26, 287-290;
Chomczynski, P., Sacchi, N.: Single-step method of RNA isolation by acid guanidinium thiocyanate-phenol-chloroform extraction. Anal. Biochem. 162, (1987) 156-159;
Graziano, M. P. FreissmuthM. Gilman A. G.: Purification of recombinant Gs alpha. Meth. Enz. (1991) 195:192-215;
Graziano M. P., Freissmuth M. and Gilman A. G. (1991) Purification of recombinant Gs alpha. Methods Enzymol 195,192-202;
Guo Q., Xie J., Dang C. V., Liu E. T., Bishop J. M.: Identification of a large Myc-binding protein that contains RCC1-like repeats. Proc. Natl. Acad. Sci. U.S.A. 1998 Aug. 4;95(16):9172-7);
Jin Y. (2002) Synaptogenesis: insights from worm and fly. Curr Opin Neurobiol 12, 71-79.);
Julius and Basbaum “Molecular mechanisms of nociception”, Nature, volume 413, 13. September 2001, pp. 203-209;
Kassis, S., and Fishman, P. H. (1982) J Biol Chem 257(9), 5312-5318;
Meller S. T., Gebhart G. F. (1997), intraplantar zymosan as a reliable, quantifiable model of thermal and mechanical hyperalgesia in the rat; Eur. J. Pain 1, 43-52;
Nair, B. G., Parikh, B., Milligan, G., and Patel, T. B. (1990) J Biol Chem 265(34), 21317-21322;
Patel T. B., Wittpoth C., Barbier A. J., Yigzaw Y. and Scholich K. (2002) Functional analyses of type V adenylyl cyclase. Methods Enzymol 345, 160-187.
Snyder S. H. (1985) Adenosine as a neuromodulator. Annu Rev Neurosci 8, 103-124.
Ruppert C., Goldowitz D., and Wille W. (1986), Proto-oncogene-c-mycis expressed in cerebellar neurons at different developmental stages, Embo J5, 1897-1901;
Schaefer A. M., Hadwiger G. D. and Nonet M. L. (2000) rpm-1, a conserved neuronal gene that regulates targeting and synaptogenesis in C. elegans. Neuron 26, 345-356);
Schaible H. G., Vanegas H.: How do we manage chronic pain? Baillieres Best. Pract. Res. Clin. Rheumatol. 2000 December;14(4):797-811;
Scherr M., Morgan M. A., Eder M., Gene Silencing Mediated by Small Interfering RNAs in Mammalian Cells, Curr Med Chem. 2003, February; 10 (3): 245-256;
Scholich K., Pierre S., Patel T. B.: Protein associated with myc (PAM) is a potent inhibitor of adenylyl cyclase. J. Biol. Chem. 2001, Dec. 14;276(50):47583-9;
Scholz and Woolf “Can we conquer pain”, Nature neuroscience supplement, volume 5, November 2002, pp.1062-1067;
Snyder S. H. (1985), Adenosine as a neuromodulator. Annual Reviews of Neuroscience 8, 103-104;
Trajkovic V, Samardzic T, Stosic-Grujicic S, Ramic Z, Mostarica Stojkovic M. Muramyl dipeptide potentiates cytokine-induced activation of inducible nitric oxide synthase in rat astrocytes. Brain Res. 2000 Nov. 10;883(1):157-63;
Wan H. I., DiAntonio A., Fetter R. D., Bergstrom K., Strauss R. and Goodman C. S. (2000) Highwire regulates synaptic growth in Drosophila. Neuron 26, 313-329.)
Wittpoth C., Scholich K., Yigzaw Y., Stringfield T. M. and Patel T. B. (1999), Regions on adenylyl cyclase that are necessary for inhibition of activity by beta gamma G(iα) subunits of heterotrimeric G proteins. Proc. Natl. Acad. Sci. USA 96, 7723-7730;
Wood, J. D. “Pathobiology of Visceral Pain: Molecular Mechanisms and Therapeutic Implications II. genetic approaches to pain therapy”, American Journal pf Physiological Gastrointestinal Liver Physiology, 2000, volume 278, G507-G512;
Woolf and Mannion “Neuropathic pain: aetiology, symptoms mechanisms, and management”, The LANCET, volume 353, Jun. 5, 1999, pp.1959-1964;
Woolf J. and Salter M. W. “Neuronal Plasticity: Increasing the Gain in Pain”, Science, volume 288, Jun.; 9, 2000, pp. 1765-1768;
Xia Z., Storm D. R.: Calmodulin-regulated adenylyl cyclases and neuromodulation. Curr. Opin. Neurobiol. 1997 June;7(3):391-6;
Yang H., Scholich K., Poser S., Storm D., Patel T. B., Goldowitz D.: Developmental expression of protein associated with myc (PAM) in the rodent brain. Brain Res Dev Brain Res 136, 2002, 35-42;
Zhen M., Huang X., Bamber B. and Jin Y. (2000) Regulation of presynaptic terminal organization by C. elegans RPM-1, a putative guanine nucleotide exchanger with a RING-H2 finger domain. Neuron 26, 331-343);

The literature listed above is incorporated herein by reference.

Standard Literature for Laboratory Methods:

If not indicated otherwise, laboratory methods were or can be performed according to standard methods listed in the below standard literature:
Sambrook et al. (1989) Molecular Cloning: A Laboratory Manual. Second edition. Cold Spring Harbor Laboratory Press, Cold Spring Harbor, N.Y. 545 pp or Current Protocols in Molecular Biology;
Current Protocols in Molecular Biology; regularly updated, e.g. Volume 2000; John Wiley & Sons, Inc; Editors: Fred M. Ausubel, Roger Brent, Robert Eg. Kingston, David D. Moore, J. G. Seidman, John A. Smith, Kevin Struhl.
Current Protocols in Human Genetics; regularly updated, e.g. Volume 2003; John Wiley & Sons, Inc; Editors: Nicholas C. Dracopoli, Honathan L. Haines, Bruce R. Korf, Cynthia C. Morton, Christine E. Seidman, J. G. Seigman, Douglas R. Smith.
Current Protocols in Protein Science; regularly updated, e.g. Volume 2003; John Wiley & Sons, Inc; Editors: John E. Coligan, Ben M. Dunn, Hidde L. Ploegh, David W. Speicher, Paul T. Wingfield.
Molecular Biology of the Cell; third edition; Alberts, B., Bray, D., Lewis, J., Raff, M., Roberts, K., Watson, J. D.; Garland Publishing, Inc. New York & London, 1994;
Gene Targeting: a practical approach (1995), Editor: A. L. Joyner, IRL Press
Genetic Manipulation of Receptor Expression and Function; D. Accili, Wiley-Liss., USA, 2000; ISBN: 0-471-35057-5.

Antisense Therapeutics, S. Agrawal, Humana Press, USA, 1996, ISBN: 0-89603-305-8.

Abbreviations Used:

AC, adenylyl cyclase; Gsα, α subunit of the stimulatory G protein of adenylyl cyclase, Gsα*, constitutively active (Q213L) mutant of Gsα; Giα, α subunit of the inhibitory G protein Gi; Gβγ, βγ subunits of heterotrimeric G proteins; PAM, protein associated with Myc; RCC1, regulator of chromosome condensation; TED, 50 mM Tris-HCl, pH 8.0, 1 mM EDTA, 1 mM DTT; ODN, oligodeoxynucleotide; RT, room temperature;

TABLE 1
Basal AC activity is elevated in spinal cord lysates of antisense
treated rats. Spinal cords lysates (20 μg) were assayed for AC
activity in the absence or presence of 80 nM Gαs or 100 μM
forskolin as described above. The mean AC activity ± SEM of
spinal cord lysates from at least three rats per group, each
measured twice in triplicates, is shown (ns = not significant).
Table

| | |
|---|---|
| | AC activity |
| Condition | (pmol/min/mg |

| | | |
|---|---|---|
| basal | | |
| sense | 102.7 ± 6.6 | |
| antisense | 124.0 ± 6.2 | (p ≦ 0.01) |
| Gαs | | |
| sense | 398.8 ± 16.9 | |
| antisense | 432.6 ± 17.2 | (ns) |
| Forskolin | | |
| sense | 283.9 ± 25.7 | |
| antisense | 316.2 ± 24.3 | (ns) |

1. A method of alleviating pain, the method comprising administering to a subject protein associated with Myc (PAM).

2. The method of claim 1, wherein the PAM is human PAM.

3. The method of claim 2, wherein the PAM comprises the amino acid sequence of SEQ ID NO. 2.

4. A method of alleviating pain, the method comprising administering to a subject a functional fragment or derivative of protein associated with Myc (PAM)

5. The method of claim 4, wherein the functional fragment or derivative is of human PAM.

6. The method of claim 5, wherein the functional fragment or derivative of PAM comprises a functional fragment or derivative of a polypeptide having the amino acid sequence of SEQ ID NO. 2.

7. The method of claim 5, wherein the functional fragment or derivative of PAM comprises a polypeptide selected from the group consisting of amino acids 400 to 1400 of SEQ ID NO. 2, amino acids 446 to 1062 of SEQ ID NO. 2, amino acids 499 to 1065 of SEQ ID NO. 2, amino acids 1028 to 1231 of SEQ ID NO. 2, amino acids 1000 to 1300 of SEQ ID NO. 2, amino acids 1000 to 1100 of SEQ ID NO. 2, and amino acids 1028 to 1065 of SEQ ID NO. 2.

8. The method of claim 4, wherein the functional fragment or derivative of PAM comprises a polypeptide selected from the group consisting of SEQ ID NOS. 17-23.

9. The method of claim 4, wherein the functional fragment or derivative is capable of inhibiting adenylyl cyclase.

10. The method of claim 9, wherein the adenylyl cyclase is adenylyl cyclase type I, V or VI.

11. A method of screening compounds, the method comprising the steps of
a. providing first sample comprising protein associated with Myc (PAM) or a functional fragment or derivative thereof;
b. providing a second sample that does not comprise PAM or a functional fragment or derivative thereof;
c. contacting the first sample with a compound;
d. contacting the second sample with the compound;
f. determining PAM activity in the first sample;
g. determining PAM activity in the second sample; and
h. comparing the PAM activity according to c) with that according to d).

12. The method of claim 11, wherein the first sample comprises human PAM.

13. The method of claim 11, wherein the first sample comprises a polypeptide comprising the amino acid sequence of SEQ ID NO. 2.

14. The method of claim 11, wherein the first sample comprises a functional fragment or derivative of a polypeptide comprising the amino acid sequence of SEQ ID NO. 2.

15. The method of claim 11, wherein the first sample comprises an polypeptide comprising the amino acid sequence encoded by the nucleic acid sequence of SEQ ID NO. 1.

16. The method of claim 11, wherein the first sample comprises a functional fragment or derivative of a polypeptide having an amino acid sequence encoded by the nucleic acid sequence of SEQ ID NO. 1.

17. The method of claim 11, wherein the first sample comprises a polypeptide selected from the group consisting of amino acids 400 to 1400 of SEQ ID NO. 2, amino acids 446 to 1062 of SEQ ID NO. 2, amino acids 499 to 1065 of SEQ ID NO. 2, amino acids 1028 to 1231 of SEQ ID NO. 2, amino acids 1000 to 1300 of SEQ ID NO. 2, amino acids 1000 to 1100 of SEQ ID NO. 2, and amino acids 1028 to 1065 of SEQ ID NO. 2.

18. The method of claim 11, wherein the first sample comprises a polypeptide selected from the group consisting of SEQ ID NOS. 17-23.

19. The method of claim 11, wherein the functional fragment or derivative of PAM is capable of inhibiting adenylyl cyclase.

20. The method of claim 19, wherein the adenylyl cyclase is adenylyl cyclase type I, V or VI.

21. The method of claim 11, wherein the first sample comprises PAM that is recombinantly expressed.

22. The method of claim 11, wherein a modified cell having a lower activity as compared to its unmodified state is used.

23. The method of claim 22, wherein a PAM knock-out cell is used.

24. The method of claim 11, wherein the step of determining PAM activity comprises determining PAM activity directly.

25. The method of claim 11, wherein the step of determining PAM activity comprises determining PAM activity indirectly.

26. The method of claim 11, wherein the step of determining PAM activity comprises measuring adenylate cyclase.


SEQUENCE LISTING (30 sequences)

SEQ ID NO: 1 - DNA, 14807 bp, Homo sapiens
tcttggagcgttctcagtttctcaacagatcttcacttgctaggcagccagaagccggcggcagtggcggcaccgcctcctcctcacattcccggggtggcggggttagatgagcggccccagtcgcggcgccgggggcgctgttcatgccggttcccgacggctccgtggctgctgcggggctggggctggggctacccgccgcggactccccgggtcactaccagctgctgctgtcaggccgggccctggccgaccgctaccggaggatttataccgctgcgctcaatgacagggaccaggggggcggcagcgctggacacccagcctccaggaataagaaaattttaaataagaagaaattgaaaagaaaacagaagagcaaatcaaaagtgaagacaagaagcaagtctgaaaacttagagaatacagtaatcataccagatatcaaactacatagcaatccttctgctttcaatatttactgtaatgtacgccattgcgttctggaatggcagaaaaaggaaatatcattggcagccgcatctaagaactctgtgcagagtggagaatcagatagtgatgaagaagaggaatccaaagagccccctatcaagcttccaaagattattgaggttggcctttgtgaagtttttgaattgatcaaagagacacgattttctcatccatccctgtgtctcaggagtctccaagccctgctcaacgtgctgcagggccagcagccagaagtgctccagtctgagccacctgaggtcctagagtctctcttccagcttcttttggaaatcaccgttcgaagtactgggatgaatgacagcacaggacagtccttaacagcactttcctgtgcttgcctctttagtctggtggcttcttggggagaaacaggaaggacacttcaggccatctctgctatcctcaccaacaatggaagccatgcttgccaaactattcaggtgccaacaattctaaattcgctacagagaagtgtacaagcagttttggtgggaaaaattcaaattcaggactggtttagtaatggcattaagaaagcagctttaatgcacaagtggccattaaaagaaatatctgttgatgaagatgaccaatgtctacttcagaatgatggattttttctttatctattatgcaaggatggattatataaaataggctctggatacagtggaacagttaggggccatatatacaattctacatcccgtattagaaacagaaaagaaaaaaagtcttggttagggtatgctcagggttatttattatatagagatgtgaataaccacagcatgacagccataaggataagccctgaaacactggagcaagatggtactgtgatgttaccagattgccacactgaaggtcaaaatattttattcactgatggagaatatattaatcagatagctgcttcaagagatgatggctttgttgtcagaatatttgccacaagcactgaacctgttctacagcaagaattgcaacttaaactggctagaaaatgcttacatgcctgtcgtatctcattattcgatctggaaaaggacttgcatattataagtacaggatttgatgaggagtcagcaattcttggtgcaggacgagagtttgcgctaatgaaaacagcaaatggaaagatatattacactggcaaataccagagtcttggaatcaaacaaggtggtccttcagcaggaaaatgggttgagctaccaattacaaaatctccaaagatagtacacttctcagttggacacgatggctctcacgcccttttagttgcagaagatgggagcatattctttacaggatctgctagtaaaggagaagatggagaatcaattaagagcagacggcaatccaaaccttataaacctaaaaagataattaagatggaaggaaagattgtggtatatacagcctgcaataatggaagtagttctgttatttctaaagatggagaactctacatgtttggaaaagatgccatttactctgatagttcaagtttggtaactgatttgaagggccattttgtaactcaggtagctatgggcaaagctcacacttgtgttttaatgaagaatggagaggtgtggacatttggtgtaaataataaaggacagtgtggacgagatactggtgccatgaaccaaggtgggaaagggtttggagttgaaaatatggcaacagcaatggatgaagacctggaagaagaactagatgaaaaagatgagaagtctatgatgtgccctccaggcatgcacaaatggaagctggagcagtgcatggtttgcactgtctgtggagactgtacaggttatggagccagctgtgtcagtagtggacggccagacagagtccccggagggatctgtggttgtggttccggagaatctggttgtgctgtgtgtggatgttgcaaggcctgtgcaagagagttagatggtcaagaggcaagacaaagaggaattcttgatgcagtgaaagaaatgatacctttagatcttcttttagctgtcccagtgcccggggttaacattgaagaacaccttcagttacgacaagaagaaaaacggcaacgtgtaatcagaaggcacagattagaggaaggaagaggcccccttgtatttgctggtcctatttttatgaaccatcgagaacaggctctagccagactcagatcccatccagcacacgtaaagcataaacgggacaagcacaaagatggaagtggagaaagaggcgaaaaggatgcaagcaaaatcacaacataccctccaggctctgtgcgatttgactgtgagctccgggcagtccaagtcagctgtggatttcaccattcagtggttttaatggaaaatggagatgtctatacatttggttatgggcagcatgggcagctaggacatggagatgtcaactccaggggatgtcccactcttgttcaagcattgccaggccctagcacacaagtcactgcaggcagcaaccatacggcagtacttttaatggatggacaggtcttcacatttggaagtttttctaaaggacaactgggcagaccaattttggatgtgccatattggaatgcaaagccagctcccatgcctaacattggatcaaaatatggaagaaaagctacttggataggtgcaagtggggaccaaacttttttacgaattgatgaagcacttattaattctcatgtacttgctacatcagaaatttttgccagtaaacacataataggcttggtacctgcttctatatcagaacctcctccatttaaatgccttctgataaataaagtggatgggagttgtaaaacttttaatgactcagaacaagaggatctgcaaggatttggtgtgtgtcttgatcctgtatatgatgtaatttggaggtttcgaccaaatactagagagctgtggtgttacaatgcggtggttgctgatgccaggcttccctctgcagcagacatgcagtccagatgtagtatcctaagtcctgaacttgccttaccaacaggatcaagggccctcactacccgatctcatgcagctttgcacattttaggttgtcttgataccttggcagctatgcaggacttaaaaatgggtgttgcaagtacagaggaagagactcaagcagtaatgaaggtttattctaaagaagattatagtgtggtaaacaggtttgaaagtcatggaggaggctggggttattctgcccattcagtagaagctatacgtttcagtgccgacactgatattttacttggtggtcttggtctgtttggaggtagaggagaatatactgctaaaattaagctgtttgaattgggtcctgatggaggagatcatgaaactgatggtgaccttcttgcagagactgatgtattggcttatgactgtgctgctagagaaaaatatgcaatgatgtttgatgagcctgttctcctgcaagctgggtggtggtatgtggcatgggcccgagtgtcaggacccagcagtgactgtggatctcatggacaggcatctattaccacagatgatggggttgttttccagttcaagagttcaaagaaatcaaataatggtacagatgttaatgcgggtcagatacctcagttattatacagacttccaaccagtgatggcagtgcttcaaaaggcaaacagcaaaccagtgaacctgtacacattttaaagaggtcttttgcaagaactgtctcagtggaatgttttgagtcattgttgagtattcttcactggagctggaccaccttagtcttaggagttgaagaacttagaggattaaaaggattccagttcacagctacactcctagatttagagagactgcgctttgtgggtacctgttgtctgaggttattgcgtgtctatacctgtgaaatttacccagtgtcagctacaggaaaagcagttgtagaagaaactagcaaattagcagagtgtattggaaaaaccagaactttgttaagaaaaattttatcagaaccacttgatcactgcatggtgaaattggataatgatcctcaaggatatctcagtcaacccttgagtcttctagaagctgtccttcaggaatgtcataatactttcactgcctgctttcattctttctacccaactcctgccttacagtgggcttgcctttgtgatctgctgaattgtttggatcaggatatccaagaagcaaacttcaagacatcaagtagccgactccttgcagctgttatgtcagctctgtgtcacacgtctgttaagctgacttccatcttcccgattgcgtatgatggagaagtattactacgatcaattgttaaacaagttagtacagagaacgactcaacactagttcatcgttttccccttttggtggcacatatggaaaaactcagccagagtgaagagaatatctcagggatgacaagcttccgtgaagttctggagaaaatgctggtcattgttgtgctaccagtcaggaacagcctgaggagagaaaatgaactcttctcctcccacctcgtctctaacacctgtggattactggccagcattgtcagtgaactgacagcgtcagccctgggatctgaggttgatggacttaattctcttcactctgtaaaagctagtgctaaccgatttacaaaaacaagtcagggcagaagttggaacactgggaacgggtcccctgatgcaatctgtttttcagtagacaaacctggaatagttgtggttggtttctctgtctatggaggaggtggaattcatgaatatgaattagaggtgttggttgatgatagtgaacatgcaggagattcaactcattcccacagatggacatctctggaattagtgaaaggaacgtacacaacggatgactcacccagtgatatagctgagatcagacttgacaaagtggttcctttaaaggaaaatgttaaatatgctgtgcgcttgaggaactatggaagccgtacagccaatggagatggaggaatgaccacagttcagtgccctgatggtgtgacattcacattcagcacgtgcagcttgagcagtaacggcacaaaccaaaccagaggacagatcccacagatactctactataggagtgaatttgatggagatttacaatcccaacttctgagtaaagccaatgaagaagataaaaactgtagcagagcattgtctgttgtaagcactgtcgttcgagcctctaaggacctcctgcacagagctcttgctgtggatgctgatgacattccagaactgctgagttcttccagtctgttttccatgctgctcccccttattatagcctacataggaccagtagctgctgctattcccaaggtggctgtagaagtctttggccttgtccaacaattgcttccgtcagttgccattttgaatcagaagtatgcaccgcctgccttcaaccctaatcagtcgacagatagcaccacaggaaaccagcctgaacagggcctctctgcttgtacaacctccagtcactatgctgtcatagagagtgagcacccgtataaacctgcctgtgtgatgcattacaaggtgacattcccagaatgtgtgaggtggatgacaatcgaatttgaccctcagtgtggtactgcacagtcagaagatgtccttcgtttgttgattcctgtcagaactgttcagaattcaggatatggaccaaaattgacatctgttcatgaaaatcttaattcatggatagaattaaagaaattttcaggatcctctgggtggcctactatggttttggtgttgccaggaaatgaggcccttttttcattggagactgcatcagattatgtgaaagatgacaaagcttctttctatggttttatgtgttttgcaattggatatgaatttagccctggacctgatgagggagtcatccaattggaaaaagaattagccaatcttggtggggtttgtgcagcagctctgatgaagaaggacctagcacttcctattggtaatgaattagaagaagaccttgaaattcttgaggaggctgcattgcaggtgtgcaaaacccattctggaattcttggaaagggtctagctctttctcattcaccaactatattagaagcacttgagggaaatttaccactccaaatccaaagcaatgaacagtcttttctggatgattttattgcctgtgtcccaggatcaagtggtggaaggcttgcaaggtggcttcagccagattcatatgcggatcctcagaaaacatctttgatcctgaataaggatgatattcgttgtggttggcctaccaccataactgttcaaacaaaagaccagtatggggatgtggtacatgttcccaatatgaaggtggaagtgaaagctgtccctgtttctcagaaaaaaatgtctttacaacaagatcaagcaaagaaacctcaaaggattcctggcagtcctgcagtaacagctgcatcttctaatactgacatgacttatggagggctggcatcaccaaagctagatgtttcatatgaaccaatgatagtgaaggaagctcgatatattgccataacaatgatgaaggtttatgaaaattattcatttgaagaactacgttttgcatcaccaactcctaagagacccagtgagaatatgctgatccgtgtcaataatgatgggacttattgtgcaaattggactccaggggctattggactctacactcttcatgttaccattgatggcattgaaatcgatgctggtctggaagtaaaagtaaaagacccaccaaaagggatgataccaccaggaactcagttggtcaaaccaaagtctgaacctcagcctaataaggttcgaaaatttgtggccaaggacagtgcggggcttcgcatccgtagccacccttcccttcagagtgagcagataggcatagtgaaagtcaatggaactatcacttttattgatgagatccataatgatgatggtgtgtggctgaggctgaatgatgagacaataaagaagtatgtccctaacatgaatggttacactgaagcctggtgcctctcttttaatcaacatcttggcaagagtcttctggtccctgttgacgaatctaaaactaatactgatgactttttcaaagacataaactcctgctgcccacaggaagcaacaatgcaagaacaagatatgccattcttgcgaggagggccaggcatgtacaaggtagtgaagacgggaccttcaggtcacaacatcagaagctgccctaaccttagaggtatcccaattggaatgttagttctgggaaacaaagtcaaagcagtgggagaggtaaccaattctgaagggacatgggtgcaactggatcagaacagcatggtagagttctgtgagagtgatgaaggagaggcatggtccttagctagagacagaggcggaaaccagtacctccgacatgaagatgaacaagctcttctggatcagaattctcaaactcctcctccaagccctttctcagtgcaagcttttaataaaggggcaagttgcagtgcccaaggatttgattatggactcggaaatagcaaaggtgatcgaggaaacatctcaacatcttctaaaccagcctctacatcaggaaaatcagagctgtcctctaaacacagcagatcgcttaaacctgatggacgtatgagccggactactgctgatcagaagaagccaaggggcacagaaagtttatctgctagtgaatccctcatcttaaaatctgatgctgcaaagttgaggtcagattcccacagtaggtcattatcccccaaccataacaccttgcagacattgaaatctgatgggaggatgccttctagctccagagctgaatccccaggaccaggttctcggttgtcatctcctaagccaaagactctcccagccaataggtctagcccatcgggtgctagttctccacgctcctcctcaccacatgataaaaatctacctcaaaaaagtactgctcctgttaagacaaagcttgatcctcctcgggaacgttctaaatcagactcttacacacttgatccagataccctccgcaagaagaaaatgcccctcacagaacctttgagaggacggtcaacgtcaccaaaaccaaaatcagtaccaaaggattctacagattcccctggatctgaaaatagagctccctctccccatgtggtacaggaaaacctccacagtgaggtggtcgaagtctgcacctcaagtactttaaaaacaaatagtctaacagacagcacctgcgatgacagcagtgaatttaagagtgtggatgaaggttcaaataaagttcattttagcattggaaaagcaccactgaaagatgaacaggaaatgagagcatctcccaaaataagtcgaaaatgtgctaatagacacaccaggcccaaaaaagaaaaatcgagttttcttttcaaaggagatggatccaagcctttagagccagccaagcaagccatgtctccttctgtggccgaatgtgccagagctgtgtttgcttccttcctctggcatgaaggcatagtacatgatgcaatggcttgttcttctttcctaaagtttcatcctgaactttccaaagaacatgctcctataaggagtagtttaaatagccaacaacctacagaagaaaaagaaaccaagttaaaaaatagacattcattagaaatatcatctgcactgaatatgtttaatattgcaccccatggaccagatatatctaagatgggtagcatcaacaaaaacaaggtattgtctatgcttaaggaaccacctctgcatgaaaaatgtgaggatgggaaaaccgagaccacttttgaaatgtccatgcataacacaatgaagtctaagtctcctcttcccttaactttacaacatttagtggctttttgggaagacatctctttggctactatcaaagctgcttcccagaatatgatttttccaagtcctggttcctgtgcagttcttaaaaagaaagagtgtgagaaaggaaggaataagaagtccaaaaaggaaaaaaagaaaaaagaaaaggcagaagttaggcccaggggtaatttgtttggagagatggcccagctggcagtaggaggaccagagaaagataccatctgtgaactgtgtggggagtcacatccatacccggtgacctatcacatgagacaagctcacccaggttgtggccgatatgctggtggacaaggttacaatagcattgggcatttttgtggaggatgggctggtaactgtggtgatggtggcataggaggaagcacttggtatctggtatgtgatcgctgtagagaaaaatacctccgcgaaaaacaggctgctgcaagggagaaggtcaaacaatctaggagaaaaccaatgcaagtcaagacccctcgtgccttgcccaccatggaagctcaccaggtgattaaagccaatgcactcttcctgctgtccctgagcagtgcagcagaaccgagcattctgtgttaccatcctgcaaagccattccaatctcagttgcccagtgtaaaagaaggcatttctgaggatcttcctgtgaaaatgccttgtctgtacctgcagacattagctaggcatcatcatgaaaattttgtgggctatcaagatgacaatctattccaggatgaaatgagatatctacgttcaacatctgtacctgccccgtatatatcagtaactcctgatgcaagtcctaatgtatttgaagagccagagagcaatatgaagtctatgccaccaagtttagaaaccagtcccataactgatactgatcttgcaaagagaactgtcttccaaagatcatactcagttgttgcttccgaatatgataaacaacactccattttacctgcacgagttaaagctattcctagaagaagagttaacagtggagacactgaagttggttcttcccttttgagacatccgtctcctgagctttctcggctaatctcagcccacagctctctttctaaaggagaacgaaatttccagtggccagttttagcttttgttatacaacatcatgatctagaaggtcttgaaatagcaatgaaacaggccctaaggaaatctgcttgtcgagtttttgctatggaggctttcaactggcttctgtgtaatgtcatccaaaccacttctctccatgatattctgtggcattttgtggcatcactgactcctgcaccagtggaaccagaggaagaagaggatgaagaaaataaaacaagcaaagaaaattcagaacaagagaaagatacaagagtatgtgaacatccactctcagacatagtgattgccggggaacgtgctcatcctttaccacacacctttcaccgcttgctgcagaccatctcagaccttatgatgtctctccccagcggcagttcattacagcaaatggccctgaggtgctggagtctcaaattcaagcaatctgatcaccagttccttcatcagagcaacgtctttcatcacattaacaatattttgtcaaagtcagatgatggcgatagtgaagagagttttagcatcagtatacagtctggctttgaagctatgagtcaggaattatgcatagtaatgtgcttaaaggacttaaccagcattgttgacataaaaacttcaagccgacctgccatgattggcagtttgacagacggctccacagaaaccttttgggaatcaggagatgaagataaaaacaaaactaagaacatcaccatcaactgtgtaaaaggaatcaatgcccgctatgtgtctgttcacgtggacaattcccgagatcttgggaataaagttacctcaatgaccttcttaactggcaaagcagtagaagatttgtgcagaataaagcaggttgatctggattccaggcacattggctgggtaacaagtgaacttccaggaggggataatcacatcataaaaattgaattaaaaggcccagaaaatacactgagagttcgacaagtcaaagtcctgggctggaaagatggtgaaagcacaaaaatagctggccagatttcagccagtgtggcccagcagaggaactgtgaagctgagactctgcgagtattcagactgattacgtctcaagtatttggaaagctcatctctggagatgctgaacctacaccagaacaagaggaaaaagcactattgtcatcacctgaaggagaagaaaaagtatacaatgcaacatcagatgctgacctgaaagaacatatggttggaatcatattcagcaggagtaagctgactaacttacaaaaacaggtgtgtgctcatattgtccaagctattcgcatggaagctaccagagtccgtgaagaatgggaacatgctatatcaagcaaagaaaatgccaattctcagccaaatgatgaagatgcctcctctgatgcctactgctttgagctgctctctatggttttagcactgagtggctctaacgttggccggcaatatctggctcaacagctaaccctgcttcaggatctcttctcgctgcttcacacagcctctcctagagtccagagacaggtaacctctttactaagaagagttttgcctgaagtaacccctagtcgtctggccagcatcataggagtgaaatccctccccccagcagatatcagtgatatcattcactcaacagagaaaggagactggaataagctgggtatcttggacatgtttctaggatgcattgccaaagcactcactgtacagctaaaagccaaaggaaccaccatcactggaacagctggtaccactgtgggcaaaggagttacaacagttactcttccgatgattttcaattccagttatctccgacgaggtgaaagtcattggtggatgaagggctcaacccctacccagatctcagagatcatcattaaacttatcaaggatatggcagcaggtcatctgtcagaagcttggtcccgagtgacaaaaaatgctattgcagaaaccatcattgccttgaccaagatggaagaagaatttaggtctccagtgagatgtattgcaacaactagactctggcttgctctcgcatccctatgtgttcttgatcaggaccacgtagatcgtctctcctcggggagatggatgggaaaggatggacaacaaaaacaaatgcctatgtgtgataaccatgatgatggtgaaactgcagcaatcattttatgcaatgtctgtggaaatttatgtacagactgtgacagattccttcaccttcatcgaagaaccaaaactcatcaaagacaggtcttcaaagaagaagaagaagctataaaggttgaccttcatgaaggttgtggtagaaccaaattgttctggttgatggcactggcagattctaaaacaatgaaggcaatggtggaattccgagaacacacaggcaaacccaccacgagtagctcagaagcatgtcgcttctgtggttccaggagtggaacagagttatctgctgttggcagtgtttgttctgatgcagattgccaggaatacgctaagatagcctgtagtaagacgcatccttgtggccatccatgcgggggtgttaaaaacgaagagcactgtctgccctgtctacacggctgtgacaaaagtgccacaagcctgaagcaagacgccgatgacatgtgcatgatatgtttcaccgaagcgctctcggcagcaccagccattcagctggattgtagtcacatattccacttacagtgctgtcggcgagtattagaaaatcgatggcttggcccaaggataacatttggatttatatcttgtcccatttgcaagaacaaaattaatcacatagtactaaaagacctacttgatccaataaaagaactctatgaggatgtcagaagaaaagccttaatgagattggaatatgaaggtctgcataagagtgaagctatcacaactcctggtgtgaggttttataatgacccagctggctatgcaatgaatagatatgcatattatgtgtgctacaaatgcagaaaggcatattttggtggtgaagctcgctgcgatgctgaggctggacggggagatgattatgatcccagagagctcatttgtggtgcctgttctgatgtttccagggctcagatgtgtcccaaacatggcacagactttttggaatataaatgtcgctactgctgttcagtggctgtttttttctgttttggaacaacacatttttgtaatgcttgtcatgatgattttcaaagaatgactagcattcctaaggaagaactaccacactgtcctgcaggtcccaaaggcaagcagttagaaggaactgaatgtccactccatgttgttcatccacccactggggaagagtttgctctgggatgtggagtgtgcagaaatgcccacactttttagaacacgcagatcctttgtctacagagagaaaaattgccttcatcccccaagaggatgcggtgaagtttaaactctgctcaccataaggacgggaccatttttacatccatgaaaatgaaccattcacagtgcaagaaggataccaaataccatgtacataattcttgctatgaaaagtttccccattattttggtttatcttcttttgaacaaatgacatcaaacttgtgaggtgtttgcatgtggccattaccgtcattggcctgtgaagcattggacatttatagataattgatataaaagaatcgccatgcccatggactaagaacgatgctggctttcaagcaaaaaagaaaaataatcattgtttattgtatactgcctttttgtaatcctgtacaattgcatcacgggtggggataaaaagaggaatattctggtttatttcctagactgttatttaaaaaaaaaaaaaacattgtgttaggacagcatataaatgtaataagtatcacactgtatataaacatatcaatgtttgtcctgtataagaattactaaattacaaatgcaatttcatttaaacttctaggttaagtttgagcctgaaattttaatgaagtgcaatactgagtgtgcctcattatcttgcagctgtaaacatattggaatgtacatgtcaataaaaccactgtacatttttatacagtgataaagtctaaaaaaaaaaaaaaa

SEQ ID NO: 2 - protein, 4641 aa, Homo sapiens
MPVPDGSVAAAGLGLGLPAADSPGHYQLLLSGRALADRYRRIYTAALNDRDQGGGSAGHPASRNKKILNKKKLKRKQKSKSKVKTRSKSENLENTVIIPDIKLHSNPSAFNIYCNVRHCVLEWQKKEISLAAASKNSVQSGESDSDEEEESKEPPIKLPKIIEVGLCEVFELIKETRFSHPSLCLRSLQALLNVLQGQQPEVLQSEPPEVLESLFQLLLEITVRSTGMNDSTGQSLTALSCACLFSLVASWGETGRTLQAISAILTNNGSHACQTIQVPTILNSLQRSVQAVLVGKIQIQDWFSNGIKKAALMHKWPLKEISVDEDDQCLLQNDGFFLYLLCKDGLYKIGSGYSGTVRGHIYNSTSRIRNRKEKKSWLGYAQGYLLYRDVNNHSMTAIRISPETLEQDGTVMLPDCHTEGQNILFTDGEYINQIAASRDDGFVVRIFATSTEPVLQQELQLKLARKCLHACRISLFDLEKDLHIISTGFDEESAILGAGREFALMKTANGKIYYTGKYQSLGIKQGGPSAGKWVELPITKSPKIVHFSVGHDGSHALLVAEDGSIFFTGSASKGEDGESIKSRRQSKPYKPKKIIKMEGKIVVYTACNNGSSSVISKDGELYMFGKDAIYSDSSSLVTDLKGHFVTQVAMGKAHTCVLMKNGEVWTFGVNNKGQCGRDTGAMNQGGKGFGVENMATAMDEDLEEELDEKDEKSMMCPPGMHKWKLEQCMVCTVCGDCTGYGASCVSSGRPDRVPGGICGCGSGESGCAVCGCCKACARELDGQEARQRGILDAVKEMIPLDLLLAVPVPGVNIEEHLQLRQEEKRQRVIRRHRLEEGRGPLVFAGPIFMNHREQALARLRSHPAHVKHKRDKHKDGSGERGEKDASKITTYPPGSVRFDCELRAVQVSCGFHHSVVLMENGDVYTFGYGQHGQLGHGDVNSRGCPTLVQALPGPSTQVTAGSNHTAVLLMDGQVFTFGSFSKGQLGRPILDVPYWNAKPAPMPNIGSKYGRKATWIGASGDQTFLRIDEALINSHVLATSEIFASKHIIGLVPASISEPPPFKCLLINKVDGSCKTFNDSEQEDLQGFGVCLDPVYDVIWRFRPNTRELWCYNAVVADARLPSAADMQSRCSILSPELALPTGSRALTTRSHAALHILGCLDTLAAMQDLKMGVASTEEETQAVMKVYSKEDYSVVNRFESHGGGWGYSAHSVEAIRFSADTDILLGGLGLFGGRGEYTAKIKLFELGPDGGDHETDGDLLAETDVLAYDCAAREKYAMMFDEPVLLQAGWWYVAWARVSGPSSDCGSHGQASITTDDGVVFQFKSSKKSNNGTDVNAGQIPQLLYRLPTSDGSASKGKQQTSEPVHILKRSFARTVSVECFESLLSILHWSWTTLVLGVEELRGLKGFQFTATLLDLERLRFVGTCCLRLLRVYTCEIYPVSATGKAVVEETSKLAECIGKTRTLLRKILSEPLDHCMVKLDNDPQGYLSQPLSLLEAVLQECHNTFTACFHSFYPTPALQWACLCDLLNCLDQDIQEANFKTSSSRLLAAVMSALCHTSVKLTSIFPIAYDGEVLLRSIVKQVSTENDSTLVHRFPLLVAHMEKLSQSEENISGMTSFREVLEKMLVIVVLPVRNSLRRENELFSSHLVSNTCGLLASIVSELTASALGSEVDGLNSLHSVKASANRFTKTSQGRSWNTGNGSPDAICFSVDKPGIVVVGFSVYGGGGIHEYELEVLVDDSEHAGDSTHSHRWTSLELVKGTYTTDDSPSDIAEIRLDKVVPLKENVKYAVRLRNYGSRTANGDGGMTTVQCPDGVTFTFSTCSLSSNGTNQTRGQIPQILYYRSEFDGDLQSQLLSKANEEDKNCSRALSVVSTVVRASKDLLHRALAVDADDIPELLSSSSLFSMLLPLIIAYIGPVAAAIPKVAVEVFGLVQQLLPSVAILNQKYAPPAFNPNQSTDSTTGNQPEQGLSACTTSSHYAVIESEHPYKPACVMHYKVTFPECVRWMTIEFDPQCGTAQSEDVLRLLIPVRTVQNSGYGPKLTSVHENLNSWIELKKFSGSSGWPTMVLVLPGNEALFSLETASDYVKDDKASFYGFMCFAIGYEFSPGPDEGVIQLEKELANLGGVCAAALMKKDLALPIGNELEEDLEILEEAALQVCKTHSGILGKGLALSHSPTILEALEGNLPLQIQSNEQSFLDDFIACVPGSSGGRLARWLQPDSYADPQKTSLILNKDDIRCGWPTTITVQTKDQYGDVVHVPNMKVEVKAVPVSQKKMSLQQDQAKKPQRIPGSPAVTAASSNTDMTYGGLASPKLDVSYEPMIVKEARYIAITMMKVYENYSFEELRFASPTPKRPSENMLIRVNNDGTYCANWTPGAIGLYTLHVTIDGIEIDAGLEVKVKDPPKGMIPPGTQLVKPKSEPQPNKVRKFVAKDSAGLRIRSHPSLQSEQIGIVKVNGTITFIDEIHNDDGVWLRLNDETIKKYVPNMNGYTEAWCLSFNQHLGKSLLVPVDESKTNTDDFFKDINSCCPQEATMQEQDMPFLRGGPGMYKVVKTGPSGHNIRSCPNLRGIPIGMLVLGNKVKAVGEVTNSEGTWVQLDQNSMVEFCESDEGEAWSLARDRGGNQYLRHEDEQALLDQNSQTPPPSPFSVQAFNKGASCSAQGFDYGLGNSKGDRGNISTSSKPASTSGKSELSSKHSRSLKPDGRMSRTTADQKKPRGTESLSASESLILKSDAAKLRSDSHSRSLSPNHNTLQTLKSDGRMPSSSRAESPGPGSRLSSPKPKTLPANRSSPSGASSPRSSSPHDKNLPQKSTAPVKTKLDPPRERSKSDSYTLDPDTLRKKKMPLTEPLRGRSTSPKPKSVPKDSTDSPGSENRAPSPHVVQENLHSEVVEVCTSSTLKTNSLTDSTCDDSSEFKSVDEGSNKVHFSIGKAPLKDEQEMRASPKISRKCANRHTRPKKEKSSFLFKGDGSKPLEPAKQAMSPSVAECARAVFASFLWHEGIVHDAMACSSFLKFHPELSKEHAPIRSSLNSQQPTEEKETKLKNRHSLEISSALNMFNIAPHGPDISKMGSINKNKVLSMLKEPPLHEKCEDGKTETTFEMSMHNTMKSKSPLPLTLQHLVAFWEDISLATIKAASQNMIFPSPGSCAVLKKKECEKGRNKKSKKEKKKKEKAEVRPRGNLFGEMAQLAVGGPEKDTICELCGESHPYPVTYHMRQAHPGCGRYAGGQGYNSIGHFCGGWAGNCGDGGIGGSTWYLVCDRCREKYLREKQAAAREKVKQSRRKPMQVKTPRALPTMEAHQVIKANALFLLSLSSAAEPSILCYHPAKPFQSQLPSVKEGISEDLPVKMPCLYLQTLARHHHENFVGYQDDNLFQDEMRYLRSTSVPAPYISVTPDASPNVFEEPESNMKSMPPSLETSPITDTDLAKRTVFQRSYSVVASEYDKQHSILPARVKAIPRRRVNSGDTEVGSSLLRHPSPELSRLISAHSSLSKGERNFQWPVLAFVIQHHDLEGLEIAMKQALRKSACRVFAMEAFNWLLCNVIQTTSLHDILWHFVASLTPAPVEPEEEEDEENKTSKENSEQEKDTRVCEHPLSDIVIAGERAHPLPHTFHRLLQTISDLMMSLPSGSSLQQMALRCWSLKFKQSDHQFLHQSNVFHHINNILSKSDDGDSEESFSISIQSGFEAMSQELCIVMCLKDLTSIVDIKTSSRPAMIGSLTDGSTETFWESGDEDKNKTKNITINCVKGINARYVSVHVDNSRDLGNKVTSMTFLTGKAVEDLCRIKQVDLDSRHIGWVTSELPGGDNHIIKIELKGPENTLRVRQVKVLGWKDGESTKIAGQISASVAQQRNCEAETLRVFRLITSQVFGKLISGDAEPTPEQEEKALLSSPEGEEKVYNATSDADLKEHMVGIIFSRSKLTNLQKQVCAHIVQAIRMEATRVREEWEHAISSKENANSQPNDEDASSDAYCFELLSMVLALSGSNVGRQYLAQQLTLLQDLFSLLHTASPRVQRQVTSLLRRVLPEVTPSRLASIIGVKSLPPADISDIIHSTEKGDWNKLGILDMFLGCIAKALTVQLKAKGTTITGTAGTTVGKGVTTVTLPMIFNSSYLRRGESHWWMKGSTPTQISEIIIKLIKDMAAGHLSEAWSRVTKNAIAETIIALTKMEEEFRSPVRCIATTRLWLALASLCVLDQDHVDRLSSGRWMGKDGQQKQMPMCDNHDDGETAAIILCNVCGNLCTDCDRFLHLHRRTKTHQRQVFKEEEEAIKVDLHEGCGRTKLFWLMALADSKTMKAMVEFREHTGKPTTSSSEACRFCGSRSGTELSAVGSVCSDADCQEYAKIACSKTHPCGHPCGGVKNEEHCLPCLHGCDKSATSLKQDADDMCMICFTEALSAAPAIQLDCSHIFHLQCCRRVLENRWLGPRITFGFISCPICKNKINHIVLKDLLDPIKELYEDVRRKALMRLEYEGLHKSEAITTPGVRFYNDPAGYAMNRYAYYVCYKCRKAYFGGEARCDAEAGRGDDYDPRELICGACSDVSRAQMCPKHGTDFLEYKCRYCCSVAVFFCFGTTHFCNACHDDFQRMTSIPKEELPHCPAGPKGKQLEGTECPLHVVHPPTGEEFALGCGVCRNAHTF

SEQ ID NO: 3 - DNA, 290040 bp, Homo sapiens
cttcagcttggagtactaaatatattctatgaaatatactttattttaaagtgcaatatatttgtcaagaatgcactatcttattacccttaaaaagagaaggtcactgaagagtaaagcatgtcttcactcttcaagcctaagctttttcttttttttttcccaaattgttctcagctaccaattttgtttttaaattccccaatacattgttgtacaaatccaggaaacaaacgcagactttaccacagcattttcagcagtctcgatgtacctaataatatacatacttcctgtgtttgtagtcttgtagctttagtacaatattaaccctttaaatgaaacaacactaatctgtcctcttatatacacgttatggttcctgccctgttatttactacatcagcacgggtagcttcctttaaaaacaaaatcgttaaagatgagagtaggaaaaatgtaaatgtgataagagttcaaaaaactgtggtccaggtttgcaatgttaagtggaaaagaacgactaaatctgtgatcacaaggggaaagatgaagccatagtcttattacatgggcacaggacagagagatctcttttgcgaaatgagcataccaaccacaaagaataaaacatactacgcccgtaataaaatgggcacaggcaggatttgccgtagttcaatgctattctttgagggcgaatgtgtcgtgggtgaactgctgtctagcccattccgacacccaagccgcccaggcggcccccacgccccccggctgcagccctccctgtacacacaaatgcacacacgcccagcccggccgctggaggaactaagatggcagcgcagaaagcacgggggccgagctcggtggggtggggaaagaaaagcaaagccctctgtgttttttcattctgtataggtccccctctttccccaaacacaagggctgcgtaaggcattcggtagtagagctcgcaggagcggcgggaggggacgcttgctttgcatactgccagaaacccacgtctccgatctgcagcgacggttcaggcgaccctcgcgacttgtgaccacactctccttcacgtccccttgacagtagaagtaagggtgactccaaatgcctttttctttctggcatgcgccccactcccccgccatcccaggggagccttccaagttgcccagctgccaaggcggtagctgtggcagttgcagggaccggggagcgcgccgtgcggaggcaggaagaggaggaggaggagaaaaaacgctgcactggctatcaattttgaggagagcgaccgctgccgctgccgtggaaggaaacgctgccaaccgcaacagaaatgccactctcggatacctaagtgagcatgtgtgcgactctgcgcacgcctagttgcgcagctcaatgacatcgggcttcttagcaagttatccagtttccgggttttgagtgtacccatcaggctccgtaggggcctgtctgggactgaacagcagtaaagattgaagcgaaattaatgaatggtgggaagagcccggagaccagggcagccacgcgggtcaatgtccactagaaactctaaatgggcagtgactgcacattccaccacctgagcctgcgctgtaaatgttttctgtgcaagcaacattcattcattcagccaacatctatttccctcctgttcagggctagaccctgtgctaggagcctgtgatggttgaacagacaccattgctaccctacggaaaacctagtgttttgtttagtatgtgctaccatttgtgaaaattcatgtgccaagttggacatcctagatcctttaagtcctccctcaatctcatgtagtgggtattcttttgagcattattcagaggagataatttgcccaaagccagacaagtattaagtagcagagtcaggatccaattccagggctgtctgatttcagagccttttagctaccctgctaggtcttgcaatagtaatgggccctctcattgcttacctgtcaggccttagaattcttagaatttagctgcctctacaccattgtcttggttctcactccagggttaggggctgcctggtcaacactgaggtctttaactgtgctgaagcattaggatagaaatttagtctaataaatacttactggatacctactacatgcaaagtcctgaagtggctgaaggagacacaaaagcagcaagctctgaaggaacttaggtaaggaaacaagacaaatacaaaaacttaacagggctaattacagtactaaaatactatgattccacatgtcttgtcctttgttcctatgtttatcccactgctaattatctttaaaaaaactaaatgaaatccatccattatttaaagcttcagaaaaaaatgcattgctttcagcaaccttccaccctaaaactagaattaatttactttcctattccttgttgtttgcacttctcaaacactaaggaggattttttaacatttattttatttttttttaagagaaggccatccgaagaagaggcacagatataagaactagtacatagatgaaagatacagtactgggatgacagaacttataaattattttgtcaggactgtaaattaggaaactcttcaaattgagagtcaaaggataaaacgtagtaggaatcaaagtgcattggcacgtatatttctagagcatatacttagatggggttcgtaggttagcttggaacaaaggactgagcaaatgttacttttttatagacaacaacaagatatgtgatggatctgacattttcagacgctatacagtctccgtcacaattattgtttcagatcgcattcaagaattatctttaaaaatgtaagtttgggcagaaaaaaataaaccattaaatgaccactgccaagagataaaagtgcactcttaggtgagtgaggagtcaataaaaaagagtaatttaaaaaatattattgctccgttgagaacaagaatttgggaatgactctggcttgtgaatactagcctatctgttatactttcacacacaaaatactttcacacacaaaaaagaatgtgctagattactacctgtctttgtaagtaaaagctgtgttaaacaagcgtaaatcctaatgacaaaaaccatataaaataagatcgctaaaggcacttctaattgggatggttttctctgccttaaactctgaattaaccagcacaattcacagtaaatcttcctgttggcagcagagccttggagaagaggtagcagaagagcgtgccactcctcctctggcgatgggcacgttcccccttgctttctggtcctcgttcttccacctggagggaacctgaagtcgaacgtcagtagctgacagcctccccagcactttccttactcttcttcaagctgccagtaaaacccggagaagtggactacttcagattccgcacaacccaacagccccaaacccaaagccccgaggcggagggagtggtggcggagagggggtggggaggaaaaggggcggcagttactgagcatgtgcgaggagtggcgcatgctctgtgaggccggcagcttcccattgcgggtagccccggcggtggtggcggtggtagcggtggtggcggcggcagtggcggcaccgcctcctcctcacattcccggggtggcggggttagatgagcggccccagtagcggcgagggcggcgcgggggggaggaggagaagaaggaggaggagaaggaggtcgctgtctttgtagtctccctgctgcgggagccagaggccgccgccggagccgtcgtcgttggaaaagggctgtgtgtgcgcgcgcgtgtctgcccgcccggcccgcggggacgaggcggcggcggcggcggcggcggcgaggatgatgatgtgcgcagcgactgcctcccccgccgccgcctcctcggggctcggcggggacggattctacccagccgccaccttctcttcctccccggcgccgggggcgctgttcatgccggttcccgacggctccgtggctgctgcggggctggggctggggctacccgccgcggactcccggggtcactaccagctgctgctgtcaggccgggccctggccgaccgctaccggaggatttataccgctgcgctcaatgacagggaccaggggggcggcagcgctggacacccagcctccaggtgcgtccccagggtgcccttccttgcgccccatgccgcccttccttgcgccccatgccgcgcgtgcacccgcgtgtgtgtgtgcttgcgtgtgtgtacctgcgcatttattgagctttcagtccgctctggatgtctgttggcaggagcacttatcgagataggagggtgcggtggctgcagtttctcgtgtgtgtgtgtgtgtgtgtgtgtgtgtgtgtgtgtgtgtgtgtctgcctgcctatgtggggggatagtgaggggtggtaggggaacctgtgatgctggaaaaggcgatggtgctggtcggctgtgacacgtgtgtgtatcgttgaggtaggaggtggcatgggtagagagggtaagaggatgatacacgaagagaattgcaacagtggcaagggaagtggcgttttcagtgtctctgccgcaatgttatttgcacgtgccgcctctcccatgacattccttccagccctttccacctcccttcaatctgcctttcaccaagaggagtcatggtatagattggttgagttttggataagcaggcccgagggataggcgtgaggtttgctttaaaaagatttatgaataagggacgcagatggagatctgtgtggatgcgaaaggtgtctgatagagatttttagagctgtactttgataaccgggccttttggtcttctagaaatgaggttgcagttactcttgtgatctgatgtcccatcggccatttctcttcatcctctgttcgatttcctctgttacttacaggctttagagtagagagggttttgtagggaaaaggggaaaactgcagaacttcagaatatgacagctggtacagcgttcaactgccactagctagaaaatctgaatagcgcgtagatttgtctcttttacttgggttggctacaccttcagcttccttgatgtagatgttcagttggtttcattttctttttcttaaaatgtgggtgcttttttttgctgagggctctcctattgttttcttttccatgcccttacatgccgcataccctgaaggttggtgactgacttgtttcaccagctttgacagtggtaattgggagttggcttcatcttttttttcccacagcttcttgcagtacatggggaaaacatagtaggaaactgtcaaaatgagttaaagagtttactgttatttattgaatgtgcaatttattaggcaattgttaggtgtctttttctcccattatgaaagggacgaggctgtaagtcaggtatttgtagcttatcactaggtatcatggcttatctgttggagacagttgtaaagctggagctttgttcacactttttctaggttaaccagttttctcatgtctgtgattgtttacaggattgggcacagcagtagttttaccatatggatgtaaacgtgtgtaactattttttgctatctgaactgaaggccagaatggcacaagtctgtttcttgctaatgcttttgaaaatagcacaaaactacgtgagacagtttgccctgataattaggtataattttgatctgtaaatctctaaattatggcaccgcaggaacatctttacaaactcctctcaaagaatttaaatttattttgtgtatggtttccagacttatgcaataatttttgacttgtatattcctgttttgacttcctactactagtgtaatatgatttctgtgtatctagaactatattaacggttttagacaaaattaacatagattaatcatataacatttgtgaatgattttttagcccttggtccatttttgtacaagccgtcaatgaaaagtaatgaagaattagttcaagtatttcaagaactactcatggaaatcagcattatgttatgatattgcctgctaatgcttgttcaacgcaacattattgtttatttaagttcacattattttattacaaaaagtagcctgcaactactctattgatgatttgattaagaattaaatcacatttcttattaaagatgttgaggaaagtagacttggggtttcggctctgaatgaatgatagaattatttgtatagggacacaagttaaaggagaagatgatatcttttagagagactgaatggagcaataatagagaatgaagctctagtgagcatcaggtttaaaggtgtgagagaaaggagacagtgaaggaaacggaatcaaaattaagaagaaaatcatgaaacagtagcctctgtggctctaggagagtggattgaaaattctcagaagtggtaaatgttgaagaaagtgagaaccgttttcaggtcattgataacttttgaggaagtggtcagaatagagggatgtggtgaggagtgaatggaatgtgtttttggtaactcaaggggatttcaaagttgagggaaggatattcaagtataggaggttgggtgggatatgcaaatatgggaggctaaatggagagacagagtgaagaagtgagagaggggataattgataggaaaaggtcaaagggatctcagggacaggaagaggtcaaagggatctcagggatagagttaaggtgacaggtggagaggttagccattgaaaaaaggagcagttaatatagaaggggtggaggtgagtacttgtaaatataaagactgggagtttattgaggataggcatcatatcctattcgttttgataatcccaatgtctagcacattttttggcatgaattctgtaaacatcaagtgcctgctctgtgccaggcagtattgcagatgctgaagatgtagcggtgagcaagatagacaaagtctctgccgtcttggaaaatccactccaatgggagaaatagacaactcataatttcacttagtaataagtaactgtgaaggcaataaaacctatttcatgtgatagggcaatttgtagtggggctattttaggtaacatggtcaggaaatgtctctttgagagttggcatttaaattgaattgccatgatgaggaggcagatgtagaggaagaatgttctgggaatttataaaggccttgaaatgggaacaggcttgtgttcaagaaacaaaaacatgatgagtgagttatcagtcaatgtcaaatgaattaattactctttgctttaggtgtacagttgagaagttgagagtttcaaacctaatggactcagtgactttgatgacagtctaacaaagtggttaagagcttgggcacagaagctagactggttttgcatcctggttttgtcatttgctagctgtgtaactttgagtatattacataacttctatgcctcagttttttaaatatgtagaatggggatcataatactaacatgaattgttttgtagattaaatgaatcaatgtatgtgcttggcatgttgactgctatgaaatattagttactgttttttgtctgtttgtttttgctgagaatgaggggtgtggggacttgaagagaaggcaaggatcatgtcttactcttttatttttggtctcagtgactgtgtagaatgtgggaataactctttcatttatccagtgactgaatagatatatttttggatgaatgaggccaatcaaaatcggaacattctgttggggaaaggggttggacaggaaatgtatgaaggaatgtatgatctgttctgaatgtgaatggtggtataaccatgcacttgggaggcaccatagcacagtggctaaatgttagagcactggaggtagactcccttagttcacgtctacctttttctacttgtgttaaattgggcaagttaattagactttgtctgtttctttacttgtaactttgtagtgttatcaaaaggacattatgtccgcacctggtaagtgcccatgaaatgttacctgttgttattattaaaatcacatttaaaattccaaccagcatggcactgtatgtttttccacacaaaatagaaatggagaaaacctagttgagttgatttagggttaaagaaaaaagaaatggaaggttttggggacagtgtttgactatatggtaaaaaggaaagtgatgccaagagggaactgatagactggaagataaagaggagtcagagtatctgggtcctgaagtcctagaagaacaatgttagtgaaaacaatagaggggattgtgaccaaaatggggaatctaaacttaacccccttagttttttcagctgctttatctctttcttgttagaaatggtatttgagtccatccttcagatgcatgggattttggttgtaagaggttaagtagtcattttagcaaaagttcagggattattttattttacttattttgaggcaggctggagtgcaatggtgtgaacatagctcactgcagcctcaacctcttgggctcaagtgatcttcctgcctcagtctcctccagtagctgggactacaggcatgcaccaccatgcctggcttgttttttattttttgtagacacagggtctcaccatgttgtccaggctggtcttgaactcctgagcttaagtgatcctcccacctccacctcccaaagtgctgggattatgggtgtgagccaccatgctcagcttgctcagggattattacttggcactaaccattggccctataaaaatttataagacacaaattctagacttgagtttactgtctatctggaagtgcaagatacaaaggcagatacttatagaagtgttaaatgaaatgaccaagaaagtataacctactatgacaaaggcatggcattctaggctggcttaaaagcatcagcagagacaggaaagcacagtttgtatgtaggtaacagtaggaattctgttagagctagaatgcaggtgtcatgaaggacagtagtggaattcattctggaaatggaggctttggctaggatggggcatcttgaagaccaaggtaagaattagattttattcagtagacactgatcacccttgatggttttaagctatgaagagacgttatttattgtaggaatatgtcaggaagtaatgcgggattgaatctgtatgtgtacatctgtatttgtgcataggtagtaattttcttcatgtagctgttatatcagtggaatcctgtatagtgtataaacacaagttaaggcaaaaactgctctattggtatactggtaggtagaaatgtgaaatgaattagtttcctaactcaagagaagaaaaaaatcttgcctctgggcttaaaaggaaaataaaaatgtcaatctaaaaatacttttgtcctttaatatatttgtttaaaaaatcaacagaatagtctcacttttttctcaaaaaccagattaaacatttaggttttctgaggaggggttaggttaattgaggtttaatttatatacaataaaattcatcctctttaaatgttcatttgaagagtcctgaaactatcaccacagtcaagtatagactgtttccatcacttcatataaaaagttctgtcaggccctcctacccctggttactgaccgctgcaaatcagttttctgtcactaatattttaccttttctagaatgtcatataaatctagctccttaagggcagggcccccaggtattgttcatctttctgtcttgtatctgtatttcctgtaagtggaagctagaccttaagggtttattaggtgttcattagaaaattttttagcaagaatattgtatatggcgctgggtactgcatgttgcatcccatccagaggttgtgaggttgtctcactatttaatatgttaaatctgattacttgggtaagacggtacctactagatttcatctctgtaaatgtatgctttttccatttgtaaagaacaagtaatcacagggtaaaaatttgagcacattgtgaatgccttttacctaatgcttttagcattctttgataatccttgtctgagtcaataatttcattaggggttgcaaaatgatgaattaaaaaataattctgttatttcttctatgtttattagctggtactcttctgcaaagaagagttggccttcactgactgtcagtgaaatgtagtttctcctaatccagcagcataaatgcttaattctcttccctcattagccaaattggtgtaatagtccagtggtggtgtggcaggtgctgtggttaggctttcagatcccgctttcaggcacatgttcccctagctcttggcaatattggcagctaaaactgattgcagctgagtcccactccacgcattgccctgagccaagagttgcctcccccagagtcatgcctcctcttcaggggcagtttacatctaatatctatacagtgtagggatataaatgcctagcgcttttgctataagacaagccaaccttgaagtctatcccaacaccagagagctctcccatgggtctggctgagggcttcctgggactgcattgcagatcaactccttcctctgttcagtcttacgtctttcactgccccacagttgttggttcggagggcactccgcaataaacttccggtgtgcacatctgcatttgagagtctgtttcctgggaaccctcctacccaaagcagttggtgccaggagtggtctcagaaagtagactttacagtgaggttttgaagctggattattggctggctggctggcaatgagaatcgattctggtgctagatggagcactaatactgataacgcttggcatgcactgtagctatgcaacttaaaactttcactggtggtgagttggaatgctattcctgttgaagggaatgcactggtgagtgcactaggcttttgagaagttcagagaaattaattatgaggacaatcatcagatggcttttggtaaataacattgatagattggagaaagatgatgcaaggtggaagataattgggggcaaaatgtaaaaggaaatttatttagcatcatgtaaagaaacacatatacagtaagaggaaaaatctaaggcttcaacccaaaacttaattttaagggtagcagagcaccaaggtaggtagaattctctctcagctatgctaaggtcagtgttctggttgggcaggaatcctgacccttaggatggggcatctgagtgcacttgaaaaccctgaaaccccaaattcctcttaaccctctgggcctgcagaagtggccttcttctactagctagaggggttgcttgcagactgtgctgtccctcttacccccagctcctcctctctcgcctactgactatcaccaaaccaacaactagggttaagtcacaagaacacctagttcggggtgctggatctgcgaagggaggaagggactgtacacacaaacacacacacacacacgttcccaaactgcaccaaggcaccccaaagcaccaccacagacacaaaagaataccatgggatatttcagaattttaaaggaaacacagcaatatctgtcacaaattactagcttgaggtagttcacggttttgacattagctcatgccacatttcttttgatgatgtcatatttttgcagggttggatttttactagttgtgatacagatcaagcactgtgcaaaaatcagtgtggaacaggaaaggagggtggcatgttcagtctgattccaagtttgagaagttgttctgtgtctaacaggcacacatgtcatattatcaagtaattgaagtttttgtttgtttgtttgttttgaggcagagtctcgctctgtcacccaggccagagtgcagtggtgcgatctcggctcactgcaacctccgcctgccaggttcaagtgattcttctgcctcagcctcctgagtagttgggattacaggcgtgtcccaccacactcggctgatttttgtatttttactagaggtggggtttcaccatattggtcaggctggtctcgaactcctcacctcgtgatccgcctgcctcgttctcccaaagtgctgggattacaggcgtgagccactgcgcctggcccagtatttgtagtttttaagaaggaaataaaaatatttttcttccaatttatgtgttattttttcaaatgtctactcatttgttaggacatgaattcttgttatttggatctaactacttaataaacactactgtttggtatttcttttggcctatgagtgcatggaaaaattaccgatgatggctgtgcatgggcccaataaaagggttttcattcaccagtgctgatctagcaactgctgctgctgaatatccagcctgacagcagcagagatcaatcatccttcaagcaggccaaccagtccttatacagtggaaaggaccatgattcattttgattggatttgagacatattctgggtctgaatttgtctttccagcagggtctcacccaggactactgtttgagggcttgcaggatgtttgatcctctactacagggatcatttcagaataagggacccactgttatgttatgttatgttatgttatgttatgttatgttatgttatgttatgttatgttatgttatgttttgagacggagtcttgctctgttgcccaagctggagtgcaatggcgtgatctcagctgactgcaacctccgcctcccgggttcaagcagttctcctacctcagcctcctgagttgctgggactacaggtgcacatcaccacgcccagctaatttttgtgtttttagtagagacggggtttcaccatgtggaccaggatggtctcgaactcctgacctcgtgatccggccacgtcagcctcccaaagtgctgggattacaggcgtgagccaccgcgcccggccgggacccacttttatagaataggagaatcatttgtcccatcttgcaccacaccgtccagaagctgatgacttgatagagcagtaaaacagtcctttgatggcacagctgatgatgagatcctgcagcttgggcatgatgccctgcaagggtgaggcgcccattctccagtgttcccagtaggaagatcacatgaaattaggagtggctccgcatagcatcactctgtgaatcacctgggcagtttctgcttcccatctccatagctctgatggcaaggggttaaaagatcctggttctcagaagagaaacatttccaacagagggcgctatgagaatcctgttatactttaaagtacagctgccactaggatttggggctccttttctaaataaagaagtcaccaggctgggcacgatggctcacgcctgtaatcccagcactttgggaggccgagatgagtggatcacgtgaagtcaggagttcgaaaccagctcagccaacatggtgagaccctgtttgtactacaaatacaaaacaattagccgggcatgttggcgcgtgcttgtaatcccagctaattgggaggctgagtcaggagaactgcttgaacctgggatgtggaggttgcagtgagccgagattgcgccactgcactccagcctgggcaatagagcaagactccatctcaaaacaaaaaaaaaacaacaaaaaaagaagtcaccatttggacagggttaatgtccttgttcatgtggaggaggtagggctgtgttccacagtggagcatggaggtttggcacacaggtgatctataaggtgtctcttggtacttccttgtccagtgtttgtggtaaatggacacgagaaaggcccagtgaccaccccatgaagtcagccacctggaccaacagaaatactagccatagattaaaggaatctagaatggctagtagagaagagttgttgatatcaattttaccgtctgtagtggctggggctgtggtttgacccactttccttcctctggaaagtttctccaggaaatgacactcaccagaattctggaggaactttttccatgccttatttgaagcaagtgaatcctaattgtataataggcgaaatatagtgaatcctgtattgtggaacccagagcccctgcccagcactgatgtgctcaaaattcccccacgactgctgggaacattggttggctacagactcttccctagttccaatatgggcattgcctttggcaataggtgatggccctaggtgatggatcattcttgggggcagcctgaatgaactggttgatcggggcattttatggtttttagtgctactgtaactagtttagtttttcctatttttcagttgtttgatatgaatatataacaatacagttgcttttgaatgttaaccttgtatctctgatcttgttaagctcatttattaactttaatagttttgtaaattccctgggatgttctgtgtaaataatcatattgtctttctttctgatctctagattctcctacccctcagcccctactttattccaattacttaattctcccttgtttttaatctgagagaaaaacaactacaaattaatcttacagttttttaaatcattaagtatgatattagctgtaggtgtttcaatggcctctatcaatttgaagatattccctagtattcctactttgctgatagtttttattttttatattttaattttttttttttttttttttgagacagagccttgctctgttgcccaggctggagtgcagtggcataatctcggctcactgaagcctctgcctcccgggatccagcaagtctcctgcctcagcttcctgagtagctgggattacaggcacacgccactatgcctgactaatttttgtatttttagtagagatgagatttcaccacgttggccactctggtcttgaactgctgacctcaggtgatccacctgcctcagactctgaaagtgctgggattacaggcgtgagccatcgcacccggcttgctgatagtttttaattataaatgtgtattgagttttgtcaaatgcttttcctctacctgttaaaatgatcatatagtaagagaacaaaaggaaaattgaattacattaattaatttttgaatgttaaattaactttgcattcctgggataaaaccatcatatttggaatgtattgctgaatttgatttgctggtattatgctaagaattttcaggtttatattcatgagggatattggtctgtaatcggttttttttcttttcttttcttgctaatcctgtcaggctttagaattagttatcctcgtctcataaagtgagttacatagtaatcctaccctacccagtcctacccaatttttaaaatgtttgcgtaagattgttgttatttcttcttgaatgtttgacagaattcaccagtggcaccatctggtcctggaattttattttggggaagcttttgacaaattcacgttctttaatagttagagcgttattcagattttctgttattattcatgtcaattttggtaacttgtatttttttggagaagttcattcatttcatacttctaagttgttgaagtttttagcatgaagttatccatagcattctttcattatcctttaaggtctatgggatctctgataataactccctccttttttaaaaaaaatatttttttaagagcagttttaagttcatagcaaaattgagaagagggtacagagattttccatctatccctttcccccactcacgcatagcctcatccatatcaacatcccccaccagagtggcatttttgtggccattgtttaacctccactgacacatcataatcactctcaagtccagagtttacattagggttcactcttggggttatacattttttggttcagaaaaatgtataatgacatgtatttatcactgtagtatcatacagaatattttcactgccctcaatatcatctgtgctccacctattcctgtttctctgccactcaaccccaggtaatcacttactgtctccatagttttgccttttccagaatgtgctatagttgaaatcatacagtatgtggccttttcagattggtttccttcacttagtaatgtgcatttaggttcattctttttttgtgtatgagacttgatagctcatttcttaccactgaataatccaccatgtagatgtaccagaatttgtctgttcacctacaatttggtgagtttggaggacaattgggttgcttccaagttttggcaattatggataaagctgctctaaacatctgtgtgcaggtttttgtgtggacatacattttcttttttagaaattgacaaatgacattgtatgtttttataatgtacagcatgatgttttgaagtacatatacattggggaatggttaaatctagtggacatgttttcagctcctttgggttaataccagaaacagtgattgtgggatcacgtgctaagagtaggtttagtttttgtaggaaatcaccaaactgtcttacaaagtggctgtttcatttttcatttccaccagcaataaatgagagttcctcttgcattaaagcctcgccaacatttgatgttgtctatgttctggattttggccattctgataggtgtgtagtagaatctcgtttttttttttcatttctctgatgacatatgatgtagaacatcttttcataacgcttatttgccatctatatatcttgttcggtgaggtgtctgttaaggtctttgccccatctttaattgagttgtttgttttcttattgttagagttttgagagttcttggtattctgtgagggtgtaactgaggagatggggaaaaaagttcttggtattttggataacagtcctttatcagatgtgtcttttgcaaatattttctttcaatctgtgccttgtcttctcgttctcttggcattgtcttttgcagagcagaggtttttaattttagtattcagcttattaattatttctttaatggattgtaccttggtgttgtagctaaaaagttatctaggttttctcctgtgttattttctaagagttttatagttttgcattttacatataaagtccgttttccattttgagttaatttttgtgaagggtgtaaggtttgtttctagattcatttctttgtatgtgatccaaagaaccaacttttggctttcttaattttctctattattgatctgttttctatttcattggcttttccatcatcattgtaatttatttctttctactttctttaagtttactttgctgttctaatttcttaaggtggaatcctaggccattgattttaaatttttcttccaatataagtatttaaagctatgaatttttatctgggctgtgctttagttacatcccacaaattttgactgctatatatttgtcatttagttcaaattattttccaatttattgtttaaaattattacaactttcagatatggtgttttcttacatattaatatattattgttattactttctaatataaaccttgtatgattttgatctttaacatttattgagatttgttttattgcccaggatatgttctgtcttggtgattgtattatacacacttgagaagaatgtgttcggtcatctttggacatgaagttctataaatgtcagttaaagttggatgataagtgttgtttggatgtttatgtttttatatttaaagtttctcctcttaggcaactagtatagaaccttgcccttaaaaaaagaaatccactgtcaatctttgcctttcaattggagtgtttagattattaatatttaatatgattaattattgacatatttaccatatcaacagtgataggtctgccactttattgtcttctgtttctctttttttgttcctttcttcctccttttctgcttctttgggattatattttgggattattgggagtatttcattttagttgctctattggtcttttcagcaatatctctttgtatttactttttttatgccctagggattacagtatgcatacctaactttttacaggctacttagggttattattatactaattcacttaaaacacagaaaccttgcaaccctacagatccttttattctcccctcccccagagacctttatgttatagttgtcatgcatactatacttggcaaacatcaccaaacaatgttatagtttttgttgtcgacagtcatgtgtactttatagaacttagaggaaaaatctagtattttgtgttttcctatatatttattgtttctattgctctttcttcattcctgaagatgcagcatttcctttcagcctaatgttgaccttagcttttctagcagatcagtctgctggggatacattctctttgtttttctttgagaatgtatttattttactttcattcctaaaggatattttaggtggatatataattccgagtagatgcttgcttccttgagcacctcaatgatgccatttagctgtcttttcacttctctgatttctggtgaaaaatctttgtaatgtaaaccttattcccctctgtgtggtgtgtaatttttttctagctgctttcaaaaattttttctttgtttttggttttcagcagcttaatttgatgtgtatctagtcattttctttaagtttatcctctttgaagagtactgagcttctcaaatcggtaaatttttggctttcactaagtttagaatgttttctaccattgtgtctttcttctgaccttacaaaattctagtggcaattatgataaaacttttgagattgtttcacaggcctctaactgttcagtctcctatcaattttttttctctgtttttcagatgaggttatttttattgatacacgttcaggttcacagactcttttctcccatctccattttcttattgagtccaaccagtgaactttttatgtattgtttatttcacttttaacatttacatttggttctttaaaaaaaaaacaaaaaaaaaaaaacaaaaaaaaacttctctttccctgccgagaacctctttccattccattcagttgtccttgtctttcctcaatgtagcatatttatagtggttgccttccagtcattgttggataattacaacatgtgcatcgtcttagggttggcatcttttgattgtctcttcccttgaagttgttcacatttttcttgtgttttgcattgtgagaatttgggattgtatcttggacatggttaatgttatgttttgtaaactctagggtttattataattctctggaggatgttgtgttttttgttgtttaggggatccctttctgtgtcttactctttttcaggacttcttacccattttccatcatatcagtttcttttctcagttcctctggccagaaagagttttaacttggaattttaacttttgggttgtagccctgtaatgcagtgatctcttcctggccttcaggcaaagctgttagtgaaaggagaaaaacaaccaaactgggaaatttactcttctgtgaatcacttttccaagttttgactcccctccagaatttgcttttatttgtttttcagagtcctgaagtactttttttgtctttttaaaaattttgcccaaagtagttgtaaacagtggggaaaataggctgttttggggtttatgccaatgtactggaaccagaactctcttatctggctttaaaaaaagattttcccttttattgatttttagcagcttgactgtattgtacctaaatatggtgtgtgtgtgtgtgtgcatgcgcacgcacacacgtctgtgtctctgtgtgtgtgtatcctgcttggtatcctttcagtttgttagatctgtgtgatgtcaatttttatttaacttgcaaattttggcctttatttcctcaaatattatttcagactcaatttcttatttccttctgggaatccaattatatatatatgatactgtttgatatcatatgtgtctataatcatatatatgacatatttatatgaccagatagtaaataacttagactttatgggccatagagtctctgttgcaaccatgcagctctgttctttagtttgaaagcaaccacagacaatatatataaatggataagcatggctgtgttccaatataactttatttacaaagatagacaggaagctgtttttggcatgtagactatagtttgctgacccctgagtaggtggtaatcgttgctggagctacattgtgaattcagggaatcaaagaagtgcaaaacagaaatgttggaaataactacaatgtaggtagcggggtgcataaaaatgaggaggtaggagatatggctagaaagatatgttaggactaggttattatgttccctgtattgctttatttgaaaatagtatggaacaatcatagggtattttttgtttgttttcttttttttgtctgtttttctgagacggagtctcactccgttgccaggctggagtgcagtggggcgatcttggcttactgcaacctccgcctcctgagttcaagagattcttctgcctcagccttctacatagcagggactacaggcacccaccaccacgcccagctaatttttgtatttttagtagagatgggatttcaccatcttggccaggatggtcttcatctcttgacctcgtgatttgcccgcctcgacctcccaaagtactgagattacaggcgtgagccactgcgcccagccaatcataggtttttaagcaatgtaatggtagagttaaactttcaatttataaagataatctctagtagtgttatagaggatgaatgaaaaggagcagatctgcaaagcagggctacctttaaagagacagttgcagtaactctgacaagtgaaaatactgagaaaagataatgggaatagatggaataaagtggatttaatgatatatttagcaggtgactagatctcaagtgcaaatataataatgattttcatctagatagaaagggaggaagagctagtttctcaagagtccattcttgggattcttctctctgaattttcttctttggttgtctcaggcttatgatttcagatatgcatttgacttcctggtctgtggtttttaggtaaaacctcttttcttaagttctagcctgtattttgaaaagtctactgctaggattctctcagtcctgttcagatacattatgttcagtatattcaaagtctcctcgtgaacatactgtcactcacaaactgcctcttttcccttaggccagtgaaaccgaagtctgagataggttcttgaggttggcaataagaaattataatgagccaacacccactcacttcacctccttttttcaggaggcagggaaaagtgggaaagtgaggctggacatgggagtatttagaaatagtgtttaataatgtttaaagggcaagaaggaaggcagtagcttaaggaaggagacaaataattaatcttttggattagataacctgtaggagatgttcttagtgtcctagccatatccctttggcatccatttgcatactgtcttctgcaaacatctatgactatctgcctgaaaagaaacacacttggcctgtgtgcagggcaactcacatgcgccagagagttaatgccttcagaaaaactttccaccagtgatggatggggagttggtagataaatacctttgtctcacctgcatttaggataactgagccatgttttctgctgtctaccagagttctccactggttcaggccctagttccccatcagggtaactggcttcataatatgtcctttcttgacttccttcttttccctgttttacttttgttttccctactggtgtttttcctgggatcacctctcaaaataactactgcacttgaattcttgaattgacgtctgcttctggggaaacccaaaccaaggtagacatttgaatttggcatgggaataataataaaaatgtgtttagcacttactaaataccaggcataattctaagtgtgacatcatccagtgggagaggtactgttttcaccctattgtatggatgagagaactgaggcacagagagattaaagtaataccaagatcacagtgctggtaaatgacagagcttgtatttgaacccaagtcatttgtttctaaattctgtactcttaacctatcttgacatagcaggctgaaggaaaagggcaaggatagtaaaaaagagagagagaatgggaataattaatagagtaaggccctgggttagagatcatttcaaggtgggtggaagaaaagcaaagggagtacatttgtaatggtgtctctattttattggttaaatagatggttagattctctgctctgagttgatgggtaggggagggattttgaagtagctggaaaggttctagatttttatggtagatgtaccttgatagagaacttaactggctctagaccagcagtctaatgatgttcagtaactccaatagctattattgtttagggttgttatagcttattgaacaaggtttgtaactaaccagtatagaagtgaatatagaacatgagcagatagctcacattagaagtgatatatgtagtaaagacttaagggacaggaagaggaagaaaaaggagccagtaaagaagactgagaaggaaccaccatcagaggtagaaggaacactaaagtagaggttctagaagctggcgggacagggggcagggatgtaaagaagtaggcactgtttggtagttatcgtatgtaggagaatgtgaaggataaagactgaagatgggcagttgaattggctactagaaagtttgtggtattgagaagtctcatagacttgcatgagtttgatagtaaggtgttaaaaaacttcaatacaatgaagtttttttttttttttttttttttttttttgagacagggtctcactttgtcatccaggctggggtgcagtggcatgaccatagctcagtgcagcctctaactcctggaccaaagcgatcttcccacctcagccatctgagtagctgggactacaggcacatgccaccgtgcctggctaatttttttttttttaatagagacggggtcttgctatgttgcccaggccaggaccatttttttcttcaagaaatttagtgatgaagagggatagaggattacttaaacttttttccccttttgaattggagaatgacagtaggcctctaaggttagaaaccacagaaaaaaagcataaagggtaaacaaagagagggatgtatggttgagcaaaatctcaaggatggtgaatgaaatgggatctggtgtatgagaggaagtgcatacttggatagagtaagagctgtctcttttgggatgggggaaggaggaagtttcagatttgaaaaggtctcagggacatatttttagcctttttatgtttctgtgaagaattccagattctttttctgatctgttttctagatcttaaattttctattcagctgtgtcctgtacacttaaatccacctatggagtctgtagtgtcagttatttttcatgtctagtttgattttttttcaaatgtgcttggttgttccccattccctgcagatattttaatgatagtcttttatttctttaaatttgatacacatgttcttgttgcaatctttccgaaaatcccaccatctgaagtctatataggtgtctttctgttgcctagttctgctgttctcactcagagtgccttgtatctctgtgttcttagttatttgcttgctctcattcctcttgttgttctctgaggcctgggatgaatagattgtattcatgatatcattagaaagtgttacagttttacaattgcttcaaggcttgagtttccctggcctacctagcctatgtttacttaagagtacaaatatgcatgaggcagggtctatagccaaagcttttcagagagttctcttcccctcacttatgctcccttccatattcccctaccctcttttctttttccttctgctgctctgcccattgccaaggtagctttatttatagtcctccccccatgtgaagacttccaaataagatcaagagactgaagctatatataagaaatctgttttcatctctcccctaaatcaagttgaactgttaaaatctctttcttattactaattagccggctaggttaagtaaataaaagttttctacttagtaatgagataactattcctccctaatttattcattgatgcttgaacatgataggagtatcattacattaattacacagaatgaatctggcttctatggaaaagcttcagtttacataaggtgcttcatagattgatttttaaaagttggtcttgtatccagtgaccttgataattcccttgttaattctttatttgtggattattttgtattttctataaatataattatctacaaataaaaaatgctttacttttaatttttaaccattatgacttttatttcatttgcatcacttactgaccttcaatattgtgttaacagaagtggtgatagtacaaatccttgcattttcctcctatgcctaagtggaaatacttaatttttcaacattcagtatagttgaatacactgttaaaagtaggttttttatagataatctgtattagattaagtaaaatctgttcctggttcaccaaggagtttcatcactagtggttgttgagttttaaaaatactttttctgtttctattgaagtgatcatatgttttgtttcttttgttctgttattatattgaattatgttgattatgtatttattctttccacatgttgttagacaaaattatttgtaggtaaatggctctctctttttttaaaaataaaaggggtaatacacataccataaattcacccttttatacaacttattggtttccagtatattcacaaagtcatacaactaaacatttttgtctctccataaatcccaaagctattagcagtcagtttctattctcctttctatcagtttttggcaactactacttcctgtttttatgaatttgcctacctagacattacatataaatggaattatacaatttatgaccttttgttgccattgtttctttcacttagcataatgttttcaaggttcatccatgttgtagcatgagtcagtacttcattctttttttatgggtaaacaacattctcttatatggaagtgctacactttatacattcattagttggtgagcatttgggttgttgctattttttggctgtgcagtatttttgcttctatgttcataggagatattggtctgaaatttttgtattttgaaatgttctcttcaaattttgatatcagagttatgcttgtcttatgagttgggaaatgtttcttctttttatattgtctaaaagagtctaacattgataatgtttgaaagaactaccagtgaatttttaaagtatttggaagaattcaccaacaaagccatccgagtctagagttttctttgtggatagattttgattagggatccagttttttttcaatatatagaagactattcagatattacattttttttcccttgtttaatgttgagaagttacattttttcaaggaatttatccattttatttgtcaaatttatgggcagcatctgtagtgatgtctcctttcttatttctcatattggtaatttgtgcttttacttttttcctgaccatttttgctctaggtttattatatttatttcattttattttaaaatatttgctatatatttaattcacaaataattgtacttatacatggggtataatatgatattttgatacatgtttacaatgtgtaatgatcaaatcaggataattagcatatttatcacctccgacatttatcattttatcatttctttgtcatgagaccattcaaaatcctctcttctagctgtttgaagttgtataatacattgttgtttatgatagtcaccctgtggtgctatataacactcaaacttactcctactttctagctctgattttgcatccattagccaacccctggctactcttcaacaactcttctactttctacttttatgagatcaacttttttagcttccacatatgagtaaaaacatgccatattcatctttctttgcctggcttctttaacttaagataatgtcctccaggctcatccacggtgctgcaaatgacagaattttactcttttttaatcactaaatggtatttcattgtgtatatatatcacattttctttatccattcatctgctcatggaaacgtaggttgattccacatcttggctattgtgaatagtgtagcagtgaacatgggagttctgaatcttttcaacatacggatttttttcctttggatatacacccagtagtggaattgctggatcatatggtagttctgtttttagttttttcaggagcctccattctattttccacaatggttgtactaattcacattcccatcaacagtgtagttctactttctccacatcttcaatagcatttattttttgtctttttgttatgtatactaatcttttcagagaaccaatttttggctccctgataacctctattgaatgtctgctttccatttcattggtatctactcttatttttatttcctttctactacatattttgcatttaatttgtggttttctttctagcttcttgagttgtaagtctagataatttactttcagtgttttttttttcagtatatgcatttatgtgtatgcaaaaggagatgcattttgtggaagtactgcgttagcagcattctaccgattttgttatgtcaaatttcattttcattttatggaaaatattttctgatttccattgtgactagttctttgacccatagtttatttagaagtcttgtttttttgattgccaaacatttgggaatttcttagttgtttgtttttttttaatttctcgtttaatttttccatgaccagagaattaccttatcattcagtccttggaaatttgtttagacttggtctgtggtccagcatattatctattttggtaaatgttctatatgcacttgaaaagaatatgtattctattgttgttggatatgtttctgtatgtatgccaattaggtcaggttgattaatcatggtttgtttgtttgtttgttttgagacggagtctcgctctgtcgcccaggctggaggacagtggtgtgatctacgcacactgcaaccgccacctctcgggttcaagcgattctcctgcctcagcctcctgagtagctgggattacaggtgcgtgccaccacgcccggctaatttttgtatttttagtagagacggtatttcaccatgttggtgaggctggtctcgaacacctgacctcatgatccacctgccttggcctcccaaagtgctgggattacaggcatgagccaccgcgcctggccgattaatcatgttttcaagtttttctatgtcagtgctgttttggtctctacttgttctatcagttacagagaggtatgttagaacttactctttcaattgtggatttgtattttcccatttaacttctgtcaattttactttatatattttgaagctgtgttactggatgcattcaaatttaggagtgttatgatttcctaatgatttgacccttttgccatttagaaatgctcttttcatttcttttaatactgttgccttaaaaattactttgtcaaatattaaaatagccactccaactttttttgggacactggagagtgtgcttgagatacatctttttctgtccttttagcatgtatcctttcatttaaaattatgatattacagttattaaaggaagactttccttcagtatttcttgtaatattggtctcttgacagtgaattatgtcagctttcatggatctaaaaacatctttatttttgtcttcattcttacaggatacttttagaatcctttatgtgaattatatcagctttcatggatctaaaaacatctttatttttgtcttcattcttaaaagatacattcttaaaggttacatgattccaggttactttcccccacctctaacccccatccctccactctaccacctccccctccccaccccccagcccttttcaacacaaagaatggaaactatcaattttattgttcctttaagcatatctcttctctggctgctttaaagattttctctttatgctttgcttttagcaatatgaccatgctgtgcctaggtatgcttttctttggattttcctgcttaaagttccctgagtttcttgaatctctcagttgatttctttcttcacttttgggaaattgtcagtaattatcttgtcatatattgcttttgttccattcttttttctctctttctgggattccaattatatgaatatgagaccatctgactttgtcccacatgtctcgttctctgtttggttcatcccatttttgtttccgtgattgaattctgttatttatttatttatttatttttggcctgtttgcaagtctgtgaaacctgtttttctgtaacttactctgcttttaaaatcatatgagaagttcttaatttcagatgtttatttttcagtttcagaatgtccacttggttctttttttataaattatgttgaaaattgccaattgcattattttacacatattgttaatctctgcatctcaggtacttgcctcctaacgttaatatctgggttttctgtgagtctgactaattttctggttttctcttgattatgtgtctgccacatattttgcttcttttgatgtcccttaatgtgtttacttagtgtccccgaactagactaacactgtgtatgaatgaacatgattttctttcagatcatgtgttttctcctagagaggtgtgctctttgctgtgtcagacagatagaatgaggagtttgattatctcaatccagtgagggattacatagaccgctccctccctctactaggattctgtcttcttacggaagttttctcttcccttttgacccagcctctttttttttttttttttttttttttttgagatggagtcttgctctgtagcccaggctggagtgcagtggcgcgatcttggctcttggctcgtcacaacctctgccttccaggttcaagcgattctcctgcctccgcctccctagttgctgggattgcaggcgtgcaccaccatacccagctaattttttgcatttttagtagagacggggtttcaccatgttggccaagctggtctcgaactcctgacctcaagtaatctgcccgcctcagcttcccaaagtgctggtattacaggtgtgagccaccgcgcccagccttgacccagcctctctatcatccttcagtgttccagttacatttctgaggagatataccactgggaaagctgtctatgtatctagagcttttctagattccagtctgtcatttcagcccacatgggcatctaaagcactactagtctcttgtttcctttattagagttcctctgcttaagccaagctcagtccttactcatgcccaagctctgcagttacccccatggaagaacaagccagtggtctcagctgccttggaagtgctcttccacctctggattttttgttcttttagtcctcattgcttccacagctttcttacgttggttagtctggtctttgaagcaggcgcattggcctgcctcctatctaatcacttactttagggaagtggaagtcagaagtttatctttataaaaagaaaaggcagataattttaggagtgcttatttttataagcatagtgatgttttgaatagttttatctgtgttgcatactcatagctataaagcatttttttcctttctgatatgggcctgtttatcaaaatatgccccagatctggggcgagcaaacaaagacccacagaccaatgaggcccaattcttgtttttatgtagcctgtgaactaaaagtggtttttacatttttaaaaggttgaaaagaaaataaaaaaggaagagaatgttttgtgagcattatgtgaaatttggaagttttattgtaatatatgcacactcctctctttatgtgctgccgatggctgctttcgtgctgtaaccattgagtagttgcaacagagatcttgtgactcacaaagtgtaaaatatttgctgtgtggccctttgcagaaaaaatttccctattcctgctttacatcacaggctcaggaattttttatttcttattactaagagaaattaagcaaaagattgaaaccattttattccagaagttctgtaaatttggatttattaaaatcttagcattttagaggatttacaaggaacctcagtctccttcatctctttattttgcaaatgaagcagatttgagtgaaagaggttttaaaattatgactacatttttggtataaagtatactcagtaataagctgatagagaaactattgatctatatcactgaataatttgcaaaattgatttgttcttatattgcagaagtatttgatttttcttaagcagtgtagctcttaagaaaatatttttggaatttttaaagtaaatattttaagataaatatttaaggcatgttaatagccttttccttttattgtcaaccacaattctttcaaatctgatggatatgaagttatttttgtgagaagactgaaagtcttacaggaccaattccagacattcttcattaagcttcgattgatttagaatataaagccctaaatcatcagaaattccaagttaaattgttaatgtgtaagactggtttttagagtttctcttttatattcccatcctcagatattgtgaaaggtttaagcttagttctcaaatatagagatataatggtgtgttatattactagcaaatggcatttttaaattttccaatgttgaatacaaattttgtttatataattttcaacttattactggctcttttagaaaacttctacctcttcctctgcaaaccaaatcaacaacaacaacaaaaaacacagtaaaagtaatttataattaatttgggaatagctagttcttggatggcctgtacttaagaattgatgggttcagattcataagaattgttacaaccaattaaatgccatcatgcatagaaagaagtattttagaaatgtacgtaaagtcatttgaactttaaagaagttccattagataaaacagacacaaacattggtctctaattgggaaaatttgagtcagttataacttgtctttaggaaatctatctgaatttggatttatatattctacttctctgaaaattggtttggaataaattttgatgcttttataagttatatagaaggggctaacctcttagtagttatgtattcctgttgctcaaatacctgatttcactcaggtaatctttaatataaaggaaagttggtttcctaaatagtatgaagaatacgtattgtgttttatttatttatttgttcatctgatatttattgaccatccctgatgcttcacattgatgcaggtgctgaaagtacacagttgaaatagacattcatcatgccctcacaatgctagtagttgagtagattctataagcaggtatatgtatttatgtttagcatttggcactgttaatctttctctttctctttacttttatggcatactatcttgtggaaatctcatccacatacatagtttccatgacctcctttgtactaaagtctgtgcaaaataatcccaaggataactccttagtgggcctttctggctagagatgctttaaatgcactcatgtcgataccatagatgtagataccttattttaatctctgagtaaggcatgtagctgtagctctcaacttcctcatttctccccttcctcactctttctcttatactttctctttctcttaaaatactcgttgagtactatatcccaggaacttttctggacagaggctagagtagtgaacaagaaagacagggcccctgcactattggagcttttagcttggcgaggaagcaatacattaaacagttccatacataattacagttattggctgtacaatgaaacataaatatacgatgaatgagagtgtatattagggaaactcatccagtgtggggagttagacttaagtgacacttatgctgagacctcgaagataagtagtggttagcaggagaagacgtggtagagaatttcaggaagagggaaaacctttgtgaaagttgtgatgaagcttaacaggttcagacaactgaaagaatatccttcttgctggagtatgatgaatcaggtggaaaagaatgcaaaatgaggcaggagagattgatggaggtcagttattggaagaggatcttttaaacgatgtcggtgcttttggactttatcttaagagaattgagaagatactgatttttaagctggggaattatccactcaggtttgtgtctttaaaagttgagttaggttcctatgtgaagaatggattgaagggtatggatgtgcagaaatgagttaagcttttgcagtggtccaggtaagaggtggtggtaacctagcttattagcattgcagcagtaaagatggtgtggacagattcaataactgtttatgagatataattgaccagattttgtgattattttgatttcaagagtgaaactgaaggcaatgtcaaggataacattttcatctgcttgttactcttgtctatttgttgacattttggtattttgaagttacacatcttcaaaatcaaacccattactttttttccagcaggctaactcttccttctaacttctctatttttaagagtgacattgtctacctccaagtgacctaggcttaaaatttttctcttttgtcactgctgttactaaattagttaccaaatcttaaggacttgaccccttcagcatctggaacaacctcctccccttcgttctccctgcattccctcttccccccgacttcatccttttttctttattacagcagtagtatcctcttgttagatcactaaactgtgctttccattcaagtctctgagctccttggggacaagaactgtctttttcattttgtgtctttactactttgcatctcagcatggtgaacataaaacatattaagtgaaatgaaaataaaatgttttccttgcttcttgtttcttgcaccataattcttattctttatcacatatttttttctgtttctcaaaagttattcaggggcttccttttgcttaaagaataatttggtcttaggactatgtgataatgaagtatccagactggctgctggagacataaagtatatgagctggggattttcctatagtagtttagtaaaaagtggctcttaagtccctttgcaccgtagccctcactaccaaaaagagcagattttttttcttggaaacatcactgacatttgtaaacatcccactatggataaagtataaaacacttcatttgatgcctttttaatgttttaagtttatgtttttgcagaattcaaatttgtattatcatttaatatatattgtatattgcttagctgccccatgttatcttctcaatagctactttatatgttagtatcttatatgtgtatgtaaagtatattgtttcacataaagaaattttgcaagaaaaagatccaaatgtctactttggaaatacccatttttgatagctttgtatgtcctaatatgaacattcatatttttttcccatttttcttttaatatttaggaataagaaaattttaaataagaagaaattgaaaagaaaacagaagagcaaatcaaaagtgaagacaagaagcaaggtaaagctgctggtaattgataaggaataggactttttcatatggtgatttgaataaaaatgccaagattaaaaaatataattaaaccattttattagtatgtgctgaagtaattttgtaaaagccctttatgatttggttcctcttttagtttcatctttcttttgggtgtttcttttctttcttgccttttttttttcttttttgaaacagggtcttgttttgtcacccaggctggagtccagtagtttcctgggctcaagtgatccttccacctcagcctcctgagtagctgggactacaagtaaatgccaccacaaccagctaattgttaaattttttgtagagacagggtctcacggtattggccaggcttgtctcgaactcgtgggctcaagcaatcctctcacctcggcctcccaaagtactgacattgcagacatgagccactgtgcccagcctatttctgggattttcttgaccagtagcctggaagttaatatctggatgttgaaaattatttgctatttattcttattatctttgatcagtcctttggggaacatatccatttctgttctcttttttcaatctgactacaggtactagaaacttgagtaaaattagcttgtaaactgggacttggatttgggttcatcatatgcctttcttgacaggaattagggaaacttggtgtgtccatattgttctgagcctcttttgactgttggtagctctgcaaaagaaaaggcaatatggaatatttgaattaggtgagaccttagaaaccaagaatctattttcacctaatgcagagatctctataaaatccatcttttttatactgaacattttcagtgaccagagcacttgatataacagtctcttttctattgaacatctagacagaaggaagagaggagtcgtaactatgattcctgaatagcttcactttgagttaaagaaacaatagatttgctgggcctgtatagcaactatagaggcaattgtttcctacacttttaagttagtgtttttactattgtaactgagagaaatctacttcaaagtagcaaacctaaaataaaatgaaaggttgggggatggcatttatttggtggctctatcttgagacacagatggatccggggatgaaatgatatcattggctgggtgcagtggctcacgcctgtaatccccgcactttgggaggccgaggctggcagatcacgaggtcaagaggtcgagaccatcctggccaacatggtgaaaccctgtctctacgaaaaatacaaaaattagctgggcgtggtggtgggtgcctgtagtcccagctactcaggaggctgaggcaggagattgcttgaacccgggaggagcaggttgcagtgagctgagatcgcaccactgcactccagcctggcgacagagtaagactccgtctcaaaaataaataaataaataaaaataatcaaacccatattttcagctcttgcttctgccttcctctgtgtgtggacttattacctgtgactgaggctgaggctgccagtagctccaactccatttgctctgagcaagaggcaatctcttatactagtcatagcagaaagtcttgggtaggattttcggtttacctgcatcatttgctttgaccacggagatacgggatactttggttagactgagttatatttttaaccctgagatgggcagtgggtgagcaccctggtgaataggccacatgaaatgagggaggaatagttaccctaaggaaagtgttgccgggtaggcagaaaaataatctgttcactgtatcccttctcattacttctgcttctgggatagaggagggaagtggagcgcattgtcaatatacagaaaacctctaacaaatacagtcattccaacctctatcccttttcaactttttggtgctatgtgcaaatcggagtatgggaatcttgaatatcttttaagtttttttaaggtttcatttggtgattttcctgaagtggtggcatatgatatccaggaagaggtgaggagaagtcaaggtcttttgtccccttcaattcggcctccaactagctttgtgcccttagtaattcagtttttttcctcttgctctcagtatttctatatcttctttttaagagtagtttgagataacagtgatgtaagtaagttcattcatttattcctcaaacattaattgcatgctgagtatgtgcaggcattatgctggatgtttgggatacaaagatgagtaagaggcattttttgctcatggaaagcatataaagtagtaaaatggtgttgttagtctccttgacctgtattactttttccagattatttttcatctaccctctttctgaaagttatttttaaaatgccatttagctggcattttatctactttatatctactttatatatattttatatatatgtatatatatatgtatatatatatatatatatatatatagccattatatctactttatggctatcatcaacccctctatttctcctcatccatcagtcatctcctttatttaatttccatcttacccaggttagataccagtctttctttttagaaaaactcttatttaatctgtaacaaattacaaaaacttgatggtttaaattaacactcgttatcttacagttctggaaatctgaagtccaaaatggatctcactgagctaaaatcaaggtgtcagcaaggctttattccttctggaggctctaggagggaatctgtttttttgcctttccagtttctatagtagaggctgctcagatgtcttgggttgtggcccccttccatcttcaaagccagtggcggttggttgagttttctcatgcggcatcactgatagttttctgttgttttatctcctcctcttactgtccagcctccctctttcacttgtaaggacccttgtgattacattggcccatccagataatctgtgcatttcaaaatccttagtttaatcacatatacagagtcccttttgccatgtaaggtaatattcacctgggttctggggaattagggtatagaagtttttggaggctattatttacctaccatatctcccttgtatatttgttgcattaatctgacaaaactcaatcttgtgtgaatccagttattttgtccctcccttaaggtgagttccatgtgctgccaaagagagttacacaagagttaactgataatgatacacattcatgatcatcaatctcaagtataccatagccagactatcttattattttttctggtcaaatagttctcttttcttctgtgattatttcagacctttgccatgctcctcaaacctctgataaattctaccttccagcacatgacttcactttcttacaggagaaatcaaattcattactcagaaaagttcctcaacttcctaatcccaaatgcgtctgtcttcaggcttctcagtagctgagcctgacatagagatttgaagtttgcagttcagtaagttttgaaagttaacattcatgcatagaagcactactgcagtcaaagttcagaacatttctatcatctccaggggttttctttttttttctttctttttttttttttttttttgagacggagtcttgctctgtctcccaggctggagtgcagtggtgtgcgatctcagctcactgcagcttctacctcctgggttcaagcaattccctgcctcagcctcctgagtagctgggattacaggcaccagccaccacgtccagctaatttttgtatttttagtagagacaagatttcaccatgttggccaggctgtctcaaactcctgacctcaggtgaaccacccgccttggcctcccaaagtgctgggattacaggcacaagccaccgtgcctggccggttttcttaccctctgctgattccatccctccctataaccctggctgtagacaaccacagatatattttctgtcactgtagattagttttcattttctagaacttcatataaatgtgatcatattgtgagtgctcttttttttttaaatctggtgtctttccctcagcataatgatttgtgttaatgttgttgtatatcaatagttcattccatttttttgctgattttatatacatatatatgcacttgtgtgtgtatgtgtgtgtgttcagttttcttgggtgcatactgaggagtggaatgtttaggtggtttttcctttgatttgccagaaggactcataaatctcacaagcaatttatactcatggctaagatttattacaacaaaggatacagagcaagcagcaggaaaaaggtatgtgtttgtgatgtataggaagttatggcataggcttcctagttcttcattgactgagctttcatgtcagaaagaaagacatgctttctctctggcagtgaactacagagaaatgtgtatattgtttttgctcaggtgagttttagaattcaagactttgtgggtttggtcacacagacatatcttgttagtcaatcagacatggtaagtgaaactcaggtacacattgtaaagcttgatgttcgtacatgcagagcagtctgacaggccaggaggcatggtccattgctctgtgtgttcataacacaatcatcaatcactaccacaaagaaaactctaaacatccacattcccaaaggttagccaagggtcaatcatggtttccttggggaaatacaaggagtaagcaatcatgcctgcagcgttaactttttcctcagagtgggcacggtaaatgtacgtttagtttagctttttttttttttttgagacagagtttcgctcttgttccgcaggttagagtgcaatggcacgatctcggctcattgcaacctccgcctcccaggttcaagcaattctcctgcctcaggctcccgaggagctgggattacaggcatgcaccgccacgcccggctaattttgtatttttagtagagacggggtttctccatgtggaggctggtctcgaactcctgacctcaggtgatctgcccgccttggcctcccaaagtgctgggattacaggcgtgagtcactgcacccggcctgtacatttagctttttaagaaactagaaaactcttttccaaagtggttgttccaatctgtagtcccaccagtggtatatgaccaatctagttgcttcatatctttgcaaacatatggtattcgctttttaaattttagtcagcctactaagtaggggtttcccattgtggttttagcttgcttttctctaatgactaatgatgtgaaatcttttcatgtaccttttgccattcttatttttttgatgtgaaatgtctgttcagatcttgcctcttatttttgtaattgtcttcttcttattgaattgccagagtttctcatatattctaaatacaagtcttttgtcagatatctgcattccaaatacatttctcctgttctgtggcttgcttttgggggaagaattgccattttaatgatattgaattttccaatccatgaacatggtatatgtctccatttatttaagtctttaaatttcactcagcaacacttttttgttttcagtgtgtagggcttacacatattttattacttttatttacaattattatatatttttgatgttactgtaaatgcagttatttttgaaaatttattttgctttgagatgatttcagtatgaatgcagtgtgtatgtgtttgtgtgcacctaagtctgtgtaattttatcacatgtgtaggtccatgtgactcttaccacagtctggatatacagcagttccatcacaaggattctctgtgatatcctttatagccacaggtatcttccttcctcaccctctccttagcttctggcaaccactgatctgttcagcatttataattttgccattttaagaatgttgtccagtgaaattgtacagtatgtaatcatttaagattggtgctttttttccccacttggcaaaatgtctttgagattcacacacattgttgcatattgtttgttccttgatgagtaagcatttcatggtatggatgtactatagtttgtttaaccatttacccattaaaagatatctgagttgtttctagcttttgactattttaaaagaatgctattacgaacattcgtgtactggtttttgtgtgaacataagtttttatttctctggtgtaaatgtccaagaacataattgctggattatatggtaagcacatatttagttttgtaggaaattgctgtactttcttccagagtggctgtaccattttacatgaccatcagcaatgtataagtttctcctcatccccaccagcatttggtgttgtcactatttattttagccattctgataggtgtgtactatatctcattttaatttgcattttcctagtagctaatgatgttgaaattcttttcatgtgcttattcaccctttgtgtaaatatcttctatattgatgtgtttattcctatcttttgcccatttgttaattggattccttgtttgtttttactgttgacttttgagaattctttatatattctgaatactcatcctttgttggatatgtggtttgcaaatattttctcccagtctgtgacttcaatgctattatttttgaaattacattttttcagttgttcattcctagtatgtggatatactactcatttttatatagtaattttatatcctgtgattttgttaaatctgcatactatttctggtaacttttttgcagattccttaagattctctacatgtttttttacaagtaaaaacatttcttttttagaaaatacttcttgcctttttcccttggctggataggatattttccttgcactagataggatatccagtacaatgttgatgagttgctgagagtagaccaatttgcctggatcacaatcttagagggaaagcattcagtctattaccatgttagctgtggttattttgtagataccctttatcaagttgagaaaattcccttctattcctaacttagactttttttttttctatcatgaatcgatactggatttttgtcaagtgatctttctgtgtttatttgaagtataatatattttttctctgttacaacagtggttgcttttccagtgttgtgccagcattgtccctgggttaaacctcagttggtcataatgtattacttttttatatattgttggatttgaatagctaatacttgtaatatctctggatatttggatgagatatttgtaatatctcttatacatatattcataagttatatgccatgccaatttaactttttgatgtatttcttatttaacaagtgtattttttttttccaattctttatggaagaagagtcgaatatttgggaccaagtgtttttggtacttgaggtatcattttgtttatcttagatacttcctggacatactctaattattggaagttgaatttctaaaatattttaaaacagctttttatatttataatataaatgttgatcttgaaattgtagttgctgtctgatgaataaaatattgggcataaaagagaaactgtttagtcttaacaaattaccaatcacatgatttttagcctttatcaatactgataggtgagagagagcttagggaagcatctacagtttacttggcattactgttacttgaatgagaatgaaatgagctgatgaaaattaagtgtttttttgggaggccctcatttctgtgtaaacttctatatctacttttaaaagcattcaaaatgcaatctaatgtttgtagtaggtcattgagactctacagtgtgtctagagtctcttaggaagtcgtaaaatgaatttcctttgatacagaactctaagagttaagctttgttgagtctattcctgtcatggcgatacaagaatatttctaagttttttgcccatccttttccagcccttgtcagattggttggttattgctgcattgctcaaaaaatagtgaggtatagaaaaggggactagaagttgggtccatagacttagactgtctctgctgtgtcacataatgtgtcttctgcaagtcagttagtgtgtctaatctttacttttatgtaaaatgggacttgatgatagaaaagagtgtgaaatggaaatattctgttttgtatttcagtctgaaaacttagagaatacagtaatcataccagatatcaaactacatagcaatccttctgctttcaatatttactgtaatgtacgccattgcgttctggaatggcagaaaaaggaaatatcattggcagccgcatctaagaactctgtgcagagtggagaatcagatagtgatgaagaagaggaatccaaagagccccctatcaagcttccaaaggtaagccactgagttctattaatatttagatgtgtaacctgcaggtgttctggctataatgcatatatacgcattgctaaaatactttgctttatgtaaaattgcacactaaaaataacacggcttatgggcaaaaatagatttggagcagaaaaggaaaactctgcaactttataactaaggtgctaacaaagtcaataattgataattcactggaaagcgcaggaagggagatcagtgggttggagtctgttataggggcaattaaaaaatgtacaaaagcagtagggtggaaatgctagtatagaggcattgagccatggcagatggaagtagagctctgagccagtgagccgagagtaaggtgggcacaagagaaagccaagagccctggaaagctgggaggtgctcctcacctgggatgcaggtatgcgtttttatttttcttgaaatagaactacaaagcctctcagctgtgtagtagcagggctgagagccttttcctttctgcctaagcttataattgtactcttgatattgtggtttcccttgattaggaaaaaaaaaaatcacctatgaaccaactgaacttctgcattattctgatattactcccttatttaccaggagcatataaactagttggtatttctataatagaagcatgtattgtaggccgggcgcagtggctctcacacctgtaatcccagcactttgggaggccaaggcgggtggatcacctgaggtcaggagtttgagaacagcctggccaacatggtgaaaccctgtctctactaaaaatacaaaaattagcccggcattgtggcagtcgcctataatcccagctactcagaggctgaggcaggaaaattgcttgaacccaggaggcggaggttgcagtgagccgagatcatgccattgcactccggcctgggtgacagagtgagactgtctcaaaaaaaaaaaaagaaactggctgggcgtaatggctcacgcctgtaatcccagctctttgggaggccaaggcgggcggatcacgaggtcaggagatcgagaccatcctggctaacacggtgaaaccccgtctgtactaaaaacacaaaaaaaacttagcccgtcacggtggcgggcgcctgtagtcccagctactcgggaggctgaggcaggagaatggcgtgaacccgggaggcggagcttgcagtgagccgagatggcgccactgcactccagcctggacaacagagctagactccatttaccaaaacaaaaaacctgtattatagaactactctcagtttactctgttctgtttaagtattggtatatttaagactggaaggtctatatatatatatatatatatatatatatttttttttttttttttttttttttttttttttttgagacggagtctagctctgttgcccaggctggagtgcagtggtgccatctcggctcactgcaagctccgcctcccaggttcaggccattctcctgcctcagcctcccaagtagctgggactacaggcacctgccaccgcgcccggctaatttttttttgtatttttagtagagacggggtttcaccgtgttagccgggatggtctcgatctcctgacctcgtgatccgcctgcctcggcctcccaacgtgctgggattacaggcgtgagccactgcacccagcctgttatatatattttttctatatagtagatatagttaatatttacaacaaaagaataggtgaaaatatagacatttttaaatggtgcctttatatgacttatgcatattgcttttttctaaacagagctattcaaaatgattttataattataaatgatttatgactattgcctttttactaaactgataccttaaaattatgccttgaaagcttatacatatccacaagggattgtgtaaatattttttactacttggaaatagcagcataaatggaacctggtctgagttcttttgcacattttatggtggctgttcctaagtacaccaccatccttaatcatattgtcaggttcttctcaccttattctcggcagatggcttgcttcatatttcatagagtaatacaagttttgtgaacttactcagcttcttgatcagccatcttcacatgtcctttttgcctcctgttttagagtgggacccttttctttttcttatttcctttgtctgcaatgtcctaactgtacctactctaatagctccttccttccttcctttgctttcctttgctttcctttcctttcctttcctttcctttcctttcctttcccttcctttctccttttttcttttccctttccttttcccttttcctttgcctttccctttcctttcctttctttcacggagttttgctcttgctgtccaggctggagtgcaatggcatgatcttggcttactgaaacctgtgcctcctgggttcaagcaattctcctgcctcagcctcctgagtagctgggattacaggcatgtgccaccgcgcctggctaattttgtatttttattagagatggggtttctccatgttggtaaagctggtctcaaactctggacctcaggtgatctacccaccttggcctcccaaagtgctgggattacaggtgtgagtcaccgtgcctggccaatagcttctttctttaacctaaaacaaacatgtttatctttaatatgggagctgctctacatagaaattatggtctctaattgtaatttcttatgcttaactaggagagattgagataaaaaacatagttgagttaggacgtagggctggaggacttgtataatttactttttagttagaataccaattctgtgtatgtgtgagtcacactgtattatagtaattgtgctacttaagttcaatattgtgagagaaaacaaaagcctgggtaaattttttacacaggtatgcagattttgaatagtaaactgtagttcaaattaaattgcataaacaagaacaatggattcctagggtgtttaaaaacatcaggattagtgagtattaaatgaaatatcaggcattcttaagataaccttttgcagcataattaacagagtcaaaggggtatctttcaaagaaaattaaagaagatcagatagcccaagtgattaatactgaattttctaccaagtactgacctgtctgcaaacagggttagcatgggttatggagccaaactgctggggttcaaacaccattaaaattactagctatgtgacactggaagttatttgacacatccgtgcctcagttttgtcatctatagattgggggttgcatctacctcatagggttgtagtgacaatttaaatgaattaatggatgttgagtgcttgagagaacagtacctggcagaaagaagtgctcggtaaatgatatatatagttgtaagtgtcagtttaggtgcaaacaagaaaatgtgtaggtgcaaatgaatttgagttacttgtcaaggaactcttcagtttattaaaaaaattatgcaagcaaatgaaacttctagagaatcagtgcaatcacctgatgaatgagaggtaaagagaggaagaagtgatgagatgcctctatgcagaggcaagcagatactatgggtggggagtgaaactttcaggtgatattttttggcagccatttccatgacatgaaggattgacactgaggacgaaattagtctcttaaggtttgaggtgatgggatttgaaggcaagtgggaaatgttggcatttttcttttccctttgaccttctctgtgcctcagccccaaatgctgctattatcctagtttgctccttttctttgttcctctgttgctcctcctaagtgtgtggctgtcccattccttcttgcctgaggtttctgtttcttgtactcttttttttttttccttaacatgattaagacagtttttaatctgttgttgaagtgttgttgtaatacagttttatttttcccattcttgagttttaaattattatttagttactaataaattgagtttgactgttatatagaccttgattgtcacccttttgctttttgcttttgtttttgtgttttgcggatcagtaatcattgatgtatttaaacgagctttctattccctgaatgcttctacattaagttttttaaccagctcaaactgtcactaaaatctacatcctctaaaactttacagattttatagctacataatttttattatttttgtttttattttcattttgagacagggtctggctctgtcactggggctggagtgcagtggcgtgatctcagctcaccgttaacttccgcctcctgggctcaagtgatcatcccacattagcctcccatgtagctgggactacaggtgtttgccaccaccccaggatactttttgtatttttagtagagatggggttttgccagattgcccaggctggtctcgaactcttgagctcaagcagtctgaccgcctcagccttccaaagtactgggattacaggcaggagtcaccgtgcctggtgcataatttttaaatagtaacgcttagaagtcatatttcccatctgaacccatttgaagccagtcatgccacattattaaaagatttagacttacctaggatcatttctttcttaattcagtatattctttttgaatttgtagccagcattatattaggtcctgagaacaaaaggatgagacataattcctgccctcaagaaagcattgtcttgtagggataagaaccaggtcaataaatattctctagtgccttaaggattgatagggagtgctagatatagtggaacacgttagagaaaggactcaacactggctgagatagagcaagagagagagagcaggaagctaaatcatgaagggccttatgtgccatgtaattgattcagatgttatctcataattagtaaatattgaagattttaagcaaaagaatatcatcagacttgcatgattgcatcagactcaccaccctgtagttatgaatatgccttactaacactggagtaactttgtactccagtaagtaagtaactttgtactccagttagtaagtaactttgtactccggttagtaaggcatatccataactacagggtggtgagtgagactggagacagaggaaccatttaggcagttattgcagttgtctagagagtgttaatgagggttggaattatggtagtgacagtgttttgaatataggaaatgttaagcagaattgacaggaattagtaataaattggctataacgactgaaaaagaaggcagaatctaaagatattcttaaaggttttgtgtggtgagggcctttactgaaagagggaatactggatgaggaacagtgcttggctgaaatgatgatcaagtttagttcacatgctgattctgaggtaaatgtgaagtatccagtgtgtgctatgtagggctgaagttcaaaaaataagtgtcagctagaaaaatagatttgggagttatcactatgtaggcacaagttgaaacaaatttctgtgagagatcggagggggtgggatcaagaacattgtttgaaaaaatagctttgaaaagttgaaaagttttgaaaagttctcttcctctgagtgtgaagagaagggaagaagatggtagtagtcatagataaatttgttaggcaaggaactcagaaacttcacactcatactttctttcttttttttttttttttcaagatggagtttcactctgcttgcccaggctgaagtgcagtggcacgatcttggctcactgcagcctccacctcctgggttcaagtgattctcctgcctcagcctcccaagtagctgggattacaggcatgcgccaccatgcctggctaattttgtatttttagtagagatggggtttcaccatgttggctaggctggtttgcaactcctgacctcaggtgatctcccaaccttggcctcccaaagtgctgggattataggtgtgagctaccacacccggcctcacattcagaattctccatcttagccttctcctcctctctttctgctgctgcctcttccccctccttttttctctgtctctagatttttaaattttttattatttgtagatattatctctgtaccaaatatttgctttatacaaacaattttaaaagacagtatacttcactctatggtaatatgtaaatataattttgatttccttttgagctttttcaaatataaaagtataggaagtgattttaggaccttttaactaaacaagatacaatttttaagggtaaagaattaattattaaatccaaatgtgatgtaactaaacttttatgatcacacttagtcatgaaataataaagtcacgtttggaaatatatgatttgagaagggcaaaataaaaccttgaaagactctttatatatcatttttctttggccaaatatatttttatctcactttcctagcatattagactggctgtatcaggtaattatttgggcttcatgtttctttatctttcaaatatgatagataaccttcaaaatttaattatttgagagcagcagtaaaggtaaaattcagtaaatttacaaaagactattttctgaagagaagtgcgaaagcatgtgttgggtaattagttatagttcgttatggaaagtattgaagcttcttgtgttcttttattgttataattattatttttattaaatatcacatttatttattttgctaattatacatatttattgtagatgatttgaaaaatcagaacatttttctaaagaggtattaaaaatcggaagtaatatttgaaataccattaaccaaaatataccccaatataaacatttttatttcttttgtcattttttctatgtacacattaaaatgttttttacacacaaatgaaataatactatattggcaggactatattttttctccttacactcttccttcctgatactgaaggagggagaaatatgtgttttcattgactataagacttcaaactgttttaaagtctactttcaaagttcaaacttttggtaaaatactttatacttagccaaacagcagaaaaagacttatttaagaacatgtctgttacctctgtgcaataatccatagctaaatattaaaaattctttaatagttgcagaacttgaggttgatttttgttgtgtattgaatatatattactattctgaaaatgaatgttttcccatttgttaaacaaaaagtctacaatgaaccaagatgtggataattagtgagactctcacagtgtttaacccagacagatgaagagttccctatcatcattgtgaccaaagttttatgggccagtctcactaaaaccggctagctctgtttctaggcagcccacctgagttgcctattgccattcacttgttattacaccttaaacttaccagagagatcaagaacctgttcattaggcagtcttactgtttctggcaaggttcaataattccgttgtcaccgtaggaaactgtatctataataaatgaaccttagccaaaaatgtcatctgtgaaaaatcctcttggatttatgaaaaatcatggttggtagcagagtaagcaatctgaaagaataacagaatatagtagtcttccattggcttgagaatagagccccatattttgtactctggagatctttgacttcacatttctctttcctgtaagtacataagcagatggaaatcttggacagcatgttcttgttttctagtccattccaggaaaagaaactatatcaaatgtgaaagtttaatcacttaatgtgtttaatcaattgaaactctatgcagactcttctgtaatattagctataatcaagtctgttagcattttagataacttctccaaagacagtcctcctagttatttgacttcttagctatatctgttgtttccagaaaacttgtaaatgttaatcagaactacaattgacttaccttcagccgacttgcttctggtttagaaaatgtatttctgagatgcagtggttttttagcaaacaatttagtgaataaattaaacaaatatttaatgtgtgcctaccatatgctaggtaattggctaggcatgggaggtattaagccgtttaagacatgttccttgtatattggaagataatacaagccgtttaagacatgttccttgtatattggaagatcccaacatggttgggaaaatgaaaacaaaaatagttatactgtaatgtaataaatgctataacagaggtctgtattgagtatgaattatatagcatgactaatttaccctattagattataaactcttagaggacagggtctagtataccttcatatttcctaaagtgagtttgcacaatatgagtgcttaggaggtatttatgtaatatatgaattaatgcaaaaatatcagcagagagaatattcttccactgttgtgttcaatagaattaatggttttgggagtttctactaattagtcttatttcaatttttctgttacttctccaaacaatttaattaatacttcttgaactggttctcagaatgttgacattgcctgagaaatggaatttgtcttttgttttatcagtcttatgaaggtatatccttaaactcattcattgcaatttgctctctcaatctttagtaacaaatttatctcttttgctggatatagagtctgatttttcttgcacctgatgaggaatagtaagataattaaaggaaaagtaccagtgatatttttagctaagcagttttaaattgttttgtgatcatcctacaagaagggataccaactataaataataatatagtgattgtaccattatagaaaaggctaagacgaaaagctccttgtaaaatctttttactatatgtgctgtgttgactttatttctggttataggtaacaattaacatgacttttgacaaaaagagtactgaattttccttgagtacaatcaaatgaactttgcaaattaatatagtttatttcttctgagtgaatgcaatctagtctctagttcattgttataagccattttcatgatgttttattatggctccattatacatatacattatcatttgctgtggaagtaacttgtaccaactaaactgcttagaaagtgttactagcttacatgtcataaagtatatatattttttccttttaaatagattattgaggttggcctttgtgaagtttttgaattgatcaaagagacacgattttctcatccatccctgtgtctcaggagtctccaagccctgctcaacgtgctgcagggccagcagccagaaggcctccagtctgagccacctgaggtcctaggtaagagccaaggctcattcagtgaagcatttaaaagtgaatataattaataatagagtattgtacacttcaaaattgttaagagaatatttctaaagttctcactacagaaaatgttaagtacttgaggtgatagatgttaattagtccacattttattcatgaatcataacatcactttataccccataaatttatataattataaattgtcaatttgcagtttttaaaaagtgaagagcttctcttatgaagcacacacaatagcctgtaatgcagtctttatttaattgtggtacggaaacagcaggggtataagttctagagtatttttttctgtgttccactgaagttatggtttaagagttgaagttcgtgcacacacatttctcaattagtatgatgaagcggggggcaaacaaaacataaatccctggaatccagaaataatcctcaaaccttaaggtcactattaactgaaatcgttcttattaaacttttggccaaaatagctaacattcactttctctaacttgttactctcaatagttttaaaaaacaggggaaaaggaaataaatgccacaaagccaaaacagtacaattagtttctaaattgaggggacttttttttgtattcatcataaataaaaatctatttgtgttatggattaaacttctgacttagcaactttcattaagtaaataaagtgttcgcttttcttaaagtatgttccgattcttgcctttcatatcagtgacccagactcaacatgtaatcctttaaataagataggagcttttacttaaaaggtactaaggaatagaaatatagatggaagcataatttttaactggttaattgctttttttctccgatgctaactatgggtctttaaactatctaaagttttctagatccttctattattataggaaaattctcagaagtactagagctgtggttctcattatgtggttctcaattctcattagcatgtatcagagttatcggggacttccctatggtgtttctgattccatagacttggggtggggcccaaaagtttgtatttggaattagtttcttggtgatacagatgctggtttgaggagtacactgctggtttagactaaaacttggtcctttttttccgacttggctatttagattttgtaggacccacctgtgagaaccaaactgcatgaacctaactacatgtcagatttttcttttttggtcgcatgtggggactcagttattgtgtttgacttttgtttgttttaatgagagggaggatgctggtcattgaatcaagctgaggattttttaaaaccacccccctcacatacacagttattaaaagctattaccaaatagaagctgtcatgttggcaggcaagcaccgtgttggttattgttatgatagcaccagtgatcatattaatattgtgaagtgctgcttggcttgacagcattttactgaagatttgatttattttatttaacaatattttgctcggttttcttggatttggtttgttagttagcaaataattggaatgttgaaatacccgcgaagacttggatgtcgtatttcctactttgaacatgttatttctttcctcctaagtcttctaccattctcatgtctcatgtcagttgttatatactttcctttgtatccattctgtacatactttctttcctttattcttttaaaactagaacttgttcttaatactggctttggcatataatatgtgctttagtcttatcttccatcaaaactttcacattattggctgggtgtggtggcttatgcctgtaatcccagcactttgggaggctgaggcaggaggatagcttaaggctgggagtttgggaccagtctgggcaacttagacccctatctctccaaaaaaaaaaaaaaaaaaaaaaactgggcatggaaatgagtgctactcaagaggctaaggtgggaggaccgctcgagcccaggagtctgaggctacagtcggctgtgatcatgccactgcgctgcactctagctgggtgacagaatgagacctcatctctaccaaaaaaaaaacccccaaaaaatcccacaaaacccaaaaatatttcaccttattaatctgttcatttttattttcttgtttaaagtttcaaattctgctgtactgcctttcgtctgtcttccatcctttcagtgtttactgctgatctcgtcactgctttggcttatttttctatgtaagggatttattatcacttcatgttttctattttgttaaattagctcaaagataaaagcactgatttggttcttttatactaaatttcttttgaaacttgcctctcctcctttttttaatctaatatcacaatcttattggttatcctttttttcccattcggttgttttcgtgttattcatgaaaatatgtgtatatttatataactcttactgtggaacatcttgatgattagtatacaccaagcttcttctctttgcaaatagattttgatttttaaattatctatttaaaaatatatttccttatatccatatattctgttcaattagattatattctcgagtactcactgaaatgtaatagcaccccctttgttatgtgctaagaatgtgcgatactattgctgttattatggaacttagcatcagagagttggatatgtgtataattacaaatgtggataaatcgtatggaggaaagtagaaggagttctgagagtccttttttgttttttgagacggagtctcgctctgttgcccaggctggaatgcagtggtgcgatcttggctcactgcaacttccacctcctgggttcaagctattctcctgcctcagcttcctgagtagcttggactacaggcgcgttccacaatgcccggctaatttttgtatttttagtagagacagggtttcactatgttggccaggctggtctcaaactcctgacctcaggtgatttgcccacctcagcctcccaaaatgctaggattacaggcatgagccactgcgcctggccaagattccttttttaacttaaaaattttttatttatttatctttagggacaggatcttactatattgcccagggtagcctcaaaccctggactcaagtgatcctcctgcctccacctcccaagtagctgtgactataggtacacactgctgcacccagctccttctgagagtctttaacaggctccctgaagatgtgacatatgagatttagagggtgaataggagggagtgaattcgattataagtgctctaggcagaaataacagcatttagtaaggctctgagacagaaaggagaatgaatgaaaagaagagcaatatggctagaatgtggagagtgaaggaaacagtcttgtgacaggtgaggaaggaggcagggacatgcagagctttacatgccatgttaaagaatttgattagtgggctaggaggaatggaaagtcattctctatttcattacatgaagaatattgttttttaaatagccttattttcttaaattatttgtttaaactattgactattttctttccctggattcttaacctttttgaaagggtagagacattctttgtcatgtttgccaataccctagtgtagaatagttgccagtgtgtgagagttcggtatatatttgctgaataatcaacagatgtactcactgaaggataataattggaggaaggatgggtagatggaaatatggacaaacagggatagatgaaaggatggagggaaggaaggatagctggaggaaaaggtagaggaaaggataaatggaaaaaaatggaagtataggatgtgtacagacaagtgaagagaggaagggggaaagaaaagaagacagaagggaggaagaaattaacaaaggagtgaaagaattttaagtagacaatcagatttttggtttgaaattctccctggctattggggcaaaagcgaatggtaagagataatttcttaacactttttatcaaagagatgatgatagcatggattggaaagatgatgatgagatagggaaaagtggagttatccattaggaagtaaaataaacaggacttaatttgggattagctatggaagatgagggattggtaggggctgcctgggtttctgtaagttaccagataacccacaggtggcattttagtttttagagaattgcagtacattactcctctttctcttgttctcctccttctctcccttccctttccctctcttcatgtttctcagtttttctttttctaactctctttttccctctccatctgattattcactctataaggattttcaagtctccgttctatggaagaaatacccagatgtctgttgattgaatcttagatggaggtgttcagggaattttgtgcagagacctctgctggttgtaaaacatttgtgtgtgtgtgtgtttcatcttcaccactaaatgactttatgatttaggaaaaatcttttgataccacatatctcaatttcattattaaagggggagaatatctgtcctattgacttggaggtttttctagggtataaatgaattacatcctaagtgtgaattgctattatgttaatgatatagccaattttatattaaagcacattattgcatatgttttatgtttctattactttgaaaatatatacataatttcctggacacttaacattttaagaagcctcctaatctaaaaggtaaaataagtatgacaagtagtctattacttgataggatgagtttcttgaatatgtgaatgcctctaggcaagcttgttttaaaagaccttagattgtctttttttactaagaaaaataaggagttgaactgaatgttctttaagacccttgttacataaatattttaacttttggggaagagagtaactagaaagaaactaaagcaaagtaattgttctagattccttaaatttcgtttataatgagacaaagtaaaaataaataaatatacgcatataatgttccgtatgttggatgaatataattgaattctattttatccacagagtctctcttccagcttcttttggaaatcaccgttcgaagtactgggatgaatgacagcacaggacagtccttaacagcactttcctgtgcttgcctctttagtctggtggcttcttggggagaaacaggaaggacacttcaggccatctctgctatcctcaccaacaatggaagccatgcttgccaaactattcaggtatttcatatttatatctgcttagaagtttataagatgacaagttacagtttcgtctcaatttctgtcaataggaattctgctttttctcactcattacagtagcttaatctgtccaagattgatacataaataatctacagtatttcaattaagtgatatgtatgtttaactctgaagctttaaatataataaaatgaattttggccagacctcttaaatctttaagctggtctttgccttaacaccatagccttctctttccttgctgttttcttgctgtctgcgttctcccttcttcccatctttccctgagttttttagtatggaattcattcatggttttaaatatcttttctctattgaaagaaaaagatatggtatcaatctgatatatttttgattggttgattatgatttttttatgaattgattcgtttctgagttacttcagctgtgtataaaacattttaggcttttaatattcagttgtggaacaaacaagctcatcaccattttagtggtgggaaaaataatccttataggcaaacttgacttaattatttgggaaggctctggaaattgagaaagaagttatgatcttaagacctgactgtctactcttctctggccccttgggcaaccctagggcaggtggaagggatgcctcaatcctgttattagcagaccaaagatagcaaaaggagtatattggtcattttctgtcttattcagtgattttgaactctcctggtcaaacaagacccattcccttactcaagatttctgctcttttcttcctttctgccttgcttggaatccttttgtccctttttatggtccttaattattttgaagttgctctaatacagtggttttcaagctgtgttctgtagacatgctttcagggtttggcaaatgtttaatttaaatatatgtttaaaatatgcaaatattttaaaaattgtaataaagtaacacaaacattcagataccatataccagattttaataggatagtgaccaccaacatatgagcttatgaattttgatatgatttgcttttatatagagacttaggaatgccattagagctcatagcaggagtataagaactacgttcatggaaagcctcccagaggatatgaggtgagatctaaactaatgagttgaaatcagccaggagaaatttttggtgttggtggtgtagagagagaatgttctaggtatccacatgctaaggcatggaggtgagagtaagagtgaaatatttgaaagctgaaagaagtttagtatagctatagcatagagagctgtcaaatagcgtagccacagcatacagagctggcaaataacagagacaggactcgagggcttatctgtctccaatatttatgctcaatcactagacaacacaaaactacttgatcctctgaatcaatagctgcagttctctacagacctgccacatcaccacatccccaactctagcctaaactccatattaaagcccattaccttaacatggcttgcagtgctcttcatgatttggccttttatctaccaacttcatttcccaaaactctcctggcctgactgttggtagagggagaaaagctttccctgagaatgtgtgcattaagtccgttttcacgctgctgatagagatgtacccgagactgggcaatttacaaaagaaagaggtttaatggatttatagttccacatggctggagaaacctcacaatcatggtggaaggcaaggaggagcaagtcatgtcttacgtggatggcagcaggcaaagagagcttgttcagggaaattcctctttataaaaccatcagatctcgtgaggcttattcgctatcatgagaacagcacaggaaagacttgcccccatgatccagttacctcccactgggtcccttccacaacacgtgggaattcaagatgagatttgagtgggaacacaaccaaaccatatgagtatgtgtaactcttcacatctcagatttgttttggcacccaaatgcatactacttgtccatttcataaagcttaacagagagtttaaagcagggtagacatggtagtactgcaagatgctgtattgaaagacactgaaatcctttctggagggttgaactcttgacttcaaaggctttctgcaaataaaacactatttattgcatttgagctcagaaacaaattaataaaaaaagcacatggaaaaaggcaccattattgagaattagcagaaacaaatatggtagaatcaatcctgccaagtccttagatattgaaattatttgatatagaatgtgtatgtgtatgaatatataaagtatggttaaagtaataaaagaaaatatcaaaagtatgattaagtagtgagaatataaaaagtagtcagacattaggaaaaaaaacaaatcgaactcctagaaatgaaaaaccaaataattacaattagaaacttaaaatgggttaactattgtataatatggttgaagatggaaatggtgaactgaaagataaagaggaagaaattatctagaatgcaacagagagacaaggagagaaatatggaagataaaataagaaacatggatcgagaatgacaaaatctaatgtaatacaatctgagttccacaaggcaataatagatttgtggagatagagtacttgaaataatggctaagattttacagaattggtgaatgacatggaatcccaagagtaaggaagtctaccttgcttgagatttgcttcttatagatgaacttttagttggtactttttgctggcatgcgaaatatcattctgaacaatttattgtcttatgaatgatgatgtattaccaaagtatctttgttctgtgtttgtaacatttaaaaatgttttttcattatttttcttctcttttgattaggtgccaacaattctaaattcgctacagagaagtgtacaagcagttttggtgggaaaaattcaaattcaggactggtttagtaatggcattaagaaagcagctttaatgcacaagtggccattaaaagaaatatctgttgatgaagatgaccaatgtctacttcagaatgatggattttttctttatctattatgcaaggatggattatataaaataggctctggatacagtggaacagttagggtaatgtgattccttacagttccttaattatacagagttataaccataatgaattgtgttctgtgtgtttctagtttcatttctagaatatgatctaattttagtgtaataatatttatttgaaaaccataattgaaatacatgcattaaatgtcattcacccatttgtattatttttcattgattattaattatgaaaccactgatatactaacttttgttttttttgcagggccatatatacaattctacatcccgtattagaaacagaaaagaaaaaaagtcttggttagggtatgctcaggtaagaaactatccacaataaaccaaaattttcttatcttttacaacatgtgatttgtccttgtttaatcagtaatatcacttctatttaaacagaaatgataatatatttttactaatatggccataataactgaatagtgtagcgtaaagaacactgtctcagaactcataagaggttcaattttagttctggccctgcttcctactagctgtatgactggacaactcagttaacttattgggtcctctgttatttagctttaatatgggattaaacataccttctttagagttcttgtgtaagtgagcctctagataagaattgaagaccacctttatcattagcacatttttttttttaattgagacagaatctcgctgtgtcacccaggttagagtggagtggcacagtctcggctcactgcaacctctgcctcctgagttcaagtgattcttgtgcctcagcttcctgagaaggcccatgccaccatgcctagctaatttttttgtatttttagtagagacagggttttgtcattttggccaggttggtctcaaacccctgacctgaagtgagccacccgcctgggcctcccagagtgctgggattacaggcctgagccaccccacccagcctcattagcacatttaaattggaaatgtatacatgtcctgaggccaaattagggtttcttaccacagactctttcattttagtctagattcagtactttctaggaaacagtgtttggcctactcagtagacagctacctgcatgaagaatatgcaaagaatcacaagagagaaagaaaagccctgggtttttgtgtcttaatgctgtgatcttatctcctgaccaacatagtcagataggtgttagccttttacaaggtcagcagccagatggatacattactcctcttatggagaaaagcaaatgaaggatggacaagagggactagaaatatttttttccatacagtacctacctgaacagtgaagcccaagttcctttgtataaggataaagaataaagatttccctgcatgcctgccttgctgagcttgtgagatttgttgtcaggatcaaatgataaaagatatttgaaagtcttttgaaaactatggaaattactattgttacatgaagaataattctactgtatcacctattggggagaatttgaaattaaatttttttttttttttggagttgtagttttgctcttgtcgctgaggctggaatgcagtggcgtgatctcagctcactgcaacctccgcctcccaggttcaggcgattctcttgcctcagcctcccgagtagctgggattacaggtgcccaccaccacgtccagctaatttttgtatttttagtagagacgggtttaaccatgccggccaggcttgtctcgaactcttaatcccaggtgatccacctgcctcagcctcccaaagtgctgggattacaggcgtgagccaccgcccccagcctgaaattaaattttagataacaaattagtttctcagtaattgtctcttaaaaattgaacttagtttaaaatatctcatcagatttttatttgccactctattgtgttttatctaagaaataatgccgtggaaagtatattattatagcagtgtgttcaatggcataccacaccttctgaaaccactatgctattcattttcaaatagcaaactcaatacttgttatttttcttaaagtacttgttaaagtaacagtgatcattcatattatttacttggcaagtggtatggtcattttgaacaaaatgtttgtaactgtactgtcctgtttagtatacctaattatgtttctgggaatatgttacatcaattttcaataatgtatgacttttcctctaattgagaggaatgttcttatatttttaatcattaatatcttttagacatcaatattgatgtattattgtacctcaaaaacctgtaatatggagctgtatggctgctgtttctgctgaattagtaataaatattttaacaggaaaattttttgctgttatcagggttatttattatatagagatgtgaataaccacagcatgacagccataaggataagccctgaaacactggagcaagatggtactgtgatgttaccaggtatgtttcaagtagtcattttttctccacaagcaattttaagaaatgtgcatgttaagctatttagacacataaagaatgattagcaagggatagtgcttgcttataaaagggttttaaaaatcttgacatacaaagcattttatagtctacgtgaagttaatacatatcaagagaatagacatagaaatataacaagttttaatatttgttttcaaacttggccgtctatggcctaatctctgacctcaccccaccactgctgaattagtggggtgatgtaactgtgttgctcttaataaaaatcagaactctggctctcatgtgactcagtataaagaaccttctgtttatttttactttaacaacagtgcagttgggttttcctccttatttaatttaatttaattttatttaatttaatttaattttattttattttattttatgttaaaggggtttcagagacagagagtttagggtaggtcaggaatttttttttttaatttcatcatttatgcttcttatgcttcttaatactttgataaaggtctttggacacagtttattttgctttaattttgaggcttttatatattcacttaaatcactgctaaattatcaggcagtaggtctccaattttgatgaatgctggaaaaatgcatattctttattgtaagatctttgaatttgtttataatgttgatactttctgaggctgtttatattaaaatcttgagtgtctaatgtgtctgtcaataatttcatagtagcaatattacttgtaagcgtttgaccatctgaaagtgagagctacttctgaagagtgcattgagaagaataattgctcctgggctgcatgtttatttttaaatataatcttaataatttcatcaaacatttatttgaaccttactacatatacatagatacatattagtgtacatgacactatgttaaagtaaatatgacatttcatgtccatttcagaaatagaggagctttggaaagcggatgacttaataaaatcattttgaaaataaccatctattttcagatatgtctacagggttttttttttttttttttttttcattacaagtcctggaccatgtctttggtcataataataataataataatgacaataatattaaaaaatacttttaataacatgcacactccccccacatatttatacatatattgaaagtaatcatgtttgttgtataaaaatcatatgaacaaaaaagaatgaattgaaataattataatcccacttcccatatataaacactatgaacatattggtaaatatccttctcgtatttttcctttgtgtatatgtccatacttgtaaaaaagtgtgatcatatgctacatattgcttttttaatctatctctttcacttaatattctgaaaatatttctaggtcattaaatagtcttctacaatgtcacttaaatgttacaaaatatattccattgattaatgtttgagaatgaaagccttccagggttttgctatataaattacactgtaatgagcgttcctatcgtgctgtctttgtgccagtcattttaggataatgacatagaagaaaaattctaaattgacacatcccatccgctataacacatgggtactaatatcaaggatttacttctgagaaaccctgcacatttaatagagactgtgacttattgataagacaatttgaaaatatgtgcctacagctgttttatttattctgccttgataaaaaaattactcgttaatgtctttattcagtttaaatctcgttgctttgtttaacttgtgaaatgcaactttgaacaatacaagtgtggctttctgtgtttcataagtgtgtgctgtattagtggtgaagaatgaaccaaacttcaggaagatagtcccatatttgtcttcagaactggcagatgttgcttgatatattcctgaagatacaagcttaaaatattacatgatggataaagaaaggataaagagaaaatcttgaaatcacgtggcagaagactcctccctcaagcctgaagaagccttttgtaattttcttgtttctttgacctggactgaattttcatccaatagcactttttgtttttcgtgggtaggaatagctctgctctgtagccttccaatcaaggagcagaagtaaattgtcaggtttatagatggtaatgaataccatgtttcaagttgacataaatcaggtaattccttgagcttcatgttccagcttgtcttacagtatagtttatttaaaaagtatttcaaccttgttttgatgaacttaaaacatactttttttatctgataattgtcattttattctttaaacctcggcttatcttacagattgtatctttatgtcattttctctccatgtctggtgacaggttgcctcagttcatgaggctttttgctactcttcttgcgccgcttcactcctcacccctgcgtttatctgaaattataatgagtttttcttctttgctttttagtttaaaatcttactgcttagagcttgagaagcatctttcttgcgcctctttgtgttctgtacagtggactaggaactctttcttttgatttctcctctctttgattaccaagtctcttgggctaccagtgggccttgtctcccagtttctatttccagtttgtgatccctgaattctctatttggtaagggtctgcctgtgttttgcatatttatgataaggaagtatgctagtatgaagctcctgggcaaagataaagaataatccagcttattcgggtgggctccattactgcatttcaaaatcagaattttatgttttgttgattcaggtgcaactagaaatgaaatgttattttttgttttggtattgtgattaatggagagattcatcctagttttctgcagttattttgggaagtatgtgtgtgtgtgtgtgtgtgcttttaacttgcttgctgaaaatttgtttttccaaaaggacagaatttatccttgatgttgattttgcttgtagttttgtgttatttagtagcaatcactaacacgtgttcctgtggtccttctgtaatactggtatcccttgttacaattgagcttgtgcattattttgatcaaagtatatggtgtactattctatggagatacatgcagtattgatgaaatatggtattctagccaagatacatgactgctttgagataaaaataaaagcataagcatatatttgtctgctcttgttggacaaacatttaaaaagtttagagcacactaaatgctaaaaatgctgtgaataaacattgaatttttaagtgggaaatgtacaactggttttgaatatccattttacaaagcaagggatatctgtagttggacacaatagatgtcaaatacagaagctttaggtcgtatttttctgtgacactaatgactgtgatgtatgtctgagagtagagtgttgcctccattgcatgtgattgtattgaaatgattgtaaccaacattatagtttgttcatcgtggtgcatgtttactgtcagactatatctaatactaattcattttgaaattaggtgggatctcaggtccttggttatggaagatttataaatataagcaaaaatgtaataagtatgattagttgaattagtttctgctaatttataattctttattaaaaagcacagtctccttttgagtttatccttattgcgtggagaactctcattttgacttcaacagaggagtaaattcattttgcaaggtgtttgcttgtattgtaatataaatactttttcttttgcacagattgccacactgaaggtcaaaatattttattcactgatggagaatatattaatcagatagctgcttcaagagatgtaagtatcctgaatcttaagtagctagtgtaaatggaattccttttccttaaattatttagcttttattagatagactgtagagccttgaacgacctctttatgtaatgtaatgctgtggatgttttatttttcttaggatggctttgttgtcagaatatttgccacaagcactgaacctgttctacagcaagaattgcaacttaaactggctagaaaatgcttacatgcctgtggtatctcactattcgatctggaaaaggacttgcatattataagtaagatagcaaattaagtgttttccctatattttaattttcaatttttcatactcttaaaaatgaacattgtgttttcaccagttcagcttatgttgtagcagctattttgtgtctgttactattaatataaaggataattttgaattaatatgaataaataactgggaaacacagtctttaaacaaagtatgatatttgaagaccattctaaggaattgactaataatttctcttttttgctttgaatagagcaaaagaaaataaaatttaatgagaattatggatggatattggtgaatgtcattagtttatatgctgcatcatttttcaactatttaaaaatttgaaaacttacgttgtaaatgttattacaacaatatacatagttctctcgttgaaatttttaaagtttttacttgaatagattttcaaggtaactatagaatgatctcatagattgtcaaaatggtaattggccttacacacggatcagtaatctttcacttggatagagaaatacagataaccacagagtatttcttgggcttagattatttacttttgtaatgttgtaaaagtaattaggatcatgagttctcctgagagttttagttttggcttgccatgccactccaggtttaatagtgaccttccgtaacttgtagggttctttgcaagatccatcttttttgactgttatctcattacatttcataaacttggcattagtgctttactttctatattctaattatgtagtatttttataattcttccctagaaaaatcttgaattgaaattacagatttaaaatttttaaagcccataaactagttagtagtttgtatagctggacaaagtcataaaatgtaccatatgtaaagctacaattactcatagttttacaatttgaaaatatgcataaggatctgtgtgtgtatatgcatatttacatatatatatatttgaattgatggagtagctatataagatgtctttctgtagaaaaagacataggcagagaatctgaaatactttaattcaaatatctgcatggcattgttccaagcttgaatgactctgtgtctggactaacctctccaacctttatatatgcttatttgtaaaccaagtggtatagttgtgagaatcaaatgagataaattttgtgaattgtataatgcctggtacctaataaacgctcaataaaactttgagcatttatacattaattcaaggaatattgctgacattgaatttttaaatttttttgtcgtatatttaaaaaattaatttggtactggtttggaacaagagaaacagattatgataactttatctttccttggagtactatacctatggttaatatttttaaaaatttgagcacatgacattttgtcatccttgttttttagtgattttgaattgacattgcaatcacttaaaatagtgcatgttgtatttagaattcttcatattagaaatgcaatagtatttattagtctcgtatttcaggtacaggatttgatgaggagtcagcaattcttggtgcaggacgagagtttgcgctaatgaaaacagcaaatggaaaggtaaattattctctttggattaaaaatgagcattctctgatttaagagattaaaatacaatctgttgattagtttatatagtttgcatgtttaagaagaatcatcttttaaattttgatttaaaattattttttataagataaaattttattcatgtagggaacagtacaggcatgcctcattttattgtgcttagttttgttacacttcacaaataattgctctttttacaaattgaaggtttgtgacaaccctgagttgagcaagtctgttggcatcatttttctaaccgcctgtgctcactttgtgtctctgtgtcacactttggtaattctcgaaatatttcagacttctttattattattatatctgttatggtgatctgttatctttgatgttactattgtaattattttagggtgctgtgaattgtgcctatagaagacaggaaacttaatggataaaagatatgaaaaagctattatgaactgtgtcttcttggcaaaatatgtgtatttcttgctcaactcccagaagctgaatagtgtaaaaaagagtttgtttttatgtgaattttatttttcaataaaatattgatgtccttttttttcctcatgtgaaacctgatgcactattccaaaaagtaccataattcctttttttttttaatacataagatatattacactggcaaataccagagtcttggaatcaaacaaggtggtccttcagcaggaaaatgggttgagctaccaattacaaaatctccaaagatagtacacttctcagttggacacgatggctctcacgcccttttagttgcagaagatgggagcatattctttacaggatctgctagtaaaggagaagatggagaatcaagtaagttgagtgatcaactatgcatttaataaaaataaatgctttattttagtaaaaataaaactttattaagataaagtttatgaatttcaataacaccacaccaactttatatatatatcattgacttgatgtaactgcttaataaatacatattgaatgaatgaggaacttattttactttccaggatctaacaagaatacatgtgaattacaacttgggaaaaaaaggagattttagatatgtgttagtagaatggtattaataatgcaatagctaatttagagattgggccctccaggagtttggtagaaaactaatattactcactgagaatttgaatgtacttcagggaatgaggatctagcaatgatagcgtaaatattaattgaccagatgaggctagcaggttcactgattttaaatgacatgttgggaagcccaggcctttgtgaaaataaacaaatagtgtcataaatgcaaataatatgggttttttttttagctttatatacaaaccattattattattatttgtacatagaggtcttgatttaatctaatttcttttttaaagctaagagcagacggcaatccaaaccttataaacctaaaaagataattaagatggaaggaaagattgtggtatatacagcctgcaataatggaagtagttctgttatttctaaagatggagaactctacatgtttggaaaagatgccatttactctgatagttcaagtaagttaataaaaagttttacttttatgcaaagaagtttcaatactagttatgtatgacaaagacatatctagaaaatttatgttggtggcatgctttctctacattttttacatttccttccaaatgtgaaatatttcgaaagagaaattttctgtcatatcttgtcactaggagcactgtccctagtttcatgcaaagctaatcatagagcttggcaagggaagcttcgctgtagctgtgacttctgtggatgttttccctagactcaggtgcttatgggacatgtccctgggacagtggtagtatcctgaagaagtgattttcatgacatagctagtttataattgttgaagtattttatctcccccattagggtgtttggctgggatgatctgtcttatttactattctatccccagtgcccagcacagtgtctggcacattgtagggtctcaagaattattttctagtgcctgggtgactgataaaccaaaagtttgagaagtggatatgaggggtagaactagctaaatggttttatatttataaatgctaatctcaaaaacttgtcatatttggttactagattttctttctagcagctagaaatgaatataatgagtgaagcatcccacttggaaggattttatgtgtgataaatatgtaaagcctttgagtttctttgtaaaaaaggaaactagaattaatgaagtttatgattagaaatatatgtaattgtcccatgctggggaaattacacctgtagaacatttatagcaattaattattaagtggaggtgtacccagatgataagtgaactggcttaatgttgtaatatttagattctcaattggctgcagctatcagagtaaatcaaaatttgggagtgggttcttgtacagtttaaaaataatattcaatataattgttgcttgctaacatccactgggtagagctgatggaaacttgtatatttcttcaacatatatttattgagcatgggtgatacattagtgattgatggacctgacagacctggatactgcctttttgaggatccgagagacagttgggtattacagaacttaaatattggggaagtggtattgaatggttatggacatagactgagatcctagcaccaacactttttttttttgagagggagtctcactctgtctcccaggctggagtgcagtggcacaatctcagcttactgcaacctctgcctcctaggttccagcgattgtcccacctcagcctcccaagtagctgagattataggcacgtgccacaacacccagctaattttttgtattttttgtatttttagtagagacgaggtttcactgtgttggccaggctggtctcgaactcctgacctcaagtgatacactcgcctcagcctcctcaagtgctgggaatacaggcgtgagccaccgcgcatggcccttggcaccaacgcttttaattgggtaacagtttacttaatctttaactgggtaacagtttactaaacatttaagagttttctttttgtttttttctacctgcaaaaaatagataatgatagcacctgcttcatatagttgttatgaagattataggaggtagaacttaagaaatgcttagcacatcatgtaaggtctcaaagcatgtcagctagtactgttaataataataataaataactactgttttatatatatacacacacccttacctttttcaaatgtgctaaatattaaaagtggaaatttttgaggtataattaacaaaaagacctaaactagtgaaaggtgaaagtgaaggccaccttaaggtagtgatatctgagctaataatgtagaacagttttcccaaagcataatatgagattgatactatttgtaatttaaataaaacaaatacttgataacatgtttttattttaatgtatattggaaaaataataatctttgattgttaatgttaaagatgcttcaatcaatttagagttgatacaaaacatattaaatagataatatgagtggcacttggatatgacaaatataatgaaagtaatacatgaataagtgaagtttagaaaataactgtgtattggattgtggaagaaagcattgtaggttgaaaaggattaaaaaatactgttttgaaaagtctcactggtttctacataaacaatttcatgtcaatatactatggcaagtattttactttgttttgctgaggcaggtcaaagcaggtaaaataaattatttatggactcctttgtgcttagaccagaaaagtttgaaagtactgtggagaggtattaccatttggaggaagcattcaggagaagtaagcttgagagaaagctacagtaaagatatgtcccaagtctggatgataatgttttaggattcactgaagctacgttacaaagttacaaagttaattcagtgatactctgccagtcttttggctacttatttctcattttgttgagttattttgccccagaaataaacggttctgcattgaggtagttcttttactaggctcagaagggcctttttgttgtttttactaaatttgtttctttcctataggtttggtaactgatttgaagggccattttgtaactcaggtagctatgggcaaagctcacacttgtgttttaatgaagaatggagaggtgtggacatttggtgtaaataataaaggacagtgtggacgagatactggtgccatgaaccaaggtgggaaaggtaggtctttaattcttagatattaaaatttgtgttgtcttttcattagtttttcagaacaacatagtgaaaagtcttgttcttttcaaaatgagtggctcttctttttctttttgttcctatttaaattttttttaaaaattttatgggtagatagtaggtatatgtatggggtacatgagttgttttgatacaggcctacaatgtgtaataataatcacagggtaaatggggtctccatcatctcaagcatgtatcctttctttgtgttatgagcactccagttatactccctcagttgttaaagtatacaaaaaattatcgccaactgtagccaccctgttgtgctatcaaatagtagatctcttgtatctaattatatttttgtgccaactaaccatcccatttcccacccactctccctgactacccttcccagtctctagtaaccagcattgtactctctatcttcatgagttcaattgttttaatttttagctcccacaaatgagtgacaacatgtgaagttggtctttcggtgcctggcttctttcacttaacataatatcctccagttacatccttgttgttgcaaatgataggatctcagtgttttttatggttaaatagtactaccttgtgtttatatgccacattttctttatccactcatctgttgatggacacttaggttgcttccaaatcttggctattgtgaatagtgctgcaataaacatgagagtgcagatatctctttgatgtactaatttcctttcttttgggtatatccatagcagtgggattgttggatcatattgagttctagtttcagttttttggggaagctccatactgttctctccagagtggctgtactaaatttacattctcactaacagtgtacaagtgttcccttttctccacatcctctccaacatttgttattgcctgtcttttggataaaagccacgttaactggggagagatggtatctcagtgtagttttgatttgcatttctctgatgatccatgatgttgagcaccttttcatatacctgtttgctattcatatgtctacttttgaaaaatgtctattcagaactttttcacattttttaattggatttgagtggctctttgacgttatatttgtgaattaggaaaaccactgaatttcttatttatttgataatatttactgataaaatatcactggaaaaacaaatacactttagattttttatttttgagcaaatgtgctggcttactccaatcatcttctaaattctacataaaatagtgcttctttggcttagcatctttgatgactgcattactccagaaaagtcttttccaaacatttttttattagctgctgttacacgacatgttttaaattttaaccatttttggcagcaatctttctggcgtatattatcttcccatttaacagcacatgctacctgtaatttaacctgttcttgacaactaagagtgatccttatgaatatctgacactttaccaagaaaagactaaattgtagacacaccttcttcacaatatcctttgctccaaccctcaggccattgaagagaacaaagggctttctcctttgattgccctgtttgctgtggtcttgttttgttgttgtggttgtaaatagcatgtctacttcttgccctcaatttttatatctagctgctatcgttatgtttataactttcactccaatttctgattttggtcaagagattaaaccatacatatatattattagaactaagcgttgaactttggttaaagtaccaataatttcgaggattaatgctgtataaattaacaaaacctggtttttctgttttgaggctttgatgtcagactcatctggatctcagaaacccaccattctttgtctttgccttattcgtgtgtctgtgtgtgtatttaaaatgcatcactttaaaaatgagcaagttactagaatatattggcaaatgaagaaggaacaaaagaaagaaaagaaatggattacttcacaatggaaatactttgatttattactaaattagtgaacttgttttggataataactgctaggacaaaggggagaatcgggtttttttaatatataatatgaatatatattcagattatgttttataagaagtctgaactaaacaacattgtttccttattctgaaactcttgtgaaaattgagcgtacatttacacctatgttctagattcttcagtgacattttgtaataatcaaagtaaaattgttaaaagatttttttaataagaatcatacctaccctgctggtaagtaatgaattagaaagcagatgagtaaagtaagacacatacagtgcatactgattgcagagtcataacagatgctggggagaatgtggagaaaggggaatgctcatacttggctagtggaaatgtaaattagtacagcaactatggaaaacatagatttttcattcgtatgaaggtgactcaaaaaactgaaaatggagctgccacatgatctagcaattccacttctgggtatatgtatcaaaaagaaaggaaatctgtatgtcagagagatgcctgcattctcaggtttatcatagccatatccacaacaccaagatatggaatcaacttaagtatccatcaacagatgaatgagtaaagaaaatgtggtacatacacaatagaatattattcatccctaaaaaagaatgaaatattgttatttgcagaaacatggatagaactgaaggacatgatgttaactgcaataaaataaaccagggacagagacaaatatcactcatatatgggaactaaaaaattgatttcatggagatagtaaatagaatagtgattaccagagacttgtaaggatagtgggagggggagaggaagagtggttggttaataggtacaaaaatacagttagagagaaggaataagttctaatgttcaggagcagagtagggtgaatgtcgttaacaacaatatattgtgtatttcaaaatagctagaagagagaatttagaatatcccagttatcttgacttgatcccagttacccagatttgatcattagacattatatgcatgtatgaaaactcacatatatgccataaataggtataacagttatgtatcaatttaaaacgaacgtaaaggaatattttaaaagtttatttaaaccaggaaagtacagaacatattgaaatattaaaaaattgtgtctctaccatgtcttgtcattttttttcagttaaaaaaaaatcacagataagattcaagtgccccatatcccccttccaaggcttattatcatattccatttctccccagagctaaccgcgatcatgactttgataagcatacgttcaatcatgttcttatatatttttacatgttcttttatattttatgtgtaaaataatattgcataatgatgtctgttttacacataaaatatactactgttttatgtgtttttataagtgttaatctgttatttacatttttctacaaatttttttttctcatcattctgttagaaatatgtccatgttggtacatataaatctaaatgattcatgttaactaatgtgtaatataccacacagtgaacatgctatagtgtatccattctcctgttgatggatgggcttatttccaatttttcattgttataaatgagtttaaaacctcctttataaatgtttcctggaaaacctgtgagactttctctgatgtaattgctctattgtggatttcagttttactggatttttcttgaaagagattgtaacaatttatactctcaccagtagagttggattttccacatcacatctgatattgacagactgatttttgtcagtttggtggctatgaaatgatactgtaattttttatttgttacattgaagtataatatgcatgcaggaaaatatacatagtatcttgatgaattttctcaaattgattgtattcatgtaatcagcacccagattaagaaaccacatattaccagcagcccagaaagccccactgtatgttcctctctgcccttcagttaattaccataaattagttttctttttatacttgatataagtgaaatcatgcagtatgaactcttttgtgtccagtttctttttattgttttatgtttatgaagttcatacatattgttacaagtagttatagattgttcatttttgttctattaaattatatatagatactacagttttaaaattgtttctagtatttgggcatttggatggtttccagttttggttattatgaataatgttgctgtgaacattctagtaaatgtcttttggtgaaagtgtttattcatggcttttgggtatatacctaggagtagaattgctggttctctcacttttcttttaattttcattcccttgattattaatactgagtttgaacatctcttcaaactttattttacttactaaaaattgatttgtaggagcttttttacgtattatgttttatatatctttgactaacctgtggtttacagtttcatttgttatgtataattttaaaattttgatgtcaaaattttcaacctttttctttataaatttttgatgaatgtagcctggtgagagattttattttccatataaattagaaccagctttatttgcttggcattgtgattgagattccattgactgtaaagattaatttgaggataatagttccagtattggaatgattaaatgagtaatgcttatagagctagttacaaatagtgctcagtacattttagctatgattattgaaaatagtcactgaggcttccttcaggatcactttattctaatcctagatatagctagctgatgcttggttatctaatttttctgcttgcccatagattagaagtggcaaaaccccaaaaggctatatatgctatgagtttatttcttggttagatagacctgtattgagtgatagctgtatgttaaatggatgtgataggtgtgagagaataagattgactaagaataagagatattatgagcaagacttaacctcataacttagatcctttaggaaagtcaaatataaaaaataattacttgactcccacagatttgttctcctgtcatagggtcttataacactgtgttccttcaaaaccctaccagttgtagtttacatttttatttggttatttgattaatgtctgtctttcttactacattataagcttcataacagctcagtattaccgtcttcagtttctagagcagaggtctgcagagtataacctgatgtcatttttttatatagccctctaagctaaaaattgattttacattgtaaggattgtaaaaaatgtgacaaagatttgtatgggccacagagcctaaattatttactggctctttataaaaggtttgctgatttcttgccgtgaaggaatccctggagtatagtaggtgttcaatttagaaaaaaagatgaatgaattgtattgtaacaccattttaaaaagttgttaggtgaggtataaacaggcactgaggaggtagtgactgactgcctggaaaaggtagcaagtgctttaatagggaatgacttctgagctgaatttttgaaggctgaagtttctctactagatgagataggcagagacatcttgtaaatgaggaaggaagaggcagagggagcaagaatgaacaaaggccctgagcatatggaaggacacagggtgtttggagagcagagggaaaagaaatgagattggagaaaattaggagccatcttgtgcctactgcctcttgctgagttttgttcttctgaagaccttggaaggtcactggaggtttgtaaattatgaagttgtcatgtgctttgggagggtgattggtggcagtaggaggatgatctagagtggggaactcttggcttggagcatggttgggaggctttctttggtctgttgaatgaaagtgttggtagtgaggatggaaaacaggaaaagatttaaaagtatgattgacagaaggtaacagccaagagacatttgacaatagccttccttgccttcttttgcagaaatgaaatggggagagtgtttattagtggtccctaaattaaaatttatttttgttactcaaatttaagcatttctttgtataattgcttggattagcatgctgtcattttttaaactgggaaatatagtcatccatcttttatacatgaaagtaacctgtataaaaattacaaattgatctgtcaaatagggtttggagttgaaaatatggcaacagcaatggatgaagacctggaagaagaactagatgaaaaagatgagaagtctatgatgtgccctccaggcatgcacaaatggaagctggagcagtgcatggtttgcactgtctgtggagactgtacaggttatggagccagctgtgtcagtagtggacggccagacagagtccccggagggtaagagacaatgattcctactaaagaacatgtgcagaacacatttctttgcaaaatcattccggagtatactctactattaatatttttttcttaaagaccagtggcatcttcacttgatcttagccaaaaggccaagaaagtattataattcttataattgttttctgttataataatttataatttaatgtcatctctaaggttgcctttctttcatttgattcatttaaaccattatttaaaaacagaccataagtttttcaagacttggggctgtacttttatttgtattatttttcaaagcctatttaatgctagatatctattatatttctttctgttgaacatgaatcttttctaaataaaattatcgatatcataaagagtacttgattagatatatagttttgttttaaattaaatgtaccatttataaatttagaaacgttgtatgcttgcttctaaagttacatcatacctgctatatttatccttgttctcaatggaccacatttttcagggttctatattattgtcaatttacagttaatatgtaagtttggcagaataagaatgcaggttttctgctaggatgttaggaagaattttgaatctattttctacattgctagttatagccactataacatgatgggatattctttgtgtgatcagctttgtaataattggctgatttgggggagtagagaaaattttgtgaagagaagcccagaaggaagacagccatccctgcctcttgaccttcctctttgtcaactctgtaatagttgcttcttcccaaatattgtctggatctaataggagtttatttctaagtttatatttggcctttcatagttaaataggtcatatataacaggccttttctttaaggctgctgagatatgattttttttttttttttgagacggagtctcgctctgtcgcccaggctggagtgcagtggcgggatctcggctcactgcaagctccgcctcccgggttcacgccattctcctgcctcgacctcccaagtagctgggactacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgagccaccgcgcccggccatgaacttttaaaaacaaattttctttacattattagtataagcaataaagtcgtctttatcaaaggataatatttattctgtagtgctttatgtttggctctgttttctgttttttaagcacctaacttctttatttttaaatttaatttggatgcagaaagactctgattattttttaacaatgttggaattgaaatttattctcctggatctctccaacgctatttccattaaaattcagtgttagctgggtgtagtggcccatgtctgtaatcccagcactttgggaggctgaggccagaggatcacttgaggccaggagtttgaaaccagcttcagcagcatagcaacacccctaactcaaaaaatatataataaaaataaaatttaatatccccactctctgagcttaagcagccagtatatatcgtgaacaaaatctataaattatctggtcccttttgaaattaattattcttcgttttttcctccagagacttacataatttataataattattcatagacttacatggacaatactaaccaagtataataattgctttatgaatgaagcaaactgaatatatcagaatattaaggactattaagctgacttcaattattataaaattgtgactacatatcttattatgataaagtacatataaaattgaccagcttaaccatttttaagtagacagttctgtggtattaaatccattcataatgttgtgcaactattaccaccatgcatctccataacttttcatcttgtaaaactgagactctgtaactgttaaacaatgactcctcattcttccctctcccctgcctctggcatccactattcttgttttctttctgtgtctgattgactactctaggtacctctcatataaagagaatcagagtatttgtcttttttgtgactggcttatttcacttagcataatatcctcagttcatccaggttgtcacatatgtcggaacatctttgctttttgaggctgaataatattctattgtatgtatattaccacattttgcttatccattcactcactgatgaacactttggttgcttccacattgtaactattgtgagtaatgcgctgtgaagatgagtgtacaaggagctctttgagaccctgttttcagttcttttgggcatatacccagaagtggaattgttggactatatggtaattttatttttaatcttttgaggaactgccatactatttttcacagcggccaaccattttacattcccaccaatagtgtacagatgttccagtttctccacatccttcttaacacttatgatctgttattttggtagtggcatcctaatgggtgtgagatagtatctcattatagttttgatttgaatttctccagtgattagtgatgttcagcatctttccatatactttttggccatttgtagatctttggagaaatgctattcaagtcctttgctcacttttgaatcaggttgtcagtttcttttgttgttgttgatgagttttaggaattctctatatagtttgaatattaattccttatcagatatatgatttgaaaataaaaagttctgtctgtgggttgtgtttttactctgttgatgttgtcttttcaagcagaaaatttttaaaattttcatgaagtccagttgtctattgttttttggttattgtcgcctgtgcctttggtatcatatccaagaaattatttcccaatctagtgttgtgaagggttactgttatcttttcttcttctttttttttttttgattatactttaagttttagggtacatgtgcgcaacgtgcaggttagttacatatgtatacatgtgccatgttggtgtgctgcacccattaactcgttatttaacattaggtatatctcctaatgctatccctccccgcttcccccaccccacaacaggccccggtgtgtgatgttccttttcttctaagagttttttgtttttttttgtaatagttttaggtcttacatttaggtctttgattcattttgagttaatttttgtatatgctgctagagaagtgtccagatcaagttttcccagcaatgtttattgaaatgattatcctttctctattgaaccatcttggctcctttgtcaaaaatcatctgaccatatatgtgaaggaagatttctcagctgtctattctatcccattggtctgtatgactgtctttatgccacaccacattgttttgattactgtagatttgtagtaagttttgaagtcaaaaccgagtactttgttcttctttttcaagattgtttggctatttgaggtccctagagattttctatgaattttaggataggtttttctattttgtaaaaaccatcattgggattttgatagggattacaatgaatctgtagattgctttgggtagtatgaacatcttaaaaacattaagtcttctaatccatgaacatagatgtgtttctacttatgtcatctttaatttctttcagcaatgttttgtagttctcatttcacctcattggttaattactaagtgtattctttttgatgctatggaattgtttctgtaatttccttttcatattgttcattgttagtgtctagaaatacaactgatttttgtgtgttgactttgtatcttcctactttgctgaattcattttctctattggtttttttgagtgcaatctttagggttttctacttatgagattgtattatctgtgaacatagataattttacttcttcctttctgctttggatgacctttatttcttttcctgtccaattgccctggctagaacttctagtgctgtttaggattggtcaaagtgagcatccttgtcttcttcctgatattacgaggaaaagctttcagtctctcaccattatgatgttcactgtgtgtttttcatatgtggtttttatgttgagatcatttccttctattcctagtttgtctactgtttttattatgaaagggcattgaattttgtcaaatgctttttgtacatcaattgagatgatcatgtgtttcttttctttcattctgttaatgtgttacattacatggattgattttcatttgttgcattccaggagtaaatcccacttcgttatggtgtataattcttttaatatcctgctgaatttgttttgttagtattttgttgaggattttcacatcattattcataagggatattggtctgtaggggtttttttttttgcctccccctgcccctctccctcctccctttcttctctttctccttctccttcttctttcttctcctcctcctcttcctccttctccttcttttcctccttctccttctcctcctgctcctcctcctcctccttctccttcttttcctccttctcctccttctccttcttttccttcttccttcttcttctttcttctcttctttcttcttctttttcttcatcttttgtctgacattggtattggggtaatcctgacctcaaagaatgaattcttgttctctcctcttcagttttttggaaaattttgagaattggtgtcataaacactattttaaaaatagtgtcagtcaggcatattactgtgtcacagtttttactgtgtagtaagtgatcttccaaattgtttataatgcttgcttttaaaagtgacacatttcacagggtgtctagctatgtcttttaaactgctgccagatatgttcttccttttgtaccttctgaaatctgtataacctggggttcaacaaggctgtcaaaagtttcaagaactgatctgatgaactacattaaagctataccttctacaaaatatgtgactaagaagttactttccctgaccccaagtcagtttctgggagtgtctttgtcaacatataccccatatggttcacaatcgatatgaaagggctgaaggagaaaaagaacaggtaattggactctgtgaaaggagagaatgggaggtgaaggcatgtaaagaagagagggggaaggagacatatatgtgtttgtgtgtgtgtatatatatgtgtgtgtgtgtgtatacatatatatatacatatatatgtattattttttcctttccaatgtgtgaatgtttttatttcctttgtgtgtgtgtgtgtgtgtgtgtgtgtgtgtgtgtgtgttactttacgttctgggatacatgtgcagaacgtgcaggtttgttacataggtatacacatgccatggtggtttgctgtacctgtcaaccggtcattaggtatttctcctaatgctatccctcccctagcccccaccccccaacaggccccagtatgtgatgttccctttcctgtgtccatgtgttctcattgttcaactcccacttatgagtgagaacatgcagtgtttggttttctgttcctgggttagtttgctgagaatgatggtttccagccttcattcatgtccctgcaaagaacacgaactcattcttttttatggctgcatagtattccatagtgaatatgtgccacattttctttatccaacctatcattgatgagcatttgggttggttccaagtctttgctattgtgaataatgatgaaataaacgtacatgtacatgtgtctttgtagtaggatgatttataatcctctgggtatatacctagtaatgggactgctgggtcaaatggtatttctgattctagatccttgaggaattaccacactgccttccacaatggttgaactaatttacactcccaccaacagtgtaaaagcgttcctatttctccacatcctctccagaatctgttgtgtcctgactttttaatgatcaccattgtaactggcgtgacatgttttctcattgtggttttgatttgcatttctctaatgaccagtgatggtgagctttttttcgtttgttggctgcataaatgtcttcgtttgagaagtgtctgttcttatccttcgcccactttttgatggggtggtttgttttcttgtaaatttaagttctgctgcataaataaatgtcttcttgtaagaagtgtctgttcatatcctttgcccactttttgatggggttgtttgttttcttgtaaatgtgtttaagttctttgtagattctggatattagccctttgtcagatgagtaaattgcaaaaattttctcccattctgtaggttgcctgttcactctgatgatactttcttttgctgtgcagaagctctttagtttaattagatcccatttgtcaattttggcttttgttgccattgcttttggtgttttagtcatgaagtctttgcccatgcctgtgtcctaaatggtattacctaggttttcttccagggtttttatagtcttaggtcttatgtttaaatctttaatccatcttgaattaatttttgtataaggtgtaaggaaggggtccagtttcagttttctgcatatgtctagccagttttcccagcaccatttattaaatagggaatgggaaggagacatattacatgaagctggcaagcttaggctctgttgctgaaggctaccaacacctgccttcacttatctgctggacagcaacattgtttgattgtttttagccaaccaacccatctgggtcacttaatcctaaagcttctggtgcttcactaactgcaaaaagacttttaaaggattgtttttaaagtagtgactttgatgatagtcgaatctgtgtgtattttctgtagaaatataccaattatgcctattgggagtttaatcagtcttgtttttaattattctgctatttatatcttttctttaaaatgaacaatcgtgatctaaagaggtgatttcattgcttactcatttaacaatttcaaaaatgtggaaaataagttatagaaaagagttttaaagacaaattaggaatatctcagttgtcttttttttttttttttgtatttccccctaggatctgtggttgtggttccggagaatctggttgtgctgtgtgtggatgttgcaaggcctgtgcaagagagttagatggtcaagaggcaagacaaagaggaattcttgatgcagtgaaagaaatgatacctttagatcttcttttaggtaattttgattgattatactatgctacactgagttgtcctcaactcagtaagtctgacagtttaaacaatttcttttagatatatgttaataaattaggataataattaatgtatgcaatactgcttttacggtaactgacaatatgacattgtttaagggatgagattcttttttttttttttctttttttgagatggagtcttgctctgttgccaaggctggagtacagtagctcaatctcagctcactgcaacctccgccttctaggttcaagtgattgtcccatctcggcctcccgagtagctggaattacaggtgcccgctaccacacctggctaatttttgtatttttagtagagacggggtttcaccatgttggccaggctggtcttgaactcctgacctcaggtgatctgcccaccttggcctcccaaagtgctgggattacaggtgtgggccaccatgcctagccagaatgagattctttttccctttttggctaagaattattaaagtaaaaatattgtgtgttttagacaatgtttttgctagcttttactttcttgaattttatgtagttctgtcatagctattatataacatatatgttgttgttgttgttgtttttggccaatcatctagctgtcccagtgcccggggttaacattgaagaacaccttcagttacgacaagaagaaaaacggcaacgtgtaatcagaaggcacagattagaggaaggaagaggtaagatgtagctacagagaaaaagtacatgaaaatccactttcctaccctatcctgaaaccactgaaaagttcacacaataaactaatttggtaagaaatcatcaaaattaaaattagcaatattctgataactcatataatgggaaaatattcagggtattcatgcttcttctaataacatacgtttaggtatttataattgtgctaataacatacttttaattcagcagggtgtttatggcataagtcagccatattaagaactgtgccatctgcctactatcaataactattagaaatggcaactttttaaaaatagatattctcacaacttgtccttcacccggactttggactcatctatttgaccgtggaacaaaattcaatttagtctttttcatttctattttcttactcatttatgctcattatcatttggacatagtcctgctatgctgccctccagttcagcacataactatgctttcatgtgtccttttttggaaaaacctcattctagttatttacaggaagagtaagatggcaggtctgcaggtttcatttgcaaagataggtctattgtttatcttttcctcattgtttatgagcttaaaagtttcttagcttaaaaatacatttaactggccaggcacggtggctcacgcctgtaatcccagcactttgggaagctgaggcaggcagatcacaagatcaagagatcaagaccatcctggccaacatggtgaaaccccatcactactaaaagtacaaaaaaattagctgggcgtggtggtgtgcgcctgtagtcccagctactcaggagactgaggcaggagaatcacttgaacctggcaggtggagcttgcagtgagccatgatcgcaccactgcactccagcctggcgacagagtgagactccatctcaaaagaaaacaaacaaaaaaaatttaactgtggttataattggtatttgaatacgcaaagataaatatatgtattactaaagatttccttgctcagtaaattttttttttgtaggattaagacaaattgttatggaagtctatgattttattcctccaataggcaaaattttgattagaaatgtttactgatattgcatattttaaatgcttattagaaatggcatgatatttgatgcatgaaataaaccttttacccatttaaccatcattatatttttttctagctattctttttgttaatatatggaataagtagtaaaatgtataatacacatggtgaattctagttttactaaacttaaattgtaaaacatatacttggcacatgaagagtgttcttcatagcatctgataagtgatgttgattgataaggtgaatatttactattgtttggctttgcttgcataatgtcctttaaaggatattttaatgagattaatatagatactgtcagtatttacattatataagtatatgggaaaattgcatgttaaatatttcttacaaaacaggatagaattaagtttctgatgtggtaattaataattttttatgttgatataattttttcagattagttaccattctatctgtttgctacctgtttcttctttctttagaaatattaggttgagcgacatgaatgaacctacaaaatgcagctatttcttacatttaaactacagtatagcacatccttacacttaattattcacatttaattacttgcctaatgcggtgtatcttgttactctttttctcttccctctaaaaaagctattcttctctaggtatagtatatttgtgcatataggaaataaaggaacattgaatcgtgcattgataacttgacataacttggtgattgcagaatattcttttgccatttatttttaaatttgacataaacctgtgaaacataattgtaaacattaaccctttaaagttaaaaaaaaaatcctccactagctaaatatttaaagaaattatgttgttgtatctgactttccaaagatagattgaggttctaggcacttagtgtatattttgtgacaaataggtttcatctgcagtcctaaatatatagaacatcagtagaaacactttgttcagattattggtgtacataaattttatcttgtccttattttttggtagaactctttcttagatcaccctccttcatgtttggtaaaacaatatatagtgaatatcttttcttgagtggaatcttcagtatgaacctcaattatcacagtccttgctttccatggcactaaaattaacagaaatgtgtgcacattggaaccatgtcctcccctttcacagtcaccacagttacctagaatcatgcgaaaggaggacagagttcccgtatttctgtgtttcagttaacacagcactatgcaaagtgatgcctgcttgtatgttttgtttgtttgtttatttatttatttattttgagatgaagtctcactctgttgcccaggctggagtgcagctgcacaatcttggcccactgcaacctctccctcccaggttcaagcgattctcctacatcagcctcttgagtagctgggactacaggtgcccgccaccacgcctggctaatttttgtatttttagtagaggcgaggtttcaccatgttggccaggctggtttcaaactcctgacgtcaaatgatccacctgcctcagcctcccaaagtgttgggattataggtgtgaggcactgtgcccaacctattttattttaaatgaatatttttgcatttggacaagaattctaactatgtattcattttttaaaaaatcaaacttcagagtcattcagtgactttggaaatccttatactgtattaagtaattaaaaatctattctgatatgacacttttatgattttatctttatattattcttagcactccattaagcaatttttatttaaaaacaaatttttataagaaaacaaaattagatgaatatgatatacttacacttccatagagataaaacaagaaagataaataccaaaatattaacaatgctttttcctgacaggtgagtttatagatattttttattttcttctttacaatttgtttcctaaatattctccaataagcatattagtgtataaatagagtaaaagttatgttttacaactgaagccattttaaaaaaatactgattaggtttaaggcatccatactttgcttacctttgctttttaaaaaatatttttattttaattggatggaggactctctttcaggtgctgtgcttttcattactttctggccatggttctcaaggtggtctcaaagggaccagcatcacttgggcacttgttagaagtaccagttctcaggccccacttcagatctctactgaaacaaaaaactgggggagtacagccagtaaactgtgctttaatagcgttccaggtgattctgatggtcccaaagtgtgagacttagtgtgctaggtcagttgtttgcactttgcggtttgatccaatgaccttctgcatcagaatcacctgggcctaatgaatcagaatttatgggtcaagggtcttggaaatagatctgagtcacttgcacagtgttctaaggctcttctctggcactgtgctagaaaagattgtagtggctttgctcccatgaggaatagaaacatcaaataaaatgttagtgttccggctgccatacatacagatcttttatttctttggcctgttgatactttttagaaactaaaaatttagtagtatgttaataattatacttactagtaattggcttttacctgtgttaacatttcagaatttaagaattaggttatgttagattattttacttgacttttctgtctcatcttatttaccaagtctgaactaagtttttcatagatgatcagaactctgggctctagttctttttttttgagtcacagtcccactctgttacccaggctggagtgcagtggtgcgatctcggctcactgcaacctccgcctctcggtttcaagcgattctcctgcctcggcctcccgagtagctgggattataggcatgcaccaccacacctggctaattttgcatttttgttagagatggggtttcaccatgttggccaggctggtctcaaacttctgacctcaggtgatcagcctcccaaagtgctgggagtacaggtgtgagccactgcgctcggcctgggccctagttcttgtactaaagccttgctcagacagcttcaggcctattgacgtaatttagttactgttttatgacaccaggtctcttataatggaatgtgttattgaaacccaagaaagcaatgatattactgcttcttcatacatttggaactttttaattttaaatctttgtgttattaatagtctagttaatttttttaatttatctttttaggtattaatagtattcatttatagattttagttcaatggtttgtgataacttgtcttttttagattatattgggttgattgatgtattaatttaactgtgcaagtgaagtgcttattcagtgaaattcttaagtagggctcttgaagtcatcagaagaactagtggatgatcatatcaatgattttatccttatattccttttcagcactctgtttcgtaaggaaaattaaacttcaataatgttagctttttcttctttcttctaagtatccaagtttcataattgtgagctgtagatgggctaaacccatgtcctggcttacatgggagagtcccattttataggtgtccaggcatatttctttttttttttttttttttttttttgagacggagtctcgctctgtcgcccaggctggagtgcagtggtgctatctcggctcactgcaagctccgcctcctgggttcacaccactcttctgcctcagcctcccgagtagctgggactgcaggcgttcaccaccacaccgggctaattttttgtatttttactagagatggggtttcactttgttagccaggatagcctcaatctcctgaccttgtaatccgcctgccttggcctcccaaagtgctgggattacaggcgtgagccaccgcgtccggcatgttgctattactttttaagcccttaaaaccattatggttttcttgatttgacgcatcaaatccaatattagctgatattccagtgacacataaaagtctctttttaaattaaagtattatatgaagaatttagagagtataagtaagaaaaaatagatcagcataaccctttatacaaagagaaccattagtagaattttggtgtaacttctcacacttttttacttatttcatataatttcatactgtgatagatattggtgtcctgtttgtattccctttaatattatatcatgaaatttttttgtgctttattgcaaataccatattaaagtaatataggaatttaggtcaaggttttgcataaacctattccttaattcaattattgtttttatttcgttgtcatcttctaatcaagttttacttatttctagttatatcattgactgaattttgttttacatttttgcttagtgttataatacatatatttttatgttttgcttgtattgtacccctaccttcccatttccatccccccctcaaagtaaatgttaatagtctaatatcttactctattaaaatataatcatgtacagagacttacacatatgtgaaagtgtagacatatgtttatttacaaaagtagtattctgtatatacatttgcaacatctttttctcattttacaatacaagaagaacacactgtaggtcagtaaatgtggatctaccacattctatacagtaactacctaatatcataatatggctataccatagtttatataaccattatttattgatgggcattcaggttattgtaagtttttgtgggttttactgtaataaatattcttaaacttagagctacatattagtgtttgtatagtagagctttcccaaaatagaatgtgtggggcagagagtatatatctgtcttaaattttaacagataattcacattgttttaaaaatgttataggaatttgtttatactccctagactaatgtataagagtagtgttttcctctagcatcacctgtattcattattcttttatttatttatttatttatttattttttgtagaggcaggatcttgctatgttgcccaggctggcctcgaacaactgggcccaagtgaccctcctgcctcagcctcccgagtagctaggactgtacaggcatgtgccaccacacctgggtttagttattctttttaattcttggaaaacagtggtgaacaatgttatctcattgttaatttaaataacacaaaatcatattctgtgtaaaattatttcataaatctcattttaatggctgcgtattattccatttagtagatgtattatagtttttaattactttcatttgggtatttttaaaatgttgctataatgaataaagttgtagtaaacacctttgaggtgaaatcctttaaaaatattttagagttttctttaggatagattcctagggtgcagttactagatcaaaggataataatttttatgatcttgaaatatatcaccaagagctttctgaaaggcttgtactatttaacactgccacttattccatgctttttctgcctacctgcctacagatgaactctgaattacccattgtctacacatgagtgtagcttctatctaaacctggttatctcatacccctattttactcactcctctcccaatgaaagaaataaatcaccaagttctatttatacagccttcttaattctttaatagctttttaatatctacttttcagaatcttactgtttccttagctgaggcttcttttctttgcctatctgacagcaaaaagccttttaactggccttacctccagtcttatcttctaagccattagccaccttctttccagaatattcattctgcaatacagatcagatcagtagcacttctctgtttaaaaccaaaaggctattcatagtccttaggatgaaggatatacttttatctctacagcctcatctctctctattctcatttcttttcccttgcccttcctttttttcccctacctctgaacctttatgctacttgttacctcctttatttgacctcatcttatgtggtggtttcttttatctctacactgtctaccttcccaccctcaagttttgttagcttctcctctagtttgtgtgcctccttttgttatagcatactgtcataatttcttgtttagttgtgtgtaaaccctccctcccctcattagaatgtgagcttcttgaagcagagattgtatcttgtttatcaagggttcccagaacttcacatggtacctgttagtaccatgatttgtagtatatttttgttgactaattgaaagcaggttagggacttatcatctctagaatgaagttttgtggtataaaaaggttttaattatctgtttttttgcatacctctccaagcaaatttcccaagacttttgctccatcacatatactctgtaaaagtcagctacatgtatttttaatgactatacaatattgtttaatatctttatgcttttataaaatcctaatttatatcctattccttttaactggaatgccctctcttcctttccttttctcttgttaaactttttgtcattctttgaaaccttgctgattgtcctgagtttatttagtatcttttcttacttcaggacataatgtgtatagatttcttttatagcatttattgtaatatttcatagtttgtgtttttatttctgtttttttccctagtggactgaattccagtgagacctgtatctttgtcattatattcatttcacctagtgtctgctaaataaaagataatcagaaaatttggtgaataaattagtgactcaactatatttaaagaaacattattttagcccatgggatacaatagtattttcgagcccaattgctttaatttttagacatgattgcccccgtgccttcttgatttataaatcaaaagattctaaaaagattgaatttagcaagacattaaacgggtgtgggtgtgatgtattttcatgaggagaaacaatattgtaggcagaccatagaaatcagaaaatggtaatataacaatattattaccttaatatctataacttttttatatagttggaaaatactttttatagtaatttatatatctgctttgtaacaattgagaaatcatgcatgttaagaaaataaggcattttgttcatatttgttcatatttgaaatatcaattttattacattttagaaatggaactcagtttacataaccttagcctgagaaaataattggtactgcattattgagaacttattcttctgttaacattaaattagcatgtttctatctgtagtagtcatagaatatgttattcatggtttttcatacaatttgaggtatttgcatttttgtttatatctagaaacataattgaaatatggttcctttacccacttgtacatttaatgaagggtactttgatatttaatttgacatttatgaaattttattagagggcagtaggctcaatttgtcccatcattagaaaaattaaactgaagatcaatagagaatcatgaaaaaacttgttttaaataaaaatgttcagtgcttatatagtgtcataattcctgtttcagttaatggtaacctgtcaaatttagagtgttttcaaatttgaagacatttttgatgttgttaaaactcaaggaaactatagttctgctgtataagaaaaaaacctaaaattttaagttgtaactaaaatttatttttcttttcaaggtaaataaatgttaatgggaaaaatgatttattttattttattaaaataaatttggctgtatattttttccttaaataccatataagtggaaagagctataatctgtgtgggtatgtatttatatatgtatatatatatgtacatagtggcactttagaagaaatgatttaatgaaagatgtttccaaaatgttatttgggttaggtgccccctttaccatcaatataaagcccaagctcctttctctctctctctctctctcgttctctctctctctctgtgtgtatacatatatctatatgcagaatatagacatttttgttttgtttttgatcatctggttttcttaacctctatttatgatttgtagtaaactcggtaacatttttattttggttttgtagcaagattaatacaaatattaaacctggaagagcttgttaaaacttaatggttaatttaaaaattagtttttatgtttcaatggaaaagatatctgtcttcaacgaatacatggtttactgactttgaatatatatgatattaaaaaagtatttgtaagataatttgattgatttaaattgaacttcttgattatctatttgagactattagtaactacacaataattttatgagtatacaagacgggttttgtactttataaattatatttaagatattaacttttatgtgaaatggtgattaggttatgggtgggggtggggggctggagtggggaaattggaagaaaagtataatgtccaaaaccgttaagaataaatttttatgctttttagtttgaattattttttgttcattatagtgttttcattttgtgagtttttctatgcacatacaaaatgtttctcctcaggcccccttgtatttgctggtcctatttttatgaaccatcgagaacaggctctagccagactcagatcccatccagcacagctaaagcataaacgggacaagcacaaaggtatttggtcttccttatcacctaggtgggatactttcccaatttcttccactcatttgaattttgtctctgggaaaatgcacaaacctgattcttctactttgcttctatcatgatgtagcctgaggagaaagaacattctgaaagtgataccgtttgtcagtgactgtcatgcttcttgtttttttgttgtcgttagctttcataaaggagatattttacaatatatcttttccttgtccatttcttttcaaatttcaaaaagttaagggaaggtggaaaaaataattattgctttctgtcttacatcctttattatttgctctttaatgactgtgatctggccttttacaattcttttttgaatgctgtttatatgctttaggataaacaatattactaaattcagtatattaaatgatgataataagactgccattacacttttccactgtttattaattaagtttatataaacaacaaatgaatatacaaatatttgatggttgtgggttaagtagagataagtgcaagcaaagcacgaatcatgaagactttctctagtccctggtgatggtcagagggctatcctagtctgtgaggaagtaggaatctagctcggtgttgtcctttgagatctcagagttctgggttaattaaatatattcttaaagcatgcttattttcttgtaaaccacaaattatgggtcagctaagatcaggaaagaatttaatacaaggaatttataaatttatataaaatttttgccttgtatcctggcctttgaaccagtgttcttccttagctagcacaacttaaaactcccattttcttaaatgcctaagataggtcctgcaaaatagaataggaagttttaaaaacatgctttccatgtagacaaattaatctaattaatgtttggatccttttcttttttttgctgcaaaattgtcatagtctaatttaaaaaaatttcataggcaatatagtataatccactttatttttatttgcctctttcagtctgtcagtttgccactcaaaacaaaaacataaacaaaatagacaaaactcatctttagtaaaggtgtaaatagagcacaacttaagatatactctgcagaaataaaaatagtgttttaaatctaatgcaattaaccacagtaaaatttgtgattactttatttactgagtgctctaacaatgccataaatatttaaggaattttcaacctaagacagctgattccaaaaagaaaagatacatatataaggaataatatttggggtatgcaatgaaattgttatttgccagaatgtctgatcttttatgatttctactatgtcacaattagaacagattttttcagtttctttcttcccccaaatatatgaaagtgaagttttcccaatagtttaactgaggttaaatattcacatttcatgaaacagtttaaaaccttttcagctccatgttagtatctcatggacaagtatctttaccctttccagatttttaaatgaaacgttaaatgaaaaccaaggttacatatttaaagcaaacttttcccaatgtcacataatgattggctatagaaattattcagtgaggctgggcgcagtggcacatgcctgtaattgcagcactttggaaggctgagggggtggatcacttgaggtcaggagttcgaggccagcctggctaacatggcgaaacctcatctctactaaaaatacaaataataatgataacaataataagcccagtgtggtggcacatgcctgtaatcccaggtactcaggaagctgaggcatgagaacaataatcacttgagcctgggaggcagaggttgcagtgaaaaaacaagttattcaatgatagctagtcttcaagtttgcttgtcatggggttactttataacaagtttctttgtatacttgtaaccactctgtaaggacctactttgtaatatcacagtgtaggaccagttttattggattttagggttttatagagtgagatagttttatctttttactccccaaatacatttacaatgcaaataataatttatcaattggtaccctatttttttgtgaagagtattaatttactgctcactaatttccctccagccactaaccatttacactcactgtcttgtcatttaattatctctccattcactgcttttacttttacgacatgttgaacatccctaatttgaaaattcgaaatgctccaagatctgaaactttttgagcaccaacatgaagcccccaagtgggaaattccacacctgacctcctgtgacaggttgcagtcccagcacacagcttattcagtgtcctaaagtgaaagcaatcctctcagccctcttcagctatgataaagcttttccatgcacagcatgatggtgacgccaatcacagtttgtctacgtgggtggctgggtggctgaggtacctttgctttctgatagtgcagggatacaaattttcatgcaccaaattatttaaaatattgcataaaattatctttcggctatgtgagtaatttgtatatgaaatatcaatgaatttcatgtttagacttgggtcccatccccaagatatctcataatgtatatgcaaatattccaaaatttgaacaaactccaaaatccaaaacacttctggtcccaagcatttcaaataagggatgctcaacctgtacatattttcagtcttttaataagtattttttctacatatatttttctcttcaccttacccagtttatcttctagtttatctactgagaagtttatggtattgattgctatgttctatcctacatttaattatgttggtacaaattataggatggtaatagttaaaattttttagactgtgacttttttgtgagggtgtaaggagagtaagaactgtggatttattgttaacaggactaagttctaagatggaaagttggagaacttctgggtgattggagaaaattcagagaaggtcagaattcatcctatacaggcagatattgactaaaggcaaagcagagtatatagactgaatcaataagtaaaattccatataaaatacagtaaattattctaaaatgtgcctataaactatattcgaaacagaattaacatttgtttcagctattcagtaccttacacagaaaaatgtgttaatagtgtacatagttaggaacatctgaattgttctagagatagtcctaaaatctattttaaaatgactgatttgaaaatctgaaggcacactgataaacatctgagtctattttactcttactttctgtttggaaatggtgcagtgtgatgaaaatattatagcctttggaattaggcttcaattttatttctgaatttgtcatttactagaactacatggctgtgcaaaatgtcttaatctctttggcttcagttttcttattttaaaaacaagaatagtaattcttaactgattgtgttgctatgaagattaaaatatatgtgtgtagtctttgcacacccacatatgtagtctttgcacatataaatcctccatgaatactagctttaatattttgaattgaattatttgttttctctttcagaattatcttaaaggtacaatatttgagatttattaaatgcatagtttatttttacaaaggatttaaggtttcaaccatttggtggtggtaaaatgcatgagccttatttatgattaaactctaaatttgtcctaccatttatgattttgggctggttacttaaccccttaacccttcaagccttagttttttcatctgtaagatggacaaagaatacctatattatcagattgctttattcattaaagaaagtaacaatagtaaatattgattgagcacttttcatattttatttaattctcataggaacctgatgagttacatatcattgttgctatattatagataggaaagctaggaccagaaggtccagtgtccagttgtcatacaactagcaaatgagaagagccaagattccagtccaagttcttaagttccagtactcccaagcactcatccactgtggtatttaattgtttacagtgttcagttagcacacagcaagtgcttagtaaattgcaactacttttgtccttgctgatttgttttaatgattttgctccaagtactcactgtgttctgtggtagtttctgttatatgcatgactgtaggattaccctccaaaagtatagattttagtaaaaattaaaattagaagttagagtcccttagatactgaaccctcttttttctctgtgtatacaaatatgtgtatataattatatataatgtgtacatacatataatattttagttacatagttgccaaggctttgtagctactgtagtttcagtgttacatggtatacacacacacacacacacacacacacacacacacacaacatattcatcttagggaaaatacagatgtgctcatacttaaatggccaattataagattattcacatctgtcaattttttattgacaatgtttaattttttatatagatctaagctttggtcttattgctgctcattaacactttactgaataatgagcttgaacagattttttttttttttttgagacagggtctcactctatcacctaggctggagtgcaatggcatgatcacggctcactgcagccttgacctcccgggctcaggtgattctcccacctcagccacccaagtagctgggactgcaggtattcaccaccacacccagctagttttttgtatttttagtagagacagggttttaccatgttgcccaggctggtcttgagctcctaggctcaagtaatctgcctttctcagcctcccaaagccctgagattacaggtgtgaaccaccatgcccagcttgagcagaattaatcttgctgaattcacttgctggttcttgaatttggactaaatttttggccacttattaagtgccttttttctttgataaactttggttaatgtctgagttataggaattatactcaattttttgatatcaaataccatatacagatatatatataaaatatatattttaattctgccactgctgctgataaagattaagaccctcatctcaatttttttacttgtaagattttcactgtctataataattgagaagttgttttgtacagttacagtttgtttcaatgaacaaaatgtttctagtattattttgacctctaaataacagctgctacttttttagtcattgtggttgtattcactaaattaccaacctttaaaaataatctccttccttgatgctatgtctctattgtgggatttatgcctatatttaatctcctactcaagggaggagacaaaatgctaaattcaaaggtaaaaccttataactaggcatcaatacatttatataaagtggaatgatacttgaaatgtttaattaagtctggcagaatcatatgaacacaatgtattttctatgttaaatctcatttggttttaataattagacatttttaccaattgagataaagccgtaaaatttctttacatctggcgtcttattaaatagcctttggaagatatgtatttgtgtgtgtgtgtgtgtgtgtgtgtgtgtgtgtgtgtatgtgtgtgtgtgtgtgtaggcttttgcatatattttaccattccactatctttccaacatgaggaggctttgaactgagtatattttattttggatggagtgaaactaaatgttgttggttttttttttttttttttagtccatccattctttgatttaatttggcaaacccacattagataatttagcagaagaggaattatatcttcatcctattatagtaaaacctctcactaattctgaatttatgatatttgagatgcagtatttgtgatcttttttgagtaaaaaattcaaaataatttcttcctctgaattttcagggtcatacttataaggggaaacctgtctaaaatcactagcttttcaaaccaagggctcaagaaatgaaggattccttcaacgtgccttcagcctctgggtccagcagttaattcttgtatgttaaaggacttttatgtttatggtacattttaaatgtaatattggcctattttgaaatataatgtaggtaaccacactctatttattgtagcatttagtttaatctgtaatctgtactgcttgaaggtaacaaaactgaatttttacttcagtgttctgtatgattaagacgtttccagtaagccacacaggcctacctgttctatgttattccgaattagtgaggttttactgtcctagtgtcgtaaagagtttcagtcttcttgaaattataggcatttagttacggaaaggaaagtaataatgtaggtagattttgtctcacctggatgttactaaaggttaggtaaaaaaagaataaacacaaaatgatttaatattattctgtttaacattctaaaatgcacaatgaaaatacaggatagaaagtgtgtgtctgtagattcacatatgtatcttcatataatacacacttgtttagagttgctataccacttgagagtggtttagactagttgaacttgtgaaattccttccagatttacatttattctgtgtacaaggaatttgaatcttgatgatataaaggtcatatgttagtgatgtatagaagtacattgtgattagaaaaagtaagttgttgttatttacacattccaaacaaaaatttttatattctggaatgtggtttttgatgctatggttagattgttggaaaattcaaagttggattgcaaaagccaagggtaaagaaaaggcttaaagcaagctcaatgaaggaaattgtgagaagggaggaggaaagaaggagtgaaagcagaaaaaaggtttggttgagggtagtgattggggacagggagaaaaccacaaagcaatatggctgaaggtgatggtggcacaggaaacatgaataagaataggccattgaactgcaatatattgaataaagctctttggtctttatacattttgaaaagttcagttgagtttcaaaatacttatgggcagtgtagcctttctaagatctatttaggaaatctaagtcacttgtgaatccattagcaattttttaaaaatggctagtactaacaatatcactaagtttaaaataattgttaaatgactttgcatgcagtaattttttatggaaactaaattttcccatataaactgttacattttaaattttgttatttctataacttaatgacccgtttattacataaggttttatccataaccagatcttatctctgtttttattaatttagtgatatttgcaaatggaagtatatagtctatcttttcaaaattccttctttgcaggtagattcttgatctatcacttctaacagctacagattcttcttgcttgttccttttaacatttttctcttgttcttccataataaactgatttaaatttttattttgaagatgaataatggcttaatcataattcacaaattataacacttgcagatggaagtggagaaagaggcgaaaaggatgcaagcaaaatcacaacataccctccaggctctgtgcgatttgactgtgagctccgggcagtccaagtcagctgtggatttcaccattcaggtagcggctggaactttagggtataactgcgtttttacaattgtgctcagtaacttatttcttcagatttgatgaacatagcctgttaactgtgtgttaaaagctgtagcgtggcagtctctcaaatctgtaagacctttgtatttattacagaatctctttaaacaactatggcatgatggaacaggtacttgtctagagatctaacttctagtctgtcttcctgtggacttcttttataattcaagacagtctggaagatgaaataatcctttaaatctctttcagttctagaatgctgtggtttttgtaacatgttcccatattttgttaatgttcagtgataaatgtatgtcaacaaatttactttcttaaaatgtagaaaataattaaattcttagtaaattattggcatttgggttagcttttgctgtactgttcaacctttataaaaacaaaaatgttaaattccttccttgaagattatttgataaggtattaacagacattaaagttctgctgtttcattgatgttgtacgtaattgtgcagacctcctatcctagataggagagaacaatattttatatgtagttactcattattaagaagattttacagttgacctgaaccagtgtgttaataagaattaatgttattaaatggcatttatacgaatttttaagtgtgtattatgaaaaaattatacatttatatgacctacaaaaatatttttcagaactaaaattaatttctcattaaatacaaatttgtccttcagttacctatttttaatggttctataaatcttaaatagtgaaaattacactgcttttagtatagttgtgatagaaatgtaaaatgcatataatgaaaaaataaagctgtgtgttttggttgcagtggttttaatggaaaatggagatgtctatacatttggttatgggcagcatgggcagctaggacatggagatgtcaactccaggtacttgctaacttttcatttgaagatccagagttattttttcttctgttttcttttccttcttttttctttcttttctctttttaagtatatatttctagaattaaaataatattataccactgctttgcaaagtgtggcccatgaattattggtggtttctgatcttatggttgagtgcttttgtagagataaaggattttgtagtttcaggagaaaagctgatatatattttaaataggagagtgattatgattgtcacaaaatatcactatattcttagcaattatctttttaaaggctgtgcatggcaatagaaaaatagtgaattgtatttttaaaaaatgtcagcaatggattcaaacatattcctgatgtctgttttgaaattttggtctaataacatgcagtcattgaagcttttgaataatttttggacagaacctattggtctctctgattaactcttttgacttattgcaaatagtagtagagagactggtggtaatttcctaatatccctacttcatttgttttagttttagttttagttcgtgacatgatcgtgcagctaaagattccctttctcagcttccattgcagcaggatgtggccatgtgaacttaagtgataggtgccactttggaatcttgctgttaaagggaaagggtgtacctttccatttgtccttctctcttcccagcttgctggctggaatgaatatgtggaagcagaaactaaagaagtcatcacagagataagttggagactgtgtactgagaatggcagaatcataagatgggaataggctgggtctctgacaccatgaactgcctcatcagcttcttaaattgcatgcttcttgcgtgagaaataaacttgtatcttgaagtccctcttacttgtctttcattacagcagccgagtttgtattctacgtaatgcagtcagaatacttttggtcaaaagtaatagaaaatccatctttattagttcaggtcttccagaaagcagatgccaagacaggattagatatgcaaagaatttattagatacatgtagttttgaccctatgaaaggagaaagagaaggaagaatcgggtaggagtctaggttccagcataacattaagagagttttagccaggccagtgatgtgtccccaagctgaggatacctttggaagggtcctgcattgggcaggaatagaccaacattcataccttcaccatgttcagtcacagctggtagcaccccaggggacgaatgtcctcgtgcatccaaagggacagcacctgaggctgttataatagttaactctgctctctgtagcagattgtcttgaaggagatctgagtggtctgtgtctgtggttgtctcaccatggaaacaggagtatttatgaagtctggaaagatagagagcagcttcaagccagacttcactcagcaattcacttagctgcaaaaacttaccttttgaccttctactctgccttattcttcctcgcagtcacagtgtatatgccagaatcaaggggacttctatggtttctcctttacatacagtgaaagaaagagaacttaccttcacatagtcattaaataaaattcaaggttgtcactgtgtcttgaactcactctactattgtggcagggatggaattctgccatttacatggtaaagattagtgattcttaaccctggctgtatattagaattacttagggaatttgaaaaaatatcatatgtggattctacatccagcatttctgagagatgtgtgtgttgaaaggcttgtgggtgggacaggggtggggagtggtccttgataggatgccagcatcagagttttttctaaaaggtccccagattctatatgtatccaggatgagaaccactctctacccttcgagctgaggcagggtcagatctgtgggcactgcataaaaaatggcaacaaaagggaatgtcctgagttatgtttatgaatgttgttgctaatgataagagacgattgagatatcattgagttgaacttcttattaaaaacacaactgaaacataaaattgatactactatcaagaggtttgtaaaactatcacaatcataaatatttaatgactaatatcatacttgcaaagagcaaaagatgtgtttggcagaattactttatcagatgtgtctgctggatatgacataataccaacagtgttgagcaattactgtcacaggtgttagatgccaactgatattttgctttgcaattgagtaacattactgaaatgcagaggctgaatctgggtagtaattacagttggccctctgtacctgtgggttctgcatctgtggattcaatcaatcttgacagaaaacatagttaggcctacgataattgcatctgtactgcacatgtacagatgtttttgtcttgttattatttcctaaacaacagagtataacaactattaacatggtgtttacattgtattaggtattataagtaatctagagatgttttaaagtatacaggaggatgtgtagtttatgtacaaatactatgccaatttatataagggatttgagcatccctagagtttggtagctgcaggggatcctggaaccaattccccatggatgctgagggatgactataacctgtgtggggaagaacgtggcaactttttgttctgtttctcatttgagagttgggacacagaagaagagggtttatttgtatcatcgtggctattgttgttgttatttagctggtggttattttgtgagccatagaaactgggaaaggtgcattgcagttagataagatggggtctcagtcatgtatgcagcaaagagggcacttcacagaaaataattggcaaatagtatttcatggtcttgccagagtggaaagggaagtagtgaaaagcatggccatgttatagtcaggttatgttatcttactgtgcacttggcactcagaagaatgagcagcatgagcacaacatccttccacaccacactgaggtgtgttggccgctgagagggaaggtgttcatgtcagcttttaaaatgaagagattacctaaaaacattacttcgtggtcctgattataccagtgaacaccattcttcgattcccaagtctgaagagatatagggcagtttcaggccagacttcctcaggtaataatattgtagcaatatgctcaatattttaaattgcttgaatttatcgtcttcatgaaatacgatagcatttaatgttgaaacaaaatatcagggtttcttatgaagattaaacattggtaggatctacacagtggtatagtaactggtagatgctacatagtcatcgaacctggaactgtagaggcaaggttcagctcttcacttcttaccaaccatatgactataatttcttaagattaaacactggtgggatttgttttaactgcccaaagttgttcttttccagcatggaatgttttggtccttgttccattgagtggtaactgaaagactgattacatgaagtgtgggcagagctacaacggataatgcagtagtcatacctatatactgtgcctagtggattagatattcatcaagaccgcttcagtcttaagcactcagttaccctgtctgtctggacttttgtggaacttggaaagtagcaaccaagaactctgttattaattcagaaaggagttgattatgtctatagaggaacaatcttgggaggaacaaagcttctatagcatatgtattaactacaacaaagcaaaacatttacagatagttgaaataaagggttacagttggagaatatgtttgaaaagtctctagtaatcaagagctgttaactggaatttactatatagcacacattaataatgtgttgtttaagatcaatattgtggtatgcatgataatgaagatggaaaagtgttattgatattctctttatttaatgtgttatattgccagaatgactgagttcagtctctaggttgggtcctgattctgtcacttggtagctatatgatcttggccaggttacttaatcttcttgtgtctcagtttctcaagtttaaatatccataatctgatgatactttcttcttctgtgatgcagccctactttatttccctttgactttatgccccacagccctctacttttttactcttttttcaccctttttttaactttatttcctactcaagttaaagactgttgcttaatttcttcacctatactcttaccagtttcttcagttttcccactctcttggtcgtggtgctaggacatgataaaaccgcaaacatgaatcaatcttaatatttcctatgcctatacttaggcttctggaaggtaagaaatcaccatacctatgcagattgatacttaatttccagattcaaccagttcctgtacaatggtcattaatcctcctgttgtgtagtattgaactatttgacttaataccttttgagagtttgatgaaagacatggggtctctatgaaaaatacaagaatatacacttaaaaatttgcttgcaaatttaggagatcttttgaagctcactcatggacttcctagaagtcctttatatctgagttgaaaactcctgctctcggtatgtatctattccatctaccaccacaccttttctcgaatcttcaaactctccactcacctgtttcctccttttagcataggatctccatgtacattgcaacagatgtaagaaattattcagcccaccaaccctgcaaacctacttccatccataccacttctctgcttctttcctcatgttacagttaaagaggaacctgttgtgtccttgctatgaaacctagctgtcttttccttagagatctatttctgtttcctgtcttctctctaacctttgtctttagcgtctttattttgagtcaccttttaaacttcctcaaatctcttatcattaaaaaaaaaaaatcctctccttcagcttcatttttcctttagctgctggctctcctttctttttccctttagtgctgggatacttagagctactttgtgtattctctgactccttatctgctgcattgtggtgcctgcttccaccaaagagacctccttctgctaaatcgagtggttaggtgtatgtctccttagagcatttggcatgtactttattttccacgcttgaaacctactcctccaatgagtctccttgagttctttctttctgtgtggtttattctagatgtctgtgagctgttcttcctcggtctgtatctaaaatgttagaagagactcataagggaaattaggatctcttcttttctctctcaaagagtcttcctagatctcattcattctcatagcttaagtgaatattaccactatctactctgttgcttaagcaaaaaaaatcttccttgccccatatcgatcatctgccaagtttcatcaattctgttccttacatagctatcaaagtcaccatctctttcactctgcactgctcctagttaaggccttcatcataaattacccaaactactatataatttttaaaatttaaagtgtcataattacatttctcaaaagcagagctctcctgctatgtgtggactatttctataatggttgtgtttctaattacagataatggttactaaaaatggctgttaaaagtaggaaaggatactaggatgtcatttatcataatagttactgttcattaagtgttaactgtgtgccagacgctgatccaagtacttgacacgtatgggcctctttactgttcacaggaactctgtgggggtagacaccgtttgttagtattctccatgtgataaagaggaaactgaaatacaaaaagaatgaaaaacttgttcgaagtctggtcgcattattattaagttggggagccaggattcacaaccagccagtctagctttaaagcctgagcttgtagtcactgctctaatgcctctcatgatactgtgttttgactcattaactgtgattctaagatctaaacttatagcttagtgataaaagttagaaaagatgatcataaaattatttcatctagttcctggattgaatttctgttaaggcattacatgatttttatttcatccaataattatttataaagatctctaacaggccagtcagtatacagagtaccagattaaaaataaatgtagcaccagtttttcagaaattattatgtgtctataattagggtaattacatttagaagatctttttgatgatctccttaaagtcagcaactgtctttttcatctttgtttacctagtacctggaatggagataggcgtttagcacttaaatgtttactgaatattcttatgagtgccttttatctttcctactccttgttgcattgcttacttattgtttttattttagttgagttttgtaagaaattgacttactttttttttttttaacctaggggatgtcccactcttgttcaagcattgccaggccctagcacacaagtcactgcaggcagcaaccatacggcagtacttttaatggatggacaggtcttcacatttggaagtttttctgtaaggaatttttaaaacattaataatattgcattataccattgccttataatttgtctatattagctcttttttctgtttccagaatataatataacaattatattataattgttacatatgcatatttcatgccttcatccccaacacacactaaacctgaatgatattctttgaagtaattttcctctctagctcagcattagaatttattgaatttaacagctttgttagaattggacatgtttatttcagattaaagtcttttaagcattcaatagagctaattctgtcataggaaaggttatttctcatctaacttgtagagatgaatttttcttaacacatagaactatgctattttgtaaccttttaaaagcctagttttttttatttgatttgtttaaaattatactttcttttttccttttccaccctctgagtctatccgcctgtctgttacataggcgcttgtgcacattctctccctctctctgcaccaaagcttaaagagtgaaaatgctctaagaattttgtgtagtttgggcatagtagatatcaagaaaaatctttgcgagactgtgctaatactcttgtaccatttcagatgtggataattactaagaactcttgaacctaagtgtctgagatgacatttacagcttttgattttttaaaaactgtaaatgtgtacttaaaatattttatttgaaaatggtttcaaacttacagaaagattgcacaactaagaacagtgtgtagaatatctggttagtgtctttaaccagattcacctattgttaacattttgatccagcatttgctttattattgcacatgtggctgttctctttctcacacacgcagtgtgtgtgtatgcacaaatgtgtgtgtgtacgtatgtatataaaatatttttttttctcaaatcctttgacaatgtcttgtgtacattatggcccgttacctcctacatactttagtttgtattttgtgaattgacctaataattattttttggcatttctttcccctccaatctaggatcaggcattacatttgcctgttgtgtgtctttagtctcctttaatctgtaacatttctacagctatgtattgtctattataacattgacatttttgaagagtatatagtactgttttgaaaaatagaatcctcttcatttttgtttctttgataatgtcctcatgtttagatgcaggttgtgcacccaggccaagatactctgtaagttatgttgtgtttgccctcactgcatcacatctgaaggctcagaatgtccatctgctcttcattgattatggacattttgatcacctgatcaaatgtctgatttctgtactgtttctcctttgaaactataagcaacctgtaggttgacacttttaagatgcctgcttctcatcaaaatttccccctagatttagcatccattgatcattctttcctgagctagtctttactataatgtttaaaaaatggtgatttttttcaactccagtaggcacttggccttttactctaagcaagagccctcctttcctgtttacttgtttttctacttattattggtatagactcaggtatttgtattttcttcattatcatctttaattatttgggtgcctagattatctccgatttggccagcagtagccccttcaagttggctcctgtattcttgtgacatgccccttcattttttggagtgcttcctttctttccagcataacaagttgttcagggtttatcttgtacaggccctgtatgactgtcctagaatcagccatttctccaagaaccctgattctttttagtggggaatagtattagacactaactgggcactagaagtgttcattgctacaggacgtctttggatctctggtatgcatatatttatatgtccacatatatgtatgtattttagaaatcataggccaggtgtggtggctcatacctataatcccagcactttggcaggccagtgtaggaggatcactcaagcccaggagtttgagaccagcctggacaacatagtgagaccccatctctattaaaaaaattagctgggtgccgtggcacatgccagtggtcctagctgctcaggaggttgaggtaggaggatcacttgagccctggaggtcgaggctgtagtgagagctgtgattccatcattgccctctggcctggacagcagagcaagatcctgtctcaaaaagaaagaaaaagaaccccactgtgacttctacttccaatccattcccacaaggttctttcttacctgtcctgttctatatttatgtactttcttcctcagtaagaatctttcctctcaacaatatcaatatatttatttctcacttagtccccagatatgtctaaaatagttttatatttgctttgcctgtacaagttcaaaaaaacaaactcactgaaaggtcattcttgctctttcccctcccacctgctccccaccccaagtctgaaggtgtatagtcaagcactgtccatgagtggcctggattctctctctcactcctttcagtggaaattgtgattcctttggaatagagtggggctcatttgtttcagtttaagattcccctcaccctatcctttgattaagttttatttcatttttaaaatatatagaacatttacatgttgctaaaaattaagttatacaaaaattgtatactcaagaggtgtcacttcctcccatatccttgacattcctgcctccccacacctttcattccatacttggaggtaatgaatgtcattgttttacagtttattcttcttgtgtttcttcttgtgaagatatataagcatgcagacacactattttatatatatatacctatatatgtatgtatgtgtatacacagttatgcactgtataacagtattttggtcagtgatggactgcgtatgctaaggtgttcccataagattataataagtactttgactataacttttctatgtttacatgcagaaatacttactaatgggttacagttgcctacagtattcagtacaccagcatgctgtacaggttggtagcctaggagcaataggctacaccatatcacctaagtatgtagtagatataccgtctaggtttgtataagtccactctgatgttcgcagcatcacagaatcacctaatgacacatatttcagaacatatccctgtcattaagtgacatgtgactgtacataaatatatatatttcccctttcccgttttttaaaggtaatagcctatatatgctcttttgcactttgctcttttcgcttcagcatctctcctagaaatcactccatatcaattcataaagctcttcatttttttttttacctccatgtaataccccattgtgtgtatatatgatagtttattcattcagtttctcatgtttatatatttacgtggtttccaatactttgtaatggtagtagtgaatattgttagaggtgtatctttgagataaattcctagaagtgagattactgtgttgaaggttaatgcctatgtagttttgtgagatattaacaattctcctgtattttgctttcacactaacggtgtatgagattgcctgtttccacagaattgccaacagaatgtgttgtggtacctttaatttgtgccagtctgatgggtgaggaatggtcctgcttacttccaagttcctttgcctttagttggtgctctgatcctcaaagttcggatccatatttagtattttggcattgaaggttaactttctttttgggggcatgtgtttccctgtgctttttctaacttcttcacacgcctctgtcctccttcttaagaacttccctgttcgtgctttgcacatgctcaggtttgcagtagccggtgggtggagagaactcttggaatttggctcttctgcttacaggaaacatagagatcatgacacccgctgtctccttccactgctgaaggtttgagtcacatatagatttgtttgcacagcatatttttgtgtcttttgaggtggttatgtgagtgataagatttgacgtcagacagtcattgtcctccagtcccagcagtcctagttgtgaacttttatgaatttaaatgtggcttatttataaccacattctggtccttatggggttgtccaggtatctaacacatttttggactgcttttcttcatattagttggtagtaagggaatacatatttatttcaatagtcaatgaaaaagataactaatttatttttcctccaaaagcagaatatatccctgaatatgatatattttgctcactatttacttttttttttctttttgagacagagcctcactctgtcatccaggctagaatgtagtggcatgatgatctcagctcactgaaacctccggctcccagattcaagcaattcttatgcctcagcctcctgagtagctgggattacaggcccatgccaccacacccagctaattttcgtatttttagtagagacgaggtttcaccatgttggccaggctggtctcaaacacctgacctcaggtgatcctcccatctcggcctcccaaagtgctgggattacaggcgtgagctactgcacctggcccctgtttacatttttgtgctctcttatttttgtcaaaaaataaggacagatcttaaaagagtatgagaaataaatgttgagtgttttggctgcttctctaatcatgtgcttgccatgtttccttccctggttctgtccagggattgaagctcagttgcttccacagttagtttgtggctgtgttttcttgctgtgctacctggtggttggatgagaacgtgaccagagtgttcgcagtgctccaggctcccttgttccagaatgctgtgaggggagagaggtcccgagccaggggtggaagaatatctattgagtcaatgtcagcattcttttattctttttcagctacaggcttactagtgagaaacttcctattactttttaaattaaactcttacctgaccaggtccctatagagagccatacttttaaactggttaatagttatttacttaaaatatcatatcttaccattgattgtgctgtttattttaaaacataaatgaaataatttagggacatatcatgtgaaaacaacattacatatttttctaattgtattcacagtttcagtatttattttggtctttttacatggatattctcagattatatgagttcaatagtgactttcttttatactatcaacttactatgtcaaagtgactttttaaatgaacaatttggaatactaatattttaacatattgtgtgtatttgtcgtacttttcatttaaatgtaggtgaagacaaagtattttggagatacttcttgcgtggaaaatatttcaaacttttttttaaaaaataatgcactttttaagactttccaggaataatagaaattattttattactttataaatatttggtccaaattcttacatattgtatttattttaaaaacccaaactaacaattggtgaaaaataaggtttcatagattgatttttatctactgcctccttaactttatgaaaattagattgtttacacagcaatattgctaatatggtttatttaatcttttagaaaggacaactgggcagaccaattttggatgtgccatattggaatgcaaagccagctcccatgcctaacattggatcaaaatatggaagaaaagctacttggataggtgcaagtggggaccaaacttttttacgaattgatgaagcacttattaattctcatgtacttgctacatcagaaatttttgccagtaaacacataataggtaataccagaaataaacaaatgccccttccaaactcttgtcttgattgatagtaaatggtttatctttccctttaatgaaatgtacttttgtagctttttgttttctttctgctcaaagaaatttaaacaaagcctgtaaatgtctccgtttatatgtgtctctatgctgaaagaatggaataattgggagatctggaaaccacagtatttgtgagctagacagtggtagaactataaactaaattctagtaggataaaatccatattagagtaagagagttttatgcattgtttattgctaaataaatgttagaaattaatttttatgggaggttatataaatccaactttgaaatttcacgaagaccaagttatcaatctgtactgtgtccatgatactgtgaataattatataatagagtggtatagcaagagattaggagcatgtgttgtggatttagacatcttgaggttttattcctaattctcccacttaaagattttttaattacttagcctcttttattctctgcttattcacctgagaaatggagataatactactttatgggttttggagaatttaaaagggtaatatatattaaaattatatataaatatttttttcttaaggcttggtacctgcttctatatcagaacctcctccatttaaatgccttctgataaataaagtggatgggagttgtaaaacttttaatgactcagaacaagaggatctgcaaggatttggtgtgtgtcttgatcctgtatatgatgtaatttggaggtaagcatctcagtttataaaatagtcaataagttttgatgcagttatactcttatggtaatataaactttatttacaggtggacttttcacatgtgagtactctcctgtttatctaataactgcaattctgtctcagtgattcacagggacctttatactgtagcatagtggttaagagcatgcattttaagcaacactacctaggtttatcccagctccacaacttgctttctgtgtcaccctggagaagttactttgactgtgtgcctcagttttctcatttatgaaatggggatgctaatactacagaggtgcatactaggtgccatataagtgtttgctattatcattataattattaattttactgttgagagttctgttgttatttttagtctgtcatcaatcctgtttatattgtatagaattcattctattttttgttctcataattattattaactatgaggaggaaattatgccaaagacaataaagaataaagaagataattgagcttgccctttaagaatgtgcattaaaaaatactcttactggtagagaaatatacaggattatatgcattctggaaattataaaaacatagagataaataaggcacaaaagagactcaagaaattatgttgcatttgtttgaaatttattgtggtgctgggccgggtgcggtggctcacgcctgtaatcccagcactttgggaggccaaggcgggcagatcacgaggtcaggagatcgagaccatcctggctaacacagtgaaaccctgtctctactaaaaatacgaaaaattagccgggcgtggtggcgggcacctgtaatcccagctactcgggaggctgaggcaggagaatggcgtgaacctgggaggcggaatttgcagtgagccgagatcgcaccactgcactccagcctgggcgagactctgtctcaaaaaaaaaaaaaaagaaaaaaaaattattgtggtgctttatatacaaaatgaataaataagttacccttcttagtgtttctagctttggatactaataggaggtgtaccctgccatcaccacagtggctcatcacagggctgagcacttctttccttttgcggccggccttagtaggcaagattccagtaagttgaaaggttgaagtagtgtgagtgaggagactttttctctgtcttccttcttctacccatgcctgcttgcattctcatcagacagccaagttctaagaggccatgtggtagcaatgggatagatgatctcaaatgtcttgccaactccttggtaattttcttccgtgttccaaaatagagattaataacgaggcttgcattcagagtgaagagactgctgcacttcccaggttgctggccagcttcctctcttatttgggcattttatgagaaggcgtagtggccagtattgagttgagtacatctcaaatcaattacatctccctggcagcattgtgcagggtagatgaaataacaagagactatgggtgagtggtcttttgtgatcattcttatgaagacatgaggtattaagaaccaaagttagaatggtcacagtgggatagaatggaggggatagagaggagaaaagatgtaaagatagaattggctgcagttacttaccgattagaagtcaatgttcaagagacttagttaccaaagagcaggctctggtttggacttaggtgtaaattgatgttttactgttttgtagttcttacatgttttatttttaaatgaagtaaatttgcattagcaaaacaaaaataaaaattggcagtgtggaatctgtttctggatgcattcactaagttagcaataacagtataactaagcgtaacagtaaaaacttacttgaactagaaccatttccgtggggtcttaacagaactctcaagtatgatgttcagctggttgaaccttgtcttttccatttatggtagtagcaatgggacagatgatctcaaatgccctgccagctccttgatcaatttctccattgttggaaaacagagattaagagatgttttggtaaaacagctgagaggtatttttctttaaatcaggatcagaatttctgttatcctgacttctggctgctttgtgtcttttgtgtttttcaatggggcacttaaattttataattatcatatgcattgcacatccaatattagcttactttgactttccttctggcctgcatctgcttcattttatattctttaggagccaactttgcctgagtaattagaaacaacatctataaagagcagataaatgttaagaacacctgttttatttcccacatacaaatttacacaaataatatgtgaatttgtgttattttttaaaaaacaaagcaaaaatacatacaggaaaaaggatggtcatactctgtatctgtttcttttcccataccgaattccactagccttctctgtggatcacaagtatgaagtttgatttatatcatctgacaccttttaaaatgcatgtatttttctttttatgtaaataaaaacatacctactctaaaagttagattttttgctcaaaaataatttagagatctttccaagtcagtatttaaagaacagcttcattattaaaattgtggaattctgtagtctgggtataccaaatatacttaactgtttgcccatcagtgaacattttagattttttttaatattacgatactacctacaatgctacagtgaacaaatacacaggtatctacacacagagttttacacacatgtggaagtatttctgcaagctagcttttaaaatgtagaattgctgagttaaaagataggctcacagaattgtaaaatgactgaggtaagtctgaatcactttagaagtttatttttccaaggttgaggagatgcctgggaagaaaagacaagccacagcaggatctgtgccctgtactttttctgaagaggtttgaggccttcagcatttaaagaggaaaagagagcaggaggagaaaaggaaaagaaaaaatgagaggatgtggtcacattcttgtaaggttttgattaggcttactgaatccacatgttgcacatgaaaaggaaggggtagagggaacagtgaattttgtatttggagttaaagtaaacatagagtagaggaagcagtcaaatactcattcatctggggctggggcgggtggggcttggtggggggatgggcagataatttctagtatcttcttgtctcttaccatgaattttccagaaccaaaaagagatttgaatccatggatattagaaactatgtgtttctattatttaccatcaagagagttagtgaaactgttgaacactaaagaccaaggggaaatcttaaaagcaaccagagagaaaagaatgattacctaaacaatgagatcaacagaacttcccaaaagcaataatgcaataatattaatgctactagttatagaagctaccataatatattttctcccaaatgctattttaagtgctttttaaaaataatttaaaattttttaattgtggtaaaaaaacagtataaaatttaccatcttaatcattttgaaagtctgcagtcagtggtgtttagtgtatttatgttattgtgaaatagatcatcagaacttttttttcttggaaaactgagactctatagttattaaacaatagcttgcatccctcacttcccgacccctaaaagaggtaaagaagtggagatagttttttgaagtttttgttacaaaagagatcagagaaatgggatgatgaagacgttgtgggttcctaatagtttttaattggtttttactgtttattcatatggttgaacatccacatccattctatctgtgcttcctttctatggatagcattccatgtccttgccaatttttgctatggattgattgtttctagctcttatacattgaggatattgtagctgtgtctcacaggtacacttcacatttttcttcaacttgtcctttgtctttaaacttttttaatggggtatttgttgagtttcatatttatatgtatgtatcaaaactttatacagtcaagtattataatctacattactattttctgtattaagtattataatctacgttactattttcaaattatttcacttttagtgatatagtttttttatatgtttagcttagatccagaagataatgagctacatttgtcctctttaagctgactatattatttattctctttgtatcatcagttatgttttaggaattttttttaaaaacaaaaccacattgaggtgctgtaattattctttttctttaaaggtttcgaccaaatactagagagctgtggtgttacaatgcggtggttgctgatgccaggcttccctctgcagcagacatgcagtccagatgtagtatcctaagtcctgaacttgccttaccaacaggatcaagggccctcactacccgatctcatgcagctttgcacattttaggtagggttgcgatttgatgtaccattaattcacattaatgtgtgctgtccagggtttttttttagagtatttaaatttatatgtaattctgtcttgtttgttaaataataataatgtacaaaataagttaaatcctctaagaggctaattccacagaaaacaatacatgagacctgtactaacactatcaaagatttatgtatgcccaattatttaattttcacagaggtagacccttaaaaataatgtttttgctcaacttgaatgtataaacttttaaagatatgataaaattttcaagtatataatatttaatttatgtatattatgttaaataaattctataaaatgtaatgaattttatatacatcttctgtttcttcttatataacacagttttccacagcatatcaacttcattcttatttaattactttgagatgtagtacttatttaattactttgagatgtagtgcaatatattattttgtaactggaaattttatatcactctatgtaactatgtattttgttgcttttaaaaatgatccttaaaaagactcattttcagactctagcacttgcaggtgtgaaatcatattcctaaatataattaccatatcataaatataactctttgtttcttttttctcaaatttcattttggtttctattgtagacatccaaaactagtgttgattaagtttgtgtgacactaatgtgtcttcctcaaatagcactttaagaatcaaactaatttggagtttcataaaaggaagaaccttgtaagaattcaaaggttaagtgatttcaacttttcagagatccagttttgtgtaaaaggttgtcgtatggcaagtttaaatatgatcattaatcagacaagggataatttgtattggttttaagcattcttccatgaaatgatgtttaaagcttggagtaacattctgagtttatttattttaatttatcccctatagcatttaatgtcatcaataccataggacattttaatttcaaggagttaaattttgtttctttgttgttttaggttgtcttgataccttggcagctatgcaggacttaaaaatgggtgttgcaagtacagaggaagagactcaagcagtaatgaaggtttattctaaagaagattatagtgtggtaaacaggtttgaaagtatgtatacttcggtttaggaaatgttgtcttacaactgaaatatatatggatcttttaaaacatagattatgatttatatattctaggtcatggaggaggctggggttattctgcccattcagtagaagctatacgtttcagtgccgacactgatattttacttggtggtcttggtctgtttggaggtagaggagaatatactgctaaaattaaggtaaagttcatcaacaatgttgtccttttttgtttgaacatactgattttgacttaatttattattttattatttaatgtctaatttttttcctttttaactgggtttattttgtttttaatgcttctttttaaaaagcgatagagaaaacattgctaaaatcgaatacagtaatactaaattttttaaatttatttatttattattattatactttaagttttagggtacatgtgcacaatgtgcaggttagttacatatgtatacatgtgccatgctgatgcgctgcacccacgaacttgtcatctagcattaggtatatctcccaatgctatccctcccccctccccccaccccacaacagtctccagagtgtgatgttccccttcctgtgtccatgtgttctcattgttcaattcccacctatgagtgagaatatgcggtgtttggttttttgttcttgcgatagtttactgagaatgatgatttccaatttcatccatgtccctacaaaggacatgaactcatcattttttatggctgcatagtattccatggtgtatatgtgccacattttcttaatccagtctatcattgttggacatttgggttggtttcaagtctttgctattgtgaataatgccgcaataaacatacgtgtgcatgtgtctttatagcagcatgatttatagtcctttgggtatatacccagtaatgggatggctgggtcaaatggtatttctagttctagatccctgaggaatcgccacactgacttccacaatggttgaactagtttacagtcccaccaacagtgtaaaagtgttcctatttctccacatcctctccagcacctgttgtttcctgactttttaatgattgccattctaactggtgtgagatggtatctcattgtggttttgatttgcatttctctgatggccagtgatggtgagcattttttcaagtgttttttggctgcataaatgtcttcttttgagaagtgtctgttcatgtccttcacccactttttgatggggttgtttgttttttttcttgtaaattggtttgagttcattgtagattctggatattagccctttgtcagatgagtaggttgcaaaaattttctcccattttgtaggttgcctgttcactctgatggtagtttcttttgctgtgcagaagctctttagtttaattagatcccatttgtcaattttgtcttttgttgccattgcttttggtgttttagacaagaagtccttgcccatgcctatgtcctgaatggtaatgcctaggttttcttctagggtttttatggttttaggtctaacgtttaagtctttaatccatcttgaattgatttttgtgtaaggtgtaaggaagggatccagtttcagctttctacatatggctagccagttttcccagcaccatttattaaatagggaatcctttccccatttcttgtttttctcaggtttgtcaaagatcagatagttgtagatatgtggcattatttctgagggctctgttccgttccattgatctatatctctgttttggtaccaataccatgctgttttggttactgtagccttgtagtatagtttgaagtcaggtagtgtgatgcctccagctttgttcttttggcttaggattgacttggtcttgcgggctcttttttggttccatatgaactttaaagtagttttttccaattctgtgaagaaagtcattggtagcttgatggggatggcattgaatctgtaaattaccttgggcagtatggccattttcacgatattgattcttcctacccatgagcatgggatgttcttccatttgtttgtatcctcttttatttccttgagcagtggtttgtagttctccttgaagaggtccttcccatcccttgtaagttggattcctaggtattttattttctttgaagcaattgtgaatgggagttcactcatgatttggctctctgtttgtctgttgttggtgtataagaatgcttatgatttttgtacattgattttgtatcctgagactttgctgaagttgtttatcagcttaaggaggttttgggctgagatgatggggttttctagatatacaatcatgtcgtctgcaaacagggacaatttgacttcctcttttcctaattgaataccctttatttccttctcctgcctaattgccctggccagaacttccaacactatgttgaataggagtggtgagagagggcatccctgtcttgtgccagttttcaaagggaatgcttccagtttttgcccattcagtatgatactggctgtgggtttgtcatagatagctcttattattttgaaatatgtcccatgaatacctaatttattgagagtttttagcatgaagggttgttgaattttgtcaaaggccttttctgcatctgttgagataatcctgtggtttttgtctttggttctgtttatatgctggattacatttattgatttgcatatattgaaccatccttgcatcccagggatgaagcccacttgatcatggtggataagctttttgatgtgctgctggattcgttttgccagtattttattgaggatttttgcatcaatgttcatcaaggatattggtctaaaattctcttttttggttgtgtctctgcccggctttggtatcaggatgatgctggcctcataaaatgagttagggaggattccctctttttctattgattggaatagtttcagaaggaatggtaccagttcctccttgtatctctggtagaattcggctgtgaatccatctggtcctggactctttttggttggtaagctgttgattattgccacaatttcagatcctgttattggtctattcagagattcaacttcttcctggtttagtcttgggagagtgtatgtgtccaggaatcccttatccatttcttctagattttctagtttatttgcgtagaggattttgtagtattctctgatggtagtttgtatttctgtgggatcggtgatgatatcccctttatcattttttattgcgtctatttgattcttctctctttttttctttattagtcttgttagcggtctatcaattttgttgatcctttcaaaaaaccagctcctggattcattaattttttgaagggttttttgtgtctctatttccttcagttctctgattttagttatttcttgccttctgctagcttttgaatgtgtttgctcttgcttttctagttcctttaattatgatgttagggtgtcaattttggatctttcctgcttcctcttgtgggcatttagtgctataaatttccctctacacactgctttgaatgtgtcccagagattctggtatgttgcgtctttgttctcgttggtttcaaagaacatctttatttctgccttcatttcattgtgtacccagtagtcattcaggagcaggttgttcagtttccatgtagttgagtggttttgagtgagattcttaatcctgagttctagtttgattgcactgtggtctgagagacagtttgttataatttctgttcttttacatttgctgaggagagctttacttccaagtatgtggtcaattttggaataggtgtggtgtggtgctgaaaaaaatgtatattctgttgatttggggtggagagttctgtagatgtctattaggtccgcttggtgcagagctgagttcaattcctggatatccttgttgactttctgtcttgttgatctgtctgatgttgacagtggggtgttaaagtctccctttattaatgtgtgggagtctaagtctctttgtaggtcactcaggacttgctttatgaatctgggtgctcctgtattgggtgcatatatatttaggatagttagctcttcttgttgaattgatccctttaccattatgtaatggccttctttgtctcttttgatctttgttggtttaaagtctgttttatcagagactaggattgcaacccctgcctttttttgttttccatttgcttggtagatcttcctccatccttttattttgagcctatatgcgtctctgcacgtgagatgggtttcctgaacacagcacactgatgggtcttgactctttacccaatgtgccagtctgtgtcttttaattggagcatttagtccatttacatttaaagttaatattgttatgtgtgaatttggtcctgtcattatgatgttagctggttattttgctcgttaattgatgcagtttcttcctagtctcgatggtctttacattttggcatgattttgcagtggctggtaccggttgttcctttccatgtttagcacttccttcaggagctcttttaggtcaggcctggtggtgacaaaatctctcagcatttgcttgtctgtaaagtattttatttctccttcacttatgaagcttagtttggctggatatgaaattctgggttggaaattcttttcttaaagaatgttgaatattggcccccactctcttctggcttgtagagtttctgctgagagatccgctgttagtctgatgggcttccctttgagggtaacccgacctttctctctggctgcccttaacattttttccttcatttcaactttggtaaatctgacaattatgtgccttggagttgctcttctcaaggagtatctttgtggcgttctctgtatttcctgaatctgaatgttggcctgccttgctagattggggaagttctcctggataatatcctgcagagtgttttccaacttggttccattctccccatcactttcaggtacaccaatcagacgtagatttggtcttttcacatagtcccatatttcttggaggctttgttcgtttctttttattcttttttctctaaacttcccttctcgcttcacttcatttatttcatcttccatcgctgataccctttcttccagttgatctcatcggctcctgaggcttctgcattcttcacgtagttctcgagccttggttttcatctccatcagctcctttaagcacttctctgtattggttattctagttatacattcttctaaatttttttcaaagttttcaacttctttgcctttggtttgaatgtcctcccgtagctcagagtaatttgatcatctgaagccttcttctctcagctcgtcaaagtcattctccatccagctttgttccgttgctggtgaggaactgtgtttctttggaggaggagagttgctctgctttttagagtttccagtttttctgctctgttttttccccatctttgtggttttatcgacttttggtctttgatgatggtgatgtacagatgggtttttggtgtggatgtcctttctgtttgttagttttccttttaacagacaggaccctcagctgcaggtctgttggagtaccaggcagtgtgaggtgtcagtctgcccctgctgtggggtgcctcccagttaggctgctggagggtcaggggtcagggacccacttgaggaggcagtctgccctttctcagatctccagctgtgtgctgggagaaccactgctctcttcaaagctgtcagacagggacatttaagtctgcagaggttactgctgtctttttgtttgtctgtgccctgcccccagaggtggagcctacagaggcaggcaggcctccttgagctgtggtgggctccacccagttggagcttcttggctgctttgtttacctaagcaagcctgggcaatggcgggcgcccctcccccagcctcgctgccgccttgcagtttgatctcagactgctgtgctagcaatcagcgagactccatgagcgtaggaccctccgagtcaggtgcaggatataatctcctggtgtgccgttttttaagcccgtcagaaaagtgcagtattcaggtgggagtgacccgattttccaggtgccgtctgtcacccctttctttgactaggaaagggaactccctgaccccttgcgcttcccgagtgaggcaatgcctcgccctgcttcagctcgcgcatggtgcgcacacccactgtcctgctcccactgtctggcactccctagtgagatgaacccggtacctcagatggaaatgcagaaatcacccgtcttctgtgttgctcacgctgggagctgtagaccggagctgttcctattcagccatcttgcctcctccccccctacagtaatactaaattaataactcattaaaaaaatcaaaattctgtaagaagggaagaaatgaaaggaaaattattttcaacaatttaccactgaagaaagactatatctcagtcctttctgtggaacttctggtggcctagcaataccagtgcctttttatgtggctactttcttatagccattttaatgacagaggaggcacaactgtttagggacattttgatgtagtatgtaaacagacatcttcaccccaggagtggagaccgggaggagtgagagatagtgttaggggaggattccatctggtaggccaactaaaaatcattgagagtcatatgttaccacacctattcgtatccaaaagggccaacaaattatcctaaacaaagtatatgttcattctttgaatattcactaatagcttgaatgttctgcttctcaaattacctgattttgctctaaaagtgaaagagaaaactatagtttctgcctgaatttcaatagcaggcttcacaaaaaaatctcgattaaatgtcatctgttcaggaggtattttcttagttctctcaataatatctaaaaatgatatcaatatgttaacactcttcttcataatgctggtttccaaggacattccccatggaggcccaaccagaactgacaaacttaaagaaatatttgcttatttctggttatagttggaatacatgctaaaaaagcagataatgtaagaatgaataaagtaaaaatttaaagctcctatagttatatggagtatagtgatagaataatgattaggactcttgtagcacatatattttgataataagaaaaatagtagtttggctcatttcagtgcaactgctttaaaatggttattttaggatgtcttgattcatcctcacgttagagaagcaacctcaatgtgttatgtagttctatttgacaatgtctaataagctcatcaggaggatatttttttttctagctctaagagattttttcttttcggaatagtgaacaacgtagtggaccttatcttcaaacactgtagtatatagaattatggaggccttccatatatgttgtttttatattggccctatttttctttttcaaactcaaattttacctcactcctcctattgtctggaaatgatcttgccttttgtattcaattatccagtgagtgggctaaggcctttcaacagtagtcagtctttttttttccttcccttcacctctctgtaatgtatgcatttgttaagcccttctgtatttttttgttttgttttgttttcatacctgtagttttttcttctatctctttgtaggttcatccccttccacctggccactgaatgtaggagttcctcaaagacccagccctaggccctctttcttcacatattcgtatatgcattctcttttgagcccagagttttaatagccatcagcataccataatccccaaatttgtatctccaactcagacttctttgatgcattttcatgtaaatatgttaaaggcacatcagttatcagtcataatatctcaactgaattcatgctcttccactgatctctctctctcatgattagtaccatccaccatatgcttgatacataggccggaaatctagacatttttcttgataggcctcctatatctctttcttctccacaaaaaagacttttctaccactaccctaggcaaaggtatttgtcttggactagccacctagatagtctctgtacatattctttctatactttctgtctccattctctctcttaccattcctacttgagcctactccagtcagatttttgcccccaccactgacattgctcttgtcaaggacactgatgactttcatattgctaaattcaatagtcaagcctcagctctcatcttccttaaccagtcagctgaatttaacccaattgagctcttctttgaaatactttcttctcttgtcccccacgacactacacttcatttttcttttgtcccactgactgttcttcttagtctcctgctggttgctcatcatcttcctgacttctaaatgttggagtgccaagaactcagtttttgacatcttttaaaagtattctcttcctcatagtggtccagcctcatggacatttatctctgatgacttttaaatttataaacctcttctctgacctttaaaccaatatgtacaacagcctacgctgtgtctccacgtgactctctgataggcattttggaggtaacatattcaatactatgcttctgatgatctgcctaaacctgctatcctcatgttcttccccatcccagttaactgactcagcatggagttaacttcattcttctgtttcctcaggccaaaaatcttggagtcatccttgattctactctttcttttacacacatccacttcgggagcaaatattatcgtttccactttcaaaataaatcccgtatctaaccacttctgaaaactgccactgctatcaggttgggtcatgccacagccattctcatttgtattattacagtagcctcccaacttgtgttctacgtctgtcctcaccccatttgttctgttctgagtgtggcagtgattctcttaaaatgtacaacagattgcatcattgtctcttcagaatcctgtagtgtcttcctatgtgagaatgagaaccaaaagccttagaaggacctacaaagtccccatcagaactggctgtcatcacctctctgaccttttgttctactagttgtttcactctaatccagtgacactggcctgcttattgttcctcagacattccaggcacagtcaaatctcagtacctcttcacttgctgtttcctcataactacagctctcttcacttcagcacctacatagcccacttccccatcaccttcagagcttcattccctggtaagcctacttaaaactgtaaattcaattcccaaccatagacttactcccaatagcccccaattctttgattgtattgtttgcccctttatgcccaatttctagaactgtgcttggcatacagtatgtgttcaaaaatattggttgaatgtaaatacaggagtcagattggggacaaattattaagaaccttcatatcatgctgtggaggagatgagaaaccaaggaataattttagattaagaaagtattctgataagatatttttataagggtactaagggtcatgaaacaaatacaaaaatcctcaactttgtggaatatgggttagaagaaggagagaatggagataagactaattagttgttgaaatattccagccagcaatagggaatatggttgaattgacaagctttggtaccagtaagagaatagatacagtaatggcagagaaatatggaaaaatgattatttcggactatttttttttaagagtctctttggtagcccacactggagtgcagtgatgccatcttggctctctgcagcctctgcctcctgggttcaagcaattcttctgcctcagcttcccgagtagctgggactacaggcatgtgccaccacgcccagctaatttttgtatttttagtagagacggggtttcaccatgttgaccaggctggtgtcaaactcgtgacctcatatgatctgcctgcctcagcctcccaaagtgctaggattacaggtgtgagccaccaggcctggcctatttcagactttttatctaagagattaggtgaatgaaagaccattaaccagaacagagaaggcaggaagaaggtatagaactagtaggacaaagggaatccaatgactgttgagaataagagctgagctggagaaagatttctaagtcattagcatataatttaaaaatttagacacctagcacagtacttgtccagtaaattttaatgcattttaatctttatctgcctctcttgcaattcaccttccataattatccagaggaaaaaggttttcttaaaaaaaaaaaggacattttttaaaaagacctcttcttcataattcttattttcacttaatttcataacctttttgtattggatatcttttctgtttttttcttacccagaggatcttcccctcttttttaagagccatttgtttcatgcttttcatctatctttttctcatgccatattactttgaactatattttgttacctattgtttttaaggattcttatgttgctcttctctctctgtagctaatatttctctacccttttttattgtttgtttttttaagaattctacagtattcacttactttttaaaagtactgtgagatgggaactgttagtatccacccaccttttatggatgaggaaactaggtcccaggatctcagttagtaagtgatgaagcaacattctacctcatttagaatgaggtagaatctcacttagtgagagactcacttagttaaaattttttgttggagttctctcacctttttcagatgcacatattaggtacaaaacttgtatagaactatttagagcatttgatgtaaatagatgaatatataaattaggaaaacagagattaatgtagaccataaaaccttgtttcctaatttaaatacatatgtttgtgtatttcacatagtaatgttaaaaaagcaggtactctgattttttacatttggtatggtccagcatacattcatcatgcacaaaattatgaacacattgttcatcagatatgtatagctgatttatataaatgattgtctttctcctttccttagctgtttgaattgggtcctgatggaggagatcatgaaactgatggtgaccttcttgcagagactgatgtattggcttatgactgtgctgctaggtaaataatttgtgcattattcattgtaactcttgcatacttaatattaaaaatttattataaatgaataaacatactttaaaaggaagttttaaatctgctatgaatatcaaatacttgagaaattaaattatatattaagcaatataagtattgctttcaaaaatgctgaattaaaactaatatacttcataaatttatttcataagtcaactgtttcaatattgaagataaaaatggaaatatgcatatattttatacacctttactttcatgcatctcttacggaatacctagtttgctggtgttctagtaataatacagttatttattactcctagcattacaggtaataaataattagcgtcaagtggtcatgtttgaccataattcagattacccatgtactctgacatcatgctgctagaatatatttgataataacacacttggggaggtgactagctttactgaaaccttttgggagtttataaaaattagtatgtttatttatgaaggttcattaagtaagaggatacttacactttatcagtcctctctacgtaatcctaagaattatctcaatattgtgttatgatcagtgttccctggtttgaagcagggaatattattataagaaaaattataaactgagcttcatagtagtaatagtgtgctatgtttcccaacaataactatgttaggaaaaccttgtaggttttcttgctagtgaaaatgtatattttcagaaacaaggatgatcgtaagatactgactttgcttgtacaagatataaatacttttgaaaacaaaagagtaaatctaaagaaaattttgactatttcttaggcattaactaatatcgcaagatttttttagtaacctttagtgttttatacaaatgaatattagggaaaattttaagaaagctaattgattaattttcagcaaagccttaaggtgccattgtcatttgccgtatttatttatttctttatttattcatttattaatttttatttttgagacagagtctcactccatcacctaggctggagtgcagtggtgtgatctcaactcactgcaacctctgcctcccggatttaagcgattctcatgcctcagcctcctgagtagttgggactacagtcgcatgccaccaggcccggctaatgtttgtatttttagtagagatggggttttaccatgctgaccaggctggccttgaactcctgacctcaagtgatcctcctgccttggcctctgaaagtgctgggattacaggaatgagccactgtgcctggctgccatttgtttttgaccatatgagtttgagtcgaaaatataaagcacagtttcatttatagagaatcgtctaggatttcttcattagcttatatacaaagcccagactcctaaggcattgggagcattgaaggatttccataacatccagcatcttttttgactttctaccactactcttctacagctgtcttaatgcattaatgaaattaataatgaacaccttgtcatttcctgacctcttttgtacttccatacctctacagattttttcatgacattctcctgcttctttaaatgccctttattcttctttcaattccagctacaaccaataaacattccttccactctgttacctagtactttgtaacttgtttatatcctttagaagatgcccttatttattgtcttttttcttcttctaaattaataatgtttgtatagatattatgtgacagactccaaacacctatctgacttaattcattctaaaggacaactctatgagcaggatattattttcaccacagttttcaggtgaagaagctaaggtactgagaggatagagtaacttgccctgcgttgccctgctagcaggtcacagagctgggccttgaacccaggctgcctgactccagggccattttcttaatatgctgcctttcaaaactatttagctgaatgaatgagctttaaaactaaagttcagatttttattttccagcaaaatttgaatgttttttaatatcagtttaaaacattctaattttaatatgaaagttttataacatttaaggatattgtagaattcaagaagaggaggaagaaatgtaattctatcaccataacataaataactatatttgcttgcattcttgtccagacttcttagagtgtttttaatgttgtttgataagaattttataatacagttagtttcatgtttttgttcacaagggattttgaaaaaataaatatatgtgacagagaagtaagaaaatattgtttttgactcactaattttttctcagagaaaaatatgcaatgatgtttgatgagcctgttctcctgcaagctgggtggtggtatgtggcatgggcccgagtgtcaggacccagcagtgactgtggatctcatggacaggcatctattaccacagatgatgggtaagtaaatgcccaagtgttacttaagcaatacttatttttgttagaggaatattgttttgaaaattcagagtactttcagcaggattcaaaagcttaggaagactagagcataatataactgctaaaaattaaattcatgtaacctcatgccaaattgaggtctagacagaacaaggagataataagaccactgtactttgctcctgtcagatggtatttgtatatatttctgggtattacattgtttacgagcttgattaccaaattagaaattatatagaggaaggtgacaaggaaaatgaggagtttggaaccataaaaaagaagtagatatgtgatattaaatttgatagtgcatgagaaaacagtttaaaatttcaaagcttcagcaatgtgtgttattttaataaaatatagggaaaattttgctgttttaaaataactttcacgtatagagtacttttcagttcatattcttatattttatctgatttgatgccctcaatagctttgtgaagcagttattagccccattttatactccagggactagagaaataaaaaggtgaaattatttgctatttcctgagagtcatgtggttaaaaccaaggtcagacctgtaacctgttgactctaaatccaacatttcaatattctattactatgatttatgtattctaaaggactatcatttgtaagataaattcagtttattcttgattactcctgagcataaactatttgaaatcagttgaagttatataacagtatatattccagtgcaggtcaattcataacaaagctgaccaaaaaaagaaatagacttatttgtgaggtgaatttcctgttgctgaatattcaaggagactactgtagaaaaatcacatctgttgttatgtgcatcaacttaagaatgtctttagaactaagtgactactttgaccctggactgtatgattttcatagacagttcattcagacttggaatgtcatggcacactgaggctaaagcttatcatgcagctagtcaagcagatattatactacttagttgtaagtagaaattttagaagtttactgctgtggttaaagctgagttggagagctaggagggacaggacagctactcatttaggagtcctaatcacgttcagttctttcattttactgactaacccattcagagctagaatgaaaatctagaaaatacctttttgcttttctttatgtaaaagagtgaatcattttcattgacataagatttgaaaacatgtttcttaaatttcttatttcctgtaatgatcatgttgtttttggtatcattgttttctcttcttagggttgttttccagttcaagagttcaaagaaatcaaataatggtacagatgttaatgcgggtcagatacctcagttattatacaggtatttagcatatttatactgaattagtatggtttattattttgctataatgaagtagaatcattacaaaacgtggcataatagggagaaattatttgaagcattcagcaaattataatatgctggtagaactataaatatatttaacaaaaacagttaatattttgtatatttttaaacattttccatacaggcttaactttaaactttgcctgattactcagattatctgtattataccaataacaatattttactgactttaaaaaaaatctatccattgattttcttactaatttcaaatgttgtaaaagaataaaatttgtcttggagaagtgagattgcagggaagatggggaggactatagatgcaggcctggttgatatattgtagtctcataagactgataaaatgagttggaaaattccctactgttggtaaattttcacctttagctaaaataccaaataataacttgctcatataatatttactatatttttaacattattgaaaaattttaacttacaataatttaagcatgtataaaggcatatgcctgtgtttaccttatttatttctagactactgcaatacttttagaataaagaattcaaatggatataaaccgttactgaaaaatgttgtcattcgttaagcagtaaaaaatcaaaggaaatggtctagaactgtgattctatctccctatctttctttctggtaatcactttgtggttacaaacattatttttccccaaaaggttttttcgtttgtttggtttttatgcaccgtgcaatctgttatagttactgtttgtaatatgtagctctacaaacataacgtgttgcattggactactcccaggtactcactggttgaataacttttaaataacagcattaatagtagcattactagagaatataagttatatgttatatacattatatatgttctctaagaagaggaaaaagcctctactactcggtcttgcgaaggtaggatttaaaggaagtgcccatatgctaaaaactttgtttagcttggaaaaatgtttcaaaaaagccctgtattggcattacctggcaaaaaaatttaaatatataatttattttcccattcaaaaatgtataatatactttaaaaagaaaaaatgacattggactttctaacataattttatatgtgtctactgcctttagacttccaaccagtgatggcagtgcttcaaaaggcaaacagcaaaccagtgaacctgtacacattttaaagaggtcttttgcaagaactgtctcagtggtaagccatgttttaaaaaatttcagcttttctcttcagtcctcagatacattttgatgtttagagtttagctgctcttgtaagtttatcttagcctagatttataaactcagatttggttttgtattcacatttatattctagaccacagataaagtatactttaaatcatagaaaaatggaggtgttggcctatgattttaacatgaccagatgaattggccttgctgtttttgaaacaaataacgatttgcttttctagtaaatcatatatgaatgcatatgctgaatgaattagaaaatgaaggaaaagttaaggatggtgctggcccaaaaaagcgtgattactacttgaggaactaggtggaagagttgcatatctaactaggaagtttgttgaaccacttgccatccccattgtacacatgaagaagcacagtgccattgaggttaacagtgcagaatggaaagtcaacatgatgtaaataaatgaaagtataaattaaggtcttgatctctcatgtattaattgaggattcttacgtatgttacctaagtaaattacatcatagcattttgaatttctaatgagataaggggtgtaagatgtttttggaactgtaaggcaccatctgaatgtgatttattaatatagctagtagtctttcatattagtaaaatgcataggaagaatgtttctttcccttttaaatttatttttcaacccaccaagtgaatatatattcctggtaggaatgagataaaagtatcctaaaagtatatgaaagatttttctcccattctcatgatcaccatatatacctactatttacattttggtgtgtatcttcccagacctttttttttttttttttttggtgaggcagagtctcgctctgtcgcccaggctggagtgcagtggcacgatcttggctcactgcaacctccgcgtcctgagttcaagcaattctcctgcctcagcctcctgagtagctgggattacaggcgcctgccaccacgcccagctaatttttgtatttttagtagagagggggtttcaccatgttggtggtggtctcgaacccctgacctcgaacaggctggtctcgaacccctgacctcatgacccacccgcctcggcctcccaaagtgctgggattacaggtgtgagccaccgcacccggccttcccagaccatttttatatgcaaaaaaacttattagtacgtatcaaatacaggatgctctacaacatgcttgcttgtttcttacgatacttacagttttcatcttttttagaaaatttcgtattatatatcatggacatccttccctggcagtacatttggctctgtcagtcttctccgtttgcgtaccattattctaatttattcaatctgatatctatcaaagaaccactttgattctcattttttgttatttcatcttgcttcatgagcatctatacacatacatctttaaatacttgtgtacttatgtgcatacttctactggaaatatgcccatttaaagtttggtatatcctctcaaagttgaccttccaaaagagccatggcagtttgtagagcttgtgctttgaataaaagactatgagaactttggcttcttggtaactttgcatcctattagtcttaagttgagtttggtatcttttgattcaaataaggatctttgactctgttatgctttctttagtgatctcacttttctgtgcttggtaacccctttgtatttatgtttaagtggttttgtggccattaggtaattgtagactttatatacaaaacttattcagtcatttctttaaaaggtaaactatagggcttagcacattggctcacgcctataatcccagcacttttggaggccgaggcaggaggattgcttgagctcaggagttcaagaccagccagggcaatatagtgaaacccccatctaaaaaaacttaaaaattagctgggtatggtggtgcgcacttgtagtcccagctgcttgggaggctgagggggtggtgagaggatagcttgagtccaggaggttgaggttgcagtgacccgtgatcacaccattgcattccagtctgggtgatagaggaagactctatctcaaaaaaaaaaaaaaaaaaaaattgaatctataaactcgttttcaggagctctgggatttggtgggaatggggcagtaagtgctctgctttgagattcaggtggtatctgttgtcatttaaccatgataaaaccagaagactagaactgcaaagaaaatctaccatcttaaactcatatgctagaattttaaataatcaaaataggaattttttagtattttctagtcaatttttatcttctattaatgtatttatcaaaaattgactgtagtttgtattgcaaattttaatatatagacataaaataagttatttgatactgttactgatatctaacactaaaactctattttattttttactacagtgactgtaaaaagctttatttttctttcaagtaaacatgcattgttttataagtgcttctcatttttcccttgtttatatgataagctagatagctgggtagcaggaacactgaaaaatttatttttaaataggtaaatattgaagaatgttgtttttagaaatgcttaactactttaccttttttgtaacaaacttcaaagttactgttgagggtaatttaataaatgaactataaaagtgtcttgggctcagtaaaaaaaattcaattaaaaaactcagtaaaaaaattcaaaagaaagttttgaagttgtctaccattgcctttccatttgattccgtttatggtatcatttgaattaattgttgtctgttttttcatattctggattaggaatgttttgagtcattgttgagtattcttcactggagctggaccaccttagtcttaggagttgaagaacttagaggattaaaaggattccagttcacagctacactcctagatttagagagactgcgctttgtgggtacctgttgtctgaggttattgcgtgtctatacctgtgaaatttacccagtgtcaggtatgatgcattttttttaaatgactatagggagaaaatattagagacattgagaagatttactttgagataactgagagtttatgccccccagctgcatttaagctgcttctaaaatgaggggagaaagtcagcttgctatgatgcttatgtatttgcttataaagagtaaaatagattacatgtcattctgatacaaaaagtaatagtgggccaaagttaaataatctaataaagtttaattttaaacaaattctgtcaaatagaaatattttgaacaagcaatttggacttaaatgctgaaactaggtttttattcccccttccatttagattaaaaattgggactacacttaatctctaagttgtttcatttctaagatattagtacttgtgatacttagctgtatagggaattactgaattatattgattgttttcatgcatatttaatattttgcttaattaccaatctgctttttccccctcaaagctacaggaaaagcagttgtagaagaaactagcaaattagcagagtgtattggaaaaaccagaactttgttaagaaaaattttatcagaaggagttgatcactgcatggtgaaattggataatgatcctcaaggatatctcagtcaacccttgagtcttctagaagctgtccttcaggaatgtcataatactttcactgcctgctttcattctttctacccaactcctgccttacagtgggcttgcctttgtgatctgctgaattgtttggatcaggtaatttaagtttgtaaaatgttagttgaaaatctgatatattgcttaatactcttcaagaaaataaattgcagttgccatatcttcttaacttgtataaaagataaattaggaaatcattcggtgtacatgacttatataagcaaagtttaaaattttttaaatctgcttcatcatcttttagttacaaaattggtaggcacattgtgcagttgctgattggtttttgaattggagtgagtgattctgagagagggagagaggaaaagaaccatagtgaccaggggagagcagggatagcaccaggaggcccccgccaccctcatctgtgagaacaggaaggagagagagctgagagaacagtcatactagcattgttcattcattcatgaggaagccacaacctttgtaaacctttatgcaccaagtcacagagtttcacaacacctgaggcaaaacactgacagaactacagggagaaatagattcacattgacagctggggactttaacactcctctttcagtaattaatagaggaactaggccaaaaacatcagcgatgcagatttgaacagtactgtcaaccatcttgatctgacatttacaaaacaggatatgaaactgcaaaatacatattcttttcaagtacacatgatttttaaaagaaatatttgggaccattcactaaggtagatcatatgctagaccataaaatgtcttaataaatttcaaaagattaaaatgtgaaagaatatattttcagaccacagtagaattacagtaaaaatcaatagcaaaggatatccagaaaaagcaccaaacatttggaaaatgacagcacttctaagtaagcagttgatcaaagaagaagttataaaggaaattttaaaatgtttcacatagaaagataaaaacataatatcagaatttgtgggatttagctaaagcattgcttaaaagagctctttatacccttaaatgcttatatgaagaagaaaggtttaaaatcagtgacctaagctgctatgttaagaatctagaaggaaaagcaaatctaacccaaagttagaaggaaggaaggaaataataaagataagagcagaaatcaatgaaatagaaagtagagaaaattaacacagctaaaagttggttttttgaagaaaaaaagttggtaaactcctagcaaacctgactgggagagaagagagaaagaaaaaacatgaaataccataatcaaaagtgaaataggggttcaccgggcacggtggctcatgcctgtaatcccagcactttgggaggctgaggtgggtggatcacttgaggtcaggagttcgagaccagcctaacatggcaaaactcagtctctaccaaaaatacaaaaattagccaggcatggtggtgcacagctgtaatctcagctactcgggaggctgaggcacgagaattgcttgaaggcgggaggtgaaggttgcattgaggcaagattgtgccactgcactccagcctggtgacagaatgatactccatctcaaaaaaaaaaaaaaaaaaaaaaaaaaaaagaaatagggactatcactataaacaactttatactggtgaatttttcagccttagaggaagttgacaaattccttgaaaaatataattttatcaaaactaacacattaagaaatagaaaaatctgaattgcctcatatccatttaaaaattatcaaaaactttcccacaaagaaaatttcaagtccaaatattgtcatttatgaattctgttaaacatttaagaaagaaataatgtcaaccttttacaaagtcttttggaaaatacaggaggcaacacttcccaactaattttatgagtttagctttatcctataccaaaacctaactaagatactacaaggaaaaattacagatgaatatcactcagaaacatagatgctcttaaaaatattatcaaaccaaattgaacatgtaaacaatatatcataaccaagtgggatttatcttagaaatacttagttaaataattgaaaatcgatgtaatttaccatgttaactgaaaaaagaaggcaaattgatgatttcctccatatagctacaggaacagtggttgatgaaactagcagttactcaatgatgaaaacctctcaggaaagtaggatataaggaaactcctaaatctgataaaaagatatttacaaaaaaatcttcactaacaacatcccacttactggtgaaatctgaatatgcccctaagtttggaaacaagacacgtggtatctacactcaccacttatattcagcagtgcactggaagtcctaggcctccccgccctccttcgtgaaaaaaatcattgtcatcactaatgcatcccaggcaactttgcatccatgtagcagagtagtatgttttaccactcaatttccaggggcaagattattgcctggactagtgcacaacttaattactctctcactctgaaccttcccagtctgatatggttcttttcttctttgctacagaacaggctagatttagcaaatgttggtttaacatatggaatgttactctgcttttatgcttaactttgtaacacagatgtttgaaggactaggtaatgtgtacacaattttgtgaattaggatatcatgtgttggctttaatgcttttttatgaaattttgtaagtatgctactaatttttcttggtaaattatggaatgtgataatataattttgtgaccttagtgaaatgtgcttgtaatagcacagatttataatttgcatcatttattatttgagaaaaatagtctcagtccttattgaaaataccttagatacattctaattggggcctttttggcatctccctttgaaacagtgctagagagtactgtgattgtgaggatatgaaaatataaaaatgctcatttactagaggacaggagcctgatttttcttgaccagacttaatttttcatcttcattgaatagaaacttaaacaattttttaaatatatagatgtagtattattctataacaagacaatttaaatgacagagtgacagaaaattttggtataatttaaaagatggtttaccttttatgtattcagaaacttagattatactgatgctatattttttggttggggggccatatttttgtttttaaaaggatatccaagaagcaaacttcaagacatcaagtagccgactccttgcagctgttatgtcagctctgtgtcacacgtctgttaagctgacttccatcttcccgattgcgtatgatggagaagtattactacgatcaattgttaaacaagttagtacagagaacgactcaacactagttcatcgttttccccttttggtggcacatatggaaaaactcagccaggtaggtctgtctctgaaatcttttatacagaggcactaaaactctaaatggttttatttattgcctcttttatacagaggcaataaaactataaacacaccaagctaagcgtatttagcttggtgcttattcttctctatttttaagtatatttacaaaatgattattttaaatatttttgaaaaatgttcatatttacctataaaatgaagtttttttgctctaacggtttaatcacacacctttaaaaaaatttgagagttatacaatgaatacctacatatttgtttttcctgaaccatttgaaggtaagttgcagatactttacattctaatataatattttagatttctaagatagaacaataagaaaataaaggatttttaaaatatctacttaataacactttggtatattaaaataagcactgtaaataataggtataggaaacattcacataaatcattttcatgctggaaatcagctaaattaatcatcttttcccttttttctgaaaaataaagtatttttcctttattgagactctgtgacttcttgaaatttttctaccggtaggggttggaattttcttatctccacttaggaaaacaaatacagtagaaattataaaactacataggaatcatatgtgtgtacaaaaagaattttgttaaaaaagtgatttacacagactacagttgcattacagctaaatatactagaatttattatctcagattaagaccttgtcctatagaaaaagctttttatttttgttttttaagactagctcattctgtcacccagtctacagtgcagttgtgccatcgcagtttactgcaacctcaacctcctggactcaaacgatcttcccacctcagcctcccaagtagctggggctgtaggcacataccaccacacccagctaactttttaaaaaaacattttgtagagacaaagtcttactatgttgcccaagctggtctcaaactcctggcctcaagtgatcctcccatctcagcctcccaaagtgctggtattgctgacaccacgcccagccaaaaaagctttttaaatggaaagaagttttctgtctgtcatatagaattgtaaattgaaagttaaataagagaataaaataggactcaaagctaataaaaacatttcgtaaatgtatggtactttgatacgcagagacagataattcaaaaaagaacttctaagtgaaaagtggctccacatataaccagaaagatttgtagaaattgaccctgattcaagtctgaattttaagagtatcagaaagacaaggttttcaaattgagtactcctatgaaggaaaataaaacaataccttagaactttttcttatgtatcgcagagtgaagagaatatctcagggatgacaagcttccgtgaagttctggagaaaatgctggtcattgttgtgctaccagtcaggaacagcctgaggagagaaaatgaactcttctcctcccacctcgtctctaacacctgtggattactggccagcattgtcagtgaactgacagcgtcagccctgggatctgaggtaatacattatattttgacactggataaaaagcacatgtgtaagttttttacacagtttcttttttcaactattttggctttgtggaagctaatttcagattggattagcaaccccaatgcattgtttttcttttttgcctttctctccacctcctactttctctgatttgttacttctctctcaccagtttacccagcatccacaggaatataaagtatacattttcctcatagagcaggtcgtcatctgccccattgcacaagcccaccaccatgccccgtgaaaagcaaagatagttctcactgctggaagatgagtaacattgctgaagtcaggccctactcatagtgcacacatagtgtgggaaaggtaatgaggtttagacttcagctctgcccactcaaccgaccctagagaaatacatacttaaaattggaataataatttttatttcacctctatcacaggttttttgaaattatagtaaaatatacataatataaaatttgccattctaatcatttttacgtgtacagttcagtggcattaaatgcattcactttgttgtacttagttgtacattatcacaaagttttcaaagtctcaaatattatgtttgagaaaatcactttgaaagtgcctttacattttgtaaagtaacattattccatttcactgccttactctttcttataacttaactgaatgttttgactgaatttggggaatgtttctgttttttcctcctaaaaaacagacagtagattggtttggaaaatcatgtcctaatatgaaatatcttacatcattcaaatgaatgtttaataatcagcatatattcagtggccctagctcccaagttcttgtcaaggaacttcttttctctattttacttttataattcctaatctttgctttttttaaaaaatttattttaacacctattctgaattaacccatccaatatatacacattattgattaattgtgacagtttagcctttagaatttcattgtatgaaattcatacgtaagaaattcgcatacatatggttcatcgcatacacttgagctaattttagtctctcatcctgggaagcactttctaagtttcttggttaactttgagtttggcctcagagacgattgaagcccttgtcagaaattttttaactttttacatttactcatttagtctataataccttcctttgatagtatactctgcaggatgcaggaaagtctataatgtgtagatccccttccaaagtttactaaagagttctcccctttaaacttacctttccaatgaaaaggaataccaaaagttacagggtgcctggtatacaactttgggagttcccacaggtcctggcttagagatgaatatttctttctcagagaagtttgcaatttttgttagaatcataaacttccatttttgtatctatttagtctcccaaaattctgtagccaagttagattagaagtaggctttgtaaataaaaagagcgatttcaacccaaagtaagttctctaattctctctagtgaaagtctataattagtatagttgcacatgtgtatgtgtacgtattcaaatactgaatgctgttacaaaaatatcactgtatatattcgtagtattcaatcttagggtatagaccattttcttcaaatgaagttttacttgcaaattcacaggtaagaggagagaggacatagtctggttgaagaggggaaccataaatttacctcttccttgcacacctatctctgcagaatttctagagctctgtgttgcatagtttgaaaactgttgatctatagaatttcgatgaaatttttagcatctcttttcaaagtctgatgatttattcatacattatgactgataagacaagcttgtacttctgacattataaattaagtggaaagtatagtttcatatggctaaaggaagaaaatactgtcaagtgacattgatttgtgaatttagaatgataaaatctggttttattgtgagttaatctctcagtagaacatacctttaggaatgtttatatccacttattaaataaaggttattttattagacttcttagaaacctttggatggttttgttaaaggtataccaactactgatacaattgcttttattgttcacaggttgatggacttaattctcttcactctgtaaaagctagtgctaaccgatttacaaaaacaagtcagggcagaagttggaacactgggaacgggtcccctgatgcaatctgtttttcagtagacaaacctggaatagttgtggttggtttctctgtctatggaggaggtggaattcatgaatatgaattagaggtgttggttgatgatgtaagtatcacccttttagtatttatcctgattagtgggttgtgtatcaggatttaccattgtcatagtatgttcaggggtttactagatcaatcactgatgaatgacactaaagaaattgaaaaagactggccacagtggctcacacctgtaatctcagctctttgagaggctgaggcagttggatgacttgagcacaggagttcaaggccagcctaggcaatatggcaaaaccctatctctacgaaaaatacaacgcacgcctgtaatcccagctactctggaggctggggtgggaggatcacttgagcccgggaggttgaggctgcagtgagccataatcatgccactgcactcaagcctgggcagcagagtgagacccgaccctgtctcaaaaaaaaaaaaaaaaaaaagaaagaaaagaaattgaaaaataatgattttacctgattgtttgtgttccatctttccaccttgggtcactactgcttcatattttttgtactttcagaaagagaaagatttaaagtagaattaaagactaaaagtgtttagctaagtccctgatgacaagtcttgagtagtttttttttttttttatattgtcatttttcaagaaacacaatgttgagatggactatgtcatagagtactaatgggtgattcttagtgaagacactatttgttgactgattgagctatatctttctatattctctaaaattaaagtacctgagttttattaaaatgggacagatggggtagcatgtaaatcattgcctattaaatagcaagtctatctctatctctaacttgattcctttcactagttaatttgaatttattccctacttcattaggctttataaatgtgaagaagtagaaatagtttaagtcatcagatgagaaaagcttatttatgttgtagtcttctcaagaaatcacataaaaatgtatatagtacccccaactttttctttcatatgtatgggaaaatactaatggctaattttatattcaagaaaataaattttaaaaataataattataaatcaaaattgttcatacaataaaattaagagtctgttggaatttttgtgtttcattttttttttttagagtgaacatgcaggagattcaactcattcccacagatggacatctctggaattagtgaaaggaacgtacacaacggatgactcacccagtgatatagctgagatcagacttgacaaagtggttcctttaaaggtagtttcaactgaatgtttctattgtgaataagaggtattattatgtagttaaaatagtgaagagtattccatgaattttgtttgttacctatagctatttgtaagttatcctttatatggtacttaaatatttatggagtacctactacgtacctaatgttgtaccacattatattatatttttgcataatgtttcttaaagtcaggagtagtaggaagttttgtagtcaaacagacttagctttggatacctgctgttccacatgctgactttaagacactggcagatcattatttaaccactttgagacttatctgtagattgggaagaataatacctgcattgtagtagtattgagattaaataatggaaataaaacacctgccttagtgtctagcacaatatgaaaaataaatattagctcctgtcttttcttctcttattgtgccctaactcatttaaaaaaaaaaaaaagtgtttttgttttgttttgttttgtttctttatttttttttaattattattattatttttttttagatggagtctcactctgtcgcccaggctggagtgcagtggtgtgatcttggctcactgcaacctctgcctcccgggtttaaatgattctcctgtgtcagcctcccgagtagctggaattacagccacgtgccactatgcccagctaattttgtatttttagtagaggggtttcactgtgttgaccaggctggtctcgaactcctgccctcaagtgatccactccgcttggcctcccaaagttctgggattacaagcgtgagccaccgcgcccagcctatttcctaacaatatttggcaaaatagagaaattttaccacatcaaagtaattatagatgttaaaataaaattaaagtatttctgaattcaactgaaataaatttcattatagagcagaattttggataagatcggtacacgagaggtttcatcaggaagaatttagatactgaaaaaccaaaaagcttttattgtgaaatttatatcatgggtcataaagcattagtttctatagctcacattttaaaagtacagtgcattatattttggacagcttttttcctatgggttttttttctcatgtctgtttgcctatatatttctatctctgagtttttcttaaaaatataatagactcaccatttttttcctcacatctaaaattattttaaaagttaaactagtcaaggaaaagtatcagcttatgcaattaaaatcccttattgccgggcacagtagcgcacacctgtaattccagtacttttgagacaccagggagggaggatcacttgagcttgggagtttgaggctaggctgggcaatatagtcagaccttatctctacagaaacaccctaaaaaattaactgagtgtgatggaacacaccggtgggaggattgcttgagcccaggaagaggaggccacattgaaccaaaattgcaccactgcactccaggctgggtgacagagcaagatcctgtctcagaaaaaaatatatatatataatatataaaatatatatatattatatatatataatatatattatatatatatagtatggctaatattctagcagtaaaattaagagcaggcactagttacattggttgtgttcagacctagttccatcacttactagctgcgtgactttgaacaagttacttaacttctctgaacctcagattcctcatctgtaaaatagggatagtaatgatacttaccttatgaggtcattttaaggattaattaaattccataaagtagttaggacagttgcttttatgcaacaggtgttcagtaaatgttagccgtcatcgtgtttgattcattaaaaacttatttaattatatcatctgtaaatatgacaaagaatttcagtatatttgcctattttactgctagagttctttttatatatcctctgtacattgtgaataaaagatgttttacatctttataatgaaggaaaatgttaaatatgctgtgcgcttgaggaactatggaagccgtacagccaatggagatggaggaatgaccacagttcagtgccctgatggtgtgacattcacattcagcacgtgcagcttgagcagtaacggcacaaaccaaaccagaggacagatcccacagatactctactataggtgggtgaatgtatagagataacggaaatactttacagtggtagaacatacacagtctactaccaggtaaaattgctaactggatacttataattaaattgtacagtgtatcttcttagcacatacttgtacaaattcatataaagaaatctctttttaatatataaattcaatgtttgctacaaaagttaatgctttaaaatatttttgctgttatttcttcaatatcattgaaatgtttgtgtactgggcatatgtgctgttgctgtcactctataatttctggtctgcaatttcaaatttaaaggatattcatttctttttataaaatacctcaaactaatgatcagatagactgtactcagatagtggtgttgccttgatcttgttatctagtcacagaatattttgatgtctgtgaatcttggacctgccccctggggccctagaatatataatagtgtgtttcttataatgtttttaacaaaaaccattccttatgtaaaccttggaaaataaaagaatgataacaacccttgggttaaaaaaatcttttaaataaacatttctcactgagtctgaatttttcttgtagcggcaaagctggcgtcttaggtagtcatgagtaccatttaccacatttgtcctcagcctgacttggcattaaatcactgaatttccatgtcactttcagaggcttgctgcttcctctctttccttcatgaagagtctagggatggtgcctacgtagatagtataaaataatttccctagatatagattttctgcttcctctttccatcagaaatagaatagaaatagcaggcagcaaatagctatgtagttcattgttgctgctgaaatacaaaagctgtctagtcttttgccccagtcaagaaaagctttctgttacataagcactacaataaatagtacattattctctttcttaaactatggattaaaaactctgttatgcgtaagagttacgtgtttcagaaagctggagagccactaagagcatctgccatccaaactttatatcttatacaaaatacagtgttttgagtattttcatgttttctattaagctttaaatacccttagctttacattttttataaatgaaaaatgttaaagtcctttagttttaaataccatcctgctctgatagctcccaagtttacatccctagccttcatgacctctgagccctggactccgggtctctcttctgacatcttcatttggatgacactagtggagatggtgttagttactgtgtgccaggacatgctttgagtatgttacctgtgttatttcaccttgtcctcacagtaaccccaggaggtaggtactttccttatccctgtttcacagatgatggaactgagacgctgaattgtctagagtcacacctctgttaagttacgctactgcgacttgtaggcaggaagtcagatttcaggtgatacacccttaaccagaatgctgcactttctctttaaggatgttcgttatagttggctagtagacatctgaggtttcatgtggctagaatattcatctcccttccacagttacagccagacaccttggagtcattctggacttctctttccctcatactttacacctagttcatcaagaagtcctgtcagctgtacttctaaaatgtcctaaatgtgaccttttcttctacttccatatttaatagtctagaccaagccactgcaacctctacctgctaactgtaataagcctctaaactagtctccctcctttcactattggtctcctacaatcagttctctacagcacagccagagtgatctgataaaaatataaatcataccaagttccacccttgctccaaacccagcttcccattctcataccagtataaaatgtaaagtcattatgtggcctacaaaccttacacaatttgactcttgtttcccttcccacccatcacgcttcactctttctccttttcttccagccatacatgtcttcattctgttcctttaattcaccagtatgttccttttgccctgacagttttccctcagctcttcgcgtgggtctttaaaaaaaaaaaaaaattcaggtcagctgggtgcgcggctcactcctataatcccagcactttgggagtccaagatgggtggatcacgaggtcaggagtttgagaccagcctggccaacgtggtgaaaccccatctctactaaaaacagaaaaattagctgggcgtggtggtgcatgcctataatcccagctatttgggaggccgagggaggagaatcgcttgaacctgggaggcggaggttgcagtgagccgagatcatgccactgcactccagcctgggcgacagagcaagactctgtctcaaataagtaaataatttaattaattaattcaggtctttgttaaatatgagttcctcggagaactgatcttatttaaaataacacccttcactttatgccctatgctgctttgtttttcttcaaagcccttatcattccctattattgtatagttgcatgtttacttttttaacttttaaaaagctataaattaaatgaggatctcagatctagatatgatttctggcactggatcatgttttacttaccattgtaaaataatctcatgtttgctcatgctacaggagtgaatttgatggagatttacaatcccaacttctgagtaaagccaatgaagaagataaaaactgtagcagagcattgtctgttgtaagcactgtcgttcgagcctctaaggacctcctgcacagagctcttgctgtggatggtaatattttttctttctcagtagtcatattttagattctttattcactaaatttttgcatagaattattgaagaggagccttctattatacaattttatagaatctaagtgttatttatagatccacccatggattaattttggataggtcacactctccatactgtaatgcagaattatttagaatgctcaaaaggagttaccatatattcttattttcttagttaatttagtaaaagaaaaattgtatttgagttaaactgggtctttattcttcttttagtaatcaaaaagctttttcttcctctctgttctttttccagtaatgtatcgctatccacccaaaactctggggtaccctaaccatctcctcagaggcacttgtctaaattcagctctatgagtttttccattggaatctctctagtttgtattctcctctctttcacttcttctcctgctgtcatttagactgccatggtgcaagtacatttcattttttaatatagggttaatatcccgataatctcactgctgcctttcccccattctccatctagttgtttagttattcttcacactggcagcagagttatcattatgaaaacatggttatggccggacatggtggctcatgcctgtaatcccagcactttgggaggccgaggaaggcagatcatgaggtcaggagtttgagacgagcctgagcaacatggtgaaagcccatctctactaaaaataaaaaaattagccaggcgtggtggcaggtgcctgtaatcctagctactcagaaggctgaggcaggagaatcgcttgaacccaggaggcggaggctgcagtgagccgagatcgcaccactgcactccagcctggatgtcagagtgacactccatctcaaaaaaaaaaaaaaaaagttacctccacctccttattgtataacattaattcctaaacctagtttaacatcagaaccacctggaaagcttgctgaaaataaagactattagatttatccccaggtcctgaagctaggctaaaaaactattttttaaataagtgtgcagcctgttttgatataacagcagacctaggaatcagtatttgggagaataattggcttataaaataaaggtcttcacagtataggctcagcttatctttccagcaccatgttcatggataactgactaaaatgttaatcagcaaagtgcttttcttggaaaaacagaaagcaatgtagcttaataacagtaaacatttttggagtgctgaatatgtgtaggtgttgttctaagtgttttacatatattacctcgtttaatcttcccaacatccaaatgagaggaatactgttgtgaacctaagttacagagtaagtaatttgcctaatgtcactgatacagccatgtttggtgccagaatggtaatttaaaataaagactccattaccctaggttctgattccagctctattattttacaggtacatgtacttatgtattaagtattttgggtaagatacttaaaccctatgtgccatggttttcctttctgaaaattggggataataaaagtatcttattgtgagtattagttgagataaggcatgtattctcaaaagcctttagaatagtaccaggcccttgacaagcacccaattggtgtcaactatagtttcgctcaaaagagcagaatatagatttagtacagcatgtatgcagtacaatgaaattaccttctgactatagttttaatgccttttatgcctttttttagctgatgacattccagaactgctgagttcttccagtctgttttccatgctgctcccccttattatagcctacataggaccagtagctgctgctattcccaaggtgtgtaatttaattctataactttgaatgttttttttaaatatcttttttaaaaaaagactttgtgtcttgtctttacaaaaacttgcatctttgaggcatctgggatataggtaacaagcagagatagatctgctaattattaaacttctctcttcttagtattgtttactggattgctgtgcttatttgttggctttttttttttttttttttttgagacggagtcttgttctgtcacccaggcgggagtgctgtggcgcgatctccgctcactgcaagctccgccttccgggttcacgccattctcctgcctcagcctcccgagttgctgggactacaggcgcccgccactgcgcccggctaattttttgtatttttagtagagacggggtttcaccgtggtctcgatctcctgacctcgtgatccgcccgcctcgccctcccaaagtgctgggattacaggcgtgagccaccgcgcccggcctgttggctttacatttgaagaatctgtgaatgggaaatagattttagcaattttaggaatatgatatgtaaggaaagattatgttaactgaggtttttcacatttatgaggttgatgccaaatggtcttcattaataacaatagcaatgttaaaggataagagtgttttaaaatagtgacatattcaagagatggaagaaatgataaactattgattctttggcataattaagtaaaacctgatttacttcagatcatgaaaaaacaagtttttaaaaatatagatttagatattgtcatgtaacatctaagataaatctttttttaatcaattgaaagatttctctttggacaaaaacttaactagtggaaaatttagatggaaattgttattagagtaaccaacaattattgtaatgcataattttagaaccactttcacgtgtatctcatttaaccatatttttatatgtgtactaggtggctgtagaagtctttggccttgtccaacaattgcttccgtcagttgccattttgaatcagaagtatgcaccgcctgccttcaaccctaatcagtcgacagatagcaccacaggaaaccagcctgaacagggcctctctgcttgtacaacctccagtcactatgctgtcatagagagtgagcacccgtataaacctgcctgtgtgatgcattacaaggtaggcaccggttcttagtgtgattcgaatgtaaaacttggcatgaggttctctgatgatatcttaaagaatgcctggcctgtatttacaagtgtattttcttttatagtatttacatatagagaatcagcaggtgattaggcactgttggatatataaaataatttaagatggaatttttatctcaaagatgataaaattttattggtttatgagagttgttcttaaatcttcctacaagtatagtgtggtatataatgctaaacttcatgctgtgatttgcaatagtagtagatataagcattaacaattttaaattgtattttttgaactaacattatttgtaccacagcctgagctctgaaaatttacaaccccaatgtaagatttaaagtagtctatacataatgttaaaaaaactatgtataaaatgtttataaatttaatgttattagagagtattcaaaatccgtattctttgttttttcattgtatcatgcaaattaaggaagagaaggataaacttgtattttgtgtgcttgtaaaatattccacttttcatggtagaaactattaagcatatagaagtaaacaagctagtacagtgcacttgtttatccatcatcccagattcagcagtttcaactgttagtcagacctgtcttatttattcccaacctacttcctaacctcctatattgttttaaagcacatttatgatatttattgatacaatgtctccaaaatataaagacccttaaatataaccatagtattatcacattgaagaaaacaatttaatgtctgcaaacatcatgtaaatttttaataatttgagttagaatccatataaaatccatatattgtgattagtatgtctcttaagtcccctttaatcttaagtccctttaaatctataagtctccctctgtctctctctctttttgttcccttgagttctatttgttgaagaaactggatcctatgtatttgttagatgggagcctcttcagcttacctgctggttctttcagacacccctcttgcagtcattcctagcttccttgctttctggtaggataagatgttccaggctccctttatatgtttgatgcctcagacttggaactaaccattttttcaaagtgtcctgatttctgttatacatacacacacatgttatatttatatatgtatatacataaatgtatatatgtttatgtacagatagatttatgtgtatagttttttaaagataaaatacctcatgagtttatactgttgatactaattcaaacaaggaccacagacacacctcagtctcctttctcttatatctaaaattcctattagtgacactgggaatatcagaattagaaaatcattaaattatttcatttggtgtgtgtgtgtgtgtgtgtgtgtgtgtgtgtgtgtgtgtgtgtgtgtgtatgtgtgtgttttgtctttgaagtatatcccatctaagtgtatgtcaaattactgtgtttaaagtaagttggaatgctccctctgcatgattatgcattcattggatatacacttagagagtcatttattttatcttagattttaggaattgctttttaaatttggattttgttttataattctttacataattacatactttgaagtcaaatttatgaaacaaatctagcttttatctctgtcctctttactctgttcctccttctctgtcataggtaacattttattctattttaattttgtactttatcctgttgtgtcatttaataaaagcatagacacacacacacatacagatgcagatgtgtatgtttgtgtatatatatatctgtattcatgtcactttgcctgttgacctcaggtgacattcccagaatgtgtgaggtggatgacaatcgaatttgaccctcagtgtggtactgcacagtcagaagatgtccttcgtttgttgattcctgtcagaactgttcagaattcaggatatggaccaaaattgacatctgttcatgaaaatcttaattcatggatagaattaaagaaattttcaggatcctctgggtggcctactatggttttggtgttgccaggtaagttttttttttcttctgttttatgtggtaaaataagtgcatttatgtatatttagtaagggtgtatcattgaaaatgtttatctaatttttagagaaatttattaaagacctactatgtatctggattgcattcgaggaataaataaggtaccctgggcaaggggcacagcctgatctgagttttggagaagacaatgttttaaggattttgtgaaaaaaagattagcttgagcaaagcagagtgttaacgttgaggagtagctagctgtctgtgtagttgggtccaaattatagaaaacaatggattagatcttttattttcccaggaagtgctaaattttcagaacttacctaaggactgaagtccttcattgtaagagaattcgtaaaccactttcttgtgtgaagagaaactatgttttaaaacttatggtttgttgcattcttaaccagttaaccaggactactcttgtgatccttcaaactaataggcaatttcatttcagaaacttcttctttccactctgacctgcctatttgtttgtttgtttgggaaaagatcagaaaaagccacactccagggtagaggattgcccactttgaaaaataaaaaggctaacctcagtatttgacaaataattgaaaaatgattgcccatgtaactgaagtactttattaagcaggggagtttgattatgaaaaaaagtaacaattcatttgcgatagacctgttgtttctagtgttatttttggtacagtttcctcaagtagatataggaacgctccccgccgcaagtctatttaagttatgtagtatatgatttgtgtttttacctatcttgtgataaaagagaaagtccattggataaaagacagggaaatttttataaaattgttttagagtttgaactcaattctggcatttttattaggaaatgaggcccttttttcattggagactgcatcagattatgtgaaagatgacaaagcttctttctatggttttaagtgttttgcaattggatatgaatttagccctggacctgatgaggtaagaatgaattatcttttcattcttttaaggaagattgtgtttctaagggctagtgagttgtgcttctaactgacttaaattgtactgttgcctctattcgaaaaattggggcctggtgcagtggctcacacctgtaattgcagcactttgagaggtgagagaatcccttgagacagtttgagaccagcctaggcaatatatcgaaatcctttctctacaaaaaaaataaaaagaaattaattttaacctaggggtgtgcaattctaaatttttgtagtttatgtgcagtggtctgttttgctacttttaatacatgaagaggatggagtttgtgcaacatgacccagcctttgaagtatagttatacctgccctgaacctgctcagttggctatacctttctgagtaaacagtagaacctgttttcaaaaaggggctatcatcttgtctaaatgttttaaatcatacaataagataaaaccaaggacaaagtgggccaaagtcatgaatgaacagttatttgaacaatgaacctttaaacctgaaccttataaccagcctgagccacaacattgagtactgaatctcatctactctcaatttaaccattagcttcacgtgcttccttctcatttcaaaagtaatgcttgaattcaaatcacattttgttgataatccgtattatcattcaaacatttttaaagcagcaaattttatgcaattccatagggagtcatccaattggaaaaagaattagccaatcttggtggggtttgtgcagcagctctgatgaagaaggacctagcacttcctattggtaagtgtggccaatactggatgttttttgatagggttttgttttgttttgtatgtattataaaagggccaaaaatagatttgcaaaatgaattttgtaatctcatatattcatgagcaataactgcttcatgtactggtaatttgagtttgtttagagactgaagggtatcctctgaccttttttaatttacacacgcacatggacaccctttttatgcatatacatgctttgagccaggcagtgggtatagtgtgtatattcagccaagcactgaatatatagtattaaaccaaaagttctagatatttcttaaatattctagtggaaatatcaagtagacaactgaatactcattgtctagatcagagaacaggtcgatagaaatgtaattgaatatgatcgttatttaaagacatgggactgtatgaaagatattctacagcaagtagataagaaagagaagatagcttaggattaaagctgtgacttatcaaaatttcaaagatgcacagaacaggaagaggaggctgcagatgagactgtgaaggaacagtcagtgaagaggagagaaaccaggagaggggatgctcctgggatttttgcatgctctgagaagtgaattaaaatctatcattggacttgaagacttgttggttgcttgtatatgcaataagaaatatgttagcaaaatgagagaactgtaagtactttcagagtagagagggagaatggaaagtaaggaagggcggtccatttgaatataggtagctctttagaagaatttttcattgaagcagaacagagatgaggcagtggcagaaatggtacttttttatatggagcaaatttgtatgatatatagattaatctataatcaagggagtaaagttgaaagtaaaaatgcttactgattctctaatatttatacatggtttaaagttttattttcttacccccccaaaatccatctttactccttcattgaagattactcttcccactgttatttttttcctcccttttcttcctctgcctttccttctttattcccttttctcttcctggtcccttttattcttctccatcccttttctcccacttaatattggtatatatgtatggaaatgagaccttgtattctagccacatgaggaaaattgctagcaacctatttttgtcaatagctgttaaattttacattatatatactttttgacatagaaaatttttatacatacttgcacaaatactcaaaaagtgtatgttgaagggtggattaatatagcagtgcttgtgattgtaaaacctagaaacaaccaaaatgtctttcagttgggaactggttaaataagtaatggcatagccattcaatggtagtatatgcagctctttgaaaaagtagacctatacagcctaacaaggaaagctgtccaaattatattgagaaaagcaagttgcagaagaaggtcccatttttgcatacattactctgtatattacaaaaaagtatgatcatgcctgtaatcccagcactttgtgaggccaaggtgggtggatcacaaggtcaggaaattgagaccatcctggctaacacagtgaaaccccgtctctactaaaaatacaaaaattagccgggcatggtggcacatgcctgtagtcccagctactcgggaggctggggcaggagaatcacttaaacccatgaggtggaggttgcagtgagccgagatcgcaccactgcactccagcctgggcaacagagtgagactccatctcaaaagaaaaaaaaagagtatgtttatgtgtggttgaaacacatatacctttttggtttgtttgcacaccaagtttctagaacaacacataggctgttgacactggatagttttaagggagcacggcactggttagaggtttagaattttttgttctacactcctatacaatttgagtatttcccattgaacaagtattactttattagtaacatttaataattaaactatcaaagaaagatcaataaataaaatatgttctatattaactaataaaggagaaaatggctaggggatgcagtcactgccttatatttttctgtgagtccctgttgtttactaaagtcatagctcttctgtaaggcaaccaaaatataaaggcccagatctgaaataatttgttccctctttttttagacaaaacgaatacactaagttcagaaagtatacatttatattcataaatgtaggatatatctttggcaaatcacccatggatgcttcttgagtattgccttgagaattgtagtatattcccactggaagtatcttatatattaaactgtatgccactaggattttttccagaatcacaaatcttttcaatctggaatagcagaattatttttttaaaaaaagtgtaatgaatgcaaaggactagaaaattaaactaaactaaatattgacatattttagtaaaactctttatttacacaacaggtaatgaattagaagaagaccttgaaattcttgaggaggctgcattgcaggtattgtcccaaatgttttaaggataaactttgtacattaatttaaaaagtattgtcactcagatcagcctatttgtcttgaccattgggttcattcattaagtacttcactttattcattggcattatagagataaataacttatcattgctccctttggtgggagaaacccatgtgagccctatgtatttggtgggtaattgactctggagttagacaaaaatccttcgggagaaaaaaacaaatcaagtgccaagggtgcggaggatggcttgcagaactgtggccatttaaattgcatcttgaaagaatttgtttttctttttttgcctgctagagaaaggggaaagacatttcacaaaggatgtatagttgtgttataagaagcagcaaaaggtgaggttggtacagttggtgagctcagtctgtgacaaaccagtatgtcatattaagtggttagggctagttgcctgtagacaattgggagccatcatttgttgtttttattattgtttgctttaccttttttgctcgtttaggcattaactagcacatttggcactgaatggaaatacatgagtactatatttcagtgcaataccccctttggatctgttacgtcaacctaatatttctcaaaattgcctagagatgactacattgttttcttacttcattagtctgacttcagctagctagtaatctgtaataggatggaaatatcagaatctgtagacagcatttgtcaaattgtaatacctacttaggccaaaccagagtttctgatgtactgctaagtgagccccattggataccacacactgagaacatcagtgatggggaaaattttgcatctagcaatggtgtgaagatagaagaaaagttgagagtctctcaactggatcagtgttactcagaccctaggatgtgatagatttgtaaaattttccaaaaatatttttcctgagaacttattaagtaaaaacactctttaaaaatgtactactttttatcagcaatgtcttataaaataaaagacattttaatagccccagatggaaaggtcttagaatggaagactctgttatagccttagtttttctaattctgccatggatcagagaactttgggcccacatttggaaaacaccaatttagatcatacttattttctatactaggtatatgtagttagtaatagtgatttaaataatatctgtagagcacttcataggtttcagcatctttaatattatctctctgtttttataattacacttcgaaatcagtattaggtcttgcatcttacaattataataactgaagttcaaagaagttttgtgacttcttgaagactcagaagggaaacaaaggctggtacccagaccttgtaattgtgaaggctagtgctttacattttacttatttcctgaggtgaattaatcaaagcggtaataatgaaaacatttatttcatctttgtcaagcactattttaaacattttgtgcatttttaactccccacagcaacccaatgaattaaaaagttttatctccatttacaaatgaggcatagagcatttaggcagttcgcccacgatcatacagctagtgagcaactgcactgggatttgttcgctggcagtttggtgacaaaagaaaccccgtctcattagctgggcgtggtggcgcatgcctgtaatcccagctactcaggaggctgaggcaggagaatcgcttgaacccgggagtcggaggttgaggtgagccaagatcatgccattgtactccagcccaggcaacaagagtgaaacgccgtctcaaaaaaaaaaaaaaaaaagtttgtgattttgaacactgtcttctgatcatagggagctgtagtgttgtcaataatgttaagaaacgtctgagttttaaaaagccattggcatcttatgtgtattccttttttaaagttaaatctttgtaccttaagattcaaaataaaaataaattcaacagatatataataaacaatatgtgtcaggcaacattctaggtgaaggagaactatcaaagagtttctattagtccattttcacactgctataaagaactacctgaggctgggtaatttataaagaaaagaggtttaattgactcacagttccctacatggctgaggaggcctcaggaaacttacaatcatggcagaaggcaaaagggaagcaaggcaagtcttacatggcagcaggagagagagaaggggaaaggcctacacacttatcaaacaaccagatattgtaaggaaaacagcaagggggaagtctgcccccatgattcagtcacctcccgccaggtcactcccccaacatgtggagatttataattcgagttgagatttgggtggggacacagagccaaaccatatcagagttcatatatggtttataatttcagggattattatggtatataaggtgtcatctagaccagatatggagagatctttattatgttattgttcaagaaaaaaaaaaatggtgtcccaaattgcctctttttggcatgtaaggtagttattctgataatctaacatgtattaaacacttactatttgttaggcaatgtattatatactttatatgcattatttagtttgaattttttaactgtgtaagagagattatttgatgaccagtttatacattttacagattcagggatggtaactttttagcttaagagcacacagcaaataatttgtccaaagccaggatttgaatttaagtctatttgattccaaagatgattatttttgaaactgtataatttcaaatttgatagtactagaaatgtaattattttctatgaaaaaatcttactggaatttattttatagtcagtaaaaagttttcagagttccagcatttaaaacaagtctctccaaaaacataagacttgcttgtcagataggtgaaacctaaaatttgttttatctgcctcttcttcttaactttagtaaggcttaggcaggctaatgagatgtgtgtgctgccacaatcaggcttccacccactttttacccaagtcaagaccttggtgcccacccacccaagagaacagacacaccttactcatgatagctgcctgtaagggggactgaaaaagagggaagccggatgcagtggctcaggcttataatcccagcattttgatagggcaaggtgggaggattacttgaggccaggagttcaagaccagcctgggcaacatagcaagaccccatctctacaaaaagttttaaaaaaaattagccagccatggtggcatacgcctgtaagtcccagctacttgggaggctgaggcagaaggatcgcttgagcccaggaggttgaggctacaatgagctatgatggcaccactgtactcctgggtggcagagtaaggccctgtctcaaaaagaaaaaagggggagacatccacagttgctgacagaagggagctctggccctgaaaaatgaggacataacatatagaggactggcatggaaggagatacaccattttatccagcatgcagatctctgacaaggcatttatttttgagggaggaaagagactggaaatattgtctcatatttttctctcctaggaatttcctattctgtgggttaaaagaaggaacagtggggtacgtggagaaaatgatttctccgtaaaggcctatctgaaagtatctgcatgttgaaaaattttatgtaattaaagttttatttttaagaatatataaatattcttttttacttaacaggtgtgcaaaacccattctggaattcttggaaagggtctagctctttctcattcaccaactatattagaagcacttgagggaaatttaccactccaaatccaaagcaatgaacagtcttttctggatgattttattgcctgtgtcccaggatcaagtggtggaaggcttgcaaggtaaatgtactttaagttacttactttattttagggctttcatctttcttcatttctgaaagaggggagtcattagaatattaaaatactactttaaaaaagtctggtttatgtatgtttaacttgctgaaatataaaatgatagttaatatttttgtttggcggttgtttgtttgtttgtttttgagagagagggtctcactgtgttgcccaggctggagtgcagtggcatgatctcagctcactgcaacctccacttcctggactcaagcgattcttgtgcctcagcctcccaggtagctgggattacagatatgccaccacacccggctatttttcttttttgtattttttgtagaaacagagtttccccatgttgcccaggctggaatagttatgatagtatatgataagaccaggtgcaatggctcatgcctgtaatcccagcactttaggaggctgaggtaagtggattgctagagcataggagttgaagacaagtctggccaacatggtgaaatcctgtgtctacaaaaaatacaaaaattagccaggcatggtggcacactcctgtagtcccagctacttggaggctgaggtggaaggattgctcaaaccctggaggcagaggttgctttgattgtgccagtgcaatccatggtgaaaccctgtctctgcaaaaaatacaaaaattagccaagcatggtggcacacacctgtagtcccagcttcttggaggctgaggtgggaggattgctcaaaccctagaggcggaggttgctgtgattgtgccactgcaaccagcttgggcaacagagcaagaccctatctcaaaaaaaaattaactaattaaattaaatttattatagttgttaattgattatttttgttgctttttgtttttaggatgaaaagggaaaaaaatatgctggcctgcatgattttactagtaaaaaaaaaatcccaacttttttctggttttaattttaaaattgcagagattactctttagtttttaaaaatcacaaaatagtgattggagaggaatgtcaaatcacttaagtcagcctcactactatactatgcagacgtttttatgtaatgcatggtagaattacttattactcagctcacaggacaacttgaatgtgtagatcacagaatgaattcaggaatgaacatcacaaattacacaacattgatgcattttaggtggaaagcaaggtaatgaggaagatagttgagaaactgtgaaaaaaagatgggcagtacaccttgaacctggctgctgtcaggcaagtagagggccatttctacccctgcttttacagagacactgctttttccaggcttgcaggatggcagagtaaggtggagctccttcagagagaatgagcctgctcaggacttcaaattatgcacaacctacattcgtgttcttggtgattttaaattagccatagttaaaatttcaagtttcctggttttaaaaagtatttttatactgtatttaaatatcagaaatctcctgtgcttaggcatgttttttaaatgaatttgtccttaacagtttctgagggagacactcatgattatcactgagttggaaatgaagataatgttttagcattttgtttgatacaagttttgttcatttggcttaattttatattaattgttgaagatgctgatcagtgtttggggttttgtttatttgtttttcattactgttaggtggcttcagccagattcatatgcggatcctcagaaaacatctttgatcctgaataaggatgatattcgttgtggttggcctaccaccataactgttcaaacaaaagaccagtatggggatgtggtacatgttcccaatatgaaggtaattataactggattaaattagcagacatctatatactggctgcaatgactgataaaattttagaaatgccaagtgctgagagtccatttgttctaccctctttatataaagggtgatgctgaaagtttgtttaaatgacttgtttatattaattagtccccaagtgtccaagttacacctgttttttttgtgagtttgttctttacattttgctacctgttacggggactcaaaggagggataagaaagtatccatctaaagagtgctagacacatacagtgaagcccctcaatatgtattgattgaataaatgcatgaaagaatacatttttaaattttgtgtatagttttgaaagactcaagtacgttctgtgtttggtattactgaaaccacattttaaaaataacactcattaagttagaaatatatgagtttagattgtaaaagaatgaggaattgaaatagttgtataccatattgatgaatatagagtttttaggatacctcttacctgaaatattaataataatgtttcagagcatattatacataattatttgtgatttaatctgttaatatgaatatctcatttaaaacttttatttctgaaaaaattatattgaataaaattttatataggcagtccccagccctttcctccttcaaagttgtcttatagagtgattggttgtttgaagcttagagctgattaagccacaaaatcctttttgctcattgggtagctaattttgcctcaatattattttcagtagaactatttcaactacttcatgcattctttcatttatagcgtgaagaagtacaatgccaatgtagcatcttacctaagtaaaaaataagaacacattgcttacatgacttaagaccagtaaataaaactgtatggaaaaggtttcaaccctgatactaaatcaaaatcagtcttttagcagtatctttcaaatatttggccatgatccatagtcagaatttttattataacccaaagatataaatataatatatgatggaatagtgtctatgtaatagcatatgttaagtacatataatatactttatatatacatacacacatacatacataatatgctatactctgatgctttctctatcttattttttaaggcaagtcatacccttcacaaactgagttcatgcctacttggaattattctatacaatgcaaaaagcatcacaaattataagaaaaaaagatttaaaaataaagttatcctgatagtgatatgaaaaaaattactactcaaggacagatttttgatcacagtacaatacacgttaagatgttgaagggcttgtatattgtgtattcactgatgaatttctcaatgtagtgagttgttaaatccaatctttgaaccctcaagattaagttttttattcatgtgacaacattctaataccatgttcatactgttacgcttctgatatttctttagttgtattttctttcttgtgttttctcctttgcaccttttattatctcttttgatatctggtatgtttatatcttgcatttgaaagtcagacttcatctttttttcttttcatcttagagttctttttttaaccatatgatggatctgcttttttttttcctctcttcttccctctcctctttccctttcacctccttctcctttttccttcttttcttcctcttctttctcctccatatatttgcttctcctactatctttccttgacttaaacctcttactcagtgtgctctaaattcatgtcagatttatccaatttagaaactttctagaaaaccactctaaatactatctagaagaagtgatttgtgctcctactgatgtttaatgtattaatatcatattatattagctggaaacaaaattaatttaacctattttactttcagcctattagtatatgaagtcttaggagcactttagactgttcataaattgttaaaatctttatgcagtaatagctaattatttgaagcataaaagttgatttgcagctaatatttcaatgagttccaaaatgctttcaaaagcaacaagtgttattgttataattttttacttataaaatgactgttccctacctgtgataaaatgagttagaactaaacacacatattgtaccaatgtcaatttcctggttttggttcagtaccatcagtgtctgacagtgttggataaaatgtagccattgggggaacctgagtgaagggtacataggacatttcatctatttttgaaacttcctgtgaattaaaaaaaaaagtttattttaaggaaatcttatttattgacaagagaaagtcacataggaaatacaagaataactttttttttaagtaacacagtttgtaaaagaaagcagaatggttattctataatgtctcagttttcttaaagtataaaattattcagtgatttacttcaaggttcatggtgcatacacacatagactctaatgataatttattaaatacttagggtaaatatagttgcttacagaaaggttaaattgaaatagtatgtataaaaatagtgtctgtaatttaatatcaaatatgtttgagacaggagagttcccttgacccctttgtgggacttgggacgtgggtgtcgtgtggctcatttacttgactgctgcactcaaaccccttgcaggatggcgagcactgaggcaagcaggtgccagggcccaggcaagcacctctgggctccagccccacagcagcatctaggagtgtgtcacaattaatgcttttttagctttgttgtctgtggatggctaagtgttaaacagctcagtgaagaatcagcatgacagcctttttgggttcctgcacccagtgtgtcctgaattcttgtctggcattcaggaagaatcaggtcatatgaatggtttgaaacgcgatgaatgcgtgaatacagcagattttttttttttttttttttttttgagacagagtctcgctctgtcgcccaggctggagtacagtggcacgatttcagctcactgcaagctccgcttcctgggttcactccattctcctgcctcagcctcccgagtagctgagactacaggcacccgccaccacgcctggctaattttttgtatttttagtagagacggggtttcaccgtgttagccaggatggtctcgatctcctgaccttgtgattcgcccgcctcagactccaaaagtgctgggattacaggcgtgagccaccgcgcccagccacggcggattttatcaaacggtggaagtggctctcagcagaaggggagctagaaagggcatggtgtgagaagaaggtgatatttccctgaagcccagccgtctccagccatgctcctctcctaagtggtgccatctgaggttaagccgcgcgtctcatcagttgctgcttctcctcttggtattcgccacttgtctctttgctagctgaagtctgggatttataggggcatagggtaggggggcagggctggccaaaaaggcaacatttaggcggaatacaaggataaactgttctcatttagggtcatggtttccaggcttgagagtggggcctttgccagggaaccaccttctcctatccagtatatccctgactcctgtctgtatcatcttcagattttttaattgtttaaagtttaaagctttttatgctggcacatgtgtttgagttgaaattttaaccatatacctgtaaactgccattttttaaatataagaatgggtttttaatatttccaggggtttgtgtcagtccaaatactgatctcttgtaacctcacctgaagatagtggtcacttaatggaacatatttgtcacctgtgcctaacattatataaatagcgaaagtctcaaactactgattccatagaatcgcgttgttaagaacaataaacataaagacagtctgtagttctgaggtagctctcaattacttaaaaatactatcacttttatctctgcactttttctatttctgtgacaattctgttgaattacctgttcttgccaaccacaaaactattgtctgtagggagcagcagaaactttgagtaaagctcagaatggtgatacctgctgaagaactaaagtggtattcaaattgtgtgtacatttgtggttgcctaggggtcaagtgtttttctgccaatgcataaacaaataaaaacaatgttttcttataatttctgggaaatatttctaaaaagtatttttcagaaatagtagttagtagatgaaatagtagagttagtagatcaaagatagtcccgttttgttgaagaaattgagttgcaaagttataattcctggaaaatatatctaaaaggtatttttataagacaatttgtattttcagtatattaacatccattaacgaatctgttttttgcatctatactgtagagttagtggacgaaagatagtcccattttattcttaaagaaagtgagttgcaaagtttaattatttaatagattgcttaaatcaaatgttgatgttggtgatagggctgtctaataatattctttcttcacccgagtaaagaaacatatttgggctttaggtataagatttgttttattaagtgttaatttttgaaacttctataaatagagtacattttcttctgtagaacagtgcagctcaaactgacattagtgctttaaaatgtgctgagattgtgggaaagcatttacacttgcattacctgataaaataatttgttagtccctgcaataacttaatgtagattcattccactatcttttcctgtactttattagtggtgcatttgtttttatgttttgatatctctgattatgtattccaaaactatgaggccttgtttttacaccgtgttcatggttcatattgtcaacccatcttgaggtcatttgaactcttttcaataagcctttgaagggaatgccagagacagaccaatgctaacataaagtatacataattatgagtttataatagtaagttgcttattttgatgtcagtgacaattaaataataatggtatttggtttctataataccatgtttaggtggaagtgaaagctgtccctgtttctcagaaaaaaatgtctttacaacaagatcaagcaaagaaacctcaaaggattcctggcagtcctgcagtaacagctgcatcttctaatactgacatgacttatggagggctggcatcaccaaagctagatgtttcatatgaaccaatgatagtgaaggaagctcgatatattgccataacaatgatgaaggtaatttattttacttatcagaaaaattttagttttcagcatttgtttatttaaccaacatttcagtgattcagatgcagacatgaaattgtaagtccaaagagcatttgatggatattaaatttgcagcaacacttattaagatgatgttatcctgtcttcagagaggattttcattggtttttggcagatgtccagaggggatcaccaattacagatcaagttaatcgaatcagagatacagatggattaggctgagtttcagttcctgtgaaagtacttgtctacttctgaccaggaacagtggcccacacctataatcccagggctttgggaggccaaggagggaacatcacttgaggccaagagttcgagaccagcctcagtaatgtagtgagaccccatctctacaaaaaaataaaaataaaataaaatttctaagtacttgtctatttgcagtttactattcttgctagaatgtatctcttcagggttttggggtttacctatgcccccttcaattttgggttctctcaaatgccagatgtatctcctagaactctttgggatttttagctctctaatacctttagacatttaaaaaatatatattttggatgttttagttatcttcagaggcaatgttaatccgaattatcaaggtagtcattatttgaagctgaagtttgtaattttggcattttcagagagcagtggtgagccattgaaggcttttgggtagaggaatggcttgattagatttgtgttttgtaacgattgtggtggaatatggacagtgtaattgggcagatgatcaagaaacatgacttggttttagtctctttaggggactattgacttaatttggtgagatatgctgatcagttggactagtgagttgtcaatgaggatagattcaatagaatcagtagaacctggtagctagtggaggtggagtaaagaagaaaggtaggaaataaaagatgaaattcacatattgagcttgtgtccaattgataatggaagaggggcaggttttggttgagaaagatagcagattcagttttggatgaggtgagttttagatacttctgaaatacctacgtgaagatgtataaaacaaagttagatgtacagacctgagtgatcacagtctgacctcaagaaaaggattcaggaatcattagcacttacattacttatgaagtcatgggaatagatgataccttctatggtgaatgaagacagaagttccaggataggatcctaagaaacaccaatatcactgagtctcagactcttctctacatcagaagcatctggagcacttacttaaaaaaaaaaaaagatttccaagcacccagagatttagattcagtgggtttggagagaggttcaggaatttacctgaaacaaatatgcagtgattcaaattcaaaacctaaactttgagaaatactgatttacaaggtagataagagaaaaagaaaggaaaaggaaaaacgattcacattggtaagaatactagccaaagaagcaacacataattaagggaatggctgaagtgagtgtagataagcctttcaagatgtttgactatagaaagaaccagaggaaatggaattgagtgaaggctttggagtatggggtcttattaatagagatagagttacatatttgagtgctgattaggaggtaattgagagaaaaaacaataaagatactccgaatataaaaatagaatctagagctcaagtggtagcattggccttaaataggaggaggagtagtgcttgtttcattctaccagaagaaggaaaagaaagcttaatacagatgctagtaaatttgtaggttttgcagccaaaggaattatttaaaacagtaattatgtgttttgggatggaaatggaagtgatgaatcttagggtcaaggttttgaaacaaagcggataaaatgaaattgttattctagagattaaaagaaagcaccgatcagaaaagatttattagatgcctttgaataatcagttgaaattgaagattatttgtgaataacagtgtgagtcatatgagcatggataaaacaatcatttggatggatctagcattaaaattttgctagaccagcaaaagaacagtggaacaaggaagttaagtatatggtagctaaagagaggacagtggaacctaatttagtttggaagggaagaaatttcagagtcaaggaagtaagagggactagacgtcaaagtagatataagaatagttattgaggtaaaaaactgaacaggccattttattttgatttcagagacagaacaattctgagtaatgtcacaggtctgtgacttttggtgtgagtggctggaaaaaaagttgaagttattgaggttaaagaaatcagggaatgagaaactagagttctaggtgaattgtccactggctgttaaagaattcctggaaaaaaagttgaagttattgaggttaaagaaatcagggaatgagaaactagagttctaggtgaattgtccactggctgttaaagaattctcttctctcactttttactcaccaaattaataatctcattatgaattctatatccgaaataaatatttgtctcattttttaaaaaatccatcttacacatttggctattttagtatcaaattttaaaattatttctactattaaaaaaaaaagcaaggctaaaaattcttctctattccaacacttgcttaaggaatttagtttctagtctccttacctccctaatcttttagtttcctatgaacatgcttcagtttgtataatttcttttgcaaattttgttaccacaacggaatgcaatatttcaaacatgttctgagtagcctaggtttagagcaggtatggaataccattcccaaacagtatctgtgaaatgttaattcaggtcccaggaaaggaaattttccttatcaaccattcacagatactatgtattcactcagtagtgagccaagtattgtagaggatggaaagatgaaatatggtctcttctttcattactaagtgtgttaataatatgaatttgagggagagtgatgaaatagaaaatacattgtactgaaagtctagaaacatggatttccttttctcacctttgtatggctaaagactcagccttaacttgcgtttttctcatgtgtaacttttgagtaggtaaagtcagctagatcatttctaattcatttttatgaaaacaaaaagggtagagagaaggaagggggctttgagtttgcttccttttactgtaagtacaggaggagttagaagtagatgagcagttggacatcttgacctgaaccttgaaagataatgatttctttgctaatgttgcaggcatacagaagtattcagaataatgtgccaagtttcatgtacctacagcccatttttgtctttacattttaccataattgtttcaggtcattttgttatttttttattaagaaacagattgctacaggtacaattgaagcctgcctaaacccttcctcagacccattaccaccccttccttagaggtaaccactgccctgagtgtagtgattctcattcctgtatatatttttaggctcttactgaatacaaatttattcataagcagtatattttgggttgttcaatacgttttccagaagtttataaaataatatcgtattgtatgtatcattttgccagatttttgcttaatgttgtttttgaggttcagccgtgtagctataggtcattcattttaactgctttaaagcattccattggggatctattccataatctgtccattctcccattgatggacgcttattataaactgtactgaaataaacattctgtatttgtctccttttgtacatgagcaagagtttctgtaagatacatacttagaagtggaattgttaagttgtagggcatgcacaactacaactacattaagtattgccaaaactcacactcccacgagcaatgtatgaaagtagcaggtgctgcatgttctcctctgcgcttggttcttaccaacacaatttgatgaccatgatgtgatttattttagttttgttttaattcgtattttcctccaagtttagttaaataacttttctagtctttattgacctttcagatttcttcttcagagaattgtcttgactgtctttttttgttgttgtttatctcttttgtatggatttattctctatatgttatggatgcatatcacctttgggttatacgagttacaaatctccttctccaaggtggaagcttttcctttctgtatagtatttgttttggaaaactttaaaattttaatgtactcaaaagatgttatctgtttttttcttttgctttttttttttaagttttggtgtttacttttggatcttcaaaccttgtagaatttatctttaataaatggtctaaagtaggtaattttttttctttttctcctatatgtataaccagttgtctcacaaccatttattgaataatatattactctgttaattgagaatgtttttaaatgattttacgattgttcagactggtctctttcttaaaggtttatgaaaattattcatttgaagaactacgttttgcatcaccaactcctaagaggtaaggatcgttttctcaaagtataaccaaaacatgtgttgtcagctgcttgcaactaaaatcatcctgcatttcaactttattttctataaataaatactttatagcatttttctaggttgccgatagagatgtcatataataatgtatagatttgcagaaagaatgtgtttacattcaactgtaatactgccaatatattacataatactacctactagtgaaaataggtatgtaaagtagatatctagtatgtaaaaatgtttaaattgacttgtttagggttgattagtttgttttttgtttgtttgctatttaaaaattgttccttttctccttccaatgaattgtggagagatttcagcatgtggtatacttgtatttgattgtactgtcttaatttttcagcccgtaagtaattctgttaacttcagatttaaggactttttttgagttaatattttatttgttaggaacagtactacttaaaatagaaacttggaaaaaataattagatgcattatactttggagagatcacctccatagaatttagaaaaatctctgaagtgaatctgagatagcagaattatgtataggctggaaataccagtttgaaactcatggggaaaataaatttgggaatcatctatattgagatggcatttaaaactgaggaatggttgtcacctagatagatggcataggtagaaaaaagatgacccagacccaaatcctggggcattccttaaattaaacggtagtcagaaaagggaaagcaacaaaacactgacaaagaatggccagtgagataagaaaaaagccaggaaaatgtcacagaagcaaaaagaaggaagtatttcaagataggaagagttgtcagctatcagatgctactgagagattgctgaggatgaagtgaccattgaacttggggacgtggtggtcattggtgactttgacatcagcagttgtaaggaaatgttggaaacaaaagcctaataaggataaattgtggaaaggaagtagaagtgagagaatggagtttgatataaatcggagaagagatgtaagaccttattagagggggttgtaataccaaagggaggtgttagtgaagattctaaagtttgtgtgctaatcgaaatgattcactaaattgagagtaattgatgatgcatgagagagaacaaggtgattgcagcataaaaatgttgagggaataagattcacagcacaaggggagggactagctttggatcagatccaggacacttcctccattctaccatgagagaaggcaggggaaagtaggtttattaatcacattgcaactttgaggttgtatctgattgcttattgtctcacagaagaaagaggtgaggtcatgatcacctgagagttaggaagagaggtgatgcaggtttgaggtaagaaagaagtgaaattcaagcattttggagagtaggcaagcgtactatggcagggtggtaggattgccaggaggtatgcaaacaaattaaatgtttgctgaactatcgtaatgatgttgactgcttaaccaaaagtaaaatttccaatatccatcttatttcttttcagacccagtgagaatatgctgatccgtgtcaataatgatgggacttattgtgcaaattggactccaggggctattggactctacactcttcatgttaccattgatggcattgaaatcggtattttctttaaagccatgagctactacttacttcaaataactagagtcaaaattatttttatcttctagttttgcccattaacttgcactagacatatggataggtttcataacttatttgaggccttgttttctctttcataatgagtatttaattagtgcattaacattttgtaataatttctggtgtccgcccaggcactgtagccctcatgactctctccctccatttcctgatgttactagtcaggtgtgctctcttccacgctgctgttaccctgtctctcccctgtgggtgtgtctgtgcctctcctgactgtgagcagaaagcattcatgtttggatcttccagtgtagctcagtgcctggcacataatgacagcaaatatggatgaatgaatgcatttttaaatcgatcattgtgttaatctgggttctccagagagacagaaccaataggatatctatacagtcatgtacctcatagcattttggtcagtgacgaacagcgtatataatggtggtcctataagattttcctgtaccttttctatgcttagatatgtttagatatgcaaatccttaccattgtgttacagttacctgcagtattcagtagggtaacatgctgtacaggattgtagcctaagaccaataggctgtaccatataacccaggtaatctgtaccatctaggtttgcttaagtccactctgatgtttgcacaatgatgaaattgcctggtgattcatttctcagaacatttccccatccttaagcaatgcatgtctatattagatatatgagaggatttattagggaaattggctcgcacaattatggagtctgaaaagtcccattatgggccacacctgcaagctggagatcctgagatgcaggtagcctcagaaccagggaagcagatgatgtaactctcagtctgaggccaaaggtctgagaatccaggagggccactaataataagtcctggagtctaaatgctggggaccctggagttcaaatgtcaagggacagtagaggaagtgtttatccaagctccaaaaaatagaaatacattcacctctcctctgtttttgtttgctctgtgtccccagctgattggacagcgcctgtccacattgaaagcagatcttctccacctactccactcacactcacacgccagtcttgtctggaaacaccctcacagacgcaaaagtaatgccttaccaggtttctaggtattctttaatccagtcaagtggacacctagaagtaatcatcacaaccattaactttgaaaaacatatttcttttgttttaggcattaatcttttctttacttttaaactgtctgtataataggataacaaggagaaaactctagtacttatcagtgtgaatttcttgtaacattatttttcctgatgctcctcaaggaaaggggtttctgtgatcagataaatttcgaaaacctggtttgggatgagtatcttttatctgaaatgcttggcaccagaagtattctagattttggactttttctgattttggaatagttgtattacagttaccagttgagcatccctaattcaaaatttcaaaattcagaatgctccagtgagcactttttttgagcatcatgtcagcacccaaaggttttagattttagatcattttggatttggggttttcagattagagatgctcaacttgtatatggtagtctcacttgagacattcacacaatctacattaacatattagggattctgagaagttctgaaatcaagaaacttgtctgactatgtttaatccttatcgtgggaccccttgcaagtggagaagagagaaacagggtggagatagtaataataccaattaatatttcaccaaaatatttgggaaaccctgctttaagtagtttgctttggcagtggaggtaaagaggtactttacattgatgtaattgtatgttctcatacattaatgtaactatatagactcagatatatccagttcatctgtaccattctagagaccttcaccagcagcctttgctgcactcttctttatttcaatatattcataaagcctttagttcagcaaatgttttttggttctgagcatacattttcctgggtaagacaattcctgtcaagtgttaacactttaatgaggattgtgaaaataaaacacgcaaacaaaaatcttatgaagtagaatataagtagtgacacacaagacatacaaattacagtaggaattcgaaagtagaaatagccaagaatgacattgtgaaaattttcttttctctaaaaattctggtaaaatttcagtagatagagtttttttcatcatcttatctaatagtggggatgaagaaaactgatacaaattagataagaaaaatccacttatatttgttttttggaatgaagctgaacaaaaatacttgcttacttagaatcatccattttcttccatactcaacttccctgctcttcttcctaagttttctccgatgccatcaatgggctgcgaccttgcttgaccatagacaagcagagataataattcactagaatagttataataataatagctagcatttattgtgcatttaatatatatctaaattttctctatgtatacttttttttttttttttttgagacagagtctcactctgttgcacaggctggaatgcagtggcgcgatccctgcccactgcaacctccacctcacaggttcaagcaattctcatgcttcagcttcctgagtacctagaattacaggtgcgcgccaccaggcccggctaatttttgtatttttagtagagatggggtttcactatggtggccaggctgatcttgaactcccgacctcaggtgatccgcctgccccggcctcccaaagtgctgggattacaattgtgaaccactgcgcctggccccttgtacattttacgtcatttatttcctacaacagctcggtgagttagatactgttatttttcccattttatagatgaagaaattgaggcacagagagtcaaacagttagtgcgagatattggagtaaccaaagcagtctaacatgagaacctcagcttctcacaaccatgctacattgtcatagagaggactggtagagatgatgagtggtattctgattggatattgtcagataagatttaattctcaagggttcttttttttattattttccattttttctgtatcatttcatatgttctagttaaagatagggagccccttcttttccttgacatcctgagacattcctttagaatctcagggccatttttaaaatatttctcttgtttagcctattaaatatccatcaccttaaaaggacagaaatcagatatttgtgttaatttttaaaaatagcctgtggatgtttataagattaggtttaaggatcttccttctctttttttctcaaatgttacagttctctgatttttctgacagatgctggtctggaagtaaaagtaaaagacccaccaaaagggatgataccaccaggaactcagttggtcaaaccaaagtctgaacctcagcctaataaggttagggcagaatgaattgagagccggaatcattcaatttttacgtttgtcactgatgcttcaaaatggccagtaaacatctaacattttacttgatttcaggacttgaattctaaatatccctaaataaatttagagagtagttcatggtaatatttctaagttgtagcatgtgtacttctttatttggtacatattgtattcttcctgctgtggaacatttacaatgaacaaaagtctactgaaattaagtgtaaagtttgcaaggtgaaaatgtcctctactgtcaaaagaaaagttaatttctgaaatctaaagagtaagtacaggcattttggtaaactgcatgttaaaaaaatgaaatattggtaattgaatttatttttctaacagaaagaaatggtttttatccttcttaacacgtatcttcagtatttagaaatgagttaaaatatatataattgtctaagctttattttcagtaatacacatttcagtgcttaaaagttctgttgttagtgttgttatactgttatttgcagagggagcctctctacaaataattctggaaccttattaagaattacgatactttcaaacctgcttaattttacttcttgctacttgtaagatctctattctatgttcttcctacctcactgtttatctttgcctttctcaggttcgaaaatttgtggccaaggacagtgcggggcttcgcatccgtagccacccttcccttcagagtgagcagataggcatagtgaaagtcaatggaactatcacttttattgatgaggtaattgataaaggagcttttggtagtaaattttggacagaaagtttttgcattgtttgcataataataaggttactgagttttttgtcagaatatgttttaagcagtttgggaaatattttaaaactattttatagaatgtgttcattgagtttcaaacatttcatttattttatcattaaaaaatgactaaaattatattttgaacatgaaaagaacaatgttcgacatagtattcagaacctgtacatgtattcactgaaaatgggtatagttcttcatgacattttaaaatgttgtttgttttgaaatgtgtttcttactgctttaactttggacagtatatttttctggtaatataaccaagtaacttctgtttcttaaaatataaatataagggaaggaagactcatattcctttgggaaacatctgattttactgtttatgtatcttaaagtgtggtttccttgtgtttttattcctaagtgaaattgtttttacagtcttgccacatgaattaccatattgtgttggcactctttgctgctttttttttccctggagggtctctcagacttactttctgcaaaaactagatgcttaatctgcacatgcaccatcatttttctgctttgcagactttcagaccttgctagcctcataggacttacaggaaggcctggcttgtttctgttgcttttttctatagatccataatgatgatggtgtgtggctgaggctgaatgatgagacaataaagaagtatgtccctaacatgaatggttacactgaagcctggtgcctctcttttaatcaacatcttggcaagagtcttctggtccctgttgacgtaagtaaaaggtggttttaaaaagcattatagatacacgctgtttgttttacagagctttctttaattaaggcttttcagttctgaggtaacccacaactaagtaacctttactttacaatttcttgaattcttaagggcttctgtgtaatgaaagtgaagtttggcaaggttaaggcccttagtgcagattatgatccatcacagataggcaggaagtagtaaatttaattaattgctacttaattaaaactactgaattcctttcaaagaggtagaaaatgagagtggttacatttttttctttcttatttatacagctgtgtggaatgaattccaaactaaagtttatgaactcagaattatacattcaaaaacatgtataaatgtatgttatgttcacattttgcagatagttctgtgtgttctgtctaattttgactctcttccttctcggcttataatactattatttagatttgtggtgctccgttgtattctagttgaagttaaatcctagtctttgagagaattcattgtacctccttgttgagtatagtagaatagagccagtgttcacaagattgctacttcatgaagtataataataacccacacttcgttttatatattaaccaggaaagaatgatttttccttttttcattaccatcaaatgtgtgtgctctttaggatagagctaataaatagcacgggtaaaattttatttaaaaacttagtattttaaacagttgaatttcttaaaaatccgtttataaagttagcttattgtatcaaaccattcagtatattccatttgactttctccttgattgtccataataatgttctccaaagcagtagtttatataatacaagacaattctttggtgaagaaaacattggaatttctatttctatttattgatctcattcttttttatatgcttttgtgtatgttttataatagcacagtggagactgtatatagacagagagcaagaaagagaaggaagatatgtgatcaagaagatttagagtgttggcttactgctttacggaggttaagttgaacaatgatacatctataagtaaaatactaaattttaaattaaaaaattacatcaattacgtcttattaaattttcctcagatttatacctgcagttaaaaataaaaaggattttgaccctctatgaaagtgtattttaaagagataattatagcaaccacttttgtacagtttttttttccaatatagacttttttgttgggcaagggggagttggtttttgttttttatacagatgggagagccaggcgaggtggctcacgcctgtaatcccagcacttttggaagccaaggccagtggatcacctgaggtcaggagaccagcctggccaacatggtgaaaccccgtctctactaaaaatgcaaaaaattagccaggtgtggtggcgggcacctgtaatcctagctactctggaggctgaagcgggagaatcgctggaactcaggaggtggaggttgcagtgattcaagatcacgccactgcactccagcctgggagacagagcgaatctctgtctcaaaaaaaacacaaaaatatagagatggggtctcactatgttgcccaggctggtctcaaactcctgagctcaagcgatcatcttaccttggcctcacaaagtgctcatattacaggcatgagctaccacacctggccattgtgtgttttatttaagaaaattttcttaagtccctaataaaatgttattccagattatattatcttttctaccttcctagtccaaattttctctgatttctttgctcatttgaggagctgcttttgggagagaaagatactcacacagataaagcaagtcaataaacaaaagtagaaaggaagagcaagaagtaccatctgtcacaattctgaatcccacgattttattttaaagcattgtgttctgttacttgatgcttttgaaccccatccaagagacaatctgtagtagacagtctccttaaggtcagggaaagaagggagtaacaaaggttagaggagcggtaaggagtggagtgaggcatacagatacttgtgggtggttttcactggaatgtgagtgcggaggttgttaatgtaaacagctcaagagtgtctggtattgttaggaagctagggtttttttggtttcttcattttaagagtagtagtgacttgagagttttataagttaaaagaaagatactagtagaaaaagggagaaattaaagatatagatgacaagactgaagactgagaaacaggaggaggaaatggaattaagggcaccttagagagcttgcttctgtaaaagagaggggaatatgcatggcagtagttatgttttagaggagggagggacgaagggaagaggttgagggagactacaagttcccttaaaatagaagataggaaaacgatcagttcagaatcaaaagaatgagttaagttagggggttgagaaatgcaatgaatatttgaaatattgtttgaaggattactgggccactaaagactcaatgagaccactgtatagacccaactggaatcccctgcctagtcctttattgccctaagtgtctggaattaaaagagccaagaaagagccaagaaagcaaattatagtactagttgctgagctgctgtgacagaaatagcaattaagattatcagttctgggaatgtggactcagacctaagttagaaatgtgaaaggacaaatggaaggtccaagggatttagaagatttaataaaatcctagaacaatatagtgaatgcactatagttaaagagttggaaaaatgcaggcaataaagtagaatatttgtatttgatatcataggtagggcactttaacaaatgagatactctattacattgccatgggagggtggctgaagtgcagtgcacgtggaagtcactacaagtgcctaagtctaggaactaagaagttaggcaggtggaagatcatctgcatggacttgaagacacacagatgatgtgactcacaaagttaaagataaaaacaggctgggcacggtggctcacgcctattatgccagcactttgggaggccaagacgggtggatcacaaggttagcagttcaagatggtgaaatcctgtctctactaaaaatacaaaaattagccaggcgtggtggtggacgcctatagtcccagctactcaggaggctgaggcagagaattgcttgaacccaggaggcagaggttgcagtgagctgagatcacaccactgcactccagcctgggtgacagagtgagactccgtctcaaaaaaaaaaaagaaaaaaaagataaaaacaatgtgtttaccattggaggaaggggtccataagacacaatttaaagtcatggggggatgggaatagtttggatcaggaagaaacagagcagtttggtataaaaatgaaagaacaggtagatgacataaggatttatgttttgggtggtgatcaaagatgacagatggggtacagtatggcaaaattaattggcatagggctctaaacagatctgggataaagttacttcagtacaggtattaactgaggactcatacagagtcagaggcctggcataggcagggacaggtatgattttctgtagggactagggcagaccccttacgcagtcagtacattttttttatgagtatctgactgtgcagggctaaacatggtaggacatagaataatgattccctctcatcttctcaaggaaataacacacctaaatagttgcatcttgctgcctcttcttttgtatgtcttatacatttactgctggaaaaactatctttctcccttcttacttcagttcgtttaaagtaaatgaactttaatcatggctgttaattgtaaaacatgactcaataaaaaaatgaaagctgggagatatgaaatcaaattagaagtattcaaacaccaatgctgctaacttaaaaagaagtgaaagtactgaaaagttgccttcagcactgtttgtgtctatctcttcaaccttttctagttaaaacaattttgtattgacatcaatgtgtgcacgttatttggatcccaactcaaacaaactatagagagaggaatgggggcagagattggaagtttgaacactgactgaatgttggatgaattatttttaaaaataagattactataattacccattttagataatccttaaagtgatcttgtcatgctgtcttgtttactcatgttttagaattcgtgtattcatcgttaagtatgaactttagctcaatttggatctatcagtctttctatttaaacaccatatttgactgaaaaatattcatatcctaatttagttcaaagtgactttaactgtttttagtaatgctctagttgagagacaaaataacattagacattcatattatgactgtatcgtttccaaaataatttttaagtcaatgggaaagatagagctaaatagatgaagggttcagatcctctgtgaggcattcattatcaattatgtagaactttttgccctttttagaacacttttcctttttttttttgttatttttgttgttgttttttgttttgttttttgtttgtttgtttgtttttacagcaccttgccctgccacccaagctggagttcagtggcacgatcttggctcactgcaacctccacctcccaggttcaagcgattcttatgtctcagcctcccaagtagctggtattacaggcatacaccaccacacccagctaatttttgtatttttagtagagacgggcagggttttaccatgttggccaggctgctctcgaactcctggcctcaagtgatccacctaccttggcttcccacagtgctgggattacagactgggattaaagacgtgagccactgcacccggctgagaacatgctttccagttttatgtcttaagagtctttgttttgttgtctaatttatctagaatggtttcacttccatttttttctaatcttaagttgtccagactcaaaaattggttggtgatcttctctttattttccatttttgctatagtcattaattcattaattgctcatttagtaaatatttgagttcttattcgtagaattcttatttgcattcttgttagtaataaatactaagctggaaagtaggcattgggtggagatgcagaggggagggatacagagtaaataacatggggtgaagttctgatccttggagtactcaccatgtaatgtggtagacataccatattcacataattaaattgcattgtgataagttgtggaatggcttcacttaagtgacatttgaggaggacttgaagaaaagttgccaggaaacagtaaaggaatttctagacagagaatcaaatgttcagagttatggggacatgctatgtatgtgaactaatgaatagttgatataggtaaagcagagaattgagggtggaaaacagatatgtactggggagaatagctttctctcttccttctcaggcagcacttaatctgtagaagtagagaaggcttacttctttctccagctacccataatatttgtttttcttgccttcagcccatttgtgtttatgtacctcttatttctgtttttaagctacttaaggataaggactcattcaggatcccttctcactcacatttgtttcttcctaagcattaaaaattacactttgctcatagtaggttttcacagtttgttgatgctttacacatttgtggatgcttaattttagttctttgcatttgatatgatcattctgtttaaaataatttctaacatatatatgctccttgccttaattatctcatttaatcctcattgtgaccctgtgatgttaagcatattataattctcatttttgcaatgagtaaagatagccactcacccaggatcacaagtagaaagtagtggagctggtattggaacaccagttgatatgattccaaagcccatgcatttatgttccgtatgcattaatctagtggagagataaggataaatatagttataaaaggatgaagcaactatagctgtaataatcctgttactttgactagaactgccaaactacagaaagataaatacttataaccttaatggttgaatgaggatgaataccatatgtattgaagatgacatgagttattctttgattgcatacctggggaacattgattgattttaaattaaaattctgaagatgtggaatctgcaaaatgtgagctagtagaatgtttcaaatatcagggacaatgcaagtaaaaagatgaagagagaatttattatgcacctaccaaggaagacattagactctggaatcacaagcttaattttttggaaaacaagtgtacttctatgataggagatgttaatggtttcaaacttacacacccaattctttgggtactctgtcaaaaatgagatcttttaacattgtgaggggggttgagtggaagagaaagtttatatcaagggaagtagatgatagacaaatggaagtattctgaggtatctttatagttcccatataatctgaattcttacagaattgagtttctgtatatctccatgcatttatacagattacttcttattttaggctgatgggtgttcacatatagaaagtatactgattttccaaattttggaacctgaaagatttgaagtagagttgaagagaaagtatcatttgttaatcttgtttgtatagcttcacctgggttaattgataaaattggcaaatgtttcttgcattttaaatccaatttcattcctttttacctaataacattaaatccaatttcattcctttttgcctaataacacttagcagtcacagctttaacataccccagatttcctgattttgttaatagctttgtgtgtatgtgtatacataacccgtgttttaatctcaataatattaacttaatacaacattagtaaatgtgctgttcttatatggttaagaacagctttatttcattctttagcttagtgttaaagtttcgtaaacagaatgagattaaaacagttaaattaagattgtaataatatatatacgctattcttaattttcctgagtcatttcatcattttgctttaacaatggcaattcttagctttgtgcttatctaacacaaggaaagatttttttcttttatttgcatgagcatttggcatggttggtcacttattttcactgtgataattattcacttgttggtctcttttctacgccatgaaagattatcagcttagagttgcaatttggtgttcattaatttagttagattcccagtatgctttaattttctaattttcttgacagaacttttctgcctttaaaatagtatttgagaatgaaatgttattttatatatatgtgtgttcgtatatacacatgcacacacaagctgtgcttataataatctgtgaaattatactatatgaaagctaacatattttaatagtgagaaattggggctttggaaaaattaacattttcctgaatgttcatatctttagactaaaacatttctaaatttccaaagaacactattcaagtgtgtgtgtgtgtgtgtgtgtgtttgtttgtgtgtgtgtgtgtgtgtgtgtgtttgagatgaagtcttgttgttgcccaagctggacttgaacttctgggctcaaggagtcctgccttctcagcttcccacgtagctgtgactacaggcatgtactaccatgccgagctatatttttaaattattttgtagagacgaggtctctctgtcttatccaggctggtctcgaactcctggattcaagcaatcctcctgtctcagccttctgaatagctacgagtataggcatgcactactcacccagctattcaagtatattttatttttaaactccagctttctgttagtcaaatttctgcttcttgataaatgcagttttattaggacattgcattttgaaaataatcttagggtaaaccagtgtgaattaagaattttctacaactgcagaacaaaagcaaaaaagtataaagtttcaatttacatggaagcatatacagaattgattttattggcaatttattaccacagttagcagttaacatcattttaaaatatcattgtacatgttgttgactgagaaagcacttatgtttgcacattgacttaaaagagagacataagctttaaatattgtcaaccagtcatcacattgcttctttatcactaactttacctgcattacttttaacaattgcctttcatgttactttaatactagaataatttatgctaaaaaatagaatacgctaaaaaaaagtgatattgttttatgtcaaaaagtctgtcccactatgctttgtaagggagaaaaacccattgaccaagtactctacaaattcttatatacgtggatgaaccagtatagacatttttcccctaaaacatagcacttcaaaatgctctgcaatatgtttttcactttacctaaagagaaaggacgtattccctatccacttttaattagggggcagttggctttggttttggtttacatttatttgtaggcatattattagacactttgtttagagttttatatgtcttatatttatgaattagtatatatttatgtagatgggtgtgtacccacattcatctgtacatatatatgtatatatccatatatatatatggatatggaaaatatagaactagaaaacagtaaataaagtctcattatctaaaatgagataatgagaaaagtttcattatctaaaatgagataatgagaaaagtttcattatctaaaatgacttataattgataaggaattttgatgtttcttttttgttgaagtcttagttgatcttttttattaatattttgaatgtcctcttttttggttcaaaataaccgtttatcataatatttacataagcaataaaattgtgcaattgctactcactagaaagattttaatttcaaatcagataaatatagattgatagcatgagataactacgaaaatgtatatcatttaaattatcagtaaaacattttgtgaagatgttaatttttctaggataacgtttctgtgaattaattgttccttaacaaaacatttttagacttaaacttagagatcctaaatgctctgcatttcctaaactttgagatagagaaaagtaggcctgtcttttgataaaattgattacatttttatcttttgataaaaaattgattgtatgtattatgtttttcttatcaaatttgttagtagcattcagaaacatttccaaatgaattttttcctacttataaagggaaggaaaattgaaaaaaatgactcagactttttattttttacttttttagtttttatttttatttttgtacttcaaaaagtgataagttcacgtaaactacatgccattattattcataatgataattgcattttctatacaatcgatatcatttccatttttattataaaacagcagactggtttctgtcataaaacaaatctggcttgtattagaacaaatacatcctcatttaaagaattttaaggtttctttcttgcattgagtatcttaagctttataacaacgtctggctcttaattttagtaaatggcatgacacactatatccatggagagcttctgacaaaaacttacttatcagacagaatttcttcggtgtgttgtttactcagatggcttcagtttctctttagtaaaataatatttccatgttggtgctagagtacagaaaaatgttgttactgtgcacgcatcttctgcagatgatatcctgttccctttctttgttgatatttttctttcttaaactttgttgctcagaatatctttaatgctagccaaggagtcagggatttggacgtattttcatggacttccaaagcttttttcccccaggtgagttaactgtaaggaacaataatattttattcaggtatttcagcatgtataacaaattttgtgtctttaaaacctttgcttaagattaataaaaccttttaaccaaaaaaagatctatatgaagtgctgcaattgagttttctcactcctgccaaaaagaatgtattattagtttaagtgatacttccacatgacttcaaatttgctatatttagttttatgagtagcttttccaatgactatatctaagattagtgttttcacaactaacttatttaggtgacagtgtcctggccttatggctcttttattttttctgctgccaacactttcacattccctctttgaatgttcatctgagaatgggatgggaatggagggagaaaataataatattgaaattcatgaatttaatatgctggtacctgggtcttacatttctgtattgaataaagcatgtattttactattgcttgagcatgataggcacagttctatgactttatgtctgtcagtgttttatactgtttagcacaagaattggataaactttgttttatacttattaatatagttttattaccttttcaataaaatattattttaaatttttaacaagttttcataattgcctttcagtgactagtttgcaattataattagttgtgccaaggcttaactcccagtgttaccctgaatgatcaaaagttagaattattgttataattaattaaatgcactaggaatattattaaataccagtgaagttgggtttttttgctaaatctatggatatttgcttttttatgttgccatgtaatttttaagaatcagttgtgagtgggattaaaacttgccccttccaactatgaaatggcatgatgtatttatttattaaccatttacaaacagacattttaaaatgtttacagaaaatcttgtgagtgtaatagcaagtactatgtaaagtttagacctgtgttactctcaatttttatcttgagtgctctctgtgtattcatgtaggtttgcatatatataaattcttacaaatatatatgatgagtatataaaatgcgtattttacatttattttcggttgtaaatctaagttttttgagtgtttaaaattctcatttccgttaaatttccaatatgtaaccaaagaatattggtaaataaaatataattacagtaaatgtaaattaatttttcttagtacatatttgtataagtaaaaattggttattcatcactatagtgagattcacaaaatattatgtgaaagttaatggtgactttaacattttccctgaccagtcatgtatggaattaacaaagtagcaggtatacagaattcttgaatctacatatttttaaacctgaggattctatttttaatattctactgtgttactgtgtgtatgaacatgtatacatatctcttcattatgaggctactggtagggatagaactatttaatacaattctatcttgcataatgttagttgttaaatttttattctatttataacattattaaatatcagttttaacctaatgtttgcatgtgtgtctctttatggttatctattactctatctgaatatgaaataaattatagagttcatattactgtatttaaaaatgcagttttaaatctcttttaaaggagtaatgatgacaatgagaacacccagtgtggaccctttatttacatttagttgggaaaaagagaaaatatggaaaggttattatttctctttattacattcacataggaaacaattttatcacagcattacaatttaacagaaaatttagggaaggttctttcattttagaagtgactcttttcaataataaatttacgttatatttacttaaatccatgtttaatttctcctttctactaaaaatatagtctgtttttctgttactagcattaatgtatcactgcatagtaatggcttaacaaatctagcaagacgtttttatttgtacaatattagagagtataagttgttaaaatattgtaagcatcaatatttcattcataactaacaaatagcaagatatagctatatttttaatgcatatttttcatcatctctgcttatggatcagttttcagattcttcatacctgttttctctatttataaataattatttcaacatgggaagaaatgaatatttagacttttttttacccataacaatatacagtaatgcagtaataatgattcaaaattattcccttgtggttcacatagtatagtatttttatttttccaactttaaatattgcattaaagataaaagtaaaatcaaggatgattatatttctcaaattatggtgggcagaagccagaggttgactggtttcctgctatattttcttgccttcaactttattttaaatagcattgttgaaaaactgagattctttaattagtgctaatgactaattcttcagttatctttagttatgtgttttttttatattgacagtactgtagacttgctataaaagctattaataggaaggctttgaaagtgataatagattgtattttgtttatttttctttcctattcaaattttcaggaatctaaaactaatactgatgactttttcaaagacataaactcctgctgcccacaggaagcaacaatgcaagaacaagatatgccattcttgcgaggagggccaggcatgtacaaggtagtgaagacgggaccttcaggtcacaacatcagaagctgccctaaccttagaggtatcccaattggaatgttagttctgggaaacaaagtcaaagcagtgggagaggtatggattcttgtagcttcatgacttctaagatactcatttttaatgtaatacaaccaaagcatctcttctaaaggttgccatttctaccacaggtttttcaatgttaatgtattttatctttacttgtattgcaaatacataatttttctgtgcttctgtctaatgatttgcataattatccttgtgtaaacaatatattaacaaatgatttgtattgtatatataaatgtttggtatttgcacatattttttcattattacaaatgtactataccatcatggattatcatcacttaaaatattctatgtgatcggtatggagatgtggtcatatgcaaaaggtttcatttctttgttcccattcataggtctcactgaagtcctgagcaaataaaattactctttaggctactgtggcatttgcctcagctgataagcttgaagatgtagtgtgtcatgctatttctcagtaagggtcattttcaagttacactatggatactctcaacctaaataatagcaagtcggctgtgttaggacattttagatgcagaaaaaagaaaatgagccatgtgttcttgggtaattcttaaaattatttaggtgttatctttgtgctttgaaagttattattttaaccaaattcataaataagcagtattttacagaactttttattgacttctgtcttacctcaggctgtggccttaatttcagaaactaatttcttagtagaaatgtattggaatgattacactattcagacacgtaagatttttactggttttcagactgagttccctgaaagaatcaagccaatcaatcatgctttttgttatgtaccaggtaaccaattctgaagggacatgggtgcaactggatcagaacagcatggtagagttctgtgagagtgatgaaggagaggcatggtccttagctagagacagaggcggaaaccagtacctccgacatgaagatggcaagttggcttaaatttgtgatttattccaattaagctttcaaatacagaagtttaatgcctattgtattgcttttgttttaaagagttttttgtgtgtttgctttttaacaagcaaaactaaaagttaaaaaatcttgcaactgttaaaattggtttgtaaaatacgtgagtacttaatgcacattaaagaagaaaattaaaagaaaattcgaaacatatagactcatctttataaaacaggcttttaatttcattaatataaatttatatacctctttcttctgtaccagtccaggactgaacgttctatcccttctataataaaatgaaacttcggtattccaactaccaatctcagtacttctcccaggcaacttcatttgattgcattttgtcttagggaaagagcattgaataaatgcatagtgtctttagggtttcctgttgttagattttcaactttaaattttattttacctttttcaacatggctatcacttaataaaatatattcctttttgtccccctctctttgatatcttggtggcaaaaatttgttcttcgaagttgagaccacattatcacttgtgtgatgctttaactgatcacaacagagcaaagaatttcttgtgttcctaaagaattgtgctcaccactagaacaatagtgtcatagtgaagttcatgtttatttcttcatacccagttatgagtcccttgtggacaggcacatggtttttatctttccattcctagtactgaaacagttttgacacatactagttgctcatgaagttttgtaatacaacttgaattataaaaataatagcacatcaaaataagtatgtaagcaaacatggacttgtctgccaaatcccaaaatttataggatgaagttaggtaaataagagaacatgcatctttattcaaaagataataaatttataatacttattattccaagaggcaatttggccataaatatggattcagaaaaggtttgaaatttgcaataatgatggagtatctttggagatatctccctttttccccccacaacacacctttgaggtaggtgatataatcttaaatttataggtaggaaactcaagctctagaatttaaactaacgtgtctatggttgtacagctggtgacatagcagatgtaagacttcagccagaatcttcattcgtgttttaaatgctgtactcattacaccatgctgtacacattgagcacaacttgagaacctacctcctctccttgtttttcccagatagactttttttagtctaagatgatatggtcataaaataatgtcctgaaacagtagaactgttcatttgaaaaagataagggttccgcttgatgtaaatcagacatttactaaatgactttttcacttactatgtgcttattatatattgctgtatatttacttcatattcataaatttttaaacatttttctttatataggaatattcagatataaatgaaaatacctaaggggagatcagctttatttaagataagttaaatatgagaacagcttaggtgacttctaaaggacttttctagcttaatccttaataattttttaactggcagtacaaaaaatatgtaagagtggattctgaagcaggactgccagggtactgatcccagctgtgcaactttttactcatgtgaccttgaccaagttaacctaatatctctgtgcctcagttttcccatttataaaatgaccttataggtcataaccacataaatttgatatgaagactaaatgagcaaataacagataaagctaatggatggtaagttaggtcttattgttaaaatgcaaaatgccttccatgctaaggatcaagttttaacctgggaaattttattcttataaacttagaagatagaagcttctttcaagataaaatgcctcttaaattagagtatcatttaagaaaagtttaaatttttctctgtaattagttaatcatcttttactcttgatgtatagatacagacataaatatggatatttggtgatcatgtgaatcttactattttaatataaaacctggagtgataaggaaagaaatgatatgaagttagtgcctttaaatcactaagattagttgatgtgctgcccaaatagtctgttcattggtgataatatttaatatggaagtacaaagatttggcactatggtaatttatatgaagtcatgtgagtaaaacaaaagctttcctctttgtaaaaattctctattggtctttgaaatctgtagtaggcaaagttactgctgtggtaaaaacagacttcactctgggtgctagaccattcttagtctagagaggaagcagtgctatcttttccgttcagtttactgttcatgggtgtttttctgaagaaacattctactgctgaaatattgcatgctgaagagtattgcctaagggaagtgctaagattggttttttaaatcaagtctacaaatgctggtaaagggatagattatacagtaccactggtttagcagaattgcaatttacaggtaggaaggaaacactctaccacaccctgtctgtagttccctgttctgtttaaaggactattgacttttttttttttttttttttgagatggagtctcgctctgtcacccaggctggggtgcagtggcgcgatctcggctcactgcaagctccgcctcccaggttcatgccattctcctgcctcagcctctctgagtagctgggactacaggcgccggccaccacgcccggctaattttttgtatttttagtacagaaggggtttcaccgtggtctcgatctcctgacctcgtgatccgcctgccttggcctcccaaagtgctgggattacaagcgtaagccaccgcgaccggccaaggactattgacttttaaaaaagatatttaagatacttaaagatattttaaaaataagatatttaaaaatatacttgagatattttatttttcataaggttattttaatggaataaataccttttccccccaattttacttgacagactttattttgccttttttggctggcattaatatggatatttaactaaggtattgttttatatcagacagagtcttggagccagttgattgttcataggggattaatttagtttgcatattttataggtcacatcttgccatttgtaaatggataaaacatttgccattttctgcttgaaataagaattctgatacatttatagactctgattatgtaacatggtttattattatccttctatttgggaaaaatatctttatgtgataaaataagttaaaagttaagttttccataattttcacattcataacttagaacagttaacaaatgaaaattccttttggtactagatacttagtttttttctctattctaatactaatatgcaaaatacttttatttctggtttataggttatccattttgttttgcaaaatactcttgtataaatttgttgcaaatacaaagagttcctggtccagaaatattagtatgcattcagtaatgtatcctgccacaataaaagtaaattggcttcataagagcctatctcatttctaaatttcactgtgtatttgataaataaaaacaaagatttgtcaggtgctttgttaatgttaagatgacctgactggtatttaaattgatcaaatctagatgtgagcttattttaaaatctaaccatcataaaatcttttgcaattttctctctttgttgtatgaatagggaatagcacgtaagaaactaatgttacagctatggcttttattgtagaacaagctcttctggatcagaattctcaaactcctcctccaagccctttctcagtgcaagcttttaataaaggggcaagttgcagtgcccaaggatttgattatggactcggaaatagcaaaggtaggtatttaaaagtaagtcttttacttacctaacttcaatattttttattaccttctcttttatacactttttatttgtgttcacccagaaatctttattaaattaaaatttgttcatggaaagctaataaaaaatgtttacaaaggtgttcttttcatagtattgttttttgctgttgttttttgtttttaaccagttttggcttaagtttatgagagtgcttgcttattttttttctatttaaaaaacactcattttgatttcatatcctaaagattctatttcactaaatctagtaaaatttgatttttatcatccacttattaggatggtaatgttaatgagaaacagagaacttgctcaaatgatccatttaaaatcaaactattacttctaaataatatttttaagtaatattacacatgagctttataaaatttatgtggttttgaatttctaacccagtaatcttagttaatttagaattatgtgaaatgatctgtttggttttaatatctataataaaaccagaaagttgtaaaaagaagaaaaccctaagtcatatgaatataatatatatacagctgttttggctgcattaatggaagagaccagcatcatggactgtccaaacaatagttaattttgaaacaatttatgctggaggttcatcagaaattgcatttttatgtgaattcacacactcttgttctttcctatgtacctgatctagactccttcaaaatcagccctattgtccacaggtaacatacagctgggtcatgagtaaaagggagcagatagcttacgtggaaataattttactttaattttatttaaattaattcacaggacttgattccaagggacaaatacagtgtcattatacctgacttgaagaacaagtgaaaaaataaaacagtgaacaaaatgtataaggaatattctgtttaaaataaaactacttctattgaatagcttcattactttccacaaagtatttttatgaaggaaacctagccacaagatttttttttactcccttggactagcctatctcataggattgggtatcatctgctcgcttacttcgggtacctgatttccattaacttaagctagatctttgtacttacatagtgtaaatgcttggtggtgttttctttatgtccctttctctttacctttgtcaactcttaaaatctcagcatttgcttaccgctactatatctctatatagtaatgttttaaagccttgtttaacacaaattgtatgctttcagtctttaaactgtctgtcattgtactaatagctatcacagtaaatcagtcattataatttattaaaatatcattaaaataaatataaatgtttgagacacaaacattgccgagaaaatttgttattatgtacaaataataaagtattactttttaaacatgagattttgtgaatatcgttcaaaaatagcatgcaccatgccaggcatagtggctcatgcctatagtcccagctacctgggaagctgaggcaacaggatcacttgaggccaggagtttaaggatgtagtctgctatagtcgtgcctgtgaatagccacacgctccatcctgggcaacagagcaggatcctgtttccttaaaaaaatattaataataagcctactcttgttaacaagcttttaaaatatttagaaggagcaaagaaataataagaaacttaaacttgatgaaatatatctaactttattatctgtaacagtagttcccattttaactcttaatactgtccaagatcgaagagctgttttttgtaagtttttttttcttaaaacagtattttagaaaattcttcatgatctctaaattttttcctataatctacagcatttatttattatttcaaaaaaagatatatggctcgtgtacacagttctttccacataaatctatcccgtgtaggtacttcagttgaaattttcacatatgaaactttattatatttcagaaatatgactagaaagatactattttccagtttctgcatcacctagtctggatgtagttccctctccccacctccagcatcatttaaactatttaatgtatactcacaaataaggagttattatccatcacataatttcattatcttaaaaaaaatttttgtaaaaccttttggtacaaggaaaaatgttagaatcgatattttagatactattcaaattaggtagctcataatcctttttgtcatataggacttacagatttttaaattattcatctaagcaaaatatttcatgcttttaaagtagaatttctttggcagtaacaacttaaaaaagaaagtattaatttataactataatgtaaacacattgctatgttaataacatactttgccccattctgctttttgaagaaaaactgttaattgcttattttctgcatgaaatatactgccgatgaggcagggtggtattagtcattgttgggaactatggaaaaggcatcatttacttaaatcttcatttatatatgttcaatattgaagcaatttatcactgattcttcatagagtcttcgttctcccatacataatgagagatgttatatatgaatttaaatggatttgtttttataggtgggcttctatataagatttggtggggcgcttccattaaaaaaaaaagcttgcaaaattcggtccctaatctgaggatgattaacagtcaccaattttcactcatttctgaattggcatggttttatttagtgttcaactctatctgaatgtcataaagcaccatttactttgccattttggatgttaatgaataacgagttgtagttttgactgttttacccactgcaggtgaccaactgagtgccatattgaattccattcagtcacgacccaatctcccagctccttccatctttgatcaagctgcaaaacctccctcttccctagtacacagcccatttgtgttcggacagcccctttccttccagcagcctcagcttcagagtaagtctgtcagccttacctgcctcatcaagcatttctgaagctttgttctgagttgcttatcagttactaattagttatcaactcacctgactggttttttttaattaggatgttatcccctcccacatttttgaaatatctaagctattctgagctaagacattctgcagctaagacatttaaagttgatttaaagttcatatgcatgactatggtaagcatcacatacagggacaactttcagtggatttttcttacttcaagatgtcccaaaaggataaagccatttcactcttatcagtgggtagaagagagactgtacttgtttggacaggtttgtttgttttttctaagtgaatttggtatctatgaaatgtatgacaatttagttttcccggttttctcaaattgttgtttcctgtctttatttttatttttttgttgttgttcctaatatgttaaattgatcatttgattccagttctgtcttttaggattgctctagtccttcattcatgatagtaattcacattggtttggaaaagatacccaaaaaaattatacttaactgagtgttcttttttcttaataacctcctgaaccttccacctaaacttttcaaagctctgtccagtatgacaacagtgtgttcttcttggcattcagttctcatgttggctatacaatatataaacacattaattgaatgagctagtttatgaaagtttcagcttgtataccatttttcggattcaaatgcacaacctctttgctttggtaaagtacattcacacttaggtctaagttatttgtttacaaaaatactttaataactgttattagggtcatgtaaaagaatgctagctggtttagtttttattaaaaactaggttagtatgtagctagcaaatgatattattcattactttatccaccttaggtaaagggtacacaagagtataaacttttctgggtttccactatccattaggaaagtttaattgatgaaattaattagtttggaaatataagtactatcaatttctagcttatcatgttcgttcatgtataatattaatttgaatgtctattaagagaagaatttcaaagaaacaaaatagtgtcacatgtgcgaaatagttttttccttctatgataaatattattttagaagaatgaaacagaagtgaggagttagtttattaattttgtgttgcatcagactttttacatttagaagtactacttaggtttcaagaagttttaaaggtaaggacaaagggaaaaacaaaggaaaaatatcaggatcagtcaggtagaaaatttatactagaaaatttaaattcattcatattcaaattcagttttaaatgaaaaattgcctgtgtcatagcagtagcagacaaaagaactaaggagaaaggagaacctttttgttctagctaaagaatcattgcaagtctttacaaaaacaagcataattaccattttaattttaccaaatttaattaataaaatatttcaaaacaataaacagtaataaaagttgatttgtctcttccagggaataacagtttctaggtgacattgtgtcaatataattttatgcatatgtgttatcagtaccaccactttatttaccactatcttgaagcaaatttgaaatcatggcttagctatgtttcaattagcaattttctgaatcattacttgcctatagtccagcaatggaaattacttaggtcttgttttaaacttcttattcgcaacaggacgtctctaaattttgacttccaaattccagaaaatggaagtcttaatctttagcatcacaaaaaaaagtatggaatctgaaaatagttttaattttttaaaataattcagaattgtattttttataccgtgttgggaaaggtaaatgtggcaaagtgccaacatttaagactccccaagcaaggtattcttccctgaacctcactaccatgtatcccactagtatatcacctatgctttagaagatggcttattcttagtgaacttccctatgctatttagtaaaatgagatttatttttgttttatacctttgtgatactaaataaatcattatgtagaagaccatatttactcaactttgtttatactgatagtcgccactatgtagtaaacactgtacttaagcactttgatacagcctcatttgagatactgtctcttatgtctgttttacagagaaggaaacataggcttagagaggataaatgatttgccccagaaacatacatagtaaaatggtagagccagaacttgaggtcaattctgttgactacaaaggctattttaaatattcagccttacctcggtaaataatgcaaaatctactttaatatatatatatttttaatttatatttaaactaggtctggcatctagagtattagctttgttcagattgattatttttctttttatagtttgactttttttaatgtaacagtagtttgaatggaaaaaaagatttcctgaatgtcataactcaagatcctggttaactagaaaaattctacaaagacacacatcaaccggtgcatggtttaacttcatctagcatttctatgagcattattatctgtaattctttggttttttttttccgaatcatagtttttacacctatctttggaaatttgaaaataatctggcacattaagttaatttttgataccttttctttagaatcaggcaacattttattccataaaatagaatatttggtaatttttaaattagcaaaattatttgattttatatacgtagaaattatttcaagtgttattctagtgaagtagaatgttatagaagtaaaagttaacaaatttcccttgtcttatgtgtgtgcacacagagacatgaaaaagaaaagtatgtatatagaattttctttactgttctacaatttactttttaatttaaaaactaattatatagtttttagaattggtaagcaatttggcaaaaaaattttagtgaaccaaagtggagaatgatatctgtatctgctttctttaagaatattacattccttattttcattatcaccatttctcttgaaagcttcattttatgtcctgtaccttctgacagtgtcttacacatcacaggcattcaaggagtgtttattgactgtaggtatttaagattaccatttcatcaatttaagatcttgtgttttctattatgatattccattgtttactcagagaacatcttcaaagaagagaatttatttctttctcttagtaattaaagccaactatactgtgacaatatgatgtatgataattgtgggtttatactgattgaattctaagcaagattatccaactgaaaaactatattgcaaatggcttcatttaacatagcattttatcactttctacctactgttccagaggctgaatagttcatttttcaacttttatttatttgtttgtggtagaaaaatggcaaataaaactgaagacaatttgcttttctcccagaaactttttaatactttttttattttataatgttttatttttaattttgatgggtacatagtagacatcttccagcaatttcaaacatgaaatatatgatatatgtcaaaagacagtactttcaagcacagatatcatagctctaggttaccttgcaacaagagtttttctattatactcataagtcacattcttatgtgggacaaaaaaaattaaatgtgaccccaaaattcatttggatatctgcatgactatttatttacatttgagaatatgaacacactaaagtactatgtttacttagcttgtagaaacatagtcatggaaagactttcagctcagatttcccttccagctacttactagctctctaagttatctgacaggtctgaatttttgttacctcattataaaatgggattcccaataaatgttatcttttgatgttattattgccttttgatgcaaatacttaatacctctcatgctttgaggaaaatatagaaaaagtccttctctaaaagagagcaatctattcttggtgtggaatgagttgcaggaaggaatttagaaagggacaaatgagctttcctagttccttttgggtctatttttgcaatgtaattcagttacctattttaagattcttaatgtatttagactgcattcagatgttagtgtgaatgttagcataaaagccaggtttgtttgtaaggcttagactgagtcatttctaccccggattataatctcagcactgtatttgtcagtttgttgtttgcctgctttgaaggtatgtggatgaagcacttgatttaggaatgaggacctgtgggatttaatccctgttttagaagattagagtcagcattggttcacatatcaaagtttaataaaacttttccacttgactttataacatctgtcacaacaaccaacatatttattttagttttggggctaacatctccagcatccctacaaatacaaatcattttaggcatgggcttgaatttatcttcactgagctgagccatcttctcagtgacctaaaagctttgacccaggtattttgttttgctgcttcatcagtgatttattcatccaccctttacttcatcctaggaattcaaaccacaaagaatgctgatgttgcttacaagacattttgtcccagtgcatttgtttgctttctcctggcttgatttgaccttttatctctccctcttcccccacctctttgtttgctactgtgtttatttcacccttcattaccatctttctttagatgacttattcattcttccatttattcacctgcttttgttaagtatctactctgtgctaggttatggtgatgagcaaggttaaaaaaaaaacaaaaaaccatgatcactgatcttgaaggcttctagaatattttaagagatacaacctagttaaagaaaaaataatggcatgttttaagtgcaacaagagcagtagtcacaggatgcaatggaaacataggaaagcttctggctcagctaggagaggttagagaaacattcaaaaaagaggcaggtcctactcagagtgttgaaggacttgtgagaatttatcaggcaaggaggagtggagatgaggaaggaggttaatggagcattccaactataaggataacatgagttacattacagagatccaatgaatcacaaagtattttattgagtccacctacgtgtcaggcagtgtactaagcactgttcatgcagagggccagtttggagtaatgaaactccaaagtttgattagaataggcttaggagaaaacggggaaggagaaattggagacaatgagtatagagaagtgtgtcaagatttgatctaaagagaagatactaagagagaagatacaagatctggagaggtttttgttctattttgtttgttaatggaaaaaaaagtatatcaaggttgttttctaatgagagtaattcagttgaaagaaatattaataatgcaggagggtgaggagaattactggcacaatattcttttatagaagaggggatgcagaccaggcgtagtggctcacacctatattcccagcatttggcaaggccaaggtagaaggatggcttgaggccaggagtttgagaccagccaagacaacatagcaagtccttgtttctataaaaattaagaaaaatttttttaaagatcagctgggcatagtgatgcacacctgtaatcctagctacttgagaggctgaagcaggcaaatcacttaagcccaggagttcgaggctgcagtcagccataattgcatgccaccccactctagcctgagcaacagagtgagaccttgtctctaatatgtatatatacagatataatatataatatataaaatacattctatataaaatatatattatatattatataaataaaatattatatataaaatatatattatataaataaaacattatatataaaatatatattttatatatatataatataaagaagaggggatacagtccagtgcaaagtggaaaggttggctaagtgggatcataaatagttatccatcataacaagcaagaaagaagtatgtatattattgggagctcttttttctaagtaaaataggaagcacttatcagctcggagttaccatggagagactgcattgatagtttgaggaaagagaagatatgagattttaaaagctaacatatgtcaaatatttatcacatgccaggcaccattcatgggaatcaacttttatccctgtccaagaacatggttcacttgagaaacttcatgcatgtgcagctttaagtgacctagtgtcagtgtcttagactagagaggttggggctggatcatgaagggtcttgtaagccatacttagtttagactttatgtgcagtgaataaatggagggagcacatggaggaaattgttctgcagaattttaaataaaaagagtgatatgatcagttttacatttgggacagataactgaaaatattatgaaaaaggtcctggagaaagggagaatgttggaggcaaagaatccagagaaaagctgagtagggtttaagctaaggttaattgcagggagaatgtagaagaaatatttaggcagtaaaatgcttaggaccagtctggacaacaaagcgagaccctgtctctacttaaaagaaaaaaaaaaaaaattagccaagcattgagggctgcacctatattcctggctacttgggaggctgaagcaggaggattccttgagcccaggagtttgaggctgcagtgagcagcctgggcgaaaagagcaagaccctgcctcttaaaaaaaaaaaaaaaaaggaagtgacatgctgattaaagttaggagccagtctcaaatgacatctacttttatgctctgttttgaaaggtaatatcttatctagtaccaagaataaaacacaaaaccaccttttaaggaaaaatgagttcggttggatgacttagctccagttggctataatataacttgaaattgagaaatatatttagcattctcataattaaagatggcattgttgactgacaagttgaaaatagaaaattattcatatattacattttattaaaaaatattattaaagcattctcatgattttgcactcaatttaggaaggattaggtctgctatatctcccactttgttattctctcaaacttctgtacctgtagtatatctcattacttaagctgaacaaaacgttcattttttgtaatcagacttctcatccttatcatcagattgtctttcctcctttggtttatttaagaagtgtgcatcagcagcagccgttatgtcttatgatggcataatttcaagtgttctaggggcctaagaagttatccagctctcattttgagatgagagaattcttgctgacttgcccaaagtcatagctggcaaatctaggacttgaacatgagagtctgtattgggggaacccgcccccaatatttcaacgtaggttctttctattttccctaagcattggccagtctgagaaaaaaagagaaagagtacaaagaggaattttacagctgggcctctggcggtgacatcacatattggtaggaccgtgatgtcctctgagccacaaaaccagcaggtttttattaagcaggtttttatcaaaaagggaggggatgcaagaacagggagtaggtcacaaagatcacatgccttaaagggcaaaaagatcacaaggcaaagggcaaagcaaagatcacaaggcagagggcaaaattaaaattactgatgagggtctatgttcagctgtgcacgtattgtcttgataaacatcttaacagaaaacagggttcaagagcagagaaccgatctgacctcaatttcaccagggtggggttttttccccgccttctgagcctgagggtactgcaggagaccagggcgtatttcagtccttatctcaaccgaataagacagacactcccagagcagccgtttatagacctccccccaggaatgcaattcttttcttagggtcttaatatttaatattccttgctaggagaagaatttagtgatatctctcctacttgcacatctgtttataggctctctgtaagaagaaaaatgtggctctattctgcctaaccccgcaggcagtcagaccttatggttgtcttcccttgttccttgaaaatcgctgttgttctgttcattttcaaggtgcactgatttcatgttgttcaaacacacatgttttacaatcaatttgtacaataatggtcctgaggtgacgtacattctcagcttacaaagataacaggattaagagattaaagtaaagacaggcataagaaactgtaagagtattatttgggaactgataaatgtccatgaaatcttcacaatttatgttcagagattgcagtaaaaacaggtgtaagaaattataaaagtattaatttggggaactgataaatgtccatgaaatcttcacaatttatgttcctctgccacggttccagccagtccctccattcaggatctctgactttccgcaacaagtctgctaactcattccagtggttttttccaactgcatctcagttatcttacatagactgcaagaagtgagaaagacaagaggttatctagtccagccttgctattttatagtttaaatccctcaaccacatccctgatgaacttttgccaggccggtaattaacaatatcacaaggctgttctgattgtctgtatttctcagtgtttgttagagcagggatgtccaacccccaggccacagaccaatactggtccaaggcctgttaggaacccagctgtacagcaggaggagagcattactgtctcagctctacttcctgtcagatcagctgcggcattagattctcataagagtgcaaaccctagtatgaactgtgcatgcaagggatctaggttgagagctcctcatgagaatctaatgcctgatgatctgaggtggagcagttccatcttgagactatttccccaaccccccatcatatggaaaaattgtctcccaagaaaccagtccctggtcccagaaagttaggggaccattgtgttagagaactaaggaaaccgtcctctacctgccacataagaataaaggaaacaatggaacagtttccctactttccctaattaacacgtattcccattttgaggcagtgagttgctggtacctgctttctccttctttctccaaatagtactttaaaagtatcttatcctggctgggcacagtggctcatgcctgtaatcccagcactttgggaggatgaggcgggtgaatcacgaggtcgaccatcctggctaacacagtgaaaccccgtctctactaaaaatacaaaaaattagctgggtgtggtggcaggcacctgtagtcccagctactcaggaagctgaggcgggagaatggtgtaaacctgggaggcagagcttgcagtgagctgagatcgtgcctctgcactttagcctgggcaacagagcaagactccatctcaaaaaaaaaaaaaaaataaagtatcttatcctaaaagcacttctgtttttggtttaggttttagctgtctttgtgctgcgtaagcgttgtctctttctcaggatgtcacttctggaggcaggaaagggttatggttaatgctaatcactttatcaaaatgtctgatttctctgatgtataattaatatttttcccttgcaactaataagcaacttgtgggagtaagatttttacttttaaaagcatatccaaccttcttgtgtctcaagttaatgctcgggaagcaatacctgtctcttatattttgaagttattctttctaaggcctaagatatatacccatcaaattatgctcagaatttttgtctcagccatcacaaaccctgaaatgatacgcttttcctaagtgttattttccttaaccatatttactagagatttggaatatattcaataatatgctatgagtatgttttcttcaagttcaattatgtgtagatgtcatttagagaaacagtatattttggggggaatccactttatgtgtcccatcttctttccacattcagatcatgtggattaatgtcatagtctagtaaaaagcttgtttctattttatccttaagtttaaaatgttgttctttcattctattagcatgattcttttagtatactaacatttatgtaaataagtcttacataaatacagttatataaacaaatctttagtctaattcttttagtataccaacatttatgtaaataaatcttagtttctgttccacgtcctaagctgctttaccattcacataattccatattcctcctaagtttactacaaagaagaatattagatgtattgattatgcagtgatactgcatctaaagctgtcagtcaagaatggctgccatagctaagagtatatacaaatcatcactgttactttattttatttttttaattttatgtatttatttatttttttgagacaatttcactctgtcgcccacactggagtacagtggtgcaatctaggctcactgcaacctccctctcccaggctctagtgatcctcccacctctgcctcctgagtagtgaggactggaggcgagtgtccccacacctgcctaatttttgtatttttttgtggaaatggggtttcaccatgttgcctaagctggtctcaaactcctgggctcaagcaatttgattgccttggcctaccaaagtgctaggataacggacttgagccactgtgcctagtcctgtcactttctgaaaaatcctatgaagcattaatagaagtaaaatcacacttaattatgcctgtttttaaatctgcatattgtttgattaatcacatcttttggccaagttgaaggcataatttatatattgtctataagaatcaataaactagtttctaaatatgttttaaataaataatttaaaaattaatttctcaaatatcctcaactataaagttagacctatttactgtttcttgtaggagcctattgctgtcaaaaatatctgaagttctattcagagaaaaatagaaatgcacaatgactgggacttaagctgaggaaagtcagcaacactgttcccatcattctaccataaatgtggcaaaatcttattgtttgtgacagatctatagaattagattttcttatatctaaagaaatatatataaatacataaacgtaggtatgtatagcatgaacagaatacagtgctatttgaccattgtcacaagacttatttataaaacctcctcaagctcttgccaatatcttagttaagattgcatctaaggtgggaggaggatgaagattgaagaactgtcaggtaatgcttgattacctgggtgacaaaattatgtttacaccaaacccccatgacacacagtttacccatgtaacaaacctacacatgtcccccttgaacctaaaataaaagttggaaagaaaaaaattggaaagaaaaaaaaggaacttaaagagaagaaaaagattgtatctagtagttttactaagactataaaatttctgacggctccactacctcctcaagattgagaactaaacaacatgagaagcacaattacatgccaaagattctttttgctgtcttcatttgtagcatcccagtggaatgtctttgaaaaaaattttttttatctgtattatctcacctgctctgagtattggtgacagtgatatatgcattgtgattagacgcctagcctcctgaatataatgtatttccagtatattgtccttctgaatagcctgtttacaattctttgaacattgcctgccaataatttagcaaattgttattgaatgtcacagcttgattatagaacatagtaagaaatcaacaaattgaacaccaaactgaatttaatgctaaaaattatccttttaaaaaataatgtattactaatttttactcatgaaatttaacatttccaatcattgcttagttgttgactcaacactcaaaaataatatatgctacttttaaaatcccaaaacctccagatttgtgtactgccaccaggtgttgacaggtgccttgattcttctagtttgtggttccagagataaggtcagggtcctgatacagcaaaaccaccatttgtctttgttaaactgaaatgagaatgcgttccttattggaaagaaaagttacacggttttattattatcatcagtacttagagaagacttaaaaagtacaacttaaaatgaactcaaagtttctgtgactaggaaatgatatgactcagggaatttaatacttgggatccaaaacaaagaaaacaaaatgacccataaagaaccatgttgtttatgagggcaaactatagtaagcaaacaatattaacttcaaatgagcactggagaaaaatcctgattctttaaaattgtttttaatattattctcttaaacttagtgatacccaaaaaagaatcaaaattttgtggagaggaatataataactggttgtcttatctgtattaacaggattttgtaaaagcattattatttctgtcaattgatttttaaaacatctatcaactttttttcttttgtgtatagatatcaatgtatctcatagattgcttcccttttttaaattaaaaagtataaatctttataaaaattaactataactttatgaaagtatgtttaaacttgagcagattttgtaaaaatttaatgtgtagaacatgtgttgttttcagcaacagcttaatttctttagtcacttaaccttttttagacttttgtttttaaaaatatttctttttttcactctgaatcatacctaggaatcaagtctctctagcctttgtaatatattgaaaagacttgaaggccattttgccttcaaacttttttccaggagtttgtccttaaacctctaacatcttataagtttcctcatagtttttcacatttctgttttatgtgtgcttccaattaagatggtattcttctgatttcctattcctgtgtcttcctgctatgtggagtcttcacagatttttagcagccaggccaggagattgtattccttcctatattggcaggttggattccccctgaattaactcttccattcagaggctgccaccgttctagttcgtgtcttgaagaattgtatcaccctcctaactgatattttaacaatagctgcaaactccttattatatgacacttaaatagcataagagctccttttgcagcacctgacatcttttgaggtttatagatttgttcaaaatgaaggatgtacaaaaatattaaaaatttaactttttaaaacttagattaagatggctttatgccatggtatgttgctatctactcctcccattgtctcttgagcccactgcattccagctttagtccctatcctcggaaactgctgtagtcagtctgtgacttttgtgttgccaaattcagttgtcggttctaaggtgtcagttttacttaatctgtcagcagtatttgacagagttggtctttcttcctagaaacacattcatttgcctgccggatatttcctgccttactggctgctgaattctagtctcctttctacatcctcatctatttggtcatctagaaatgtcctagggctttggcctcttctaggcgtcactcccaaagtggtattctgcagtcatggagaatgacacatctgtaggcatatgactcccaagtttctcagcacctgatgtgatgtatacttatttgttcattccctctctcctttctcctaaatataaactctgtgagaatagggactttgttcctgttcaccactgtgtctgcaatgcatagaacagtatctaacacgtaatatatactcatactttacattgaatgatttcttaatttatgggtcattcatcattaatcttgatcataataatgaatcagccgaaaacagcactttttttaagaaaagtgtgtgttatgtctagtttatttcaataatactaagacaactatctccttactacctgaatttatttcagcacaaccgattaattttaattgctatgagttaaaaactttaaatacagtgagtttttttaaattcagatagagatgttttttagcttactgcttttaaaagactgcaatttaaaggttctgttaattcttcaaaaaaagataaattaagtgcttcattttcctgcatagtaaatcattgacgttcctaacattttttcaataacttataaattttctttgctttgtttgtgttgtgacacgttgtgtaaatgagatttgatatcagaagtaggatctacaaaacaaacactaaatactggggttagtactaacgttttcactgaagatggacccagaaaagtagccaaacacagtgtcaccccgagtccattctgagatacatactggaaaattatagtaatcggttcattcaggaaaccaagtgacaataggttaagccatgagtaatataagtaatataagtaacatttctttatcttccactattaaaaaaaaacttgtaaatctttatttaaatgaacaccaaattaaagaaaactgagccaaagaaatgtaagtctaaaatgaaatgctcaattttgatttatcagagatataagccttactaagttttaagttaacaaatttggaccttaacacatattaatctaaattaattaattcgacctttgttttcagttttagagaaatgtaaacttgttcaaaaatatagcagaatataaactatttaaaataacagtttatacttgcataccatttgaaatgctttgtttttgcttgtgttctctgctaaattttttttccattgaatctgtgctgcttctttgccaatgttttccactgtagaatctccatctcgcaaccttgcttctcgtgagcgcatttacaaaaattatggtgtagctgggcctgcctctgctctctcatctctgtctcacaaactgaagggtgggtatacatgcattcatggatgggccattcttaatgaacataaggagtgtcattaaagatgtttccgttcgtctcagtcatgttcatgccattccatttttttaattaaagtttatcattttatttacatttatatatgtttgagtaatttatgatgctcctattcagtattataatatagattatcccagtagctatgcatgtttattacatctataggttaaacattactcttacttaattttaaaactttatgagatcattaaaattaaaataggtataaggaaagaattacctaattgggggaatttttgaaatcatttttatcagaaaaagatgatatatgtgcaaggcataaagcacattttttagtcttaaacttattgttatataatataaaaatcattttactattttagtcttttatgagaaacactgataatgaacttaaagtttttaagtttaaattagttattaggactcatgtgtctatttatatttatatgtaaacctacctgtcctatctggcataaatttttctaatattgttttcctagtgactaagttttaatattttaaacttagaaatgtttaaactattgacaattgacaaaatttcagacaatttaaaaatatgtttttggttttttgaaacactacagatgcctacttaaatattttgtcttaaagtattatttaattaaatttaaatcttgtaaatcttttaaaatcttgtatttaaataaaattttatcaaaaagaacttttacagcaatgcctaacatattgtataagtttttcaactaattactgattttctttttttcttagaaatagataaaacagctatatgtttctcattatacaattgacagtataaacatataaataggaaatataaaaatatactaaatagtattgtacaacctgtgctatatagtatatagtattgtgtgtaatagcatatagtatcatgtaacctgatatatgctatatatcatgccatgtgatatatgcatatagtattatgatatagtatgatcctgataatttaagatttgggcatgaaaaaatgtatgggtcatttagtgaatgaatgcttaccatttaacatgtttagtaacatttaacttgcttgccctaagtttctatcctgtcattattatttcttcaggtttcctttaaagttagaaaaatgtttgcatattatcatttaaatgtaaaagtacagtcattaaataaattaggagatgaatgtgaaagtattacgtatagtctgaagatttttagcaaactgtttatatttgttggtattttgagcagccacaacatacaatttttagtaatatttaataacatgctgcaaaacatttgaaacacaaatgaagcagataaacataaattagcacaggggcttaccccacttaacaagcatagttttttttaatatataaaatacgttttggaaaaataaatttttcaaaaagatttagttatcatttgagaaatacatctgaaaaagaaatatttctgccattatttgtatgcctaaacttcaacaggtatttagctgtcaccacttacagatcttaagaaaatattcaccactattagagatataataagatatacaaatgcctattctttctaacactatgtaataaacttttttgctcacatatgacaagtggtgttcctggtatttggcagattccatttttcaggcaaccacagaggttgttttcatgatgagtaaataacctaccattgtaagttcatatgcattcctagaatagaggtagcatcagcaggtattcaccattcctttgcatgcagctgttgtgcaggtcaaccactaagacacatctgacacagggagtaggtaatctttgtcacgaacccttcctatttgttagaaatcctacacaagttgtccttttttataaattagagagaaaacctctttcagtttacagatggattttttggagaaagggataccaaatataaaagacttactaattgtttgtctagattcccattatgtgtggttcagtttctgaaataaatgtgatgataaaggtgataaacatgtctccagctgtcagattaataacaaaaaacaagtattccaaacatcacatactcaaaattaatggccatgactactgatttaatctaccaaatataatttgtttaccaaatttgccatatgctgttactaggtcagtaagattagatgggtagcaattttttgaactacttttgaacagtttaagggtacttattcatagatcacatctgtttatctgtgctctcccaagacagcattgatagttagaattctaaattgggatttttttaacccttttttcaaaacttagccccatctgttaatactgtatttataaaacagagcacataatatgataaaggaggttaatgattgaataatgaagctttgcaagaaactaaaaagcattatgttttagttacttttattcatccaaagaacaggaaaaattatcttaaatattggcatgttgcaattaaacatttagttcatcagtgtgccctgtataaagtacttgccatactctattatttcaccatgtaattactcagtttgcaagcatctaacccttgcacatcaataactaatgtcacctctgtcctgtggccttgggagccttaattgtaaaaagtagacttctggagaagctgaatacttgggagtagcagaagaaaaaaatcaagaaaattggaatatattactcattcaacacatatttattgaatacctactgtattcctggattcattcttggtattacatcagtgaagagtaacagacaaaaatacctgcccttgacaaagactggggcagattctccagcactactgtcctgagccatagagaaatagagcgtgactatacacacttaacacatgcacggaagcataaaaaacattttcctacatcatctgaatttgttcacatccttttttcttttaaaaaataataacatgcagttatagccagttaacaaatctaccttaatttcctaggaactgtctttgaaaaaatataaatacttccagataggtttatggattttcagttttttataccacttgaaacccttcagtcagattgcttttctcctaacaactaactggtatcacttgcactaagagtagaagtttttattttaaggaggaaaggtgatgttgaagtttgtaccatttacttgggtcacagacagcctggctctaatgcctcaaagaaaagcataaaatagaatgaaggagagacactgaatgaaggcagggtgctaaaaacaattaatgtccatgcagaaacatctaatgctttgccttagaaaagggaggtgattcttcttttcatcctgtttttctgtcattctcctgactccttgttttgagggtccacagttcagacttttaccactcattctgaggatccaccgtttctgtggtttccaagggaacacaggctctgagttctaagcagctaagcttcctgaacttttggagtgtaacctgtttataaataattaaataacagcttcagggcattcatgtgctcattgtaaactagtctaatctgatcataaaacagagtgcctcttaagcaactctttgttagcctaattgtatgcatacacttaaaaattataattgtgtctatgcataaattcagttctctaattcaacaaaacccataccgttgccctaaggatgcgaggtatcactctgctggaaatttagctctactctacatttaaaatacaagttttcacattaaataatttcttattccccaaaagattaatttctgtctctttctagtccctcactgaatttaaattctaactagttattgcactagggcagaggttgagatatcactgatagttcacactggatttgttcaacctgttgttttgcgtccatgaaaatagtttttggtggtaatgttgtgtaatgatgtgtagaaattttattagaaatatttcccccttctttcctttattcttcaagtattttagtatgtatgacaaacaatataaattttgtagctatgtttttttatttcagtgtttagtggctaaattatcagtaagttataattttattaataagtttcactgtaaaatggaccatacaggtgatcgaggaaacatctcaacatcttctaaaccagcctctacatcaggaaaatcagagctgtcctctaaacacagcagatcgcttaaacctgatggacgtatgagccggactactgctgatcagaagaagccaaggggcacagaaagtttatctgctagtgaatccctcatcttaaaatctgatgctgcaaagttgaggtcagattcccacagtaggtcattatcccccaaccataacaccttgcagacattgaaatctgatgggaggatgccttctagctccagagctgaatccccaggaccaggttctcggttgtcatctcctaagccaaagactctcccagccaataggtctagcccatcgggtgctagttctccacgctcctcctcaccacatgataaaaatctacctcaaaaaagtactgctcctgttaagacaaagcttgatcctcctcgggaacgttctaaatcagactcttacacacttgatccagataccctccgcaagaagaaaatgcccctcacagaacctttgagaggacggtcaacgtcaccaaaaccaaaatcagtaccaaaggattctacagattcccctggatctgaaaatagagctccctctccccatgtggtacaggaaaacctccacagtgaggtggtcgaagtctgcacctcaagtactttaaaaacaaatagtctaacagacagcacctgcgatgacagcagtgaatttaagagtgtggatgaaggttcaaataaagttcattttagcattggaaaagcaccactgaaagatgaacaggaaatgagagcatctcccaaaataagtcgaaaatgtgctaatagacacaccaggcccaaaaaagaaaaatcgagttttcttttcaaaggagatggatccaagcctttagagccagccaagcaagccatgtctccttctgtggccgaatgtgccagagctgtgtttgcttccttcctctggcatgaaggcatagtacatgatgcaatggcttgttcttctttcctaaagtttcatcctgaactttccaaagaacatgctcctataaggagtagtttaaatagccaacaacctacagaggaaaaagaaaccaagttaaaaaatagacattcattagaaatatcatctgcactgaatatgtttaatattgcaccccatggaccagatatatctaagatgggtagcatcaacaaaaacaaggtattgtctatgcttaaggaaccacctctgcatgaaaaatgtgaggatgggaaaaccgagaccacttttgaaatgtccatgcataacacaatgaagtctaagtctcctcttcccttaactttacaacatttagtggctttttgggaagacatctctttggctactatcaaagctgcttcccagaatatgatttttccaagtcctggttcctgtgcagttcttaaaaagaaagagtgtgagaaagagaataagaagtccaaaaaggaaaaaaagaaaaaagaaaaggcagaagttaggcccaggggtaatttgtttggagagatggcccagctggcagtaggaggaccagagaaagataccatctgtgaactgtgtggggagtcacatccatacccggtgacctatcacatgagacaagctcacccaggtaaatgatactgttgaatgtacgtataagtgcttttttctttaatgaaccagatcactttatttgagctgttctatgaattttgtcaaaggatagggaatttatagtatcttagaatgtcatttttaaagtacattatcagtacagggttttgtgaaaaacagataaattctaatattatgatggccaggtcatttagtgctatttcactttcatgtagcatgttttaaaaaatattatgtgttacccaaagggtgtgccattaatgtgtctgtctcacacagcagaaacagtggcatgcccatacacacagggcatctcctttgattttactattttcctgttcagaattgcaaattaaattagattagattgatttctataggctttttcctacggaagtgatattgttttatcagaagactgcattttcagtaaatgtaatgatttctgtaacattgactaatattccaaattcacctaatacaaatttggttaatttttttctttgtctacatattatacctcaaatgtcttatgaatatgtgatttctgaaaatatatcattgttatagaacttttatacttgtattgtctaattttattgacataattaagtatatctaaatatacctatattctatctctcttttaatatgcctgattagaaactaagtatctctcatatttggggcatcatttaattgattgctttttgatcaagtaagctatttttctgatatagttaatagcctgaaaaataagatatctacttcagtattttgctttctttgagaaggtaaatctgtcattaatgtatgagtaataaggatcaaactattaaggacacttggagtgttaaatatttatttttggtattgtttcaaggttgtggccgatatgctggtggacaaggttacaatagcattgggcatttttgtggaggatgggctggtaactgtggtgatggtggcataggaggaagcacttggtatctggtatgtgatcgctgtagagaaaaatacctccgcgaaaaacaggctgctgcaagggagaaggtattttattccatttggagctatacttttaatgatatggatacactgtataaaaataccttgttataatcttactacatcagataattccacatagttgatattaaaagatcagcttttataatggaaatcatatacaaatttaatatttgtactaagtcaaatgcttttaattttaaaaattaactatatattggaataacactagataacctctgctacatatgccttattgacaaatatttgttgtgtaactctttattattaattgtctgttattctctctatggagagctttccttttcaccttctttctcttttcctctttttttggctcctttgttttcctattccatatctactctctcttttctttattcactctagtggcacctaactaaaaaatttcactgaatacacgcactgaaatgatttactttttaaaaaaatgaattacatttagatttaactagttaggtgttaaatttttctgaagaaggaaaggaagtatagtttgtgtttgatacaaaattgtcttggtcagcccaggatgaatatgaaatgaatgttgttttttataatcttattatcttaaaatagctttacaagtgtttaataatttttattggaaactcaaaatacttagcttaagaaactaaggattttaatgtaagtcagaaaagattagtttgatttttttcttcgccaggtcaaacaatctaggagaaaaccaatgcaagtcaagacccctcgtgccttgcccaccatggaagctcaccaggtgattaaagccaatgcactcttcctgctgtccctgagcagtgcagcagaaccgagcattctgtgttaccatcctgcaaagccattccaatctcagttgcccagtgtaaaagaaggcatttctgaggatcttcctgtgaaaatgccttgtctctacctgcagacattagctaggtaataaaattttgctgttgttttttaatcacctttggattaacagcgattgtttattgatatctagggtagttcggtatttgacctacactttattagctattgtgtactataaacaaacttccaggcatttattttttgagttgccttgactgacataatgactttatggtaataggtatttaaaaaactataaaaatgcttcatcaatttgcattgtattaatacacaacatgtcttagaaaataagactatagctatcttttactctttggtaggctgacttcactctgttagtgcgaaatgctgaagtgtaaagaaagtgtgtttccttagcagtcttttatttcgtaaattattcattctgttagcatttaaatgaataccaactgggcacctaccagccttgtgctaggccctgaatgtatgaatataacaaaatcctggcttctatgtactcatactcggatgtcaagtcagacatgttagtgaaacatttgaaatgccatgtgaggagtgctacaatagaagtatgtatgtaagatgctgttagagcataaaagatggagagtatctgaatcttccccaggaaatcagaaaaggcttcccagagggtctgagcctttagtagagtctcaaaggataaatagaaatctgtcaggcaagcgacaggaagaaagcgactcttggaagaataacccaggtgagtaaagacacaaatgaaatctcattacacatttcaggaatcacgggttgttaaaggactggctactggaggaaatatgtgaaagcccgttcaaagtgattggttcacccaagtaatgtcgggctctgtagcccaggttaagattgcattttatcccatgagcagaggaaacagtggaagaattttaaacagagaaatacatgaactggtttgcaattaaaaaatcactgtggaatgtggaagctaatgtggagactagataaaagtgcattactggagatagaaacctgttagtattctgttttcatatattggacctcagtggtgcagcctgtactttcacacatcatgcactgacactccaaaagagcctttcatataccaaaaccaaatttttttcatcagctgacaaatgaaggtaatctagatattgcccactgacatggcttgccgaccaacagttccattttggaaattgtcctctttgtgtagcagagttgtatggtttttaaaattttgtgcttactattacaataaaatgaataagcaacaaaaatgaaaatatcagactaagtacatttgagactgaggtggaaatcatcagtatgccaaaatacaatagatctttagtgtgaatcacaccactcagtggacttaggttagcaactatttgttcattttgaaaaggaaagaatcaaattgtaaaacgtgaaaaatgctaacacagcataattatagatttttttctttttagctgtctggggaatgggcaccccttccaccctgaaccacagaaaatttcatatatctaatatttttcaaatatgatcttatgtatgttcctacccaaaaaagtaactgcttgtatttcagtggtctgtgcaagaaataataagcatgaaccaaggcagtgatgatagggaatgagaagagagaatggctctgagaggaaacctaatatcatgggcttgaaagatgatgatgatagagggctaagcctactacaacactggaaatattttagcttaagcaactggatggttggtgggcttattcaccaaaacactggaagatggaaaagttctcaaaaggaaagaaactgagttcaattttggatttgaaatgttcgtggtacctggaatgttgatgaataaaattatcagacttttattttcacctacctacatctacgattattttaatgctttgcctttaactatgatttacatgtgctgcttttcctgttatgaaagagattaaaaatggagaggatctgtattgccatcatgctttaagttattgggtttgatttaatgcaggcatcatcatgaaaattttgtgggctatcaagatgacaatctattccaggatgaaatgagatatctacgttcaacatctgtacctgccccgtatatatcagtaactcctgatgcaagtcctaatgtatttgaagagccagagagcaatatgaagtctatgccaccaaggtattttagttctctcctgtctgttgaatagctagtgtttggtggaagaaagactttgaactagaaaagagtcctgtagaagttctttaatacatctgtgagccagttttgctaactgctggagcagtaagaaaaagcatacatgtatgactaaggtaaggtccttgatgggagaggcagactagttggagagaagagatcaactagtgaaggaattaaagattacattatatgacattacttaatgctgtagtaaatcagagtagggaagggttactttgagttttcatgggagcagtggcattgaaaataaatcttaaacttcttggattattgtttacagtgcctcctaacttaggtatggtagtatcagagtaaagagcttctcggaaagctgaatagaaacttgttacaataatgtaatacacaagaaaatagaggccaggcgctgtggctcacacctgtaatcccagcactttgggaggccaaggcgggtggatcacctgaggtcaggagtttgagaccagcctgaccaacatggtgaaaccctgtctttactaaaaatacaaaaaattagccgagcatggtgacacacacctgtaatcccagctactcgggaggctgaggcaggagaattacttgaacctgggaggcagaggttgcagtgagctgagattgcaccactgaactccagcttgaggaaaaaaaaaaatagagtaccactgaaacttttttagaggacgaatgatgtgataaaaatgcaaaggtagtctcacagtgacaattgagaagctgacatatggattaaagagatatattttgtaggaaaaataagtgtgacttgttgactaactggataatgaaaaagataagtttaagatgacatgaagatttcagtcacgaataactgtgaacatggtatcattggaaaggaagacatttgctgaatgaaaagagaatgtgcttattaaaagagcatgccactgtgttactactatacaactataattataaactaatataataaatactttaatgagcgcaatgggacatatatatccaaatgaggttttttttttttttcattaaaaatacatgcaacttctcccctccccaactgtgtggattactcgtcagaatttctttgaagctgctcttgacattttctccagagtgcagtgcaaccttttatatctttaaaagtgataaagtatttctaaaggagatacactttttaaaagagttagaaactgttttgaggctagtctgacaagtgtgattctcattgggttactattaccccttaggatcaaaatccaggaggctgaggcaagaggatggcttcagcctaggattttgaggttacagtgagctacgattgtgtcactgcacttcagcctaggtgacagagcaagaccccttctatttaaaaaaaaaaagttgatgaataaatgaattcattaaatcagtggttctcaattgagggtgatatttgcctctctgttgacatctggaaatatttggaaataactttgtcacaactgggctagttgagtgggcatcctagtgagttgaggccagggatgttgctaaccatcctacaggaggcaagacagcctaacaaggaattacctggctcagggtagccattcaattaaagatgtcatctttgctttcttctttggattttcttgtgttgcttgaattctttcacaagcaatttttttttttttttggtaaaaagtcactttttaaatttgtgtaattttttttaagttagtagaaaacttcctctgaaaacagaacaaatgtctgtgctttatagtaacaccatatataaagtgaattaagttaatgtttccctgtattctagtcattctggaaccaagcaacctatactaacacccagggacatttactgccaatcagttatgtacagtatttatagtctattcaaacaagaatgtacataagcttagagaagtggtgagtaaagaaaacacctaggcagttagccgtctattagaaaatagataattttaccaagggacacaggatttcaaacctggcttgaatgatggaaaaaactccagaaccaacggtgagcaaaaatattccaatttgaattcaggtgacagaaaaaggagctcacgaaaagaatagtttctaaatttgttttagagaaataagaaagagaagggatttctctttttttataaaatgtgtcaataaaggaaattgtatagctctcttaataactttgtaagtatttttaaatgttgctttaataaatagaaaagctgttagtttacttaagcctgaataaaaccattaagttcttttatagagtgtaaatacaacactggtgccaaaaatcagtttgttacctctcatgacaccttgatgcagccatcctccaagggctttgggcaacatttcccaagagtgagaaacctcacagaacactgaagacaccaaaagaaaactttggaaccaaattttgcttcctgttttattaggttttcatgtgtacatatccattttctttctatttggtgcacttacgattatatctgactgtttttcctgaatattatgatacatgaattccaccccagtgcttttgattctaaaaggagcttggaaacctttcttctttttcatttaaaatacaaagtagatttaagaaaaccataagttttaaggtacaatgctgaaatcagtcaaaatgttactgcatttgaaaagatcaaattatgatttaaagagagattatcaatttggctttgattttgtttctccacatgaaatattgagtcacatattttgcatcattataagtatagttacatttcattcagctcttctgagtttgtatctgttgcattgttccatgtacagtttagaaaccagtcccataactgatactgatcttgcaaagagaactgtcttccaaagatcatactcagttgttgcttccgaatatgataaacaacactccattttacctgcacgagttaaagctattcctagaagaagagttaacagtggagacactggtaagtatgaggataaaaaagaatattgagtgatctgactactacaaataaaaaaattgttttatttgcatataatttttaaaatttaatttttgaaggaaaaggcatgggataatttctaatgtgtgattgacattccttctatatgacatagctataaatgtgcatctctaaattttgcatcttagttgaagggaaatctggagatcattcagtgattgttcgagaataaattgacctttacaatcttacttgttaggtatctaagccaaatgttacaaccattatcccttccactaaacttttgtggaccaggagttccaatgacgtacacattcacagttatctctccaccccaactgaagcttgataactcttggagctgccatcattctaagtagataataaaactttggggatataaatagcccttaatcgatttaaaaaaaaaaaaaaaacagttacatgatctggaccagttggtatcaacagccccaaattggagctggggattctacctcaagaataattttaaaaacccaagaagaacagggtaatatagtgatcaattatcagagagatcacttgctaaaatgaagatttggcctaaaaattatagtcagatgattaagttatcttactgaagacttttaagaaaaacattattgaagattgtccagcaatttataggttcattcaacaaatatttttgacctcctgttaagaaatgtttcttataaagccatcttttagaaaatgatgcagatgagatgtagatttcatttagagaaaatataaatcacttagcttttagagtgttttagaaaaggtgaactttagattcagctctgattcaaaacccacctctacaacttaatagctatatgactttcagagagtaacatatttaatctttctgaaccttggtttcctcagtaaaagttgaaattgttatgatttgtgtcatgttcaaaaaaccaatgacaaaggctttaagtgaatatattagatattattatcttaaatatatatgcactgcctatgaaaattaataattttctttttctttaatagaagttggttcttcccttttgagacatccgtctcctgagctttctcggctaatctcagcccacagctctctttctaaaggagaacgaaatttccagtggccagttttagcttttgttatacaacatcatgatctagaaggtcttgaaatagcaatgaaacaggccctaaggaaatctgcttgtcgagtttttgctatggaggtatgaagacattaatgaaattactgattcattgattatacaaatcatgtgatgattctatttcacgatgccacttttttaccaacagaaattgtgtgaaattaaaaggcattagccaattaaaagtattaagtgttcctataaaaatcagactctaaagtttatgaatataaatgaatttagaatttcatatttaagcaccctaaagaaatttatttctgtattgctttactatttgaatcttgagattatcttttttttaaaagctttttatactcagaatgtcattttattagaatgaaatgacagagtaatcatggaaagaagataaggagtagggcttttgagtggtaagttgaccagtaataacttctctgggcctttggtctctggaaataataggattactgtatattacaggcctatatatagtaaatctgtgtttttatcaaatgtgataaataacctaccacttcatggatgggaattatatgaatatatttaagtaaatggttgcttgcctagagcttgagatacactttgtgttcatccagatgcaagttggaaaaaatgtgcttgaataaattaatattaaaatgaattttcaaagtaaaatgtgatttgaaaaaatattttggtattgtatcttataaattatttttcctgttttttttaaattaaaaccccaaattattaactgtttcaagttccgtgtgctttcagtgaaataaaggtcagattttcaaggtgccaagcatagtagcccatgcttgtaatcctagcactttgggaggcggaggagggaggatcacttgagcgcaagagtttgagaccagcgtgggcaacatgtgagacctcatttctacaaaaaaaaagttttaaaatcagctgggtgtggtggtacacacctgtagtcccagctacttgggaggctgaggtgggaggatcatttgtgcctgggtgcttgaggctgcagtgagccatgatcacaccactgtactccaacctgggtgacagagcgagacactgtctcttaaaaagaaaaacaaatagtaaaactataaggcattttttttaattaaaaaagattttctagtatctttctttgaagtcttaatccatggaggtactttatttctgagtactttttaaactcatgtattttattcttgaactcatttaaccgtcatttcttcaggctttcaactggcttctgtgtaatgtcatccaaaccacttctctccatgatattctgtggcattttgtggcatcactgactcctgcaccagtggaaccagaggaagaagaggatgaagaaaataaaacaagcaaagaaaattcagaacaagtaaatgaaatttttgttttgattgtgcatatttttatagaaactggtagtataccttcaaatagtccaataatttgcatgaaatcattcgcagatctaacttgaaagcaaaaagaatattttagtgctatgggcaaagtattaataatgagacctggattttatagaaggcagcctgtataatggagagagcattgaattagggcactaggagcctgagttcagcctcatttctaccctattgtagttgtatgacttgtaggcctcggtttccatgtctgtaaaatgaaagtaatctaggtgacatttatcttattcccagtcactctgaaattatgtgaatggcattttaggttaatttccaaggatatcaagcaacattatcatagtcaaattcttaaaactcacaaacaagacaccatgagcaagaactaacaaaaacaatagacagtagaaatagaccctcaacaatggttgtaattatctatgtaaatgtagaataatgtgttaatatgcttaaagaaataaaagagaaactttaaaatatgagaagaaattgtaagaagaaatatgaagtgatgaatttgataaagaactaaagaaaacataaatactaatctaagtataattgatatttttagttttgagattttatagcatgtcagtgaaaaaattggctaaatataagagaatgatggattcgataaagaactaaagaaaatacgtaaaaattaatctaagtataattgatatttaaaaaacaatcagttggacattttatagcatgtcagcaaacaaattggttaaatataagagacctgtagaaattatctagacttcaacacagaaaaagtaaataaaaaatacaagttgctaagagcaatgaagggcaaaatgatgtacaacttacctacttggagtttcagaaggagaagacagaatggaagagagacagtgtttaagagttaatacttgagaatttcccagaattgattacagataacaacccattgagtcaagaagcccagtaaaccccaagcagaataaatacaaatcatggtggacaaattatagtgaatcacctgaacaccaaaaacaaagaaagagatcttttaagcagccacagaaaaaaatattagctttcaaaagagggacagtgagaataattgcttaatttttggtagcaacattacaagttaaaatactgagtttgtttaaaaggctgtcagttggtttttttcaacgtgctgaaagaaaatacttgttaacccagttgaagggtctttgaagaacaagaccaaagacatttccagaataaaaactgggagaaatggccatggagtatcaatgaagagaaaagaaagtgtaaatatgagggcatatcttaaaaaacattgactatgtaaaaagtaatgatatgtcatggtgatgtacaaaatgataaaagatcaatcctggatatgtatctacctaatactgtaaccttaaaaaaagtaacaattgacagactgcaaggaaaaataggacagtctacagttacaatgtgagattgtcatatacctgtaacagctatctgttcctgcataactaccccagaactcactggactaaaacagttgtttatttgctgacagtctgtgggttggcaattgcagcaaggctccactgagaggaatgactctttcctgttttggttacatagtgtgcttcaagtggtaccccctgagctcttttatcctccagaatgctggcctaggtttgttcagatgccagaagcattcaagaaagagagcacagaagctgcaaagccaaaatggcgaacactaaactcatatgccatgcttctgccaccttccattagctagtataattcacaaggccggtcaaattcaaagactgagaaataaactccacctgttgatggaaagagcgacagagaatttgtggctgttttgtgtatagtaccataacactctttttagtactggataggtcaggcagagaaaaagaatcagaaagtacacagatttgaatgattgacagtcttgatagaatatacaaaacattgcatatagttgtttacattcttttcagatacacacaaatgtctatggaaattgactgtttactggacacataaagcaagttttaacaaacttgaaataattggttttcttcccaccatgtcctcagtctttattgctagtatataagaattagataacaaaacaataacaagaaaaatatttatagggaaattaagtaacgcttccaaatgagttgcatcaaagaagaattataacagaaaatattttaaatggaatggcaataatagtacatctcaaaacttgttgtttggcagagttctagttatagttacgatgaaacagccagtgtcagcattagttcttatagtaagtaacaaaaagctgggcaaaatatttaaaacaattgttcataggctctgaggagtgaccagtacagggctgtaatccttgagagaagaaaagggcatgaggtaagccccacaattgggaaaagagcccaagcagagtacgtcagttgtgttggcagagatcaaagtttaggactgtaactttgggatgtgggaagcagggcactagtggtacagagctgcagaaagggagtctaaaatttttgcataaaaatatccctagggtcctggccactcttagactgggcatgcaatttctctggaggatctagcagtggaaatgaattgtaagctgagaactgaacagattcttagcttcagagctgggttctgaagttttagtccaaccagaggtggggtgagcgtgatgaatactcctggcatgcccttgaaaccccagaaagaccatactcaaggaatacagaccatgccttaggagtaagggcagtgcctaaaatcaagggcaaaactgaaataaccccagtctaacaaagtcgaatgccaagccttgatggaatctagatggcctgctatctccctgtaagaacaaaactacattctttagaagacaatataaaccagaacttctatatcagtagaagataatataaaccagaacttctatatcagtaatcaatatatgcacaaagaaggaggcaaaaagcaaccatcttttacttagctcagtgacatagtttaaaaagtttgatgggcttacattggtagcaagatggaagaacaaacattctttgctgtggagtagcagcttcttagagaatggtttggtaagatctatcaaaattcacattaaaaatcattcagtaattccacttttatgaagttctccttatcatatatgcatgtgtgaaaagacagatatacaagttgattcattgcagaatgttatataaaaacaaaagattggaaacaacctaaatgactgttctgaagtggactgatgaaataaactatggtatttttattcatcagaatactatgcagtgggataatatatgttacatacatatctaataagtaataatctcaaagataaattattgttaaaagaacacagatcagtttgtttccagtgctgccatttgtgtaaatgaacaatatttatagacagtccaagaaactggtaatagaaattgccagtgggagggcatctaggtggctgggctgtagagacagggagactttttatggtatttttttatacctcttgaatattttattatgatgtgtgttatctactcaaaataataaaaagttattccatagttaatattattaatggagccatctgagggttgtaatgagaaatgaaattcaacccctacttctctttcctagctgtggtccatttgcgtagaaaggagcttttttttctaattcacataaaaatgctgtatgagttagtgaccaccccaacttctaaacatatgttattttttatacataaacatacacacaaaacatacctacatatatgtatgtgtatatatccatacacatacatatatatgcacacacactacacatacacatgcatacacatgtggattcaccctacaggtatctttgtgagtacactagaattgatacatttattcatttgcatgctgattttgcttgtgactaaacttacagcttttttccctaaaataatacaagcataattagttaaaattactgtatctttctgggtacaatttggttttgttgtttgtatttctgttacatgaccattttttaagtatacataatagtcttttgtgaagaggattccttccagcacacaaactgataaacttgtttcaatttctgccttaggagaaagatacaagagtatgtgaacatccactctcagacatagtgattgccggggaagctgctcatcctttaccacacacctttcaccgcttgctgcagaccatctcagaccttatgatgtctctccccagcggcagttcattacagcaaatggccctgaggtaattttggtatccacatatcctaggtacattggacaagagagtttatggaatattcaaagtatgaaatgctccaaaaaaagctctttaaatagtaagatagaatccctacattatatagatggtgcctccctccttacagtcataatttttagaatggtccttcctcttagttaacatttgtttgccctcctgtaaaatgtggctaatgtaattcttagtgccctaaattatcactgtagcaatctccctcactttccccagtctcctacctcaaatcacttaaatgtcccaaaaagagagagggaaaatgaaatctcaaaaacaaaaaccaaaatagagttttgacttgaaaagaatcttggggaaaacagagcctggttgtatgcacccagtcctaaaagcaccttaaggagcatcacagatgctcaacagattccaatttcaatttgaaatgtgttgagaatttagcattgcttttacactgtgaaccttgttactagattcacatactggactcacacttataaataaattctcctaattaatgtatacaagaaatagattgatggaatgcttgggcttcccatgttactaatgataagaccagtggaagagagatttctctttgtcctcctcacaagatttttgaactacaagataaactataccctgatgaattagtatggcattgtaagagtaataaatttgaggggttgttgcagttggtgtaccacttgtttacacaatagattcattaccctttttgcttctgtagccttgatcttcatctgtctaattcttttatatttaaaatgggtcaactttccttagaatttaccttgttccaaagaagttacctttatttctcaaaaacaaaaacaataggaatgattttcttccctgaatttttaaaatgtctgttactagaacattaaaatttcttgagatattagctacagagtactcatctaagagttccttagatgttcctggaaaataattatttcgtatattgcttatatatcgcctttttaggtgctggagtctcaaattcaagcaatctgatcaccagttccttcatcagagcaacgtctttcatcacattaacaatattttgtcaaagtcagatgatggcgatagtgaagagagttttagcatcagtatacagtctggctttgaagctatgagtcaggtcagtcatttcagttattatcctggttgtcctgttagttaataatacatcaaaggagaaactgtaagttattcataatcaatttgatcctgtcttccattatgttttatcttatacctgtttgttttatcatcacgaaagatttgcttttaagtatcataagtacttatatttgtttctgataggaattatgcatagtaatgtgcttaaaggacttaaccagcattgttgacataaaaacttcaagccgacctgccatgattggcagtttgacagacggctccacagaaaccttttgggaatcaggagatgaagataaaaacaaaactaagaacatcaccatcaactgtgtaaaaggaatcaatgcccgctatgtgtctgttcacgtggacaattcccgagatcttggggtaagaaagcaaacgtgatttacggctctttagctcttttcagatcttagtattcatgcactttcatttctgaatttagcaacaagctttatcacctggaatttcaccaaccaaacatcattgctataatccccaaataaatcttaagtctaaaaacaaattaatatgattgggagcactgtggaattccatttctgaaaagaggtctgattacaaatataccatggctacagctaagcaaaatgaaagtctgctctacaattttctgctgccattcccttggttaagattttcccgtgacctggaagtcctccccttaaactctgctgatcaattcttccagggtctctcaaactcagcagagaaattctaagtaactcctctaaaaaattactctttccctctgctgattctttttgtattactctggtggcacttaataataatctttgacctatagtgtaatcagcttttttttttttttctgagatggagtctccctctgtcacccaggctggagtgcagtggcacaatcttggctcactgcaaccttcgcctccaggttcaaacgattctcctgcttcaacctcctgagtagctgggattacaagcacacgccaccatgcttggctaatttttgtatttttagtagagacagggtttcaccatgttgtccaggccggtctcaaacccctggcctcaagtgatctgcccacctcagcctcccaaagtgctaggattacagacatgagccactgcacccagcctatcttcaagtgtctactttgccacagtcctcatgaaataaaaaataagtttcagactgtcccaaataatcataatttatattctcttcagcctagttcatccatgtgtttcaactgaattaaatattcttttataaaaggcactaaacaagaaaaaagagtcctatttgcttgagtattaacaaccaagaaagaaatagcactaactgccagactgcctttcttctacctcatttagaatttttttcctcagaatggatgtggcttgctagcagatacagtattaatggctctccttgaaccattgcctggcttctcctgtgccatttgcatatttcctttctcaggaagagccacagtggtaggattgggtctcagataacctggatgcaggccagtctgcaatcagagcaacctccaactcctgtacatcagtgcaggttctattcctccctggtattgagtaagtaaaagaggcttagctaatataatatgagctatttcctcctcagacctaagaatttccactggtgtaactactttatcttactggaaagtcaagtgtaacaaagcaaaacatttttgtagctagaactcactgttcagtgcaaagctatctttggctcatggtacattttaagcaaggacatactctccctcagaacaatgattgcaatcaatttaacacttctgtcttttctaagaggaaataatcataacagtattcattttcaaaattcccaacatgtaagctgacatctactttccttttaacaggcttcctctatgttataggttgagcattccaaatctgaaaatccaaaatgctacagaatctgaaactttttgaacacagatatgacacttaaaggaaatgcatattggagcatttctggttttagatattcagatttggggtgcccagccagtaaggataatgcaaatagagtatttcaaaatcctaaaaaatccctaatccaaaacacttctagtcccaaatatttctaataaaggatactcaacctgtatgtatgttttccttgagttatgtacctccatgttcctcaacctgcctaccacagagccttgtacacagcatttgttgatttgagttgtatcagcctacagcactcatattatgcagctctccaaggaattaatgttaatctatttcttgatgtgctttgcgtaaaacttattcagccttaagatatttgacactagtttgaacatgtattgaacttgtactgtttgacatttattttgtgtattaatgttgccttccctagtttggcaagtatcttgggggatagaacctcctctcatatatcctacttcaaatctgtaaggaagtctgggtacattgtgacactcataagtatttgttagttcagttaccttccctctttatacttccaaaagtaatgaaaatatagcaacaagagtctgacataagtgacaagcagatttgttgaaaggcttcatgaggaaggaatctgaatatttagactttgaataaggagagggaatgtcaagctgtgagagagcacctggagagaggctgaaatgtgcaatttaagatggagtttgtgagaatgagactaaagttataggaaccgtggttagttggagggatggggtggggtcaatcaggaaggaccataagagacaggcagaagagttgatgtggtattagtaggtcatgagaaaccattgtaagttctgaagcaggaatatagcatattaactattgtgaattgtctatttcagaataaagttacctcaatgaccttcttaactggcaaagcagtagaagatttgtgcagaataaagcaggtaattgaaattttgtcattaaatgtttcgctacctagagagaaatttcttcttctcagccttcactattgaattccacccttggcacacaaccaaagtattatagggaattatctatttacagaacactaaattgacaagtggtccaaaacataggaatagtaaccaccatccctgtgctggacatctcacctgttttcctaaatgttccccagtcatgagcaattatattgtacctacagtctccctaccctactccccaacaaagactcagggtatcttaaaatttatcacagatcccattcacacttaaaaccttacttcacctatactttgagttaatacaggttattgtcctctggcctgaaaaatctgtctgttataacacttataacactgcagctatatccaggttgcaagtcagtttaaaccaactttagaaacaagttaagttttatctaggcatgtgtctgagactgtgctcaagctcccttacttgtattcatgaaactttgtcaaatctgttcctcactggctactttatctcacaaaaagttggccaatcgcttgcttgcttgcttgttttcaaatcacattgaaggaatcaattaagattttggactaaacatataataaattttgtttaaattgttgaagaatttgggaaaataatcctattcaatgagattgtgtttgtttatacttaatacccctttttgaaaattgacattattaattaaaataatatcaacttaatgtctatttaaatcacatagaaatcgccaggtcatttcttggttattaaatgccataagaacagtttttattttatgaagggaatagtgtcaagaactatttcacagtatattaacatttaatccttgaaaaaaaaagtcatattctcaatgaaaattagtgaaataaggaatcacaagtgtgaagttacatattatgtaatttttgttcaaaagtaaaacatattaagtaactgcactttaaagtcccttatctttaaatggtactttaatgtgaggatgcacaaaaaaagaacccttttctgatcttgtgaggtttccacttacagatgatatagataaagtacagtcatataaattatttgtaattatttcactcatataattttgctcggaagccatatcgtagtcaggcctggattttaattctcaattccaccatttactgaaaatcccttaacctgtattaaccttagtttcttcatctgtgtaatagctactataaataatattacttatcttcacctggttgaaataagaaaatcagtgtgaaataacctcataacctttactctgtgcaattctttgttatttgagcttgctggtatcaatgctaagtccagcacagtgataaaagtgatccagctgaatcaggttcctgcctcttttctactcaaccaggttgatctggattccaggcacattggctgggtaacaagtgaacttccaggaggggataatcacatcataaaaattgaattaaaaggcccagaaaatacactgagagttcgacaagtcaaagtcctgggctggaaagatggtgaaagcacaaaaatagctggccagatttcagccagtgtggcccagcagaggaactgtgaagctgagactctgcgagtattcagactgattacgtctcaagtgagtgtccttacaacatattctagcacaggataattgatgtaatatattttaagagtgtaacaaaatattttgtgaaacttattacagaaatttctggctataaatgttgcccattttttcttttcatagccctaaacacctgagttccatgttgcatttgtaattaagaaaaatagtggtgtatattagcaagagtagcatattataaaacctgcggaatcagctaatgagtcctctggccaatatagtgttaatgcctgtcattaattccttctcacatgcatatcttcatcaggtatttggaaagctcatctctggagatgctgaacctacaccagaacaagaggaaaaagcactattgtcatcacctgaaggagaagaaaaagtatacaatgtatttatttgtattctaaagatatttatttcccttttttattctttttttcagtgaaataattcatttattgctgtatattttgatggatatgtaataacagttttatgcagttcatatttgttcttagaaagatgaaggaactatctaaaagaaattcattattcatgtataatctagctagtattttaagtaacagcaaatttgctctataccttgggtttttgcattcatttgtaatcatttctgtgctataccccatcagttatcttctccctcctccattgtccctgtccttcacagttcagctcaaaagtactgtcttcttcatgaaacttttttacgatttatcctcttcatgaaatttgtttatgatttccccaaacaaaagcagttttctctctgctggagttcataataatctgtatgtgtctcttcttcatttttctcttatgtttatctacatggatgttttctctcttctactgaaccatagcttcttaaagaccaagtccatgtctaatttgtctttgtatccatcatggtagcatcacacacatgtttagtaaatttttattagaataactactctttgttactgttcttgaaatttcttgcaaaaatacagttgctttactttttcggtcctgactaaaacactatacctaaaacttttgtacttcatccatttgcaaaaagagaagtcaactctgactagtctcaatattctagtgtctttaaatgctacaaataatcaccttggcagccctttgaaagataaatttcaaggatttcccttgtctcaccctgtgtttcttaatacagtagtatctaatcattgtcctgtttttgtttgaggtctgaaaaaaataagatttcttaaatgctggtaacttctttcaacatcttttggagatttcatttgaatgactacaagactgtgtaactacctgttaagtcaattgaattttagcagatgtcaactctgtactaaacaattgtgagaatggcaagccctcaaaaagcttgcattctgttcttggttatgaaccctctgcattcccctcaccccccccaaaaagaaatcaacaaagatactatatagtataaaagcaagttattaaatgtgtgaaaaaaattggatcattgtgccctggagtggtcagagaaggcttcttaggggatattggactggagttagatctgaaagagaatgaagggattcaaatgagattagtagagagaatgggagcaaggactgatcagagagactggcctaagggtgggagagtgtatgctgaggagttaaggttgagtaggaaaggttatactgtcatgtgtgtgcccatgaaataacatgatttttaagagaatctttttttaagttagattattgaagcacaatttacatacagaaattcctcattttaaactgtatcattcaatgaatttttacttgtgtacactagtgaaatcattatcacaatcaagtttgttttgttttgttttgcttgagaccgggtctctctctgtcatccaggatagagtgcagtggtatgatcacggctcactgcaacctcaacctcccgggctcaatggatcctcctgtctcagcctcccaagtagctggaactacaggtgcacgtcaccgtgcccagctgattttaatatatctgcagagatgtggttttgccatgttgcccaggctggtctcagactcctgggctcaagcagtctgccagcattggcctcccaaagtgctgagattacagatatgagccaccatgcccagcccacaaccaagttttaaaacacatcactccccaaatttctctcatgttcctttgcaggcaatcccccaacctcacacttagagcctagcaactactgaactatattctgccagtgttgtgttgccttttcctggatgtcatataaatggaaccactcagaatgtagcctttttgtcttgtttctttcactttagtgtaatgcttttgagatttatccatgttgtagcatgtagccatggttcattcttttggttgctgagaagtgttccattatatggatttaccgtaatttgtttatcctttcattttttgatggatatttgggttgtttctgctgtttggctactattagtaaagctgctgtgaacactcacatacagatctttgcaggtcttttaccaattatgtgttttcaaatattttctcctagtctgtgtcttatctttcagagaacagaagtttttaagtttgataaagtccagttttttcttgtttttttgattcatgatttttatgtatggtctagaacccaaggtcacacagatattctcttcttttcctttagaaatgttatagttttagttcttttctttagaactgtatacattttaatttttatgtgcagtgtaagaattgaggcttatcttttgcatatagatgttcaattgttctactacgattcattgagaagactatcatttatcaatagtcaatttaccataaatggtaggtctgtttctggactctattctgctccattgatccatcaaagtgtcttacttactgtagctttataaaaagtcttgatatcaggttctaactttgttctttatctaagttattttggctattctagatctcttgcatttccatataaagcttagaatcagcttgaccgtttctttaaaaaaaaaaggggggggggcggtggggatggaattttgactggaattaacaccaaattgatagtttagggagaatcgccatgtcaataatattgagtcttcgtatctgtgaacatgatatgtatcaggggttttcttacatctgtttaaaatttctctcagcagtgttttgtaattcattgtacacatcttgtacatattttattaaatttattcctaaacatttaacatttgtaaagctattgtaaatggtattttttatttcactttccaattgttcatttctagtgtataaaaatagaattgacttttgtatattagatttctaccctgtgatcttaaattcacttactaatttcagaaactttttatagactctttggggttttcttagataatcatgtagtcttcaataaatttgggttttttcctttctaaactatatgccttaaatttcttttcttatattattgtgttattgcattgattaagcccttaagcgccatgctgaatagaagtaatgagaacagatagctctgacttgttctggatcttgaaaagaaagtataaaccattcattattaagtgtgatgtgaactgtagattttttgggggtgaatctttttaataagattggagaatgttttcttctattcctagtttgctgagaatttctatcagaatggattcttttttaagagacctggtcttaactctgtcacccagacaagagcacagtggtacctttatatgtttctctttcttcatttcttttattccgtaagataatccatatattgctatgcattttagtcaacatgtgatactagcttgtatacagttcattttccttcagtgaaaaaaaaaactaggggatggtagctacatgatatagtgcttcttttagaagtaataaaaatgctgtaatattgactgacgacgatagttgcacatatctgaatgtactaaaaaccattgaattctatgctttaaacagatgaatagcatggtatgtgaactatatctcaataaagatgtttttaaaaagcaaaaataaatgatgaaagaggtaaacaagatgaaagaactatctaaaagagactcatatataatctaactagtacttaagtagcaaaataaatgctctattctaaaaattcactttctttaatagataacagagctgtttcagttatctgtttctttttgggtaagctttagtagttctgacactcaagcaatttgtccatttcatctaagtttctggatttattgccataagttgttcataatatacccttatgtctttttaatatctgtagaacctgtagtgatgtgtcttggtgatttgtgtcttcattctttttttctgaaatagtttctctaaagattcatcagtttgatctcttcagagaactagctttttgtttcattgattttcctctatttttgcttcttttaataatcacaactttatttctttccctatgtttgccttgtattcatttgttcatctttttctgacttcttaaggtccaagatcagatcgtagactccaagacctttcttttctcatctaagtatttaatgctattattttcttctaagcactacattagctgcatttcacaaatttggggtattctgtttttattttcattcaattcacaatgttttcttagttttcttattcccatttgatttctttttttttttttttttctttttttttgaaatggagtcttgccctgtcgcccaggctggagtgcagtggtgcaatctcggctcactgcaagctctgactcctgggttcacgccattctcctgcctcagcctcccaagtagctgggagtacaggcgcccgccaccatgcccagctaattttttgtattttttttagtagagatggggtttcaccacgttagccaggatggtctcgatctcctgacctcgtgatccacccacctcggcctcccaaagtgctgggattacaggcgtgagccactgtatccggccccattttgatttcttctttgacccttgggtaatttagaagcaccttgtttaattcccaaatatttgggaattttcctgttagctttatattattaatttctggttcagtttcattgtggtcaaactgtattgtatacttcgtataatttcagtccttttaaaattgttaagatttggattctggcctataatatagtctgttttagtgaatgtccacgtgcacttcaaaaaaatgtgaactctgctgttcttctttgaggggttctataaatatcagttagttcaagttgatcaataatattcaagtctttaagagccttaatattcaagtctttaagagccttactggtttttggttttttgcttgtttacttgttctgttaagtactgaaagaagagttttgaagtatccaactaactggatttgtttatttggaattcattctacctggtagctgatctcaacattctaatggactccaaaaaaaaagttttggttttgtagatgatcaggtttttcctcattgttagggcgaaagggacattctcttctggctctctatcttaagaaatgtaaaaccaagcatcatttttgattcttaaacaaaagagtttcttacctcagttttcttacctgtaaagtaggcaaaatattacaatttaccttctggggttgttgcgaagattagataagatttgtgtgtgtgtgtgtgtgtgtgtgtgtgtgtgtgtgtgcgtgtgcacacacgtgcatgcatatatatatatacacacacatatatacatatgtggcacttagaagaacagcgtgcacataataagctctataaaattttagcctttatatttttccttatgatacatttctttgatgacttggtgttccgatagcttaatttaagaccactatcatcacagaggtttgttaggacactttccttttaaacaaatgttattttcctggattgttcttcaaacaattaaatgtgggatgtgaactaataatgtttacaatttagaaatagatttaatcaaagctactcaaattgactacattaaagaaatttgtgaaaggagaatatacttctctgaatttcctaagttctgagattttgttctttttatttcaaactttatcctttgtcttagtattataagcctcaatttaaaataaatttactttaaaaatatatacacaaaattatttctcaagttactttgaaatatcacatatacaaattttatttaaagagaccacttcattcttttttttttttttctttttctatgtaaaggcaacatcagatgctgacctgaaagaacatatggttggaatcatattcagcaggagtaagctgactaacttacaaaaacaggttggttaacagctatttcattgtcttcattagttttgtgtgtgtgtgtgtgtgtgtttgtttgtttgtctgtttgtttgttattagggtataaagcagggatgtccaatcttttggcttccctgggccatatttgaagaaaaagaattgtcttaggctacacataaaatacactaaggctaacaatagctgatgaactaaaaaaaaaattgcaagaaaaaaaaatctcctaatactttaagaaagtttatgaatttgtgttgggccacattcaaagccatcctgggccacaggcagtcccccggctgcaggttggatgagcttggtataaagtattttgaaaaatttatgaaggcttttttatgctcgcccttttataggtcctgcagaatgtgcctgccattttttagagaggaatgagtattttggcataccttcatcattttaaagattttacttatgaaaacttgcttaccttgctgctatttgttatgggtagattttttatttggtgtcaaatacccagctatatcagatttgatttgcatttcccatatttcttttcccccattcttctgtgtgttggctgagcaattaacagtgttaactataccagtgttaatatttttaaatataaccaggtgaggaaatagcgctatattgttcatataatggggtgctacataacataaaaatatcacttcaaaagtcaaataaaatattgattttaaattaacctattattctaacttttttttttttcgagagggagtctcgctctgttgcccaggctggagtgcagtggtgcgatctcggcccactgcaagctccgcctcctgggttcacaccattctcctgcctcagcctcctgagtagctgggactacagatgcctgccaccacggctggctaattttttttttttttttttttttttttagtacagacggggtttcaccgtgttagccaggatggtctcgatctcctgacctcgtgatccacccgcctcggcctcccaaagtgctgggattacaggcataatatttttgtatttttagtagagacggggtttcaccgtgttagccagaatggtctcgatctcctgacctcatgatccacctgcctcggcctcccaaactgctgggattacaggtgtaatttttttgtatttttagtagagacgggatttcaccatattagccaggatggtctcgatctcctgatctcatgatccgcccgcctcggcctcccaaattgctgggattacaggcgtgagccacggcacccagccctattctaactttctacactattattcttttcaatgaactacaacatgaatttatttttgctttatctttctagcagtgtttcattgaatcttagatgctttcaattgtaaaatgtcatgtataatttcagtattgtgaatcttatcagctataaagcaccttatgaacataatggtaaaccatgagaaaaagtgccttcagtgataaaatatggtgattggtttttgacgtgctaaactgtaagcataggcattaataacttgcatataatcagtttgcacatagtagttgatttaaatatttccatttttattgtggctcccttagctttacttgtttcttagatgagttaagagaatgacatatgatggagaattaactgggagaatagtattaaattgagtatcaatttacattcttgtcttattcttttgtttgcttgttttgctaggagtagtaaatcaaactaaccctatatttttacatgttctgtgttaactttaaataggtgtgtgctcatattgtccaagctattcgcatggaagctaccagagtccgtgaagaatgggaacatgctatatcaagcaaagaaaatgccaattctcagccaaatgatgaagatgcctcctctgatgcctactgctttgagctgctctctatggttttagcactgagtggctctaacgttggccggcaatatctggctcaacagctaaccctgcttcaggatctcttctcgctgcttcacacagcctctcctagagtccagagacaggtgactgatcacactacagcctttccattgcttttgttacttgaaacattgttaaaagttgcaatgcaaagagaattatctaaggaccctaagtatagatttttatttcgtattttgttatattttgtttggggcctgtattttactcccttttttactgtttatagtgagagtatagaatagtaaaatatgttccattttattaatgtgttgtgataagagagtattctggctactgattaccatattaccatgaaatatataatgtcatttagccctgaccagaaagaaaaggtattttttttcgtgctgccttgggttagtaattaccactactattaccatttgaatagtatgtatatcatattttcttccttcacaaaatgtaaaatagtgtttaaatcatatttcttttcttaaagattttgaagttacagaatggtagttaggtacattaaaaaatgaagcctgggatggggtacagtgacttatgcctgtaatcccagcactttgggaggcaaagctgggaggatcccttgagcctaggagttcaaggctgcagtgagctgtggttgctccactgcactccaacctgggcaacaaagcaagaccctgtctttaaaaaaagaaaagagaaataaaagaaaccttaaaacattagtctttacatggaattctctcattttatttttcttaccaggtaacctctttactaagaagagttttgcctgaagtaacccctagtcgtctggccagcatcataggagtgaaatccctccccccagcagatatcagtgatatcattcactcaacagagaaaggagactggaataagctgggtatcttggacatgtttctaggatgcattgccaaagcactcactgtacagctaaaagccaaaggaaccaccatcactggaacagctggtaccactgtgggcaaaggagttacaacagttactcttccgatgattttcaattccaggttagttattgcctctattttagtaccaaaacagaatagagtgagctactgttcagaaatttaagacataatttcagattttaccttgttaggttactcaccaaataaaatccataaacaactggctaatatctaaaaaggtcatgtatataataaaagtgtcatttataattagtggggaatagaaagtaggctgaatatgtgatgtcagaatatttggatatccatgttaaaaaaaattctagatacctacctctcaccatatacaaaattaaacttcagttttaggtaatatgtttttttgccacaataaaaagaaataaatatgggggaataaagtaaagttcaggtcaactaaagctttatgtacatttctttttaaaactgtactgggacaaattatagaagaatatttttgtaatcttcaatttgataagaccttcttaaacaaaacacagaacccaaaatcatataggaagatatttatagatttgactatataaaaatgaaaattacttgataggtgataatatttaaatgttttaaaatatagggaaataacttagaaaaatacttaaacctatataatagacaatgacttgttagcataatatagaagtatgactacaaatcaattttaaaaaggcaaacaactgataagaataataaatgacaagaacagtcagttcacagaaaaaaacttataaatgccagtagtcagttcacagaaaaaaacttataaatgccagtaaacaaatgaaaacacctcactactaatcagagaaatgtagattaaaataattgcaaaatatctttgatgctttggcaacattatgaggaaataggcattttcatctcatataaattgctgcagcctttttggatatctgtttataatatctaactatattttaaatatgcatatcttttgacctaccaattctacttctaaaactttcatgcatttgcaccaaggtatatatgcaaggatgttctgtaattgtgtgtaacagtagagaattagagacatctgaacagtgctacttgtagagccaatccatgacaatatgcagataacatcagaatgttaatcattgtatcacttctttcctgtagtcttgctaggaaaaaagtcagctgaactaaatagtagtgtgcttagtgatacagtgaatttatattccggttaagctccacattatccgcatgtgccctagaaataagtcgtgtgtggtccctggactgtacttctaaaagatgcacacagaaataatgtgcaaacagagaaatacttgaaagggaactcaaataaccttaactgctgtcttgggatttggggtaatggtttgatgaattttctttttaaatgttttaattgattcagtcacttaaaaatctaaaatgctgcattctgaaataacattttcatctttttcatttgttactactactgataatttattggctttttaagtgctaatttttcttgtagttatctccgacgaggtgaaagtcattggtggatgaagggctcaacccctacccagatctcagagatcatcattaaacttatcaaggatatggcagcagtaagttcccattcttctgttttggcagtgaagtgcggcaggaagctgacacaaaacagcttactctgctgaattgataggagatgtagattagtgtaactttacttaaagactatttgtagtatgttaactatcatgctgtttgtcttttcagtgagtgataatttgtcatttatgtaaaccatgttcatttcaaattttccagttaatttgtgtgggaattgttacgtgactttaaaatctaataaaataaaatatgctctttcagaccctaagaaagaacttgattcaaaaaacctaaaggaaaaaaagaggaagagagctctcgaaagtggaagtttctcattctcctcagtcctcatggtttttaaaccctttattcttctttattaatgtcatataccctctgtcttaagatggtggcttccaaaggagatctgggagtgtggaggacagggagttactttacagaatgcctggctctgattagttgctccttgtcattttccacacattgtattatttctaggagggcagcctgagaaggacaagatagtatggaatgtagagtcagaaggaatagaaggcagcttggagaaggaggaggaattgaactgccccttaaatcatgtggatatgagaagtgggaggattttctgggggaaagatgaagttctatcacttttcccaacagcgtggatattttctcaacctctgtggcaaacagactttctcagggtgttcattgaattaaaatatttaggagtaatatttaggaacacctaaattatatgatagttaaatgtttgttcattacaaatcaaggtagcaaataaaggtagttgcagtttgaacctgtttatattttaggttcctgtaatactcatctaaagcagttagcttcactgtgtaaattttatgctaagtgacaaacttctcaaacatagataaccttagtatttctgttatgtttttatcagatgtgagagtaattgcccttgatttccaggaaaagagtattgagaacttaattttttatttatttatttttaaatatcaaaatgatgattagaagttgaattttccataggatcacgaagaaattttagttcaaaagtttgtgcccataatctaaaattccatggtctcactaaaaacaaatatttcctttgataggagatgttatttaagaaaaaagagtttactatggtaatacatttctggtcacttattaaaatactctgttctgctcaatagggtcatctgtcagaagcttggtcccgagtgacaaaaaatgctattgcagaaaccatcattgccttgaccaagatggaagaagaatttaggtctccagtgagatgtattgcaacaactagagtaggttttgctttgttttgttttgttttaaatttggtgtgtatcattgcgtgttcttttttagtaatagatttaaacaaattttaatcactaagtcaacataattattttttaaccaaataccgtatttattgaaaaagtataaagggcttttatattatgaactagtcgttacttctcactgttatgtgttctgtgtcacttgtttttgttattgtttaggaagccaagggtacactggttagaaaaggctaattacattgtacattgtttgcgtgttgtctttctaatgatctgtttgctgtagtttaccacagtaagtttagtgccagcattatgctgtgatagaccgaaggtcctttggagacaagacccctgcctatgcctggttgttcctaatgtagagccctgtaacttacttacaggatttataggtcttacttctaaaaagcaagaagtgcttgtccagggcagggtcagggtggaaatctttgaggaaatgagattgcattcttcagatgacacatttatagtaagttttaacctgctagatttcatgtcccatatacttccagaagagatttgagatttccttgaaggagggcagtctgacaacgttaggaagaagtgaatgctggtaaaaattgcttaggttaatcctcagaaatctgaagaagataaactatatatccctagggcggaaaaagtcaatttatgctatcttgctattattgactgtgacacctgaaataggtatttttgacagtaatcctttcctaccactcatgtgttgctttctttcagtctgcattccccttcctccttgcatgcattatttctttttctatttttctgtccctattttaacatctcttgctttctgtatccaataacactcacatgcacttcttagagttgagctagttaagtcattttctattcttcattaatgaggggatatatcattctatataaaactgaagcttaccctttaagcctcagaaatttttaactatttctgaaaacagttgtctttttttttttttttttttttttgagacagagtttcactcttgatgcccaggctggagtgcaatggtacgatcttggctcactgcagcctccgcctgcaggtacaagcgattctcctgtctcagcctcccaagtagctcggattacaggcatataccaccacacctggctaattttttttgtatttagtaaagactgggtttcaccatgttagtcaggctggtcataaccccctgacctcaggtgatccacccaccttggcctcccaaagtgctgggattacaggcatgtgccactgtgcccggccctattttggagaacagttttctaaagcttttagacatgtatgtatttctgacaaagtgcaccaagtatagtattaggctttctggggcaacaaggggcaacaaagacttctgaggaacttttattatctagaaggatttaatgtgtacctgactactcatgggactgtacacaatttagcagttgctgtaccaccttctttggcatttaatgatactaaatggaaaaatattcagaagacattagtagtaaatattaaatatgccaattcatttattcatatgttgtttacatagtagaccaataaaggataattttatttgtcagatagaagacagggtggttcatcagagaaacagtcacttgttcagtagatatctgagtgcctcctgtgtgcatggcacataggacagtgatcgtgtatgcctgctaccacatcaggaagacttgagtgccactgagtatctatgtattttcaaaatcagccagcatttattgagctgttgtgatgtgtcacagtcagcatttcataagtctttcctaaaaattcagtggtgtaacagccttccctttcaacagctctggcttgctctcgcatccctatgtgttcttgatcaggaccacgtagatcgtctctcctcggggagatggatgggaaaggatggacaacaaaaacaaatggtaagatcagaattttgtaagaatttatcttgctttcctttacagtaagctttagcttagcttaaaaacaaacaaacaaacaaaaaacacatcaacataaacagttagtggatctcttgaaagattgtgtcccttcggccaagttcatctcacaagtctatttcagagagtccctacttcatagtatatggaaactttaggggcttttatttaagtatattaaagagctttcttcttctctgagtcatgtttgcatcatatcaaaatttatttttagccagagtctgatttagtgactagatattttactaaaatgtaatcagtcttatggtaggaaggaaataaagcaataacacaggcaacatgacacaccacctgttcacatatatacttaagtaggtttgatgggtatctttaagttcactagaaaggaaaactttatataggccctttcaccattttcttattaatgttcaagtttcaaaaataaacagtttcttcctttggcatacagaatgtttagatatataatggggcagacatttcttaagtctctaagtaacaaagtatacctgttcattgtattaatcattgaacctgcaaagcacattaatgattggatttggagttttgagagatttttatagttaagtacatttgcaataattctgtttaatattcagattctaaattcttaatagacttccgaataacggtttatttttgtaaataaattatattcaatttgtatgagagtctgagacttgcttgttaatctaagtaacgtggaaatgtcttattttagcctatgtgtgataaccatgatgatggtgaaactgcagcaatcattttatgcaatgtctgtggaaatttatgtacagactgtgacagattccttcaccttcatcgaagaaccaaaactcatcaaagacaggtgaagatttttgtctctggagcataaaatatgttcagtgaattttgaaataaatgtgtgaaagaggctttgtgtagtagggggaaaaagtggactttggaatcaaagaccttggttgagttcacagctgtctcgcttaccatctataaatagcaagttgttttatccttagaaacacttaatttctccatttataaaaaagggactgctgtgaagattaaagagaagattggaaatattcaaaacttcttagagtgcttaaatgaaagccattattattattgaataattaaattttatactctccctttcagagtgataaataagcatatttttccctgttctttaatgaaataataggtcttcaaagaagaagaagaagctataaaggttgaccttcatgaaggttgtggtagaaccaaattgttctggttgatggcactggcagattctaaaacaatgaaggcaatggtggaattccgagaacacacaggtaaaaggtttaaagattgagccatgcctttaaaacccacaaaaaaagattaaaccgtgccaactgattaaactgtgaataattttgagtgatattctgatgtttaattatgacatcattgatctatatatttactaaatatatagagtgttctatggtgaagagagcattagaccaaaagtcaagaaacctgatttgccactaactagctgtgtggtgtcgagcaagtcacttaacctctggaactcattgacttcatttataaagtgagaggatcacaactaatggggctccatagataaaatacatttctattgataattcccttctagttctgctagttgttgccccacaataaaaattgctgcactagatgatcactaagatctctatttattataaggttgtatgattttatgatatttagaggtttttgtagacctaatgcttctgaaggtgttgtgaatggtactgtttccattgatagcaataaaataattaacaaaaatgaatagacataaaacaaaaggtaacctatgtatgttaaatgtatttttatctctaaaacatagttttcattttgctgcttttctaaagtaaatcttgtatattcctagttcaaagttatgttgaatcttacaaattctcagttttcagcacatctgcagtctgccatttgactgacacgaacttccagtgttggagttaggtgtaaatccgaatcaatttaattctagggtgactttgcatgctgcaagagcatgaccttcttttctcatagttgatttcttcctcctataatctgtcagtgctgtatactgcattaataaatgttctaatgtcaaaccatccttgtaaaccatcctttttaaatagatttttctgtaaatattgctgaattcaatatattgatattaattatagttagatttttatatctgtgtttaaaaatcagcttggtctgtaattttcaaatcttcacgttctttttcttattgctctgttcttgttttattgattctgtttcttctttaagtatttcaaacatatttaaattccttttcaaattgcagagttcctgaggtaggaattctgtttttcaagtttcatactctgatgataggctttataccatgccttataattttgttatatatagttaaagatttccctaacttctgtgtatttttatttaatttttggttatcatattttttatggtattgtgcatttggagtcaggggcttgtaacagctgctcagtctggtaacttgactagaagtctagtctgtttctttcactgaaaaaaaatattagtcgagagtgtatttggcttcagataggtctttaatttctttttttgtgtgtcattatttactttataattaggacaacttagaaaccttccgttctaaaactgtcaggctgatttcccaaggatagcaacttatacaaacctagtttgctttttaaaatttagttctgttaacatatctagaaaacatccataagaaggatttttatttttctgcttgaacctaggcaaacccaccacgagtagctcagaagcatgtcgcttctgtggttccaggagtggaacagagttatctgctgttggcagtgtttgttctgatgcagattgccaggtgagtatatagtgacacaggccatcccattgtccatgttcccctgtgacacctttacttcatacagaaaagagttttagctcagcgtcactccagcttcctaaatgagtgtattacctatgtgaagcatcaaatgccttaccaaagttgaagaatgggtcaaataaggctctcaagcccaaaccacactctttttgtatgtttctcagttaccatgatcatatgtatttaatatctacttttgtactagaaacagttaaaaatttatgaactgtatttcctttatctcttacagacattattcacttatttccttaaaatacattttcaacatctaagagttctttaatcctgaacatatataaacatattctatgttgtccaaatgtttttggtttctcaattaataagaatgtggatatgctcagagagtcaaaggtacctttttaaaaaaaaaaaaaatctgcattttggtgatgcggagagtggtgactgggaaattatacattggggaactttttttattttttatttttttgagacggagtctggctgtgtcgcccaggctggagtacagtggcacgatctcggctcactgcaacctccgcctcccgggttcaagcgattctcctgcctcagcttcctaagtagctgggattacaggcgcgcgccaccatgcctggttaatttttgtatttttagtagagatggggtttcaccatattggtcaggctggtctcaaactcctgatctcgtgatctgcccacctcagcctcccaaagtgctgggattacaggtgtaagccacgactcccagccaggaactttttgctttatagctgtgcatgatgacaaatatgaaattataattcacttatcagcattccctttatagagagtggcattttttaaaaatcgcatcaaaaaaaaaaaactttgttaagaagcctaatttatattcaaacaaagattagaaataactttactttgttgttacttggcctgcatagatacttcctgtgggaaagtaatctttggaaatttatataataaggaaaattaccagaatgtgtgcttttttacatacttgtgtaacattgtaattcatctttaacaggaatacgctaagatagcctgtagtaagacgcatccttgtggccatccatgcgggggtgttaaaaacgaagagcactgtctgccctgtctacacggctgtgacaaaagtgccacaagcctgaagcaagacgccgatgacatgtgcatgatatgtttcaccgaagcgctctcggcagcaccagccattcaggttgcctcagcttttaaagtattgaatgtatccagacattaagatggggaatatatttgtcaaataaacattgattgaggccgggcgcagtggctcatgcctgtaatcccagcactttgggaggccgaggcaggtggatcacgaggtcaggagatcgagaccatcctggctaacacagtgagaccccgtctctactacaaaaatacaaaaaaattagctgggcgtggtggtgggtgcctgtagtcccagctactcaggaggctgagtcaggagaatggcatgaagccaggagacggagcttgcagtgatctgagatggcaccactgcactgcagcctggtgacagagcgagactctgtctcaaaaaaaaaaaaaaaaaaaaaagcagttgattgaaatctttttttatatcactaggcagaagtgttcttttaatctatacatgtaccaggctaatgaggagcaaattctgcctctttatgctgtgctttctatgttgcatgctcagctttaatttgtgatttgtgtgaacctctgtaatcaacagaatgtataaaatattcagaagccagggggaaataattagattatcccccagtagctaaggctcttcttaatttccaaaatccagaatggtatttttacaacattatataattgatctatacataatgatacatacttctatacatacacaagttactgtgcactaaataggtttttttaacttttgcccattgcttatattttcagttctcatgattccaagataataatagtctccaaaagaaaatcataagtcagtaatttcttcttctttctgtgagatacaaaattgaataattttactttttttcctaacctagcttactttatataagtggcatgaatgttaaaaagcaggagaagccaaaggcctagttaatagtatcccaatttactgtaatgaaatttagtaaataataatgtcccaatttcagtgtcccaatgaattttcattatagtaaatttcttgatttttctcttacctgctttaagtagtgccttgcattaaataatctcccagtcactcaactgtttatcattattatatttgcttatgtccttttcttttgtttatttaagctggattgtagtcacatattccacttacagtgctgtcggcgagtattagaaaatcgatggcttggcccaaggataacatttggatttatatcttgtcccatttgcaaggtatggaaagaatctgaaatcatgtactttctttttcttttgttctttcttttcacctttcaaaataaacatatgtcagtatctcttggttttacccaccagcccctagcagtttcttcccttctcattcatgtggaacctgagaatataatctctctgttataatgcatctcaaaacagtaattgtccatttaaaactgattacattctgttaaacatattacatgagaaagttcatttaaacatcgtgatgcattaaagggcatgcatctctactctcagaccactagagagctcagttgacataacatgacaacggctaccaaaaagaagaatgatcctctggtgtttgtagaaactgatacctgccattaaagagtgaaattggaaccctcagtaccactagaacagattcttttagagcttagttaaaacaaggagaaagttcttggcattttgacacatttgtacaaaggtagtcagcaagtaggaagttgttttggcaagctaataatgattaacaaaaagctgtttttgaagaatgtggcagtactttccctattaattcatcgcccctttttgttctattaagctctccctattttcttatcttcgtatttgcaaactaatataaagggtagaatgtggaaagacttagcacaatcattgtctagatagactgaggaataatcaaaaagattgtagtattttgctctcccctatcatatggtgctacaaatattaatagatacatgaaagttttacagcaaatactacctgttcacttctgtcacctggcgttctgccttcccctcaaggaagcaaaacacacacacacacacacacacacacacacacacacacacacacacacacacacacacaccaaacacagagcgtgtcttatttgtggcaacaaggtaactttgtttccacaatagctagggcaacaggagatatatttcagacgcaggaaatataaagctaataaaatggaattttcatgctctgtgtcccatttgccccattttccctgctcttggaaaaatatgggcttctaaagaatttacagaatgtttttcaaatgacattttatttagaatatgtgtgctgtctggttatcttctagcacaatgcttggcacttaggtgcatgctaagtgtttgttgagttggttaataaatgattctgctaaagagtgttcattttttattgcagaacaaaattaatcacatagtactaaaagacctacttgatccaataaaagaactctatgaggatgtcagaagaaaagccttaatgagattggaatatgaaggtctgcataagagtgaagctatcacaactcctggtgtgaggttttataatgacccagctggctatgcaatgaatagatatgcatattatgtgtgctacaaatgcagaaaggtatgctataaattatactgagaagttttaaaaactagagcttacctatatgattaagaattcaaattgtacagtgatatctaattattccatcttaagcctgaagttaaaaataagatagcttggtacaatgtttcccaatgtttccagtaaaacttgtttacttaacttgtttggggattctttatttaacctgaacaaacttttcgaaacatgcagtatttctgtatacaagctgcttcccatcagtaataacctgttggcccagggcaattccttctcgaataaacttctctcacttgactctgctaaccattccttccttaaaacccttaattccttggcttccaggacaacacactctttttctgtcctccttcctttctagtcatttatctttaactacttcttctttttttttttttaacctaccccttacatatttgttttcctagagctccattcttgcctacttaataaatacttccttaccttgggtgacctcttttacatctataccttcacctaccatgtatatgtttatgaccccccaaaatttttgagatttagttacaaataaccaactgcgtaccagacatttggatctctcacacatacttcagagttcacatgtcaggtattatattcatcaatcttggccgggcacagtggctcacacctgtaatcccagcactttgggaggccaaggtgggcagatttcttgagctctggagtttgagaccacccagggcaagatggcaaaaccccatctctacaagaaatacaaaaattagacaggtaggttggcatctgcctgtagtcccagctacttgggggactgaggcaggggaatcgcttgagtccagggggttgaggctgcaatgagctgtgtttgtgccactgcagtccagcctgggtgacatagtgagaccctgtctcaaaaaaaaaaaaaaagactttaccaatctctgatctttattgacaaaatcctatcaattctgtctccagatctctcttgagtccttttttctttccattacatctccagcaaacctaatcattttccttggtaattacagtagccttcttcctgagctcgggtgtagaacccctaattcctctgttgcatcccttacactgctgctaattaatacttctagatctaaaatctaactggatcactcttctacttataatacggtagcccagtctccctctccagtctcatctcccaagatgctaccatttgctctccatttgctgccacataccaaataacttgcatttcccaaactcagccctgtctgctcacccactgacacccccatgccattgcacaggccattggtgtttaaaggatgctcttctctacctccccctgcatattttactctgacctgccacccccaaccccatacacacatgctgtcatgcagagcctaatctgtcttgtcatacctcgggcttcagctcaggtttcacctcctctgtaacatacccccatctcacttccatcaacaccaccaccacatgattttggtatcttgcaggcatctagcatagaactccctattctgcattatgactactggaccacttatctctctgccctacttgataagttccatgaggacaaagagtatgttttttcatttttctatctctagtactttgcacttactagatacaaagttaagtggagaatgaataaatgagtgaaagaatgactacatgagaaaggtgcatagtctcccaatgtagcaaaaagaaaatacagaggatgaatggatgtctaagtagagagatgatagccagaaaggcagataaatacatgcacacccaatagtgcacatttatgaagcttgtattgttacaaataataaaatagtatttctgtgtgtttcatgtactaaacattataaatgaatattgtgctattacagggtagcagtcaaatgcttaggaagccaagcagaggatatagaaaagtgaaacagctggagtaagcagtccccaagacaaaccgaaatacttccagggttaggtttagctggtgaacgaccgtttataacctctgcatcaatggatcctgtctttggacctgtttttctagtatagataatagatttatattgactcagtttgtttattcccaatccggacatagctttttgactatgagacaatcagtgcatgcaaattgtcccagttccttggcctcacttaatctctgcccagggcaaatacagataaatcacccatcatgatatttttatttattcatgataattttatttattcaaattctatttatgcaagtgtctgtattggaaactgttgagttcctgcctactgcataactttataactatgagcagaattctgaacagaattatcaaatttgccttttttttttttttgagacggggtctcaccatatcacccaggctggagtgcagtggcatgatctcagcttactgcagcctccacctcctgggctcaaacaagcctcccacctcagccttctcagtagctgggaccacaggtcctcaccaccacgcctggctaatttctgtgtttgtttgtatttttgatagagatgggttttgccatgttgcccaggctggtcttgaactcctgagctcaaggaatctgtctgcctcaccctcccaaagtgctgggattacaggtgtgagccaccatgcccagccggccttttcttcttaaaaaatgtgtgatcagaggatgcttaaaattacttttttctttcccattagccttggaattataattctatactaattactttatgtatattttgttctacagtattataaattgggaactgctaagcatatttattaaaacagtagaaggggatatgtattttaatatttcaaaatgagaaaaatttctcaaactttaccctgaaaaaagcatagcttttactacttaatattctgttgatatttacttggaatattatacttttttgttttgacaggacaatgtaactgcctactttaagcttgaatccatttgagagtcgaaatagctttaggccaggcatgtggctcatgccaataatcccagcactttgggaggccaaggcaagaggatcacctgagcccaggagtttgagaccagcctgggcagcatagggagactctgtctctacaaataattaaacaattagccgggtgtggtggcgtgcacctatgatcctagccactcaggaggctgagttgggcggatcgcctgagcccaagaggttgaggctgtcgggagccatgattgcgccactatgctccagcctgggcaacagagtgagagcccgtctcaaagaaacagagagagaggtggaagaggagaagagaagagaagggaaggaagggaaggagggaaggaaggtaggaggggaagaaagaaaaagaagaaatagctttatttcttaccctgatccaccctacatacagtcagcaagcaatatgggcatgcacttggcatttcaagggatggtctagtagataatggtattttctaaaacccctagatcttactttcactatgcccatactgtatctcccatgccagagttttcttttttcctgctgtaatctagatgtgttttctccacaggcatattttggtggtgaagctcgctgcgatgctgaggctggacggggagatgattatgatcccagagagctcatttgtggtgcctgttctgatgtttccagggctcaggtaggcagaacattttattagaaacaacagtttagaaatgttaaatagtattattttttaaacaataatatggtgggaatgtaaatcaggcttttctcctcagtattttctgatatttgttcagtgttgtataatttttcacattatttcatatatattatttcctttgggcgatatttttattttataattttgtcttccatgaaatgatcaccatagttaaacaatgtaataaattttcttattttcttagtttcttaaacgtgcagtgaaacttttaggttttcccctcttcagtgaaatgttcacagtacatctgtgagattataagagaggttaatttcattaattttcagtttgaccagccataaaacattcataaagaaattaagtaccttgcttattaaatcagatagattatgatggaaatggaattagaacttggatctttgtccagaccttttggacaggccacatatttatttgttagttttcaaacatgtgtatcatcagtttttcaatcaatttttagagttatgtaggccctcaaaatatgaattaaggagctgtctatgtatatgcctatcttaagtgtgattgaaatagtctcactatgatcttgtgtctgtctcttctcttttgctatagatgtgtcccaaacatggcacagactttttggaatataaatgtcgctactgctgttcagtggctgtttttttctgttttggaacaacacatttttgtaatgcttgtcatgatgattttcaaagaatgactagcattcctaaggaagaactaccacactgtcctgcaggtatgcttttaatattttaaaatcacgattatgatctatataccatagttttatgtaaacattatatgaaagctctgtttcaagtgacagaaactcaattcaaactagcctaaagaagcaggaggaattccttgatccttgtaagcttatgactttctgggtgaattaggaagcaggctcatcatcatgagtgagaagttagctgtgacggagcagatctcatggggagtgagaatgaagtctactaaggattggtgaaaagatggctgttcagcactaaaaagtcatatgaagttcaatattacagaaccatttaaaaggattctgtgatttttttttttttttaacgaagcctggaaccgttgccataaaatgaaccaagtgatttaatctagaatggctgggaatcagtagtgtagtgagcatcagaaagtcaagggaattaaagaaaccactcagaatacaggagaaattattgtctatggaaagcaggctgaggggaaagaagtatgaaaccaaaaagaagctgaaaatactggaagaggacaggaggctagtggcaacggtgaggtggaataccaggcttcacggaagtgagagaagtggaaggagaggtccgtgcaaccataggagagaattttacataagatatatctgaaattagtgaaaatacatgcacaagtacatacaacacaatataccgtgttgtcaaagctagaaaacacacactgccatctgctgcctatcagagtgcaagtgatagacctctccggaaaacagcttaatagtgcctctcaaaattaccagcgtatcaacccttcagcactccctcttcagggatatagctacaaatgtgaggacagtaaaaaagggcaaataaacaaggttattaactacagctttgctatgataataaaaatgtataaacaatgctagtgtccagtagggtagtggctaatattacgcatcagtttaaaaaaaaaaaagagtgaggatggtgtttctgtgtgctgatttggaaagatcttcaggagatagcatcagatgaaaaaagtaaattaggccaagtggtgtgactgacacctgtaatcccagcactatgggagaccgaagcaggtggatcacttgaggtcaggagtacgagaccagcctggccaacatggtgaaaccccatctctactaaatgtacaaaaattagccaggcatggtggcgggtacctgtaatccgagctactcacgaggctgaggccagagaatcacttgagcctgagaggcggaggttgcagtgagccaagattgcgccactgcactccagcctgggcgacagagctgtttcaaaaaaaaaaagcaaattgcagaacagagtatgcagtatgctgccttttgtaagagggaaaatgagactatctagtcatatttgtttatatttacataaagaaacccctggaaagatactcaagaaactaatactattgattaactattgggaaaagcaagaaaaagatgagggtagagactagggaaagttgcttccaattgtgttatcatttttaacattttgattttaaaccatgtgaaacatatactatccatttcaaaataaagataagtagggttttttcaagtatgactactttttaccatttgctcatgtcaaataaaaattgttttactgtttctcctcaaatactgtttttttttaatagaagtagcaggctttgaaagaagcaaggccagaatgatgtgaagccaggttatggaggataatggacagaacagagatgtgagccatgcttggagggagggggtgatgtgctggaggtcagcaagtgaagggagtgtggaaagggcagtattctagatgactctagatgacttgagcttggaggaagggcaattattgaatgaaagttgtaggttgtggtctggaaagaaataggagccaagagaaaaatcagtcacacgttgtagccatatgacctggaggagcttgggagataaccagcctccagttgagaaagatttaaggcaagcagtatcttcaggggaaatctagattttacttaaagtttcctctatgttaaaaaacagaaataagaatgctaaaaataaggaagttaacaatgaaacactttagagaactcagtggtgggtagtagctgagagaggagggtcaggtttcctcacaggatcagggcagccaacacaaaggaatcacaggagggaggcgggggaggagccagagttggcagaaggaaggaatgacctcggtcctgggcattttcacacatatttgctccatttttacaatctgcgagtagatggtattatccctatgttatcaaatggaaaattgagggctagggaggttaatgaactggcctaagaacatacactgagattgaggtatactctcccagcctcacacatccctctaactcatccattcatttagagaactggaatagaataaaggagatcccacagggatgtgagtagggaagatggccatatacagtttacttagaaggacagaagaagcaattctctaaccactctcaaccctgttaattctaatatttttagataatcagtcttcatccattcaagctgctataacaaaatactgtaaactgggtagcttataaacagtggaaatttattctcacagttctggagaccaggaagtctaagatctaggaattagcagtcgaggtgtctggtgaggacccactctcagcctcataggtggcactttcttgctgtcgtggaagggcagggggcctctcctgggtctcattgataggaagagcactaatcccattcatggattccaccccatgacctcatcacctcccacagctccacctcctaacaccataacattggtgttaatgttgtagtctcagcagtgcaccaagatataacagtctctcattgtctgagataatgccaggagttctttgtcctacctccaagaagattaaggagcacagatacaaaggtgaggttagagcgaaagtttaataagcaaaagaagaaagctctctgccagcagagaggggggcccaaacaggatgctcccgtgaggctggggcccagggtttttatggactggaaaggggaaggaatgtgcttagtctgtgggctgtcttggcccgcagcgtgactcagcttggcccgggaccctggcccaggaacccactggagcccactgtgcctatgcccacaaaaggagagagccccctgactatacaaaggacaaaggcatttctatcccaggtcttgtcctttatctgattgaaggtttttctgtctgtgcagccgtgggcatgtctttaggcacaatgccctgtgctagttccctcatcggtgcctgcagcttgactttttttccccaactgctttttatgttatatggggatgaggcactgacctgtggacctggggctctctggggacccttcccttgctatctacctaaggcaaactaactcctttcattaacacgtcagtatataaatttggaggagcacaaaaacatccagaccatagcataatccctctatccatttctttctaaaatatttattgactaccttctcgtgccaggatacttaatcttcacagcaccccatgagttaggtattgctgtgtctattttaaagatgaggtatctaagaatcagaaaaaataattttacttcagtttcatatctacaaaatgaggataatcgtttctgcctcctatgactgttaaatggttaagtagctgatatactagaacagtgcctcagtagtaagcactcagatgtttgcaggtgtgttttcactatttttatttctcaccaccatgttggattgtgaacaccttgaagatgggagccatgcacttttcatctttaatcccaagtacctaagaatgcccttcatatattaggcatgcggtaaatatttgttgagtaaatgactcattttttccttgaaaaaacccaagggctgcagtttgtgtcaccaccattgatatgttgtgtatctgaactagatctgccattgagtgtccccatcctgcgcactctcccccacccactggtggggtgggcaccaatctgcagtgtgatttggagcaggagctactgaactactttctaacaggaataattaactttctagcaaacctctggcatcctcccatgctgacccatcctgaggtctgtgcctgcatttgtctgaggtggcactcattgccactctcatgcctgttagctctaacctgaagttgacttctctgagattagctgtcttcagtttgacactctaatgtcttggtcttactcttgtacagaacttactttttattcctctagatcaggcctcagtttgtggttgttcagtaatagtagtaatagccaacatttattgggtattatactgtgcagtctaagagctttataaatgctagccttttatccatcatggtttccttctcttatgtcagaaatgtttttaaacctagaaaaggtaaagtaaatgagacgtcattcctcattgatcagtgtaagctcattccatccaatctcgtgatatttttctctcaagcacaatcagaatttagatttattattttgaaaactagctgtagaaaactaatgcagagttcctattccttcatctactattaaatagggactctgctttcaagtataagcctagcgtgtttttctgacaaagtaagcctcctggtttctaaaggaactaaaaatcttttctaaatgatatgaaacccatgacgtgaagcacattaatataataattttcttttttaaaatactgtttttggtacatttgataggcctactttgattattatcaacaaattcatatcttccccaaagtgttttttccacattgtattttataacattgttcatgtttttaacctgataactttcattttcctgctaatatccttcaggtacttttaaaaattacaggaatcattggccttcccattttaccagtgtgggctttttcatcagtaaaaataaaagggaaaacatggcttctttaaacttaaatgtttttacttatagaaaaacattctacacaaaaatctccctgttttctattcagaaaaataatgttctgccagactaaattgaggggttttgtgaccttttttgttcattactctgttttaaaatgatatatctgatgcatttattaatagaaaaagatctgtagctcgaagtgatcctctctaggcaggatatagtgagtaagggaccccatcacttcttccattgtacatatctgtgttgaattttttatagtgaacattttttacttttgtgatcagaagaaaaatttctaaatgttatatttgtataaccatagtcatgttatttctttgaataacctatcagtgatttatatttctgtgaggtttatatgtgtgtattcttaaaacattattttcctttgcctccttcaaatttatacaggtcccaaaggcaagcagttagaaggaactgaatgtccactccatgttgttcatccacccactggggaagagtttgctctgggatgtggagtgtgcagaaatgcccacactttttagaacacgcagatcctttgtctacagagagaaaaattgccttcatcccccaagaggatgcggtgaagtttaaactctgctcaggataaggacgggaccatttttacatccatgaaaatgaaccattcacagtgcaagaaggataccaaataccatgtacataattcttgctatgaaaagtttccccattattttggtttatcttcttttgaacaaatgacatcaaacttgtgaggtgtttgcatgtggccattaccgtcattggcctgtgaagcattggacatttatagataattgatataaaagaatcgccatgcccatggactaagaacgatgctggctttcaagcaaaaaagaaaaataatcattgtttattgtatactgcctttttgtaatcctgtacaattgcatcacgggtggggataaaaagaggaatattctggtttatttcctagactgttatttaaaaaaaaaaaaaacattgtgttaggacagcatataaatgtaataagtatcacactgtatataaacatatcaatgtttgtcctgtataagaattactaaattacaaatgcaatttcatttaaacttctaggttaagtttgagcctgaaattttaatgaagtgcaatactgagtgtgcctcattatcttgcagctgtaaacatattggaatgtacatgtcaataaaaccactgtacatttttatacagtgataaagtctaccactgtgggaggtattgtttaaaaaacaaaatttgaacacctttaaggtctaaaagtccagttttcctcagaaagaaatttactaacacaacacattcataattttcaaaactgttagagaaaataagaatagtaatgaagtgcaatgttggaatcttaaaccctaagcagaagatcataaatatatatatatatattcgtaggtaaatatattcataccaaagacagaagaaagcatttaggaaattaacttctattttaactagcagcatttttaactttatttatttatttatgagacagggtcttacactgtcacccaggctggagtacagcggcgtgatctcagctcactgcagcctccaactgggctcaagcgatcctccagcctcagccccctgagtagttgggactacaggcatgcaccagcaccatgcccagctaattttgtaatttttgtagagatggggttttgccatgttgccgaggctggtctcaaactcctagactcaagcaatccccactgctttggcctcccaaagtgctaggattacaggcgtgagccattgcacccagcccctttttgaagttcatataaagtaataaaatcactaattttttattcattttctatagacaacggtgtgtttaggtacatctttttgacactcgagtcatgaaattgccaaattattgattgttgactcatcatacatcagattagcaaaacttttgttgttgttgttgtatttgagacagtctcactctgctgcccacactggagtgcagtggcaccatcttggctcactgcaacctccacctcccaggttcaagcgattctcctgcctcagcctcccaaggagctgggactacaggcgtgtgccacctcacccagctaatttttgtatttttagtagggatggggtttcaccaggctggtcttgaactcctgacctcaggtgatccgctcgcctccggcctcccaaaattccggtattacaggcgtgagccaccgcgcctggccaagactagcaaaagttttaatcacattttgttctaaattccaactgtgggaaatatagaacttaatttgtcaatatagaattactgacaaatttaatgacatttgtgttaagcatttatgagaaatttaaaacaatctttgccttgaaggaacttaaaaaataaagatcagatctgagcacaggtaactattttacagaatgttctaataagtttcataaaaatatccaagccaaatgcttcaggatttaggaaaaatcattattgttttgtcgttgggagtgtagctaagaggccatttaagctgccttttgaagaataagtagcatttcaaggagcagagatgtggagaaaggcatgagaaatacctaagtaggaaaatctagaatatgatcaaagaacagtaagtatgagtgttcaagcttaggattgactgaaaatgtttccagaaaacacaaagtacgtagagagactaaagctgttacaagaaaactggaggggagaatgcagtgaggacactgtcagtatatttgacttgacactgtcagtatatttgacttaaattcacaggccacaggaatgaaactaaccttgagaatgccacacagataagagactttatggacacttaatagctgcgaagaataccatcttgcgtcagttaactttgctggcaaactggtcagctcagtttgtaaatcgtagaggaaaagaaacaaactattgagatggcttggctaaaaaagccatctcaagttatgaaagggataaaatcaacaaagctgtgtagtgaatactcacaatgacttcaacttatcttcatttatgaggaagtcacttttagtcaggatctctagcacctcaaaaccagagaacataaacatcttagcaataaattatgacaaagcacaggaaggagtggctttggcaggcatttttagctcaagcatcctacacacaaggaccatccttcaaacactgcatgtaacatccaaggcaggagacagaaagcagaatcacagtggtagatgtcagtgaatcttcctggaaagaaattgtttttccgttatattttcaagcctaggaaaagccatttttcttagtattatggctagactgtttaggcctaaaatctccctattttatcaataagcttatttggttttggtttttggttttttgagttttttgggtttttttttttttttttaagaaagggtctcactctgtcgccccagctggagtgcagtggcgttatctcggctcgcagcaacctctgccttttgggctcaagtgatcctcccacctcagccccaagtagctgggagtacaggcacgagccaccatgcccagctaatttttgtaattgaaacagggtttcaccatgttggccaggctggtctcaaactcctgagctgaagcgatctacctgcctcagctaggattacaggtgtgagccaccgcacccaacctcaataagcttatttgataaaatatatgcaatgctccctttattcacttttcattcagaatgtttagtaatttgtattgtttttcagattttcagcccaatatatctccctgcccactgtgtcactgtattctacctatacatcatcacgtgtttctgctattggctgtatgatggaacactgcggctcattttcctgaaaactgccgatagtgcatagagtgctgggatggaaaccagaagctttgaattcaagccttggttctgccttgtttttgcttgggtggccttgagtcagccacataccttttaaaatctcaatttattagaaattattccaaatcaaaatcaaatgagaaggtatatacaaaagtgctttatcccacaataaactattcaagagagagcaaaggagaggacatttactcaacacctcctaaaaggcagccagtgaaattaggcattttatttaatcctcctggcaactctgagagtaaagcattattaatcccattttggctgtttaaagaaattatttgcactagattccagctgtagtttagcttcagaaaaaaaaatcctgagatgtgaattcacagctttctgggtttaaagcccaagctctatcacatcatgctattattgttacattactgctagttctatgaaaagaaatactaatttatgaaatacatcttatccaaaatggttgggaccagaagtgtttctgatttcgggttttttcaggtttgggaatatttgcattaccagttgagcatcccaaatctgaaatccaaaatgctccagtgagcattttatttgggtatcaggcatgtcagtactcaaagttttaaatttcaggacacttatcgtatggccaaggttgcacaggggacaagacagcatatgcctccaagagtgttcagtgtcgtcaggaggtacctccacccagatttcccttcaggggaggccccacctcaaggagtccagttagctgacagcctccagctttaatcacccccacatatgacccaatgtcacactcctccagggcagccccagcctgtgatagagttcagatggggttctcgggcctggccatttctgcccaagcggcactcctgcgtgcgcggtcttgcgtaagaactcaggccgactgagactgtcttacagctgccctgcagtcggaggcttttcctacccaatctttccttctctcttctttcctagatgccagacctgcatcacagtctaatggctctccctgcccactcctgctccctcatcactttatctttcatagggattacccccaacagattttctgcacttctaacttaatcttggcatctgcttcctagaggacctgcctgacacaggagggtaatacattttaaatgacttacttatggcttcatttctcaaatgtcttagggtggcttagaaaaaa

SEQ ID NO: 4 - DNA, 739 bp, Rattus norvegicus
gcttgccaaacgattcaggtgccaacaatactaaattcactacagagaagtgtacaggcagtgttggttggaaagattcaagtccaggactggtttagcaatggcattaagaaagcagctttaatgcacaagtggccattaaaggaaatatctgttgatgaagatgaccagtgtctgcttcagaatgatggattttttctttatttgttatgcaaggatggattatacaaaataggctctggatacagtggaacagttaggggtcatatatataattctacatctcgtatcagaaacagaaaagaaaagaagtcttggttaggatatgcccagggttacttgttatatcgggatgtgaataaccacagtatgacagccataagaataagccccgaaacgctggagcaagacggcactgtaatgttaccagcttgcagatgttgcttgacacattcctgaagctgctgtcttataatgtcatacacgggtaaagagcggacacgggaagttgtcagtgtgacgccgaaggaagacccagccctcagccccagccctcaagcctgaagaagcctgctgtcatttcatcacttctttgacacggactacagtttcttcaataaaatcatttgctttttccctgacaagggatactacttctctgtagcttttcatcaagaagagaagtagtaaactgtcagatttataggtggttactgaataccatgtttaaaatgaacat

SEQ ID NO: 5 - DNA, 473 bp, Rattus norvegicus
gcagcttcagagtgacagaggaactgtctcaacatcttcaagaccagtgtctacatcagcaaagtcagagctgccctccaagaacagcagatcagttaaacctgatgggcgtgtgagccggactactgctgaccagaagaagccacggggcacagaaggcttatctgctagtgaatccctcatgttaaaatctgatgctgcaaagttgaggtcagactcccatagtaggtcactgtcccctaaccataacactttgcagacactgaagtctgatggaagggtatcttctagcttcagggctgaatccccaggaccaggctctaggtcatcctctcctaagcccaagactctgccgactcccaggtctagcccatctggtgctagctctccacgctcctcctcaccgcaggataaaaatctacctcagaaaagcacagctcctgctaagacaaaacttgacccacctcgggagc

SEQ ID NO: 6 - DNA, 708 bp, Rattus norvegicus
  misc_feature (615)..(615): n is a, c, g, or t
ttttttttttttttttttcctttttcgtttttttattctctttctcacattctttctttttaaggactgcacaggaacctggacttggaaaaatcatattctgggaagcagctttgattgtagccaaagagatgtcctcccagaaggccactaagtgttgtaatgttaaggggagcggagacctagacttcactgagtgatgcatggacatttcaaaagtggcttccgattttccgtcttcacacttctcatgtagaggtggctccttaagcatagacaggaccttatttttgttgatacttcccatcttggatatgtctggtccgtgaggtgaaatgttgaacatatttagggcagatgatatttccaacgaatgtctattttttaacttgatttccttttcctctgtgggtggctggctattcaaactacttcttatgggagcatgttctttggaaagttcaggattaaacttcaggaaggaagagcaggccattgcatcatgtactatgccttcatgccacagaaaagaggcaaagacagctcgggcacactcagccacggacggggacatggcttgcttggctggctctaaagatttggctccatctcctnttgaaagaaagttagacttttcttttttgggcctggtgtgtctattagcacatttccgacttattttggggtgatgctctcatttcctgttca

SEQ ID NO: 7 - DNA, 529 bp, Rattus norvegicus
taagcttgctctgtccctgccccgtacatctcagtaactcctgatgcaagtcccaatgtctttgaagagccggagagcaatatgaagtcgatgccaccaagtttggaaacgagcccgataactgacaccgacctggctaagagaactgtcttccagaggtcatactcagttgtcgcttcggaatatgataaacaacactccattttacctgcacgagttaaagccatccctagaaggagagtgaacagtggagacacggaagttgggtcttctctcttgcgacatccgtcaccggagctttcccggcttatatcagcccacagctctctctccaaaggagagcgaaacttccagtggccagtcttagctttcgtcatacagcatcatgatttagaagggctggaaatcgcaatgaagcaggccttaaggaagtcggcttgccgtgtgtttgctatggaggcattcaactggcttctctgtaatgtcatccaaacaacgtctctgcatgacattctctggcactttgtgg

SEQ ID NO: 8 - DNA, 667 bp, Rattus norvegicus
acgaagggctgcataagagtgaagcgatcacgacgccgggcgtcaggttttacaatgatccagccggctatgccatgaacagatacgcatattatgtttgctacaaatgcagaaaggcatattttggtggtgaagctcgctgtgatgctgaggctggacaaggagacgactacgaccccagagagctcatctgtggagcctgttctgatgtgtctagggctcagatgtgtcccaaacatggaacagactttctagaatacaaatgtcgctactgctgttcagtggctgtcttcttctgttttggaacaacacatttctgcaatgcttgtcatgatgattttcaaagaatgaccagcattcctaaggaagagctcccacactgtcctgcaggtcccaaaggcaaacagctagaaggaactgaatgtccactccatgttgttcatccgcccacgggggaagagtttgctcttggttgtggagtgtgcagaaatgctcacacgttttagaactttcagatcctttgtctacaaagaggatagttgccttcatcccctgggaggatgcagtgaaactttaaactctgctcaaggataaggaacggggaccatttttacattctgaaaacgaaccattttccagtgccaggaagtgatgccccaaatacctg

SEQ ID NO: 9 - DNA, 652 bp, Rattus norvegicus
  misc_feature (605)..(605): n is a, c, g, or t
ttttttttttttttttttgacgctctcactgtgtagagcacagtggtttactgacgtacattccaatatgctgacagctgcaaggtagtgaggcacacccagtattgcactgcattagagcttcaggctcaaacgtaacctacaggtttaaatgagactgctgtgtcatttagtaattctcatacaggacaaacactgatagctttatatacagtctgatacttattacaattataggccttactaacacaatttttttttttaaataacagtctaggaaagaaaccagaatattcctctttttatacccacccgtgatgcaattgtacaggattacaaaaaggcagtatacaataaacagtgattatttttctttttttgcttgaaagccagcatcattcttagtccatgagtatggagatccttttatatcaattatctataaatgtccaatgctccacaggccagtgacggtaatggccacatgcaaacacctcacaagtttgatgtcttggttcaaagatgataaaccaaaacaatggggaaacgttcgtagcaagaattatgtacacagtatttggcatcactcctgcactggaaatggntcgttttcagaatgtaaaatggtcccgttcttatcctgagcagagtt

SEQ ID NO: 10 - DNA, 18 bp, Articifial Sequence
gaagggtggggccaaaag

SEQ ID NO: 11 - DNA, 20 bp, Articifial Sequence
ggatgcagggatgatgttct

SEQ ID NO: 12 - DNA, 24 bp, Articifial Sequence
ggtggtgaagctcgctgtgatgct

SEQ ID NO: 13 - DNA, 24 bp, Articifial Sequence
cgtgtgagcatttctgcacactcc

SEQ ID NO: 14 - DNA, 18 bp, Articifial Sequence
gactggtttagcaatggc

SEQ ID NO: 15 - DNA, 18 bp, Articifial Sequence
gccattgctaaaccagtc

SEQ ID NO: 16 - DNA, 18 bp, Articifial Sequence
gcaattgctaaatcagta

SEQ ID NO: 17 - protein, 1010 aa, Homo sapiens
NNHSMTAIRISPETLEQDGTVMLPDCHTEGQNILFTDGEYINQIAASRDDGFVVRIFATSTEPVLQQELQLKLARKCLHACRISLFDLEKDLHIISTGFDEESAILGAGREFALMKTANGKIYYTGKYQSLGIKQGGPSAGKWVELPITKSPKIVHFSVGHDGSHALLVAEDGSIFFTGSASKGEDGESIKSRRQSKPYKPKKIIKMEGKIVVYTACNNGSSSVISKDGELYMFGKDAIYSDSSSLVTDLKGHFVTQVAMGKAHTCVLMKNGEVWTFGVNNKGQCGRDTGAMNQGGKGFGVENMATAMDEDLEEELDEKDEKSMMCPPGMHKWKLEQCMVCTVCGDCTGYGASCVSSGRPDRVPGGICGCGSGESGCAVCGCCKACARELDGQEARQRGILDAVKEMIPLDLLLAVPVPGVNIEEHLQLRQEEKRQRVIRRHRLEEGRGPLVFAGPIFMNHREQALARLRSHPAHVKHKRDKHKDGSGERGEKDASKITTYPPGSVRFDCELRAVQVSCGFHHSVVLMENGDVYTFGYGQHGQLGHGDVNSRGCPTLVQALPGPSTQVTAGSNHTAVLLMDGQVFTFGSFSKGQLGRPILDVPYWNAKPAPMPNIGSKYGRKATWIGASGDQTFLRIDEALINSHVLATSEIFASKHIIGLVPASISEPPPFKCLLINKVDGSCKTFNDSEQEDLQGFGVCLDPVYDVIWRFRPNTRELWCYNAVVADARLPSAADMQSRCSILSPELALPTGSRALTTRSHAALHILGCLDTLAAMQDLKMGVASTEEETQAVMKVYSKEDYSVVNRFESHGGGWGYSAHSVEAIRFSADTDILLGGLGLFGGRGEYTAKIKLFELGPDGGDHETDGDLLAETDVLAYDCAAREKYAMMFDEPVLLQAGWWYVAWARVSGPSSDCGSHGQASITTDDGVVFQFKSSKKSNNGTDVNAGQIPQLLYRLPTSDGSASKGKQQTSEPVHILKRSFARTVSVECFESLLSILHWSWTTLVLGV

SEQ ID NO: 18 - protein, 617 aa, Homo sapiens
IFATSTEPVLQQELQLKLARKCLHACRISLFDLEKDLHIISTGFDEESAILGAGREFALMKTANGKIYYTGKYQSLGIKQGGPSAGKWVELPITKSPKIVHFSVGHDGSHALLVAEDGSIFFTGSASKGEDGESIKSRRQSKPYKPKKIIKMEGKIVVYTACNNGSSSVISKDGELYMFGKDAIYSDSSSLVTDLKGHFVTQVAMGKAHTCVLMKNGEVWTFGVNNKGQCGRDTGAMNQGGKGFGVENMATAMDEDLEEELDEKDEKSMMCPPGMHKWKLEQCMVCTVCGDCTGYGASCVSSGRPDRVPGGICGCGSGESGCAVCGCCKACARELDGQEARQRGILDAVKEMIPLDLLLAVPVPGVNIEEHLQLRQEEKRQRVIRRHRLEEGRGPLVFAGPIFMNHREQALARLRSHPAHVKHKRDKHKDGSGERGEKDASKITTYPPGSVRFDCELRAVQVSCGFHHSVVLMENGDVYTFGYGQHGQLGHGDVNSRGCPTLVQALPGPSTQVTAGSNHTAVLLMDGQVFTFGSFSKGQLGRPILDVPYWNAKPAPMPNIGSKYGRKATWIGASGDQTFLRIDEALINSHVLATSEIFASKHIIGLVPASISEPPPF

SEQ ID NO: 19 - protein, 567 aa, Homo sapiens
GREFALMKTANGKIYYTGKYQSLGIKQGGPSAGKWVELPITKSPKIVHFSVGHDGSHALLVAEDGSIFFTGSASKGEDGESIKSRRQSKPYKPKKIIKMEGKIVVYTACNNGSSSVISKDGELYMFGKDAIYSDSSSLVTDLKGHFVTQVAMGKAHTCVLMKNGEVWTFGVNNKGQCGRDTGAMNQGGKGFGVENMATAMDEDLEEELDEKDEKSMMCPPGMHKWKLEQCMVCTVCGDCTGYGASCVSSGRPDRVPGGICGCGSGESGCAVCGCCKACARELDGQEARQRGILDAVKEMIPLDLLLAVPVPGVNIEEHLQLRQEEKRQRVIRRHRLEEGRGPLVFAGPIFMNHREQALARLRSHPAHVKHKRDKHKDGSGERGEKDASKITTYPPGSVRFDCELRAVQVSCGFHHSVVLMENGDVYTFGYGQHGQLGHGDVNSRGCPTLVQALPGPSTQVTAGSNHTAVLLMDGQVFTFGSFSKGQLGRPILDVPYWNAKPAPMPNIGSKYGRKATWIGASGDQTFLRIDEALINSHVLATSEIFASKHIIGLVPASISEPPPFKCL

SEQ ID NO: 20 - protein, 301 aa, Homo sapiens
APMPNIGSKYGRKATWIGASGDQTFLRIDEALINSHVLATSEIFASKHIIGLVPASISEPPPFKCLLINKVDGSCKTFNDSEQEDLQGFGVCLDPVYDVIWRFRPNTRELWCYNAVVADARLPSAADMQSRCSILSPELALPTGSRALTTRSHAALHILGCLDTLAAMQDLKMGVASTEEETQAVMKVYSKEDYSVVNRFESHGGGWGYSAHSVEAIRFSADTDILLGGLGLFGGRGEYTAKIKLFELGPDGGDHETDGDLLAETDVLAYDCAAREKYAMMFDEPVLLQAGWWYVAWARVS

SEQ ID NO: 21 - protein, 101 aa, Homo sapiens
APMPNIGSKYGRKATWIGASGDQTFLRIDEALINSHVLATSEIFASKHIIGLVPASISEPPPFKCLLINKVDGSCKTFNDSEQEDLQGFGVCLDPVYDVIW

SEQ ID NO: 22 - protein, 204 aa, Homo sapiens
DEALINSHVLATSEIFASKHIIGLVPASISEPPPFKCLLINKVDGSCKTFNDSEQEDLQGFGVCLDPVYDVIWRFRPNTRELWCYNAVVADARLPSAADMQSRCSILSPELALPTGSRALTTRSHAALHILGCLDTLAAMQDLKMGVASTEEETQAVMKVYSKEDYSVVNRFESHGGGWGYSAHSVEAIRFSADTDILLGGLGL

SEQ ID NO: 23 - protein, 38 aa, Homo sapiens
DEALINSHVLATSEIFASKHIIGLVPASISEPPPFKCL

SEQ ID NO: 24 - DNA, 3030 bp, Homo sapiens
aataaccacagcatgacagccataaggataagccctgaaacactggagcaagatggtactgtgatgttaccagattgccacactgaaggtcaaaatattttattcactgatggagaatatattaatcagatagctgcttcaagagatgatggctttgttgtcagaatatttgccacaagcactgaacctgttctacagcaagaattgcaacttaaactggctagaaaatgcttacatgcctgtcgtatctcattattcgatctggaaaaggacttgcatattataagtacaggatttgatgaggagtcagcaattcttggtgcaggacgagagtttgcgctaatgaaaacagcaaatggaaagatatattacactggcaaataccagagtcttggaatcaaacaaggtggtccttcagcaggaaaatgggttgagctaccaattacaaaatctccaaagatagtacacttctcagttggacacgatggctctcacgcccttttagttgcagaagatgggagcatattctttacaggatctgctagtaaaggagaagatggagaatcaattaagagcagacggcaatccaaaccttataaacctaaaaagataattaagatggaaggaaagattgtggtatatacagcctgcaataatggaagtagttctgttatttctaaagatggagaactctacatgtttggaaaagatgccatttactctgatagttcaagtttggtaactgatttgaagggccattttgtaactcaggtagctatgggcaaagctcacacttgtgttttaatgaagaatggagaggtgtggacatttggtgtaaataataaaggacagtgtggacgagatactggtgccatgaaccaaggtgggaaagggtttggagttgaaaatatggcaacagcaatggatgaagacctggaagaagaactagatgaaaaagatgagaagtctatgatgtgccctccaggcatgcacaaatggaagctggagcagtgcatggtttgcactgtctgtggagactgtacaggttatggagccagctgtgtcagtagtggacggccagacagagtccccggagggatctgtggttgtggttccggagaatctggttgtgctgtgtgtggatgttgcaaggcctgtgcaagagagttagatggtcaagaggcaagacaaagaggaattcttgatgcagtgaaagaaatgatacctttagatcttcttttagctgtcccagtgcccggggttaacattgaagaacaccttcagttacgacaagaagaaaaacggcaacgtgtaatcagaaggcacagattagaggaaggaagaggcccccttgtatttgctggtcctatttttatgaaccatcgagaacaggctctagccagactcagatcccatccagcacacgtaaagcataaacgggacaagcacaaagatggaagtggagaaagaggcgaaaaggatgcaagcaaaatcacaacataccctccaggctctgtgcgatttgactgtgagctccgggcagtccaagtcagctgtggatttcaccattcagtggttttaatggaaaatggagatgtctatacatttggttatgggcagcatgggcagctaggacatggagatgtcaactccaggggatgtcccactcttgttcaagcattgccaggccctagcacacaagtcactgcaggcagcaaccatacggcagtacttttaatggatggacaggtcttcacatttggaagtttttctaaaggacaactgggcagaccaattttggatgtgccatattggaatgcaaagccagctcccatgcctaacattggatcaaaatatggaagaaaagctacttggataggtgcaagtggggaccaaacttttttacgaattgatgaagcacttattaattctcatgtacttgctacatcagaaatttttgccagtaaacacataataggcttggtacctgcttctatatcagaacctcctccatttaaatgccttctgataaataaagtggatgggagttgtaaaacttttaatgactcagaacaagaggatctgcaaggatttggtgtgtgtcttgatcctgtatatgatgtaatttggaggtttcgaccaaatactagagagctgtggtgttacaatgcggtggttgctgatgccaggcttccctctgcagcagacatgcagtccagatgtagtatcctaagtcctgaacttgccttaccaacaggatcaagggccctcactacccgatctcatgcagctttgcacattttaggttgtcttgataccttggcagctatgcaggacttaaaaatgggtgttgcaagtacagaggaagagactcaagcagtaatgaaggtttattctaaagaagattatagtgtggtaaacaggtttgaaagtcatggaggaggctggggttattctgcccattcagtagaagctatacgtttcagtgccgacactgatattttacttggtggtcttggtctgtttggaggtagaggagaatatactgctaaaattaagctgtttgaattgggtcctgatggaggagatcatgaaactgatggtgaccttcttgcagagactgatgtattggcttatgactgtgctgctagagaaaaatatgcaatgatgtttgatgagcctgttctcctgcaagctgggtggtggtatgtggcatgggcccgagtgtcaggacccagcagtgactgtggatctcatggacaggcatctattaccacagatgatggggttgttttccagttcaagagttcaaagaaatcaaataatggtacagatgttaatgcgggtcagatacctcagttattatacagacttccaaccagtgatggcagtgcttcaaaaggcaaacagcaaaccagtgaacctgtacacattttaaagaggtcttttgcaagaactgtctcagtggaatgttttgagtcattgttgagtattcttcactggagctggaccaccttagtcttaggagtt

SEQ ID NO: 25 - DNA, 1851 bp, Homo sapiens
atatttgccacaagcactgaacctgttctacagcaagaattgcaacttaaactggctagaaaatgcttacatgcctgtcgtatctcattattcgatctggaaaaggacttgcatattataagtacaggatttgatgaggagtcagcaattcttggtgcaggacgagagtttgcgctaatgaaaacagcaaatggaaagatatattacactggcaaataccagagtcttggaatcaaacaaggtggtccttcagcaggaaaatgggttgagctaccaattacaaaatctccaaagatagtacacttctcagttggacacgatggctctcacgcccttttagttgcagaagatgggagcatattctttacaggatctgctagtaaaggagaagatggagaatcaattaagagcagacggcaatccaaaccttataaacctaaaaagataattaagatggaaggaaagattgtggtatatacagcctgcaataatggaagtagttctgttatttctaaagatggagaactctacatgtttggaaaagatgccatttactctgatagttcaagtttggtaactgatttgaagggccattttgtaactcaggtagctatgggcaaagctcacacttgtgttttaatgaagaatggagaggtgtggacatttggtgtaaataataaaggacagtgtggacgagatactggtgccatgaaccaaggtgggaaagggtttggagttgaaaatatggcaacagcaatggatgaagacctggaagaagaactagatgaaaaagatgagaagtctatgatgtgccctccaggcatgcacaaatggaagctggagcagtgcatggtttgcactgtctgtggagactgtacaggttatggagccagctgtgtcagtagtggacggccagacagagtccccggagggatctgtggttgtggttccggagaatctggttgtgctgtgtgtggatgttgcaaggcctgtgcaagagagttagatggtcaagaggcaagacaaagaggaattcttgatgcagtgaaagaaatgatacctttagatcttcttttagctgtcccagtgcccggggttaacattgaagaacaccttcagttacgacaagaagaaaaacggcaacgtgtaatcagaaggcacagattagaggaaggaagaggcccccttgtatttgctggtcctatttttatgaaccatcgagaacaggctctagccagactcagatcccatccagcacacgtaaagcataaacgggacaagcacaaagatggaagtggagaaagaggcgaaaaggatgcaagcaaaatcacaacataccctccaggctctgtgcgatttgactgtgagctccgggcagtccaagtcagctgtggatttcaccattcagtggttttaatggaaaatggagatgtctatacatttggttatgggcagcatgggcagctaggacatggagatgtcaactccaggggatgtcccactcttgttcaagcattgccaggccctagcacacaagtcactgcaggcagcaaccatacggcagtacttttaatggatggacaggtcttcacatttggaagtttttctaaaggacaactgggcagaccaattttggatgtgccatattggaatgcaaagccagctcccatgcctaacattggatcaaaatatggaagaaaagctacttggataggtgcaagtggggaccaaacttttttacgaattgatgaagcacttattaattctcatgtacttgctacatcagaaatttttgccagtaaacacataataggcttggtacctgcttctatatcagaacctcctccattt

SEQ ID NO: 26 - DNA, 1701 bp, Homo sapiens
ggacgagagtttgcgctaatgaaaacagcaaatggaaagatatattacactggcaaataccagagtcttggaatcaaacaaggtggtccttcagcaggaaaatgggttgagctaccaattacaaaatctccaaagatagtacacttctcagttggacacgatggctctcacgcccttttagttgcagaagatgggagcatattctttacaggatctgctagtaaaggagaagatggagaatcaattaagagcagacggcaatccaaaccttataaacctaaaaagataattaagatggaaggaaagattgtggtatatacagcctgcaataatggaagtagttctgttatttctaaagatggagaactctacatgtttggaaaagatgccatttactctgatagttcaagtttggtaactgatttgaagggccattttgtaactcaggtagctatgggcaaagctcacacttgtgttttaatgaagaatggagaggtgtggacatttggtgtaaataataaaggacagtgtggacgagatactggtgccatgaaccaaggtgggaaagggtttggagttgaaaatatggcaacagcaatggatgaagacctggaagaagaactagatgaaaaagatgagaagtctatgatgtgccctccaggcatgcacaaatggaagctggagcagtgcatggtttgcactgtctgtggagactgtacaggttatggagccagctgtgtcagtagtggacggccagacagagtccccggagggatctgtggttgtggttccggagaatctggttgtgctgtgtgtggatgttgcaaggcctgtgcaagagagttagatggtcaagaggcaagacaaagaggaattcttgatgcagtgaaagaaatgatacctttagatcttcttttagctgtcccagtgcccggggttaacattgaagaacaccttcagttacgacaagaagaaaaacggcaacgtgtaatcagaaggcacagattagaggaaggaagaggcccccttgtatttgctggtcctatttttatgaaccatcgagaacaggctctagccagactcagatcccatccagcacacgtaaagcataaacgggacaagcacaaagatggaagtggagaaagaggcgaaaaggatgcaagcaaaatcacaacataccctccaggctctgtgcgatttgactgtgagctccgggcagtccaagtcagctgtggatttcaccattcagtggttttaatggaaaatggagatgtctatacatttggttatgggcagcatgggcagctaggacatggagatgtcaactccaggggatgtcccactcttgttcaagcattgccaggccctagcacacaagtcactgcaggcagcaaccatacggcagtacttttaatggatggacaggtcttcacatttggaagtttttctaaaggacaactgggcagaccaattttggatgtgccatattggaatgcaaagccagctcccatgcctaacattggatcaaaatatggaagaaaagctacttggataggtgcaagtggggaccaaacttttttacgaattgatgaagcacttattaattctcatgtacttgctacatcagaaatttttgccagtaaacacataataggcttggtacctgcttctatatcagaacctcctccatttaaatgcctt

SEQ ID NO: 27 - DNA, 903 bp, Homo sapiens
gctcccatgcctaacattggatcaaaatatggaagaaaagctacttggataggtgcaagtggggaccaaacttttttacgaattgatgaagcacttattaattctcatgtacttgctacatcagaaatttttgccagtaaacacataataggcttggtacctgcttctatatcagaacctcctccatttaaatgccttctgataaataaagtggatgggagttgtaaaacttttaatgactcagaacaagaggatctgcaaggatttggtgtgtgtcttgatcctgtatatgatgtaatttggaggtttcgaccaaatactagagagctgtggtgttacaatgcggtggttgctgatgccaggcttccctctgcagcagacatgcagtccagatgtagtatcctaagtcctgaacttgccttaccaacaggatcaagggccctcactacccgatctcatgcagctttgcacattttaggttgtcttgataccttggcagctatgcaggacttaaaaatgggtgttgcaagtacagaggaagagactcaagcagtaatgaaggtttattctaaagaagattatagtgtggtaaacaggtttgaaagtcatggaggaggctggggttattctgcccattcagtagaagctatacgtttcagtgccgacactgatattttacttggtggtcttggtctgtttggaggtagaggagaatatactgctaaaattaagctgtttgaattgggtcctgatggaggagatcatgaaactgatggtgaccttcttgcagagactgatgtattggcttatgactgtgctgctagagaaaaatatgcaatgatgtttgatgagcctgttctcctgcaagctgggtggtggtatgtggcatgggcccgagtgtca

SEQ ID NO: 28 - DNA, 303 bp, Homo sapiens
gctcccatgcctaacattggatcaaaatatggaagaaaagctacttggataggtgcaagtggggaccaaacttttttacgaattgatgaagcacttattaattctcatgtacttgctacatcagaaatttttgccagtaaacacataataggcttggtacctgcttctatatcagaacctcctccatttaaatgccttctgataaataaagtggatgggagttgtaaaacttttaatgactcagaacaagaggatctgcaaggatttggtgtgtgtcttgatcctgtatatgatgtaatttgg

SEQ ID NO: 29 - DNA, 612 bp, Homo sapiens
gatgaagcacttattaattctcatgtacttgctacatcagaaatttttgccagtaaacacataataggcttggtacctgcttctatatcagaacctcctccatttaaatgccttctgataaataaagtggatgggagttgtaaaacttttaatgactcagaacaagaggatctgcaaggatttggtgtgtgtcttgatcctgtatatgatgtaatttggaggtttcgaccaaatactagagagctgtggtgttacaatgcggtggttgctgatgccaggcttccctctgcagcagacatgcagtccagatgtagtatcctaagtcctgaacttgccttaccaacaggatcaagggccctcactacccgatctcatgcagctttgcacattttaggttgtcttgataccttggcagctatgcaggacttaaaaatgggtgttgcaagtacagaggaagagactcaagcagtaatgaaggtttattctaaagaagattatagtgtggtaaacaggtttgaaagtcatggaggaggctggggttattctgcccattcagtagaagctatacgtttcagtgccgacactgatattttacttggtggtcttggtctg

SEQ ID NO: 30 - DNA, 117 bp, Homo sapiens
gatgaagcacttattaattctcatgtacttgctacatcagaaatttttgccagtaaacacataataggcttggtacctgcttctatatcagaacctcctccatttaaatgccttctg